(19) United States
(12) Patent Application Publication
(10) Pub. No.: US 2023/0067811 A1
(43) Pub. Date: Mar. 2, 2023

(54) MODULATING LYMPHATIC VESSELS IN NEUROLOGICAL DISEASE

(71) Applicant: University of Virginia Patent Foundation, Charlottesville, VA (US)

(72) Inventors: Jonathan Kipnis, Charlottesville, VA (US); Antoine Louveau, Charlottesville, VA (US); Sandro Da Mesquita, Charlottesville, VA (US)

(21) Appl. No.: 17/794,338

(22) PCT Filed: Jan. 25, 2021
(86) PCT No.: PCT/US2021/014921

§ 371 Date: Jul. 21, 2022

Related U.S. Application Data
(60) Provisional application No. 63/071,241, filed on Aug. 27, 2020.
(60) Provisional application No. 62/965,763, filed on Jan. 24, 2020.

(51) Int. Cl. A61K 38/18; C12Q 1/6883; A61P 25/28

(52) U.S. Cl. CPC A61K 38/1825; A61K 38/1866; C12Q 1/6883; A61P 25/28; C12Q 2600/158

(57) ABSTRACT

In some embodiments herein, methods, compositions, and uses for modulating lymphatic vessels of the central nervous system are described. In some embodiments, methods, compositions, or uses for treating, preventing, or ameliorating symptoms of a neurological disease comprise increasing flow via meningeal lymphatic vessels are described.

### RELATED APPLICATIONS

This application is claims priority to U.S. Provisional Application No. 62/965,763, filed on Jan. 24, 2020 and U.S. Provisional Application No. 63/071,241, filed on Aug. 27, 2020. The entire contents of each of the foregoing applications are expressly incorporated herein by reference.

### STATEMENT REGARDING FEDERALLY SPONSORED R&D

This invention was made with government support under Grant Nos. AG034113, AG057496 and NS061973 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 131819-01420_SL.txt, created Jan. 12, 2021 which is 19,824 bytes in size. The information in the electronic format of the Sequence Listing is incorporated herein by reference in its entirety.

### BACKGROUND

Neurological diseases impact millions of people worldwide, and include degenerative and inflammatory neurological diseases. Among degenerative neurological diseases, Alzheimer's Disease (AD) is the most prevalent form of dementia worldwide (Andrieu et al., 2015) and is distinctively characterized by early and marked cognitive impairment (Andrieu et al., 2015; Ballard et al., 2011). The vast majority (>98%) of AD cases are sporadic (Blennow et al., 2006), and in such cases the etiology of the amyloid pathology is poorly understood (Benilova et al., 2012; Blennow et al., 2006). This is in contrast to familial AD, where rare hereditary dominant mutations in amyloid precursor protein (APP) or in presenilins 1 and 2 drive the uncontrolled formation of amyloid beta (Hardy and Selkoe, 2002). The brain's pathological hallmarks of AD are intracellular neurofibrillary tangles and extracellular amyloid plaques, the latter being a product of the amyloidogenic processing of APP and the resulting deposition of amyloid beta in the brain parenchyma (Benilova et al., 2012; Hardy and Selkoe, 2002; Ittner and Götz, 2011). Increasing aggregation of diffusible amyloid beta peptides from the ISF and the CSF into toxic oligomeric intermediates and their accumulation in the brain parenchyma (Hong et al., 2011; Iliff et al., 2012) are believed to be precipitating factors for different neuroinflammatory abnormalities (Guillot-Sestier et al., 2015; Hong et al., 2016; Matarin et al., 2015), such as the formation of neurofibrillary tangles (Ittner and Götz, 2011) and the pronounced neuronal dysfunction (Palop et al., 2007; Sun et al., 2009; Walsh et al., 2002) in the AD brain.

Organs generally function less effectively with age. For example, skin becomes less elastic, muscle tone is lost, and heart function declines. Aging is a substantial risk factor for numerous neurological diseases, including neurodegenerative diseases and inflammatory neurological diseases.

### FIELD

Several embodiments herein relate generally to compositions, methods, and uses for modulating lymphatic vessels in the central nervous system. Modulating lymphatic vessels, in accordance with some embodiments, are used to treat, prevent, or ameliorate symptoms of neurological diseases.

### SUMMARY

The present invention provides compositions and methods for modulating lymphatic vessels of the central nervous system. The compositions and methods are useful for treating, preventing, or ameliorating symptoms of neurological disease. This application is related to PCT Application No. PCT/US2020/054390, filed on Oct. 6, 2020, the entire contents of which are expressly incorporated herein by reference in their entirety.

In one aspect, the present disclosure provides a method of increasing clearance of molecules, such as proteins, in the central nervous system of a subject in need of treatment, inhibition, amelioration, reduction in symptoms, prevention, or delay in onset of a neurological disease. The method includes administering an amount of a flow modulator to a subject, whereby the amount of flow modulator increases the diameter of a meningeal lymphatic vessel of the subject, thereby increasing fluid flow in the central nervous system of the subject; and administering a neurological therapeutic agent to the subject, whereby the clearance of molecules such as proteins in the central nervous system of the subject is increased. In one embodiment, the flow modulator is a VEGFR3 agonist or Fibroblast Growth Factor 2 (FGF2).

In another aspect, disclosed herein is a method of modulating an activity of a lymphatic endothelial cell (LEC), a brain myeloid cell (e.g., microglia (Mg)), an infiltrating leukocyte and/or a brain blood vascular cell (e.g., brain endothelial cell (bBEC)) in a subject in need thereof, wherein the activity is an alteration of gene expression in one or more genes in Tables 2-29, the method comprising administering an effective amount of a flow modulator to the subject, wherein the flow modulator increases the fluid flow in the central nervous system (CNS) of the subject; and administering an effective amount of a neurological therapeutic agent to the subject, thereby modulating the activity of the LEC, Mg, and/or bBEC in the subject. In one embodiment, the alteration of gene expression is an increase in a level of gene expression of the one or more genes in Tables 2-29 as compared to a control level of gene expression of the one or more genes. In one embodiment, the alteration of gene expression is a decrease in a level of gene expression of the one or more genes in Tables 2-29 as compared to a control level of gene expression of the one or more genes. In one embodiment,

In one embodiment, the control level is a level of the gene expression of the one or more genes in Tables 2-29 in a healthy subject not having a neurological disease, or wherein the control level is an average level of gene expression of the one or more genes in Tables 2-29 in a population of healthy subjects not having a neurological disease. In another embodiment, the control level is a level of the gene expression of the one or more genes in Tables 2-29 in an age-matched subject with intact and functional meningeal lymphatic vasculature and no underlying neurological disease, or wherein the control level is an average level of gene expression of the one or more genes in Tables 2-29 in a population of age-matched subjects with intact and functional mengigeal lymphatic vasculature and no neurological disease.

In one embodiment, the level of gene expression of the one or more genes is increased by at least about 50%, 75%, 100%, 1.25-fold, 1.5-fold, 1.75-fold, 2-fold, 2.5-fold, 3-fold, 3.5-fold, 4-fold, 4.5-fold, 5-fold, or more, as compared to the control level. In one embodiment, the level of gene expression of the one or more genes is increased by at least about 50% to about 5-fold, about 2-fold to about 5-fold, about 3-fold to about 4-fold, about 50% to about 2-fold, about 50% to about 4-fold or about 75% to about 4-fold, as compared to the control level. In one embodiment, the level of gene expression of the one or more genes is increased by at least about 50%, 75%, 100%, 1.25-fold, 1.5-fold, 1.75-fold, 2-fold, 2.5-fold, 3-fold, 3.5-fold, 4-fold, 4.5-fold, 5-fold, or more, as compared to the control level. In one embodiment, the level of gene expression of the one or more genes is decreased by at least about 50% to about 5-fold, about 2-fold to about 5-fold, about 3-fold to about 4-fold, about 50% to about 2-fold, about 50% to about 4-fold or about 75% to about 4-fold, as compared to the control level.

In one embodiment, the control level is a level of the gene expression of the one or more genes in a healthy subject not having a neurological disease, or wherein the control level is an average level of gene expression of the one or more genes in a population of healthy subjects not having a neurological disease.

In one embodiment, the method further comprises determining a level of gene expression of the one or more genes in the subject prior to administering the effective amount of the flow modulator and the effective amount of the neurological therapeutic agent to the subject. In one embodiment, the determination comprises obtaining a sample from the subject, processing the sample, and determining the level of gene expression. In one embodiment, the method further comprises determining a level of gene expression of the one or more genes in the subject after administering the effective amount of the flow modulator and the effective amount of the neurological therapeutic agent to the subject. In one embodiment, the determination comprises obtaining a sample from the subject, processing the sample, and determining the level of gene expression.

In one embodiment, the method further comprises selecting a subject who would benefit from an increase in gene expression of the one or more genes in Tables 2-29 or a decrease in gene expression of the one or more genes in Tables 2-29.

In one embodiment, the subject has a neurological disease, or is at risk for developing a neurological disease. In one embodiment, the method further comprises selecting a subject that has a neurological disease, or is at risk for developing a neurological disease. In one embodiment, the neurological disease is Alzheimer's Disease (AD). In one embodiment, the subject has a risk factor for AD selected from the group consisting of: diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), age, familial AD, and a symptom of dementia; or a combination thereof.

In one embodiment, the flow modulator is a VEGFR3 agonist, said VEGFR3 agonist comprising VEGF-c. In one embodiment, the flow modulator, e.g., VEGF-c, is administered by intra-cisterna magna (ICM or i.c.m.) injection.

In one embodiment, the neurological therapeutic agent comprises an antibody, or antigen-binding fragment thereof, that binds to amyloid beta. In one embodiment, the neurological therapeutic agent, e.g., the antibody or antigen-binding fragment thereof, is administered systemically.

In another aspect, the method includes determining the subject to have the neurological disease, a risk factor therefor, or both. In one embodiment, the method includes determining the subject to have a risk factor for AD selected from the group consisting of: diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), age, familial AD, a symptom of dementia, or a combination of any of the listed risk factors.

In one aspect, the disclosure provides a method of identifying a subject that has an enhanced risk of developing a neurological disease, comprising detecting an alteration in gene expression in one or more genes in Tables 2-29 in central nervous system prior to the onset of the neurological disease, thereby identifying the subject as having an enhanced risk of developing the neurological disease. In one embodiment, the alteration in gene expression is in brain lymphatic endothelial cells (LECs), brain myeloid cell (e.g., microglia (Mg)), infiltrating leukocyte and/or brain blood vascular cell (e.g., brain endothelial cells (bBECs)). In one embodiment, the alteration in gene expression is in immune cells in the brain of the subject. In one embodiment, the alteration in gene expression is in immune cells in brain cortices or meninges of the subject. In one embodiment, the gene is selected from the group consisting of Hexb, ApoE, H2-Aa, H2-Ab1, Cd74, H2-D1, and H-2Kd. In one embodiment, the brain LECs or immune cells are obtained from a biopsy of deep cervical lymph nodes or peripheral blood from the subject. In one embodiment, the alteration in gene expression is in ear skin cells.

In one aspect, disclosed herein is a method of identifying a subject that has an enhanced risk of developing neurological disease, comprising detecting an increase in a number of immune cells in central nervous system of the subject prior to the onset of the neurological disease, thereby identifying the subject as having an enhanced risk of developing the neurological disease. In one embodiment, the increase in the number of immune cells is in brain cortices or meninges of the subject. In one embodiment, the immune cells are CD45high cells or H-2Kd expressing CD45int cells. In one embodiment, the immune cells are microglia or recruited lymphocytes from blood. In one embodiment, the immune cells are selected from the group consisting of B cells, CD4+ T cells, CD8+ T cells, and type 3 innate lymphoid cells (ILC3s). In one embodiment, the number of immune cells is determined by in vivo fluorescence imaging.

In one aspect, disclosed herein is a method of identifying a subject that has an enhanced risk of developing a neurological disease, comprising detecting one or more single nucleotide polymorphisms (SNPs) associated with one or more genes selected from the genes in Tables 2-29, thereby identifying the subject as having an enhanced risk of developing the neurological disease. In one embodiment, the SNP is associated with a gene that is highly expressed in a lymphatic endothelial cell. In one embodiment, the lymphatic endothelial cell is selected from the group consisting of a central nervous system lymphatic endothelial cell, a diaphragm lymphatic endothelial cell, and an ear skin endothelial cell. In one embodiment, the gene that is highly expressed in the lymphatic endothelial cell has an average expression in the top 2nd, 5th, 10th, or 25th percentile out of all genes. In one embodiment, the expression percentile is determined by RNA-seq data. In one embodiment, the gene is selected from the group consisting of the genes listed in FIG. 23. In one embodiment, the gene is selected from the group consisting of Dst, Hmcn1, Rgl1, Prrc2c, Sft2d2, Itga6, Celf1, Sppl2a, Golim4, She, Abca1, Nfib, Akap9, Tmem106b, Dlc1, Adam10, Serinc5, Itga1, Ptprg, Fermt2, Efr3a, Parvb, Gsk3b, Pak2, Cd2ap, Egr1, and Ahnak. In one embodiment, the gene is selected from the group consisting of Frmd4a, Maf, Timp2, and Elmo1. In one embodiment, the gene is selected from the group consisting of Crl1, Clptm1, Picalm, Psma1, Ssbp4, and Mef2c. In one embodiment, the gene is selected from the group consisting of Apoe Tspan13, and Bsg.

In one embodiment, the subject is a human subject. In one embodiment, the human subject is about 20 years old, about 30 years old, about 40 years old, about 50 years old, about 60 years old, about 70 years old, or about 80 years old. In one embodiment, the human subject has been previously identified to have a risk of developing neurological disease. In one embodiment, the human subject has been previously identified to have a risk of developing neurological disease by family history investigation or genetic screening. In one embodiment, the neurological disease is selected from the group consisting of AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items. In one embodiment, the neurological disease is Alzheimer's disease.

In one aspect, disclosed herein is a method of reducing the risk or delaying the onset of developing a neurological disease in a subject, comprising administering an effective amount of a neurological therapeutic agent to the central nervous system of the subject prior to the onset of the neurological disease, thereby reducing the risk of developing the neurological disease in the subject, wherein the subject is identified to have an enhanced risk of developing a neurodegenerative disease using a method described herein. In one embodiment, the method further comprises administering an effective amount of a flow modulator to the subject. In another embodiment, the neurological therapeutic agent reduces the number of immune cells in the brain.

In another aspect, disclosed herein is a method of increasing clearance of a molecule from the central nervous system in a subject in need thereof, the method comprising: administering an effective amount of a flow modulator to the subject by intra-cisterna magna (ICM) injection, wherein the flow modulator increases the fluid flow in the central nervous system of the subject; and administering an effective amount of a neurological therapeutic agent to the subject by systemic administration, thereby increasing the clearance of the molecule from the central nervous system of the subject. In another aspect, disclosed herein is a method of reducing an aggregate of a protein or peptide in the central nervous system of a subject in need thereof, the method comprising: administering an effective amount of a flow modulator to the subject by intra-cisterna magna (ICM) injection, wherein the flow modulator increases the fluid flow in the central nervous system of the subject; and administering an effective amount of a neurological therapeutic agent to the subject by systemic administration, thereby reducing the aggregate of the protein or peptide in the subject. In another embodiment, disclosed herein is a method of reducing a microglial inflammatory response in the central nervous system of a subject in need thereof, the method comprising: administering an effective amount of a flow modulator to the subject by intra-cisterna magna (ICM) injection, wherein the flow modulator increases the fluid flow in the central nervous system of the subject; and administering an effective amount of a neurological therapeutic agent to the subject by systemic administration, thereby reducing the microglial inflammatory response in the central nervous system of the subject. In another aspect, disclosed herein is a method of reducing neurite dystrophy in the central nervous system of a subject in need thereof, the method comprising: administering an effective amount of a flow modulator to the subject by intra-cisterna magna (ICM) injection, wherein the flow modulator increases the fluid flow in the central nervous system of the subject; and administering an effective amount of a neurological therapeutic agent to the subject by systemic administration, thereby reducing neurite dystrophy in the central nervous system of the subject. In another aspect, disclosed herein is a method of treating a neurological disease in a subject in need thereof, the method comprising: administering an effective amount of a flow modulator to the subject by intra-cisterna magna (ICM) injection, wherein the flow modulator increases the fluid flow in the central nervous system of the subject; and administering an effective amount of a neurological therapeutic agent to the subject by systemic administration, thereby treating the neurological disease in the subject. In one embodiment, the flow modulator comprises a VEGFR3 agonist, optionally wherein the VEGFR3 agonist comprises a VEGF-c. In one embodiment, the neurological therapeutic agent is an antibody, or antigen-binding fragment thereof, optionally wherein the antibody, or antigen-binding fragment thereof, is an amyloid beta antibody, or antigen-binding fragment thereof. In one embodiment, the amyloid beta antibody, or antigen-binding fragment thereof, is selected from the group consisting of: bapineuzumab, gantenerumab, aducanumab, solanezumab, immunoglobulin, BAN2401, semorinemab, zagotenemab, crenezumab, and an antigen binding fragment thereof.

In still another aspect, the present invention provides a method of treating, inhibiting, ameliorating, reducing the symptoms of, preventing, or delaying the onset of a neurological disease. The method includes administering an amount of a flow modulator to the subject in need, whereby the amount of the flow modulator increases the diameter of a meningeal lymphatic vessel of the subject; and administering a neurological therapeutic agent to the subject, wherein the neurological therapeutic agent is different from the flow modulator, thereby treating, inhibiting, ameliorating, reducing the symptoms of, preventing, or delaying the onset of the neurological disease. In one embodiment, the flow modulator is a VEGFR3 agonist or Fibroblast Growth Factor 2 (FGF2).

In yet another aspect, the neurological therapeutic agent is selected from the group consisting of a small molecule, a nucleic acid, a peptide, a protein, an antibody, a recombinant virus, a vaccine, and a cell.

In one embodiment, the neurological therapeutic agent comprises a small molecule. In another embodiment, the small molecule is selected from the group consisting of Donepezil, Galantamine, Rivastigmine, Memantine, Lanabecestat, Atabecestat, Verubecestat, Elenbecestat, Semagacestat, Tarenflurbil, and Brexipiprazole.

In still another aspect, the neurological therapeutic agent comprises an antibody, or an antigen binding fragment thereof, that specifically binds to a protein or a peptide that forms pathological aggregate. In one embodiment, the peptide or protein is selected from the group consisting of amyloid precursor protein, amyloid beta, fibrin, tau, apolipoprotein E (Apoe), alpha-synuclein, TDP43, and huntingtin. In another embodiment, the protein is amyloid beta and the antibody or the antigen binding fragment thereof is selected from the group consisting of: bapineuzumab, gantenerumab, aducanumab, solanezumab, immunoglobulin, BAN2401, semorinemab, zagotenemab, crenezumab, and the antigen binding fragment thereof. In still another embodiment, the antibody or antigen binding fragment thereof that binds to amyloid beta comprises a HCDR1, a HCDR2, a HCDR3, a LCDR1, a LCDR2, and a LCDR3 of any one of bapineuzumab, gantenerumab, aducanumab, solanezumab, immunoglobulin, BAN2401 (Eisai), semorinemab, zagotenemab, crenezumab, or an antigen binding fragment thereof.

In one embodiment, the protein is tau and the antibody or the antigen binding fragment thereof is selected from the group consisting of Gosuranemab, Armanezumab, and the antigen binding fragment thereof. In another embodiment, the antibody or antigen binding fragment thereof comprises a HCDR1, a HCDR2, a HCDR3, a LCDR1, a LCDR2, and a LCDR3 of any one of Gosuranemab, Armanezumab, or the antigen binding fragment thereof.

In another embodiment, the protein is alpha-synuclein and the antibody or the antigen binding fragment thereof is selected from the group consisting of BIIB054, PRX002/RG7935, prasinezumab, and the antigen binding fragment thereof. In one embodiment, the antibody or antigen binding fragment thereof comprises a HCDR1, a HCDR2, a HCDR3, a LCDR1, a LCDR2, and a LCDR3 of any one of BIIB054, PRX002/RG7935, prasinezumab, or the antigen binding fragment thereof.

In still another embodiment, the protein is fibrin. An exemplary antibody or the antigen binding fragment thereof is 5B8 as described in Ryu et al., Fibrin-targeting immunotherapy protects against neuroinflammation and neurodegeneration, Nature Immunology 19, 1212-1223 (2018), incorporated herein by reference. In one embodiment, the antibody or antigen binding fragment thereof comprises a HCDR1, a HCDR2, a HCDR3, a LCDR1, a LCDR2, and a LCDR3 of the 5B8 antibody.

In yet another embodiment, the protein is apolipoprotein E (Apoe) and the antibody. An exemplary antibody or the antigen binding fragment thereof is HAE4 as described in Liao et al., Targeting of nonlipidated, aggregated apoE with antibodies inhibits amyloid accumulation, J. of Clin. Invest., 128(5): 2144-2155, incorporated herein by reference. In one embodiment, the antibody or antigen binding fragment thereof comprises a HCDR1, a HCDR2, a HCDR3, a LCDR1, a LCDR2, and a LCDR3 of the HAE4 antibody.

In yet another aspect, the flow modulator is VEGFR3 agonist, said VEGFR3 agonist comprising VEGF-c, and wherein the neurological therapeutic agent comprises an antibody that binds to amyloid beta.

In one aspect, the diameter of the meningeal lymphatic vessel is increased by at least 20%.

In another aspect, the central nervous system of the subject comprises amyloid beta plaques, and wherein administering the flow modulator in combination with the neurological therapeutic agent reduces the quantity of amyloid beta plaques. In one embodiment, the quantity of accumulated amyloid beta plaques is reduced by at least 5%. In another embodiment, at least some of the accumulated amyloid beta plaques are in the meninges of the subject's brain.

In still another aspect, the neurological disease is treated, inhibited, ameliorated, prevented, or delayed in onset, and/or wherein symptoms of the neurological disease are reduced.

In yet another aspect, the molecules to be cleared comprise a protein selected from the group consisting of amyloid beta, fibrin, tau, apolipoprotein E (Apoe), alpha synuclein, TDP43, and huntingtin.

In one aspect, the present invention provides method of reducing a quantity of aggregates of a protein or peptide in a subject having a neurological disease or a risk factor therefor. The method includes determining the subject to have the neurological disease or the risk factor; administering a flow modulator to a meningeal space of the subject, whereby fluid flow in the central nervous system of the subject is increased; and administering a neurological therapeutic agent to the subject, wherein the neurological therapeutic agent is different from the flow modulator, thereby reducing the quantity of the aggregates of the protein or peptide in the subject. In one embodiment the flow modulator is a VEGFR3 agonist or FGF2.

In another aspect, the protein or peptide is selected from the group consisting of amyloid beta, fibrin, tau, apolipoprotein E (Apoe), alpha synuclein, TDP43, and huntingtin. In one embodiment, the protein or peptide comprises amyloid beta and the aggregates comprise amyloid beta plaque.

In still another aspect, the neurological therapeutic agent comprises an antibody that specifically binds to the protein or peptide.

In one aspect, the neurological therapeutic agent comprises a small molecule.

In another aspect, at least some of the aggregates of the protein or peptide are in the meninges of the subject's brain. In one embodiment, the protein or peptide comprises amyloid beta and the aggregates comprise amyloid plaque.

In still another aspect, the quantity of aggregates of the protein or peptide is reduced by at least 5%. In one embodiment, the protein or peptide comprises amyloid beta and the aggregates comprise amyloid plaque.

In yet another aspect, administering the flow modulator increases the diameter of a meningeal lymphatic vessel of the subject's brain by at least 20%, thereby increasing fluid flow. In one embodiment, the flow modulator is VEGFR3 agonist or FGF2. In another embodiment, the VEGFR3 agonist is administered, and the VEGFR3 agonist comprising VEGF-c.

In one aspect, the subject has the neurological disease.

In another aspect, the VEGFR3 agonist is administered. In one embodiment, the VEGFR3 agonist comprises VEGF-c, and the neurological therapeutic agent comprises an antibody that binds to amyloid beta. In another embodiment, the VEGFR3 agonist is selected from the group consisting of VEGF-c, VEGF-d, an analog, variant, or fragment thereof, or a combination of any of these.

In still another aspect, the neurological therapeutic agent is administered to the central nervous system (CNS) of the subject. In one embodiment, the neurological therapeutic agent is administered to the meninges of the subject's brain.

In yet another aspect, the flow modulator is administered by intra-cisterna magna (ICM or i.c.m.) injection to the subject. In another embodiment, the flow modulator is administered selectively to the meningeal space of the subject.

In one aspect, the flow modulator is administered to the subject by a route selected from the group consisting of intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the flow modulator, expression in the subject of a nucleic acid encoding the flow modulator, or a combination of any of the listed routes. In one embodiment, the flow modulator is VEGFR3 agonist and/or FGF2.

In another aspect, the neurological therapeutic agent is administered selectively to the meningeal space of the subject.

In still another aspect, the neurological therapeutic agent is administered to the subject by a route selected from the group consisting of intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, or a combination of any of the listed routes.

In yet another aspect, the neurological therapeutic agent is administered to the subject systemically. In another embodiment, the neurological therapeutic agent is administered by intravenous infusion.

In one aspect, the neurological therapeutic agent is administered to the subject by the same route at the flow modulator. In another aspect, the neurological therapeutic agent is administered to the subject by a different route than the flow modulator.

In another aspect, the flow modulator and the neurological therapeutic agent are administered to the subject at the same time.

In still another aspect, the flow modulator and the neurological therapeutic agent are administered to the subject in the same composition.

In yet another aspect, the flow modulator and the neurological therapeutic agent are administered to different locations of the subject.

In one aspect, the flow modulator and the neurological therapeutic agent are administered to the subject in different compositions.

In another aspect, the flow modulator and the neurological therapeutic agent are administered to the subject at different times.

In one embodiment of various aspects described herein, the flow modulator is VEGFR3 agonist or FGF2.

In one aspect, the neurological disease comprises a proteinopathy.

In another aspect, the neurological disease comprises a tauopathy and/or amyloidosis.

In still another aspect, the neurological disease is selected from the group consisting of: AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items. In one embodiment, the neurological disease is an amyloidosis. In another embodiment, the neurological disease is Alzheimer's disease. In still another embodiment, the neurological disease comprises familial AD and/or sporadic AD. In yet another embodiment, the neurological disease is familial AD and/or sporadic AD.

In one aspect, increasing fluid flow in the central nervous system of the subject comprises increasing a rate of perfusion of fluid throughout an area of the subject's brain, and/or increasing a rate of perfusion of fluid through the subject's central nervous system and/or increasing a rate of perfusion out of the subject's central nervous system. In one embodiment, increasing the fluid flow in the CNS increases clearance of soluble molecules in the brain of the subject. In still another embodiment, the fluid comprises cerebral spinal fluid (CSF), interstitial fluid (ISF), or both.

In another aspect, the neurological therapeutic agent is administered in an amount effective to treat, inhibit, ameliorate, reduce the symptoms of, prevent, or delay the onset of the neurological disease.

In still another aspect, the fluid comprises cerebral spinal fluid (CSF), interstitial fluid (ISF), or both.

In one embodiment of various aspects described herein, the neurological therapeutic agent is selected from the group consisting of: bapineuzumab, gantenerumab, aducanumab, solanezumab, crenezumab, pepinemab, ozanezumab, AT-1501, BIIB054, and PRX002.

The present invention provides a composition or product combination. The composition or product combination includes a flow modulator; and a neurological therapeutic agent that is different from the flow modulator. In one embodiment, the flow modulator is VEGFR3 agonist or Fibroblast Growth Factor 2 (FGF2). In another embodiment, the neurological therapeutic agent is selected from the group consisting of a small molecule, a nucleic acid, a peptide, a protein, an antibody, a recombinant virus, and a cell. In still another embodiment, the neurological therapeutic agent comprises a small molecule. In yet another embodiment, the small molecule is selected from the group consisting of Donepezil, Galantamine, Rivastigmine, Memantine, Lanabecestat, Atabecestat, Verubecestat, Elenbecestat, Semagacestat, Tarenflurbil, and Brexipiprazole.

In another aspect, the neurological therapeutic agent comprises an antibody that binds to a protein that forms pathological aggregate. In one embodiment, the peptide or protein is selected from the group consisting of amyloid precursor protein, amyloid beta, fibrin, tau, apolipoprotein E (Apoe), alpha-synuclein, TDP43, and huntingtin. In another embodiment, the protein is amyloid beta and the antibody or the antigen binding fragment thereof is selected from the group consisting of: bapineuzumab, gantenerumab, aducanumab, solanezumab, immunoglobulin, BAN2401, semorinemab, zagotenemab, crenezumab, and the antigen binding fragment thereof. In still another embodiment, the antibody or antigen binding fragment thereof that binds to amyloid beta comprises a HCDR1, a HCDR2, a HCDR3, a LCDR1, a LCDR2, and a LCDR3 of any one of bapineuzumab, gantenerumab, aducanumab, solanezumab, immunoglobulin, BAN2401 (Eisai), semorinemab, zagotenemab, crenezumab, or the antigen binding fragment thereof.

In still another aspect, the protein is tau and the antibody or the antigen binding fragment thereof is selected from the group consisting of Gosuranemab, Armanezumab, and the antigen binding fragment thereof. In one embodiment, the antibody or antigen binding fragment thereof comprises a HCDR1, a HCDR2, a HCDR3, a LCDR1, a LCDR2, and a LCDR3 of any one of Gosuranemab, Armanezumab, or the antigen binding fragment thereof.

In yet another aspect, the protein is alpha-synuclein and the antibody or the antigen binding fragment thereof is selected from the group consisting of BIIB054 (Biogen), PRX002/RG7935 (Roche), prasinezumab (Roche), and the antigen binding fragment thereof. In one embodiment, the antibody or antigen binding fragment thereof comprises a HCDR1, a HCDR2, a HCDR3, a LCDR1, a LCDR2, and a LCDR3 of any one of BIIB054 (Biogen), PRX002/RG7935 (Roche), prasinezumab (Roche), or the antigen binding fragment thereof.

In one aspect, the protein is fibrin. An exemplary antibody or the antigen binding fragment thereof is 5B8 as described in Ryu et al., Fibrin-targeting immunotherapy protects against neuroinflammation and neurodegeneration, Nature Immunology 19, 1212-1223 (2018). In one embodiment, the antibody or antigen binding fragment thereof comprises a HCDR1, a HCDR2, a HCDR3, a LCDR1, a LCDR2, and a LCDR3 of the 5B8 antibody.

In yet another aspect, the protein is apolipoprotein E (Apoe). An exemplary antibody or the antigen binding fragment thereof is HAE4 as described in Liao et al., Targeting of nonlipidated, aggregated apoE with antibodies inhibits amyloid accumulation, J. of Clin. Invest., 128(5): 2144-2155. In one embodiment, the antibody or antigen binding fragment thereof comprises a HCDR1, a HCDR2, a HCDR3, a LCDR1, a LCDR2, and a LCDR3 of the HAE4 antibody.

In one aspect, the VEGFR3 agonist comprises VEGF-c. In one embodiment, the VEGFR3 agonist comprises VEGF-c, and wherein the neurological therapeutic agent comprises an antibody that binds to amyloid beta. In another embodiment, the neurological therapeutic agent is selected from the group consisting of bapineuzumab, gantenerumab, aducanumab, solanezumab, crenezumab, pepinemab, ozanezumab, AT-1501, BIIB054, and PRX002.

In another aspect, the VEGFR3 agonist is selected from the group consisting of: VEGF-c, VEGF-d, an analog, variant, or fragment thereof, or a combination of any of these.

In still another aspect, the flow modulator is in an amount effective to increase fluid flow in the central nervous system of the subject. In one embodiment, the flow modulator is an amount effective to diameter of a meningeal lymphatic vessel of the subject's brain by at least 20%, thereby increasing fluid flow.

In one aspect, composition or product combination, further includes a pharmaceutically acceptable carrier.

In another aspect, the composition or product combination is formulated for a route of administration selected from the group consisting of: intrathecal administration, intraventricular administration, intraparenchymal administration, nasal administration, transcranial administration, contacting cerebral spinal fluid (CSF) of the subject, pumping into CSF of the subject, implantation into the skull or brain, contacting a thinned skull or skull portion of the subject with the flow modulator and the neurological therapeutic agent.

In yet another aspect, the present invention provides composition or product combination. The composition or product combination includes a first nucleic acid that encodes the flow modulator of any aspects described herein, wherein the flow modulator is a polypeptide; and a neurologic therapeutic agent. In one embodiment, the flow modulator is VEGFR3 agonist or FGF2.

In one aspect, the neurologic therapeutic agent is a second nucleic acid encoding the protein, the peptide, or the antibody of any aspects described herein, wherein the protein, the peptide, or the antibody is not the flow modulator. In one embodiment, the first nucleic acid and second nucleic acid are DNA or RNA. In another embodiment, the first nucleic acid and second nucleic acid are a DNA, and the DNA is on an expression vector. In still another embodiment, the expression vector is a plasmid or a viral vector. In yet another embodiment, the expression vector is an adeno-associated viral vector. In one embodiment, the first nucleic acid and the second nucleic acid are on the same polynucleotide molecule. In another embodiment, the first nucleic acid and the second nucleic acid are on different polynucleotide molecule.

In another aspect, the present invention provides a pharmaceutical composition. The pharmaceutical composition includes the composition or the product combination of any aspects described herein, and a pharmaceutically acceptable carrier. In one embodiment, the pharmaceutical composition is formulated for a route of administration selected from the group consisting of: intrathecal administration, intraventricular administration, intraparenchymal administration, nasal administration, transcranial administration, contacting cerebral spinal fluid (CSF) of the subject, pumping into CSF of the subject, implantation into the skull or brain, contacting a thinned skull or skull.

In still another aspect, the present invention provides the composition or product combination or the pharmaceutical composition of any aspects described herein for use in treating a neurological disease or disorder. In one embodiment, the use is in in treating a proteinopathy, such as a tauopathy and/or amyloidosis. In another embodiment, the use is in in treating a neurological disease selected from the group consisting of: AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items.

In a method of any aspects described herein, the flow modulator is a polypeptide, and wherein the flow modulator is administered by administering an effective amount of a first nucleic acid that encoding the polypeptide. In one embodiment the neurological therapeutic agent is a second nucleic acid encoding the peptide, the protein, or the antibody of any one of claims 1-70, wherein the protein, the peptide, or the antibody is not the flow modulator.

In one aspect, the first nucleic acid and second nucleic acid are DNA or RNA. In one embodiment, the first nucleic acid and second nucleic acid are a DNA, and the DNA is on an expression vector. In another embodiment, the expression vector is a plasmid or a viral vector. In still another embodiment, the expression vector is an adeno-associated viral vector.

In one aspect, the first nucleic acid and the second nucleic acid are on the same polynucleotide molecule. In another aspect, the first nucleic acid and the second nucleic acid are on different polynucleotide molecule.

In some embodiments, a method of increasing clearance of molecules (such as proteins) in the central nervous system of a subject in need of treatment, inhibition, amelioration, reduction in symptoms, prevention, or delay in onset of a neurological disease is described. The method can comprise administering an amount of VEGFR3 agonist or Fibroblast Growth Factor 2 (FGF2) to a meningeal space of the subject, whereby the amount of VEGFR3 agonist or FGF2 increases the diameter of a meningeal lymphatic vessel of the subject, thereby increasing fluid flow in the central nervous system of the subject. The method can comprise administering a neurological therapeutic agent to the central nervous system of the subject. Thus, the clearance of molecules such as proteins in the central nervous system of the subject can be increased. In some embodiments, the method further comprises determining the subject to have the neurological disease, a risk factor therefor, or both. In some embodiments, the method further comprises determining the subject to have a risk factor for AD selected from the group consisting of: diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), age, familial AD, a symptom of dementia, or a combination of any of the listed risk factors.

In some embodiments, a method of treating, inhibiting, ameliorating, reducing the symptoms of, preventing, or delaying the onset of a neurological disease is described. The method can comprise administering an amount of VEGFR3 agonist or Fibroblast Growth Factor 2 (FGF2) to a meningeal space of the subject in need, whereby the amount of VEGFR3 agonist or FGF2 increases the diameter of a meningeal lymphatic vessel of the subject. The method can comprise administering a neurological therapeutic agent for the neurological disease to the subject, wherein the neurological therapeutic agent is different from the VEGFR3 or FGF2. Thus, the method can treat, inhibit, ameliorate, reduce the symptoms of, prevent, or delay the onset of the neurological disease. In the method of some embodiments, the neurological therapeutic agent comprises an antibody that specifically binds to amyloid beta. In the method of some embodiments, the amyloid beta antibody is selected from the group consisting of: bapineuzumab, gantenerumab, aducanumab, solanezumab, and crenezumab. In the method of some embodiments, the antibody that binds to amyloid beta comprises a HCDR1, a HCDR2, a HCDR3, a LCDR1, a LCDR2, and a LCDR3 of any one of bapineuzumab, gantenerumab, aducanumab, solanezumab, or crenezumab. In the method of some embodiments, the VEGFR3 agonist is administered, said VEGFR3 agonist comprising VEGF-c, and the neurological therapeutic agent comprises an antibody that binds to amyloid beta. In the method of some embodiments, the diameter of the meningeal lymphatic vessel is increased by at least 20%. In the method of some embodiments, the central nervous system of the subject comprises amyloid beta plaques, and wherein administering the VEGFR3 agonist or FGF2 in combination with the neurological therapeutic agent reduces the quantity of amyloid beta plaques. In the method of some embodiments, the quantity of accumulated amyloid beta plaques is reduced by at least 5%. In the method of some embodiments, at least some of the accumulated amyloid beta plaques are in the meninges of the subject's brain. In the method of some embodiments, the neurological disease is treated, inhibited, ameliorated, prevented, or delayed in onset, and/or wherein symptoms of the neurological disease are reduced. In the method of some embodiments, the molecules comprise amyloid beta.

In some embodiments method of reducing a quantity of accumulated amyloid beta plaques in a subject having a neurological disease or a risk factor therefor is described. The method can comprise determining the subject to have the neurological disease or the risk factor. The method can comprise administering a VEGFR3 agonist or FGF2 to a meningeal space of the subject, so that fluid flow in the central nervous system of the subject is increased. The method can comprise administering a neurological therapeutic agent to the subject, wherein the neurological therapeutic agent is different from the VEGFR3 or FGF2. Thus, the method can reduce the quantity of accumulated amyloid beta plaques in the subject. In the method of some embodiments, the neurological therapeutic agent comprises an antibody that specifically binds to amyloid beta. In the method of some embodiments, at least some of the accumulated amyloid beta plaques are in the meninges of the subject's brain. In the method of some embodiments, the quantity of accumulated amyloid beta plaques is reduced by at least 5%. In the method of some embodiments, the VEGFR3 agonist or FGF2 increases the diameter of a meningeal lymphatic vessel of the subject's brain by at least 20%, thereby increasing fluid flow. In the method of some embodiments, the VEGFR3 agonist is administered, said VEGFR3 agonist comprising VEGF-c. In the method of some embodiments, the subject has the neurological disease.

In some embodiments, for any of the methods described herein, the VEGFR3 agonist is administered. By way of example, the VEGFR3 agonist can comprises VEGF-c, and the neurological therapeutic agent can comprise an antibody that binds to amyloid beta. By way of example, the VEGFR3 agonist can be selected from the group consisting of: VEGF-c, VEGF-d, an analog, variant, or fragment thereof, or a combination of any of these.

In some embodiments, for any of the methods described herein, the neurological therapeutic agent is administered to the central nervous system (CNS) of the subject. For example, the neurological therapeutic agent can be administered to the meninges of the subject's brain. In some embodiments, for any of the methods described herein, the VEGFR3 agonist and/or FGF2 is administered selectively to the meningeal space of the subject. In some embodiments, for any of the methods described herein, the VEGFR3 agonist and/or FGF2 is administered to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the VEGFR3 agonist or FGF2, expression in the subject of a nucleic acid encoding the VEGFR3 agonist or FGF2, or a combination of any of the listed routes. In some embodiments, for any of the methods described herein, the neurological therapeutic agent is administered selectively to the meningeal space of the subject. In some embodiments, for any of the methods described herein, the neurological therapeutic agent is administered to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, or a combination of any of the listed routes. In some embodiments, for any of the methods described herein, the neurological therapeutic agent is administered to the subject by intravenous infusion. In some embodiments, for any of the methods described herein, the neurological therapeutic agent is administered to the subject by the same route at the VEGFR3 agonist and/or FGF2. In some embodiments, for any of the methods described herein, the neurological therapeutic agent is administered to the subject by a different route than the VEGFR3 agonist and/or FGF2. In some embodiments, for any of the methods described herein, the VEGFR3 agonist or FGF2 and the neurological therapeutic agent are administered to the subject at the same time. In some embodiments, for any of the methods described herein, the VEGFR3 agonist or FGF2 and the neurological therapeutic agent are administered to the subject in the same composition. In some embodiments, for any of the methods described herein, the VEGFR3 agonist or FGF2 and the neurological therapeutic agent are administered to different locations of the subject. In some embodiments, for any of the methods described herein, the VEGFR3 agonist or FGF2 and the neurological therapeutic agent are administered to the subject in different compositions. In some embodiments, for any of the methods described herein, the VEGFR3 agonist and FGF2 and the neurological therapeutic agent are administered to the subject at different times.

In some embodiments, for any of the methods described herein, the neurological disease comprises a proteinopathy. In some embodiments, for any of the methods described herein, the neurological disease comprises a tauopathy and/or amyloidosis. In some embodiments, for any of the methods described herein, the neurological disease is selected from the group consisting of: AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CID), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items. In some embodiments, for any of the methods described herein, the neurological disease is an amyloidosis. In some embodiments, for any of the methods described herein, the neurological disease is Alzheimer's disease. In some embodiments, for any of the methods described herein, the neurological disease comprises familial AD and/or sporadic AD. In some embodiments, for any of the methods described herein, the neurological disease is familial AD and/or sporadic AD.

In some embodiments, for any of the methods described herein, increasing fluid flow in the central nervous system of the subject comprises increasing a rate of perfusion of fluid throughout an area of the subject's brain, and/or increasing a rate of perfusion of fluid through the subject's central nervous system and/or increasing a rate of perfusion of fluid out of the subject's central nervous system. By way of example, the method can comprise increasing a rate of perfusion out of the subject's central nervous system. In some embodiments, for any of the methods described herein, increasing the fluid flow in the CNS increases clearance of soluble molecules in the brain of the subject. In some embodiments, for any of the methods described herein, the fluid comprises cerebral spinal fluid (CSF), interstitial fluid (ISF), or both. In some embodiments, for any of the methods described herein, the neurological therapeutic agent is administered in an amount effective to treat, inhibit, ameliorate, reduce the symptoms of, prevent, or delay the onset of the neurological disease. In some embodiments, for any of the methods described herein, the fluid comprises cerebral spinal fluid (CSF), interstitial fluid (ISF), or both.

In some embodiments, for any of the methods described herein, the neurological therapeutic agent is selected from the group consisting of: bapineuzumab, gantenerumab, aducanumab, solanezumab, crenezumab, pepinemab, ozanezumab, AT-1501, BIIB054, and PRX0002.

In some embodiments, a composition or product combination is described. The composition or product combination can comprise a VEGFR3 agonist or FGF2. The composition or product combination can comprise a neurological disease therapeutic agent that is different from the VEGFR3 agonist or FGF2. In the composition or product combination of some embodiments, the neurological therapeutic agent comprises an antibody that binds to amyloid beta. In the composition or product combination of some embodiments, the VEGFR3 agonist comprises VEGF-c, and wherein the neurological therapeutic agent comprises an antibody that binds to amyloid beta. In the composition or product combination of some embodiments, the antibody that binds to amyloid beta is selected from the group consisting of bapineuzumab, gantenerumab, aducanumab, solanezumab, and crenezumab. In the composition or product combination of some embodiments, the antibody that binds to amyloid beta comprises a HCDR1, a HCDR2, a HCDR3, a LCDR1, a LCDR2, and a LCDR3 of any one of bapineuzumab, gantenerumab, aducanumab, solanezumab, or crenezumab. In the composition or product combination of some embodiments, the VEGFR3 agonist comprises VEGF-c, and wherein the neurological therapeutic agent comprises an antibody that binds to amyloid beta. In the composition or product combination of some embodiments, the neurological therapeutic agent is selected from the group consisting of bapineuzumab, gantenerumab, aducanumab, solanezumab, crenezumab, pepinemab, ozanezumab, AT-1501, BIIB054, and PRX0002. In the composition or product combination of some embodiments, the VEGFR3 agonist is selected from the group consisting of: VEGF-c, VEGF-d, an analog, variant, or fragment thereof, or a combination of any of these. In the composition or product combination of some embodiments, the VEGFR3 agonist or FGF2 is an amount effective to increase fluid flow in the central nervous system of the subject. In the composition or product combination of some embodiments, the VEGFR3 agonist or FGF2 is an amount effective to diameter of a meningeal lymphatic vessel of the subject's brain by at least 20%, thereby increasing fluid flow. The composition or product combination of some embodiments further comprises a pharmaceutically acceptable carrier. In the composition or product combination of some embodiments, the composition or product combination is formulated for a route of administration selected from the group consisting of: intrathecal administration, intraventricular administration, intraparenchymal administration, nasal administration, transcranial administration, contact cerebral spinal fluid (CSF) of the subject, pumping into CSF of the subject, implantation into the skull or brain, contacting a thinned skull or skull portion of the subject with the agent, and expression in the subject of a nucleic acid encoding the VEGFR3 agonist, FGF2, and/or neurological therapeutic agent. The composition or product combination of some embodiments is for use in treating a neurological disease or disorder. The composition or product combination of some embodiments is for use in treating a proteinopathy, such as a tauopathy and/or amyloidosis. The composition or product combination of some embodiments is for use in treating a neurological disease selected from the group consisting of: AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items.

### BRIEF DESCRIPTION OF THE DRAWINGS

FIGS. 1A-1G are a series of microscope images and graphs showing effects of the flow modulator VEGF-c and the neurological therapeutic agent amyloid beta antibody on the meninges and meningeal vasculature of adult 5×FAD mice in accordance with some embodiments.

FIG. 1A depicts the age of the of 5×FAD mice and treatment regimen.

FIG. 1B shows representative images of the meningeal whole-mounts of 5×FAD mice treated with different combinations of mIgG2a or monoclonal anti-amyloid beta antibody (“ABETA Mab1”) with AAV1-CMV-eGFP or AAV1-CMV-mVEGF-C. Meninges were stained for LYVE-1 and CD31; scale bar, 1 mm; inset, 300 m.

FIGS. 1C-1G show measurements of transverse sinus diameter, coverage by LYVE-1negCD31+ blood vessels, total number of lymphatic branches, transverse sinus lymphatic vessel diameter and coverage by LYVE-1+ lymphatic vessels. Results in FIGS. 1C-1G are presented as mean±s.e.m.; n=7 in eGFP+mIgG2a, n=6 in eGFP+ABETA Mab1, in mVEGF-C+mIgG2a and in mVEGF-C+ABETA Mab1; Two-way ANOVA with Sidak's multiple comparison test. In FIGS. 15D and 15G, the units for the Y-axis are percentage of field of view (“% FOV”).

FIGS. 2A-2M are a series of microscope images and graphs showing effects of the flow modulator VEGF-c and the neurological therapeutic agent amyloid beta antibody on amyloid beta protein and plaques in adult 5×FAD mice in accordance with some embodiments.

FIG. 2A shows representative images of the brain sections of these mice. Brain sections were stained for Aβ and with DAPI; scale bar, 2 mm.

FIGS. 2B-2M show plaque density (number of plaques per mm²) (FIGS. 2B-2E), average size (m²) (FIGS. 2F-2I) and coverage (% of brain section) (FIGS. 2J-2M) in particular brain regions (cortex and amygdala, FIGS. 2B, 2F and 2J; hippocampus, FIGS. 2C, 2G and 2K; thalamus and hypothalamus, FIGS. 2D, 2H and 2L) or in the whole brain section (FIGS. 2E, 2I and 2M). Results are presented as mean±s.e.m.; n=7 in eGFP+mIgG2a, n=6 in eGFP+ABETA Mab1, in mVEGF-C+mIgG2a and in mVEGF-C+ABETA Mab1; Two-way ANOVA with Sidak's multiple comparison test.

FIGS. 3A-3G are a series of graphs and microscope images showing effects of the flow modulator VEGF-c and the neurological therapeutic agent amyloid beta antibody on behavior and amyloid beta plaques of aged APPswe mice in accordance with some embodiments. Behaviors assessed include open field, novel location recognition, and contextual fear conditioning.

FIG. 3A depicts the age of the of APPswe mice and treatment regimen.

FIG. 3B depicts total distance, velocity and time in center of the arena (% of total time) in the open field test.

FIG. 3C depicts time investigating one of the object location (% of total time investigating objects) in the training trial and time investigating the novel object location (% of total time investigating) in the novel location recognition test.

FIG. 3D depicts time freezing (% of total time) in the context trial and in cued trial of the contextual fear condition test.

FIG. 3E show representative images of the brain sections of APPswe mice treated with anti-Abeta antibody (ABETA Mab1) and with AAV1-CMV-eGFP or AAV1-CMV-mVEGF-C. Brain sections were stained for Aβ and with DAPI; scale bar, 1 mm.

FIGS. 3F-3G show plaque density (number of plaques per mm²), average size (m²) and coverage (% of brain section) in the cortex and amygdala (FIG. 3F) or in the hippocampus (FIG. 3G). Results in FIGS. 3B-3D, 3F, and 3G are presented as mean±s.e.m.; n=11 per group; Two-tailed unpaired Student's T test.

FIGS. 4A-4Q show meningeal immune response in 3 months-old 5×FAD mice and WT age-matched littermate controls.

FIG. 4A is representative flow cytometry dot plots showing gating strategy for CD64⁺ macrophages (pre-gated in singlets CD45⁺ live cells), CD19⁺ B cells (pre-gated in CD64^{neg} cells) and CD11c⁺MHC-II^{high} dendritic cells (DCs, pre-gated in CD19^{neg} cells).

FIG. 4B is representative flow cytometry dot plots showing gating strategy for γδT cells (pre-gated in singlets CD45⁺ live cells), TCRP^{neg} eNK1.1⁺ cells (NK cells, pre-gated in TCR^{neg} cells), TCRβ⁺NK1.1⁺ cells (NKT cells, pre-gated in TCRβ⁺ cells) and CD4⁺ and CD8⁺ T cells (pre-gated in TCRβ⁺NK1.1^{neg} cells).

FIGS. 4C-4K show number of total CD45⁺ live (FIG. 4C), CD64⁺ macrophages (FIG. 4D), B cells (FIG. 4E), DCs (FIG. 4F), γδT cells (FIG. 4G), NK cells (FIG. 4H), NKT cells (FIG. 4I), CD4⁺ T cells (FIG. 4J) and CD8⁺ T cells (FIG. 4K) isolated from the meninges of 3 months-old wild type (WT,) and 5×FAD mice. FIG. 4L is histograms showing the expression levels of PD-1 in the fluorescence minus one (FMO) sample (grey) or in concatenated meningeal immune cell populations from WT and 5×FAD groups. Cells considered to be positive for PD-1 are demarcated in the different histograms.

FIGS. 4M-4Q show frequencies of PD-1-expressing γδT cells (FIG. 4M), NK cells (FIG. 4N), NKT cells (FIG. 4O), CD4⁺ T cells (FIG. 4P) and CD8⁺ T cells (FIG. 4Q) in each group. Results in FIGS. 4C-4K and FIGS. 4M-4Q are presented as mean±s.e.m.; n=5 per group; two-tailed unpaired Student's T test; data is representative of 2 independent experiments.

FIGS. 5A-5R show compromised meningeal lymphatic function is observed in 5×FAD mice and limits brain Aβ clearance by anti-Abeta antibody.

FIG. 5A is representative images of meningeal whole mounts from 5-6- and 13-14 months-old 5×FAD mice, stained for CD31, LYVE-1 and Aβ (stained with the D54D2 antibody; scale bar, 2 mm; inset scale bar, 500 m).

FIG. 5B is a scheme depicting the compartmentalization of the meningeal whole mount for quantification of LYVE-1 and Aβ coverage.

FIGS. 5C-5E are graphs showing the coverage by LYVE-1⁺CD31⁺ vessels as a percentage of the region of interest (% of ROI) at the superior sagittal sinus (SSS) (FIG. 5C), transverse sinus and confluence of sinuses (TS/COS) (FIG. 5D), and petrosquamosal and sigmoid sinuses (PSS/SS) (FIG. 5E). Results in are presented as mean s.e.m.; n=8 per group; two-way ANOVA with Holm-Sidak's multiple comparisons test (for LYVE-1⁺ vessels) and two-tailed unpaired Student's T test (for Aβ coverage); data result from 2 independent experiments.

FIG. 5F shows that lymphatic endothelial cells (LECs) were isolated from the brain meninges of 5×FAD mice and WT littermate controls at 6 months of age, total RNA was extracted and sequenced.

FIG. 5G is principal component analysis (PCA) plot showing segregation between WT and 5×FAD meningeal LEC transcriptomes.

FIG. 5H is a heatmap of top 200 differentially expressed genes.

FIG. 5I shows gene set obtained by functional enrichment of differentially expressed genes in meningeal LECs from 5×FAD mice. Data in FIGS. 5G-5I consists of n=3 per group; individual RNA samples result from LECs pooled from 10 meninges over 3 independent experiments; the Benjamini-Hochberg correction was used to adjust the associated P-values in FIGS. 5H and 5I (adj. P-value <0.05); grey scale bar represents expression values for each sample as standard deviations from the mean across each gene in FIG. 5H; functional enrichment of differential expressed genes was determined with Fisher's exact test in FIG. 5I.

FIG. 5J shows that human LECs were incubated with 100 nM synthetic scrambled human Aβ₄₂ peptides (scramble) or synthetic human monomeric/dimeric Aβ₄₂ for 24h or 72h, total RNA was extracted and sequenced.

FIG. 5K is PCA plot showing segregation between the different samples.

FIG. 5L is a heatmap of top 200 differentially expressed genes.

FIG. 5M is gene sets obtained by functional enrichment of differentially expressed genes in human LECs incubated with Aβ₄₂ for 72h, when compared to scramble 72h. Data in FIG. 5K-5M consists of n=3 per group; individual RNA samples result from human LECs pooled from 3 well replicates; the Benjamini-Hochberg correction was used to adjust the associated P-values in FIGS. 5L and 5M (adj. P-value <0.05); color scale bar represents expression values for each sample as standard deviations from the mean across each gene in FIG. 5L; functional enrichment of differential expressed genes was determined with Fisher's exact test in FIG. 5M.

FIG. 5N shows representative images of skull caps with attached meningeal layers after photo-ablation. Adult 2 months-old WT mice were injected (i.c.m.) with Visudyne (5 μL) followed by a transcranial photoconversion step (Vis./photo.) to ablate meningeal lymphatic vessels. Control mice were injected with Visudyne without photoconversion (Vis.). One week later, mice were injected with 5 μL of a suspension of fluorescent microspheres (1 μm in diameter) into the CSF and 15 minutes later the lymphatic vessel afferent to the deep cervical lymph node (dCLN) was imaged by in vivo stereomicroscopy. Representative images of skull cap show microspheres and lymphatic vessels stained for LYVE-1 around the confluence of sinuses (COS) and transverse sinus (TS) at the dorsal brain meninges or around the sigmoid (SS) and petrosquamosal (PSS) sinuses at the basal brain meninges (scale bars, 500 μm).

FIGS. 5O and 5P are graphs showing LYVE-1⁺ vessel total length (in mm) and branching points in dorsal meninges (FIG. 5O) and basal meninges (FIG. 5P). Results in FIGS. 5O and 5P are presented as mean±s.e.m.; n=10 per group; two-tailed unpaired Student's T test; data is representative of 2 independent experiments.

FIG. 5Q is representative frames showing microspheres flowing through the lymphatic vessel afferent to the dCLN, or cumulative sphere tracking (for 20 sec), in mice with intact or ablated meningeal lymphatic vessels.

FIG. 5R is graph with quantifications of microsphere flow (number microspheres per minute) in mice from different groups. Results are presented as mean±s.e.m.; n=11 in Vis. group and n=14 in Vis./photo. group; two-tailed unpaired Student's T test; data results from 2 independent experiments.

FIGS. 6A-6F show functional enrichment analysis of differentially expressed genes in meningeal LECs from 5×FAD mice and in human LECs incubated with Aβ₄₂.

FIG. 6A is a heatmap of top 50 differentially expressed genes in WT and 5×FAD LECs at 6 months of age.

FIGS. 6B and 6C are Exocytosis (GO:0006887) and Phospholipase D signaling pathway (KEGG:mmu04072) gene sets obtained by functional enrichment analysis, with corresponding differentially expressed genes.

FIG. 6D is a heatmap of top 50 differentially expressed genes in scramble or Aβ₄₂ at 24h or 72h.

FIGS. 6E and 6F are adherens junctions (KEGG:hsa04520) and Phospholipase D signaling pathway (KEGG:hsa04072) gene sets obtained by functional enrichment analysis, with corresponding differentially expressed genes.

Data in FIGS. 6A-6C consists of n=3 per group; individual RNA samples result from LECs pooled from 10 meninges over 3 independent experiments. Data in FIGS. 6D-6F consists of n=3 per group; individual RNA samples result from human LECs pooled from 3 well replicates; functional enrichment of differential expressed genes was determined with Fisher's exact (P<0.05); color scale bars represent expression values for each sample as standard deviations from the mean across each gene.

FIGS. 7A-7E show mass cytometry gating strategy, marker expression levels, unsupervised clustering, and meningeal immune cell numbers.

FIG. 7A shows representative mass cytometry dot plots depicting gating strategy used to select CD45⁺ live cells used in further high-dimensional analysis. Meningeal single-cell suspensions were obtained from 5×FAD mice at 5-6 months and 11-12 months and processed for mass cytometry.

FIG. 7B shows final heatmaps of the median marker expression values for each immune cell cluster identified using Rphenograph. Median marker expression values are indicated by color intensity depicted in the scale bar.

FIG. 7C is t-distributed stochastic neighbor embedding (tSNE) plots showing unsupervised clustering of CD45⁺ live immune cells.

FIG. 7D is number of total CD45⁺ live meningeal leukocytes.

FIG. 7E is numbers of different meningeal immune cell clusters showing a statistically significant increase in B cells, CD4⁺ T cells, CD8⁺ T cells, type 3 innate lymphoid cells (ILC3s) and an undefined cell population isolated from the meninges of 5×FAD mice at 11-12 months of age. Results in FIG. 7D and FIG. 7E are presented as mean±s.e.m.; n=7 per group; two-tailed unpaired Student's T test.

FIGS. 8A-8C show specificity of an anti-Abeta antibody (ABETA Mab1) and kinetics of brain Aβ recognition upon i.c.m. or i.v. injections.

FIG. 8A is representative images of brain sections from 4 months-old 5×FAD mice and WT littermate controls that were incubated without primary antibody (ab), with murine IgG2a isotype control (anti-fluorescein), anti-Abeta antibody murine IgG2a monoclonal antibody (against human Aβ protofibrils, ABETA Mab1) or a commercially available rabbit anti-human Aβ (D54D2 clone, optimal for immunofluorescence staining). Images show Aβ and DAPI staining (scale bar, 1 mm).

FIG. 8B presents images of different regions of the brain of 5×FAD mice, 1-hour post injection of anti-Abeta antibody (1 μg/μL) into the CSF (5 μL, i.c.m.) or into the blood (100 μL, i.v.).

FIG. 8C presents images of different regions of the brain of 5×FAD mice 24 hours post injection of anti-Abeta antibody into the CSF (i.c.m.) or into the blood (i.v.). Images in FIG. 8B and FIG. 8C panels show astrocyte endfeet and glia limitans stained for Aquaporin 4 (AQP4), As stained with Amylo-Glo RTD and anti-Abeta antibody staining (scale bar, 200 m). Data is representative of 2 independent experiments.

FIGS. 8D-8N show pharmacological ablation of the dorsal meningeal lymphatic vessels in 5×FAD mice dampens Aβ plaque clearance by anti-Abeta antibody.

FIG. 8D shows that adult 4-5 months-old female 5×FAD mice were injected (i.c.m.) with Visudyne (5 μL) plus photoconversion (Vis./photo.) or Visudyne without photoconversion (Vis.). One week later, mice were injected (i.c.m.) with anti-Abeta antibody (murine antibody against Aβ protofibrils, ABETA Mab1) or murine IgG2a (mIgG2a) as a control (both at 1 μg/L). Injection of anti-Abeta antibody or mIgG2a was repeated two weeks later. Another lymphatic vessel ablation step was performed, followed by two injections with anti-Abeta antibody or mIgG2a.

FIG. 8E is representative images of brain sections from 5×FAD mice stained for As and with DAPI (scale bar, 1 mm).

FIGS. 8F and 8G are graphs showing number of Aβ plaques per mm², average size of Aβ plaques (m²) and coverage of Aβ (% of region) in the hippocampus (FIG. 8F) and cortex/striatum/amygdala (FIG. 8G). Results are presented as mean±s.e.m.; n=5 in mIgG2a groups and n=6 in anti-Abeta antibody groups; Two-way ANOVA with Sidak's multiple comparisons test.

FIG. 8H is representative stereomicroscopy images of lymphatic vessels stained for LYVE-1 around the confluence of sinuses (COS) and transverse sinus (TS) at the dorsal brain meninges or around the sigmoid (SS) and petrosquamosal (PSS) sinuses at the basal brain meninges still attached to the skull cap (scale bars, 500 m).

FIGS. 8I-8L are graphs showing LYVE-1⁺ vessel total length (in mm) (FIG. 8I) and branching points (FIG. 8J) in the dorsal meninges and total length (in mm) (FIG. 8K) and branching points (FIG. 8L) in the basal meninges.

FIGS. 8M and 8N are quantification of number of Aβ plaques per mm², average size of Aβ plaques (m²) and coverage of Aβ (% of region/section) in the thalamus/hypothalamus (FIG. 8M) and in the whole brain section (FIG. 8N). Results in are presented as mean±s.e.m.; n=5 in mIgG2a groups and n=6 in anti-Abeta antibody groups; Two-way ANOVA with Sidak's multiple comparisons test.

FIGS. 9A-9K show repeated delivery of anti-Abeta antibody (ABETA Mab1) into the CSF of 5×FAD mice reduces brain Aβ plaque load.

FIG. 9A shows that adult 5×FAD mice (3 months-old) were injected (i.c.m.) with either mIgG2a (5 μL at 1 μg/μL) or anti-Abeta antibody (5 μL at 0.1 or 1 μg/L). Injections were repeated another three times, every two weeks, as shown in the scheme.

FIG. 9B is representation of different brain regions considered for the quantification of Aβ, namely hippocampus, cortex/striatum/amygdala and thalamus/hypothalamus.

FIG. 9C is representative images of brain sections from the different groups stained for Aβ and with DAPI (scale bar, 2 mm).

FIGS. 9D-9K are quantification of average size of Aβ plaques (m²) and coverage of Aβ (% of section) in the hippocampus (FIGS. 9D and 9E), cortex/striatum/amygdala (FIGS. 9F and 9G), thalamus/hypothalamus (FIGS. 9H and 91) and in the whole brain section (FIGS. 9J and 9K). Results in FIGS. 9D-9K are presented as mean±s.e.m.; n=5 in mIgG2a group and n=6 in anti-Abeta antibody groups; One-way ANOVA with Bonferroni's multiple comparisons test.

FIGS. 10A-10K show that compromising meningeal lymphatic function in 5×FAD mice limits brain Aβ clearance by ABETA Mab1 and modulates neuritic dystrophy, microglial activation and fibrinogen levels.

FIG. 10A shows that adult 3-3.5 months-old male 5×FAD mice were injected (i.c.m.) with Visudyne (5 μL) plus photoconversion (Vis./photo.) or Visudyne without photoconversion (Vis.). Upon recovery, mice received intraperitoneal (i.p.) injections of ABETA Mab1 (a murine antibody against Aβ protofibrils) or the control murine IgG (mIgG) antibodies, each at a dose of 40 mg/kg. Antibodies were injected weekly for a total of four weeks. Additional steps of meningeal lymphatic vessel ablation or control interventions were followed by four weekly injections with antibodies. Mice were tested in the open field and Morris water maze.

FIG. 10B show representative images of brain sections from 5×FAD mice stained for Aβ (stained with the D54D2 antibody) and for LAMP1 (scale bars, 1 mm).

FIGS. 10C-10F are graphs showing number of Aβ plaques per mm² of brain section (FIG. 10C), average size of Aβ plaques (m²) in ABETA Mab1 cohort (FIG. 10D), coverage by Aβ plaques (FIG. 10E) and coverage by LAMP1⁺ dystrophic neurites (FIG. 10F) (as % of brain section) in each group.

FIG. 10G shows representative images of the brain cortex stained for Aβ (stained with Amilo-Glo), fibrinogen (grey), IBA and CD68 (scale bar, 100 m).

FIGS. 10H-10K are graphs showing the coverage by IBA1⁺ cells (% of field) (FIG. 10H), number of peri-AP plaque IBA1⁺ cells (FIG. 10I), percentage of IBA1 occupied by CD68 (FIG. 10J) and fibrinogen coverage (% of field) (FIG. 10K) in each group.

Results in FIGS. 10C-10F and FIGS. 10H-10K are presented as mean±s.e.m.; n=9 in each group; in FIGS. 10C-10D, 10F and 10H-10K, two-way ANOVA with Holm-Sidak's multiple comparisons test; in FIG. 10E, two-tailed unpaired Student's T test.

FIGS. 11A-11G show that meningeal lymphatic dysfunction leads to anxious-like behavior and worsened spatial learning and memory in 5×FAD mice.

FIG. 11A shows that adult 5×FAD mice with intact or ablated meningeal lymphatics and treated with ABETA Mab1 or control mIgG antibodies were tested in the open field arena and in the Morris water maze.

FIGS. 11B-12D are graphs showing total distance (in centimeters) (FIG. 12B), velocity (in centimeters per second) (FIG. 11C) and percentage of time in the center of the open field arena (% of total time) (FIG. 11D).

FIGS. 11E-11G are graphs showing latency to platform in acquisition (in seconds) (FIG. 11E), percentage of time in the platform quadrant in the probe trial (FIG. 11F) and latency to platform in reversal (in seconds) (FIG. 11G).

Results in FIGS. 11B-11G are presented as mean±s.e.m.; n=9 in each group; two-way ANOVA with Holm-Sidak's multiple comparisons test in FIGS. 11B-11D and 11F; repeated measures two-way ANOVA with Tukey's multiple comparisons test in FIGS. 11E and 11G; statistically significant differences between groups in days 3, 4 and 7 of the Morris water maze test are indicated as D3, D4 and D7, respectively.

FIGS. 12A-12F show impairing meningeal lymphatic drainage affects the access of anti-Abeta antibody (ABETA Mab1) to Aβ plaques in the brain parenchyma. 5×FAD mice (5 months-old) with intact or ablated meningeal lymphatic vasculature were injected (i.c.m.) with 5 μL of anti-Abeta antibody (at 1 μg/L). One hour later, mice were transcardially perfused and the brain was collected for analysis.

FIGS. 12A and 12B are images of ten different regions of the brain of 5×FAD mice from the Visudyne (Vis.) or Visudyne plus photoconversion (Vis./photo.) groups showing blood vessels stained for CD31, Aβ stained with Amylo-Glo RTD and anti-Abeta antibody staining (scale bar, 200 μm).

FIGS. 12C and 12D are graphs with colocalization between CD31 and anti-Abeta antibody (% of CD31 signal occupied by anti-Abeta antibody) in each brain region (1 to 10) (FIG. 12C) or presented as the average of all regions (FIG. 12D).

FIGS. 12E and 12F are graphs with quantifications of colocalization between Aβ aggregates and anti-Abeta antibody (% of AR signal occupied by anti-Abeta antibody) in each brain region (1 to 10) (FIG. 12E) or presented as the average of all regions (FIG. 12F). Results in FIGS. 12C-12F are presented as mean±s.e.m.; n=5 per group; Two-way ANOVA with Sidak's multiple comparisons test in FIGS. 12C and 12E; two-tailed unpaired Student's T test in FIGS. 12D and 12F.

FIGS. 13A-13U show impairing meningeal lymphatic drainage in 5×FAD mice affects microglial gene expression.

FIG. 13A shows that myeloid cells were sorted (by fluorescence activated cell sorting, FACS) from the brain cortex of 4 months-old 5×FAD mice injected with Visudyne alone (Vis.) or Visudyne followed by transcranial photoconversion (Vis./photo.). Transcriptome of sorted live CD45⁺Ly6G^{neg}CD11b⁺ cells pooled from 3 mice per group was analyzed by single-cell RNA-seq (scRNA-seq).

FIG. 13B depicts unsupervised clustering and t-distributed stochastic neighbor embedding (tSNE) representation of four distinct clusters of microglia.

FIG. 13C shows frequency of microglia (% of total cells) from each cluster was similar when comparing the Vis. and Vis./photo. groups.

FIG. 13D is a heatmap with genes involved in the transition from homeostatic to disease-associated microglia phenotypes, depicting the homeostatic (clusters 1 and 2), Trem2 independent (cluster 3) and Trem2 dependent (cluster 4) signatures within each cell. Cells were grouped by cluster and genes were grouped by signature. Scale shows mean-centered, log-normalized expression values.

FIGS. 13E and 13F are violin plots showing Hexb expression levels e) in microglia from each cluster (FIG. 13E) and all microglia (FIG. 13F).

FIGS. 13G and 13H are violin plots showing Apoe expression levels in microglia from each cluster (FIG. 13G) and all microglia (FIG. 13H).

FIGS. 13I-13L are violin plots showing expression of the major histocompatibility complex II genes Cd74 (FIG. 13I), H2-D1 (FIG. 13J), H2-Aa (FIG. 13K) and H2-Ab1 (FIG. 13L). Data in FIGS. 13B-13L resulted from the analysis of 402 microglia in the Vis. group and 249 microglia in the Vis./photo. group (from sequenced cells sorted from the cortices of 3 mice per group); Wilcoxon Rank-Sum test with Bonferroni's multiple comparisons test was used in FIGS. 13E-13L.

FIG. 13M shows that flow cytometry was performed using cell suspensions from the brain cortex of 4 months-old 5×FAD mice injected with Visudyne alone (Vis.) or Visudyne followed by transcranial photoconversion (Vis./photo.).

FIG. 13N is representative flow cytometry dot plots showing gating strategy for live CD45high CD1b^{neg} (lymphoid cells), CD45hghCD11b⁺ (recruited and/or activated myeloid cells) and CD45^{int}CD11b⁺ (microglia).

FIGS. 130-13Q are frequencies of CD45^{high}CD11b⁺ (FIG. 13O), CD45^{high}CD11b⁺ (FIG. 13P) and CD45^{high} CD11b^{neg} (FIG. 13Q) cells isolated from the brain cortex of 5×FAD mice from the Vis. and Vis./photo. groups.

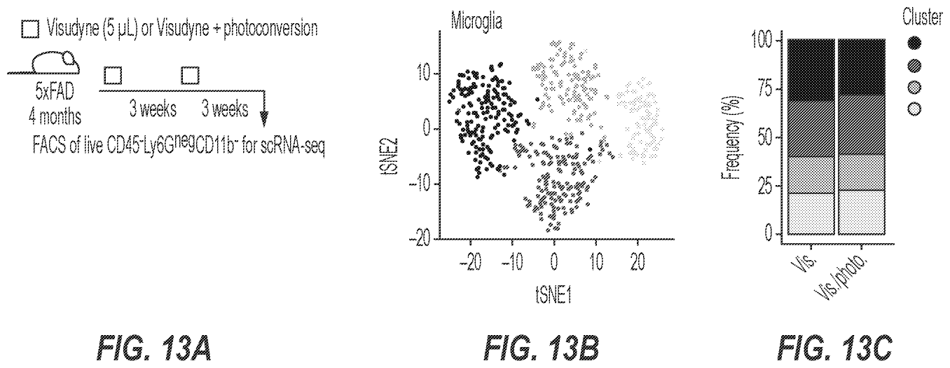

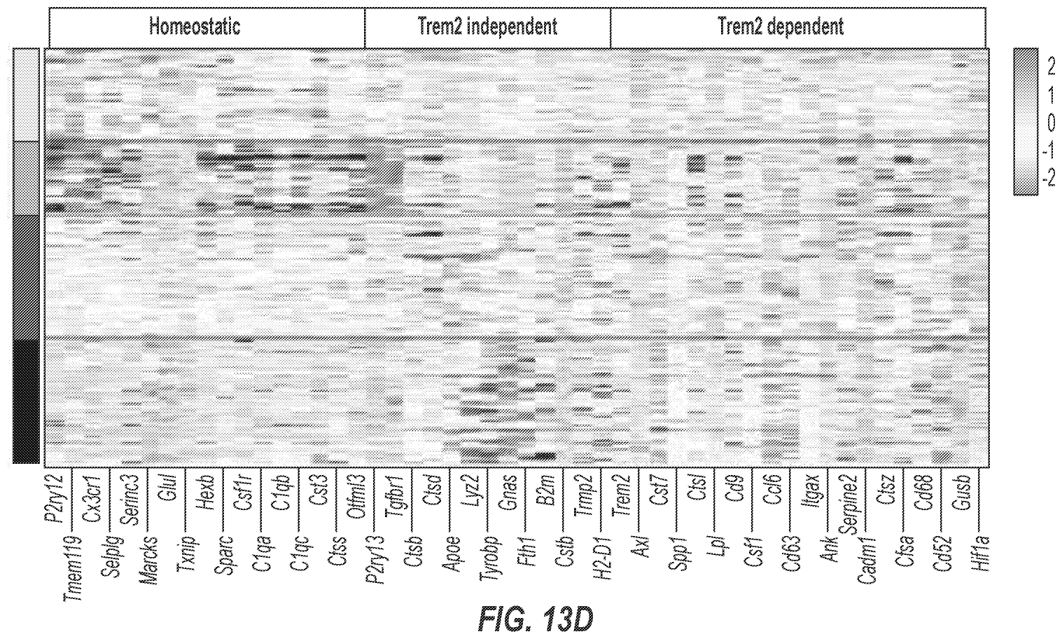

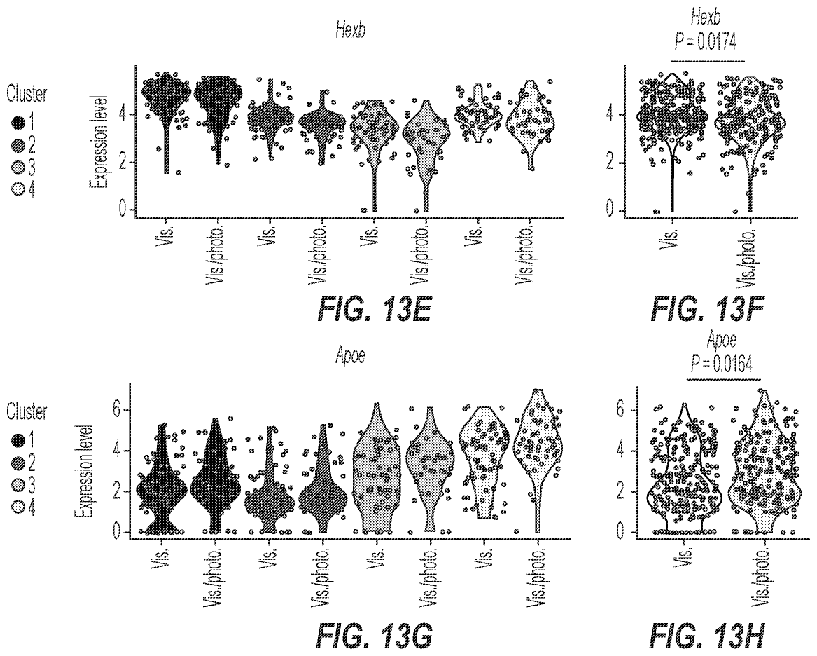

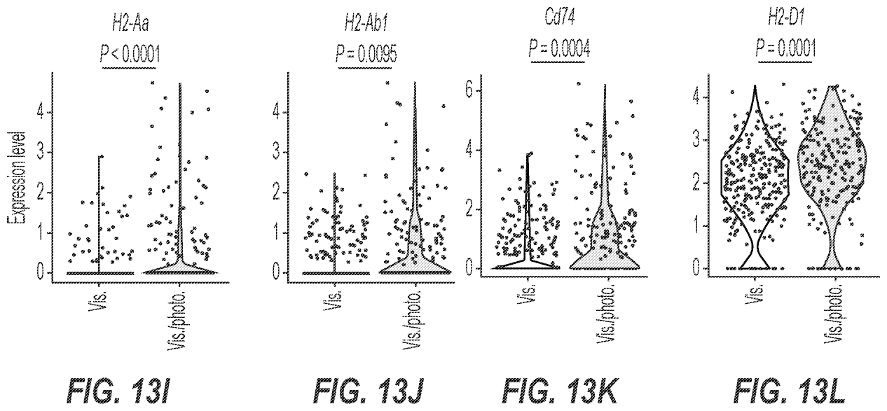

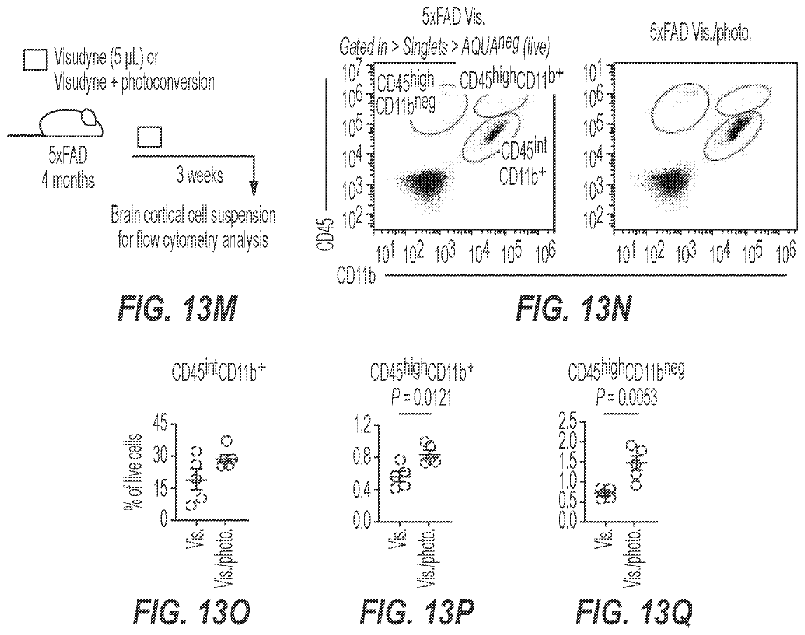

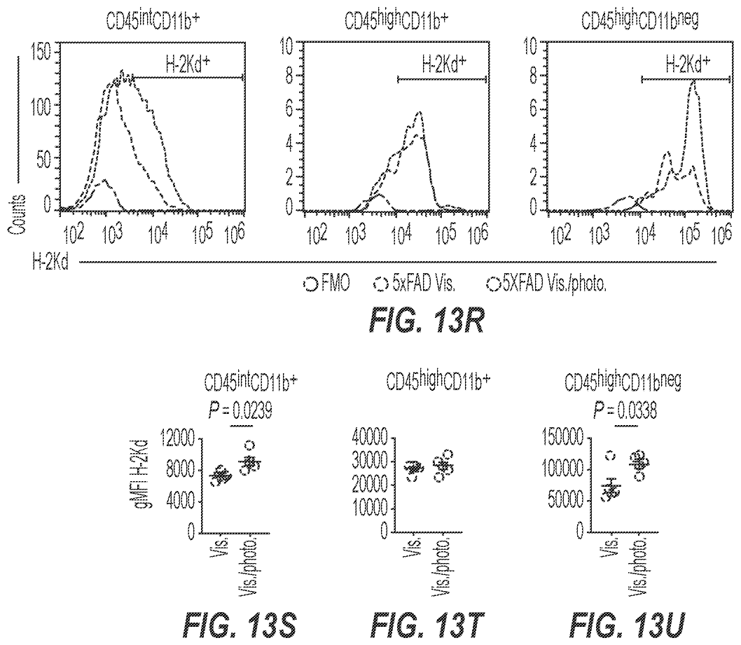

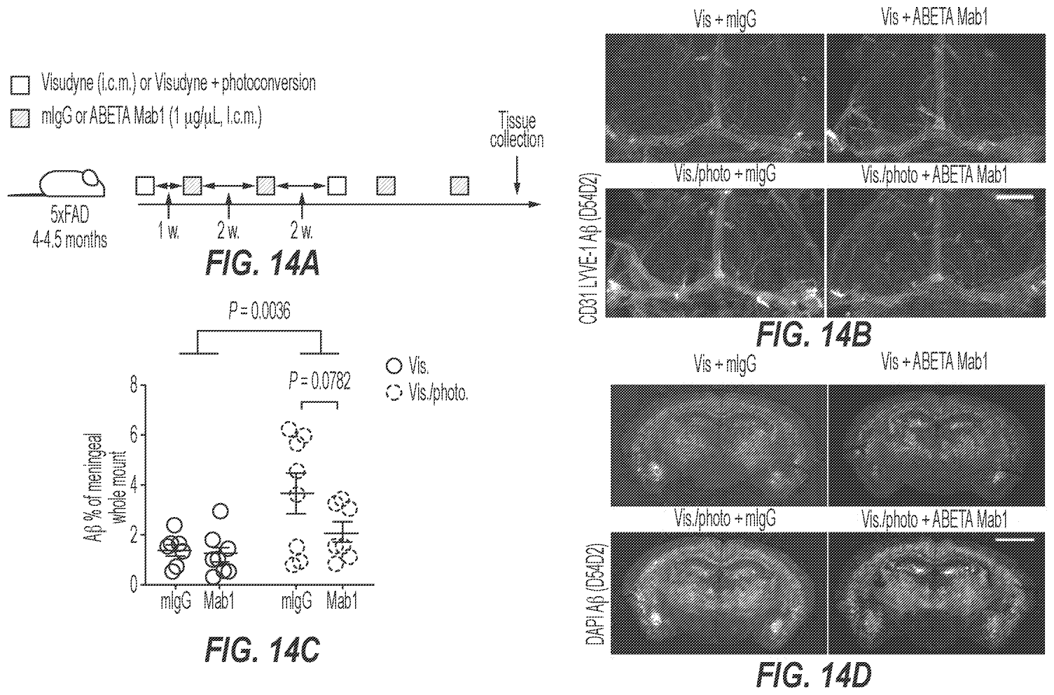

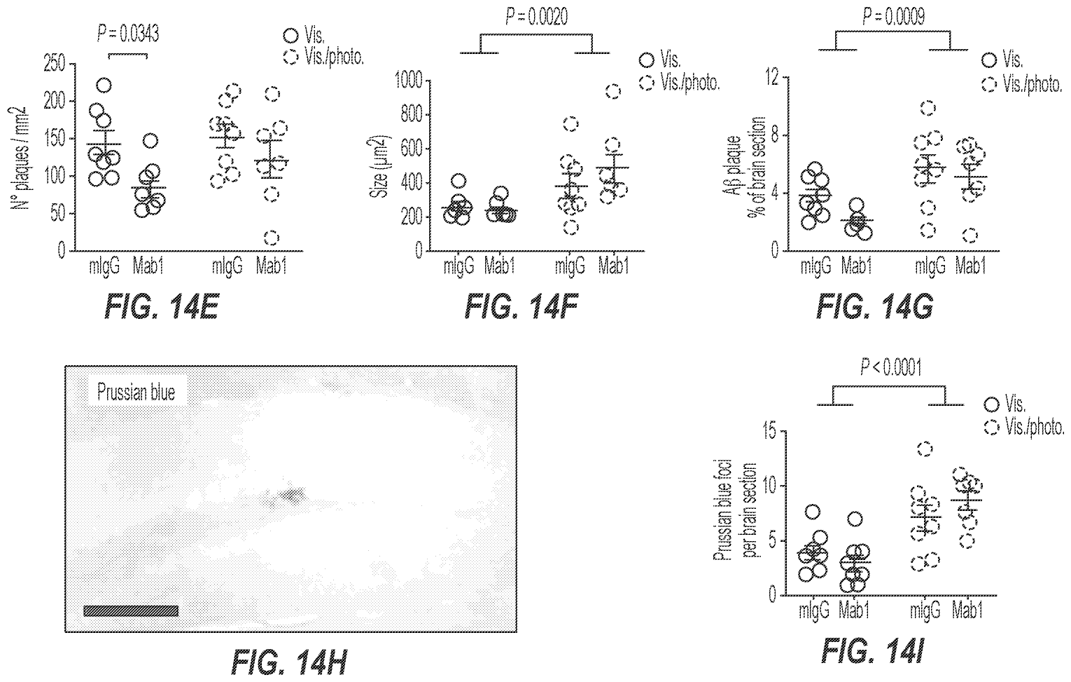

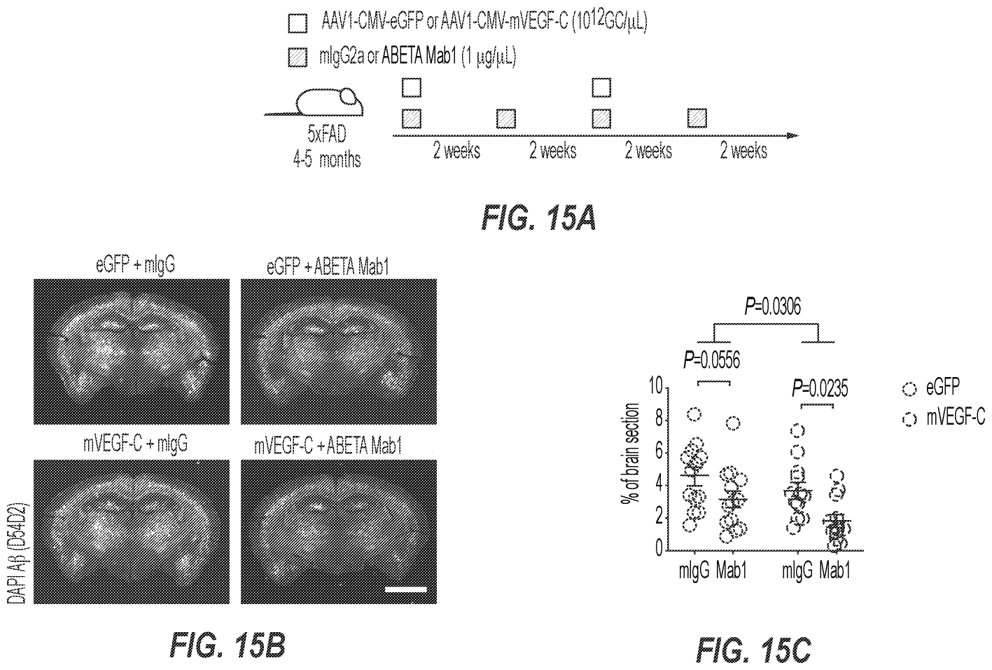

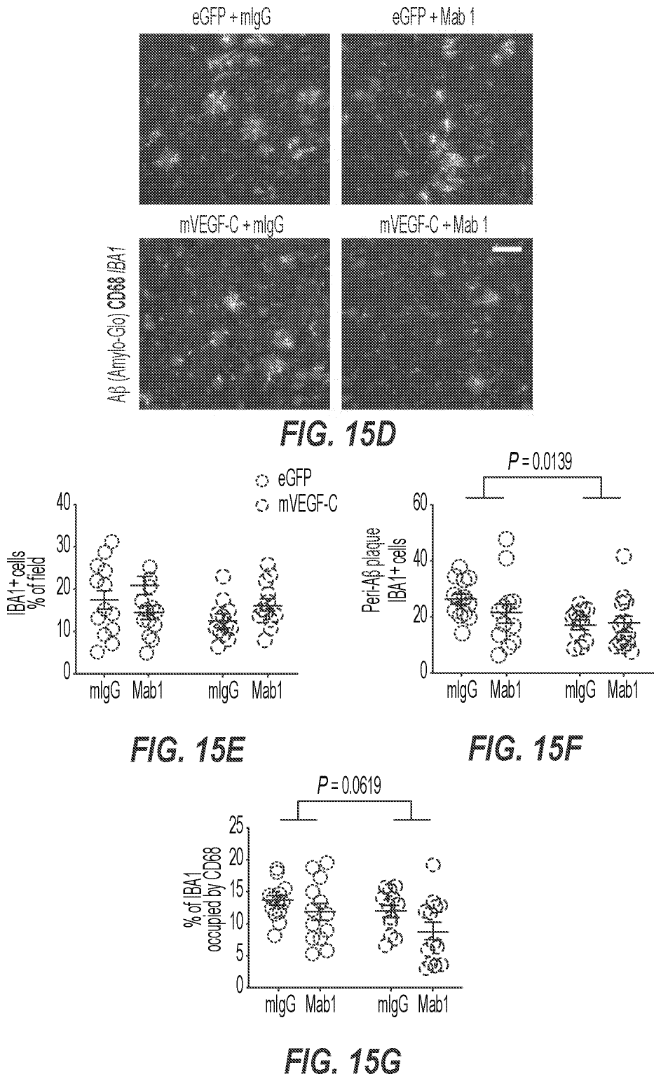

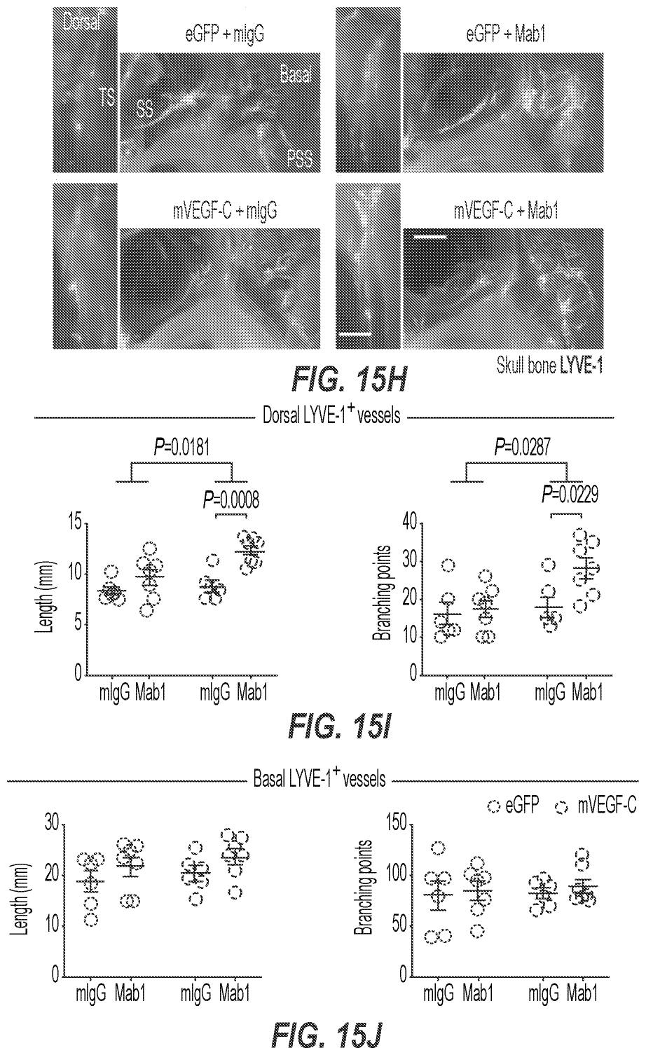

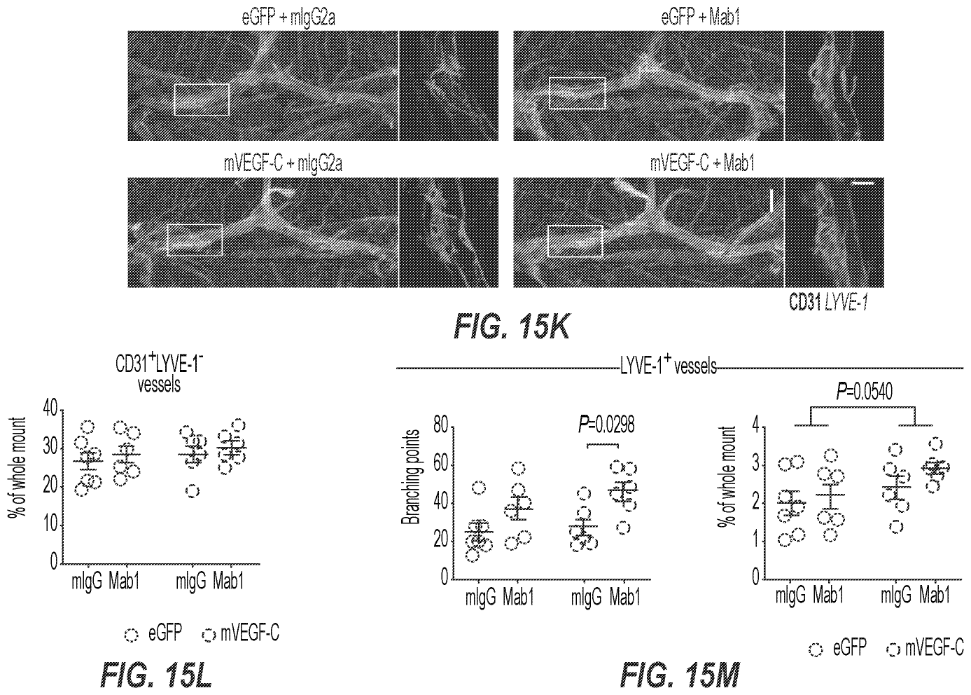

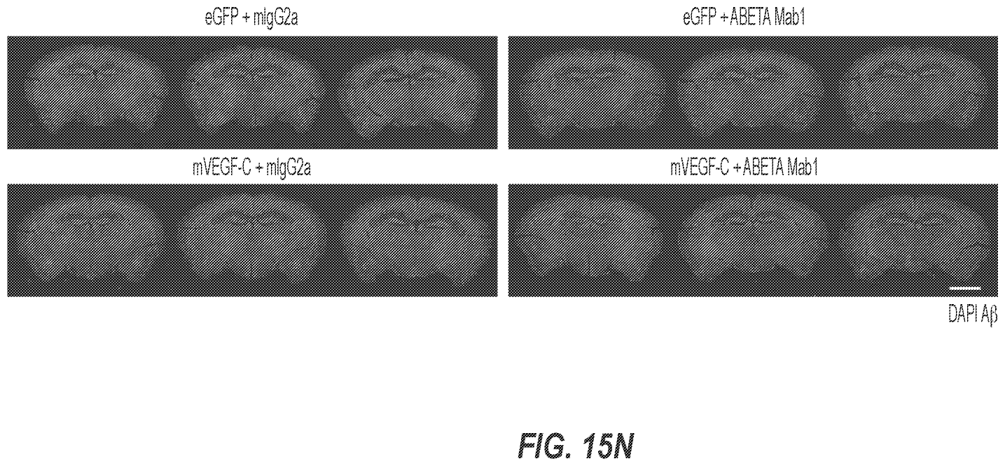

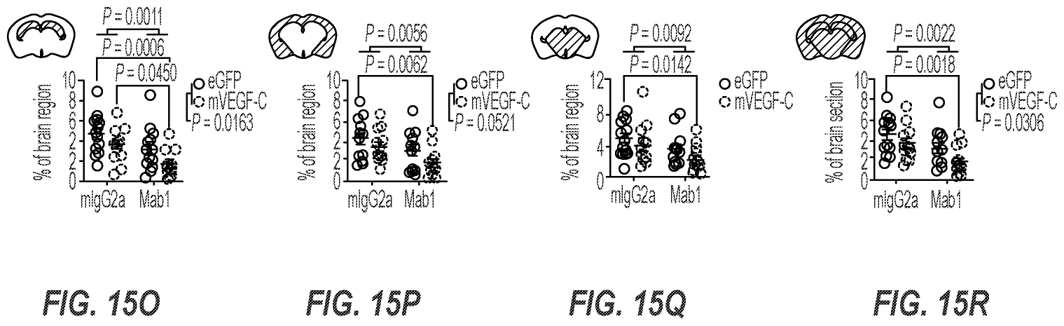

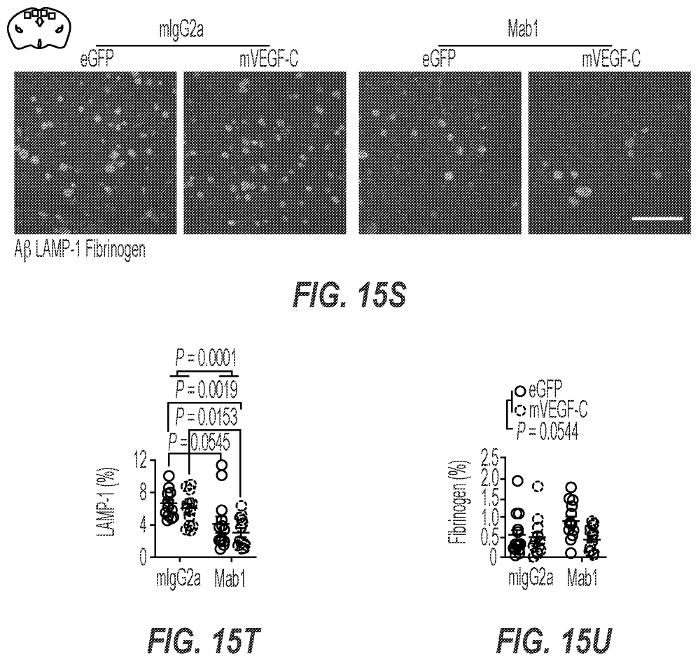

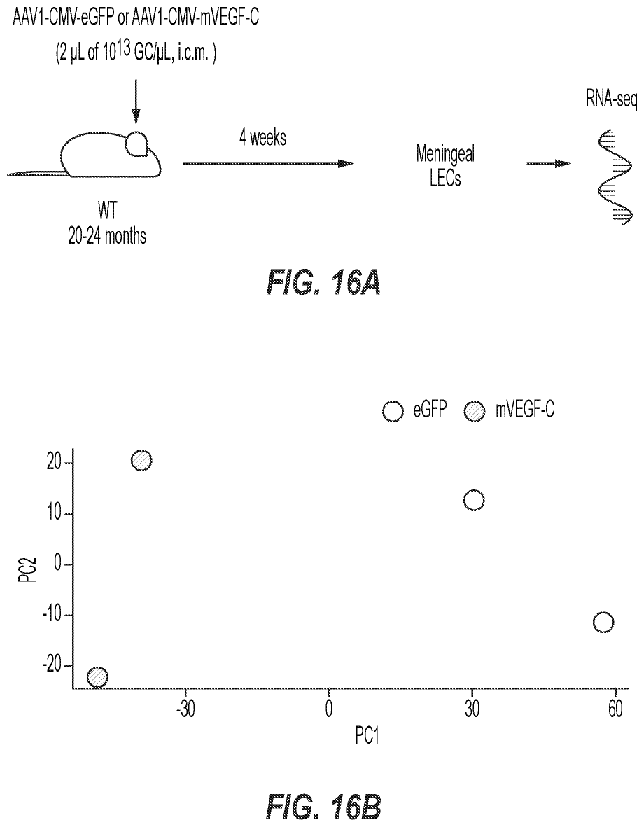

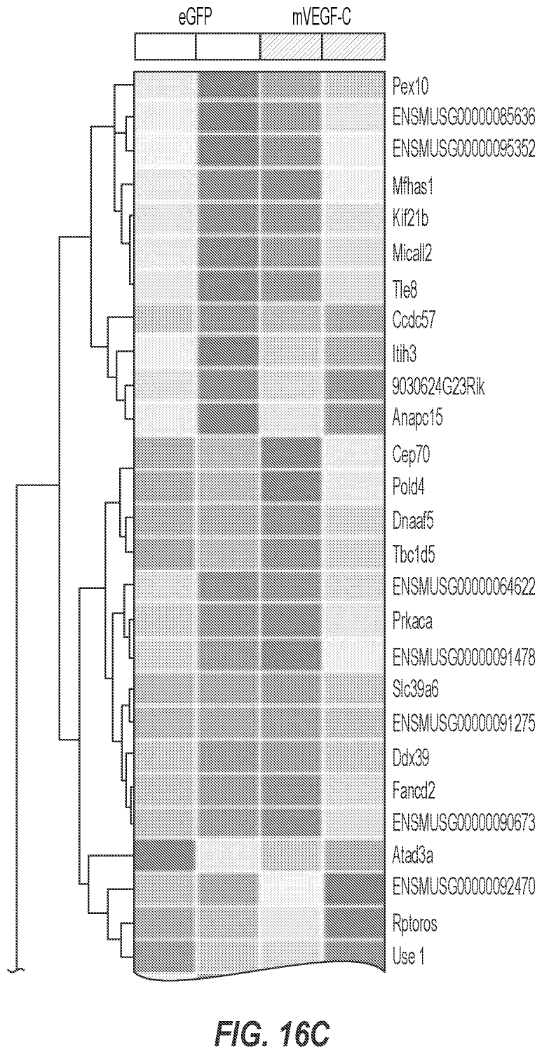

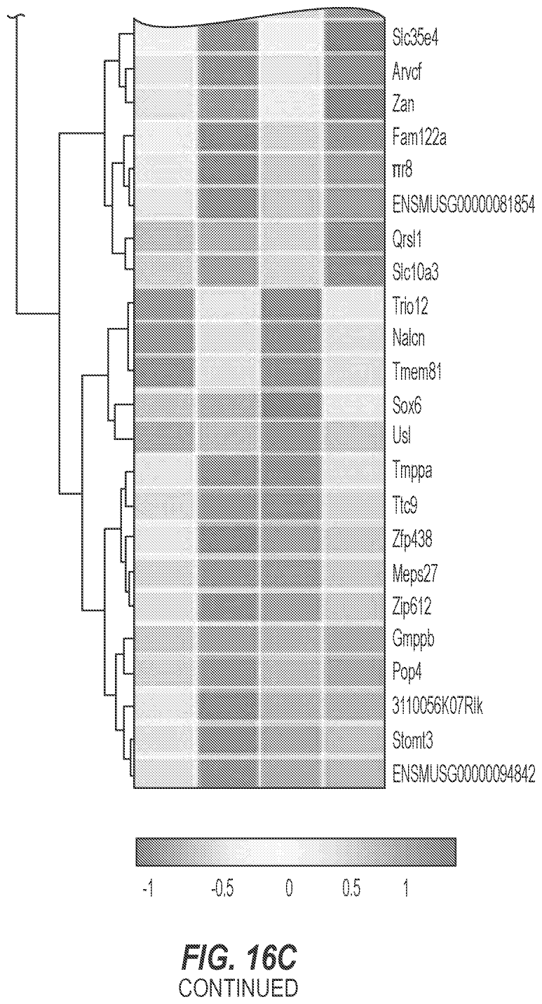

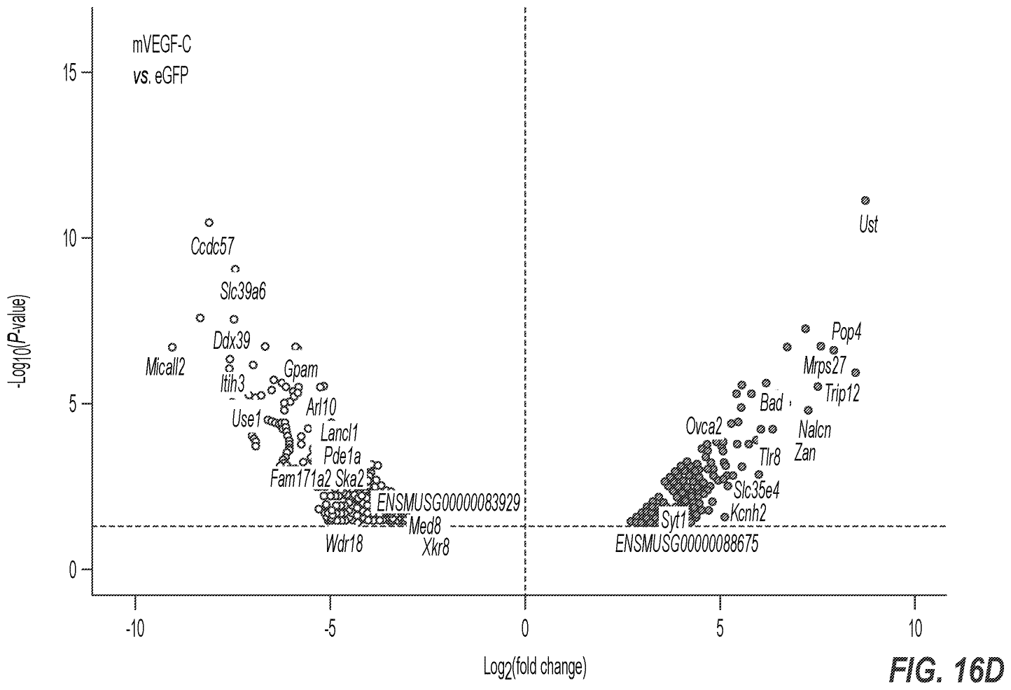

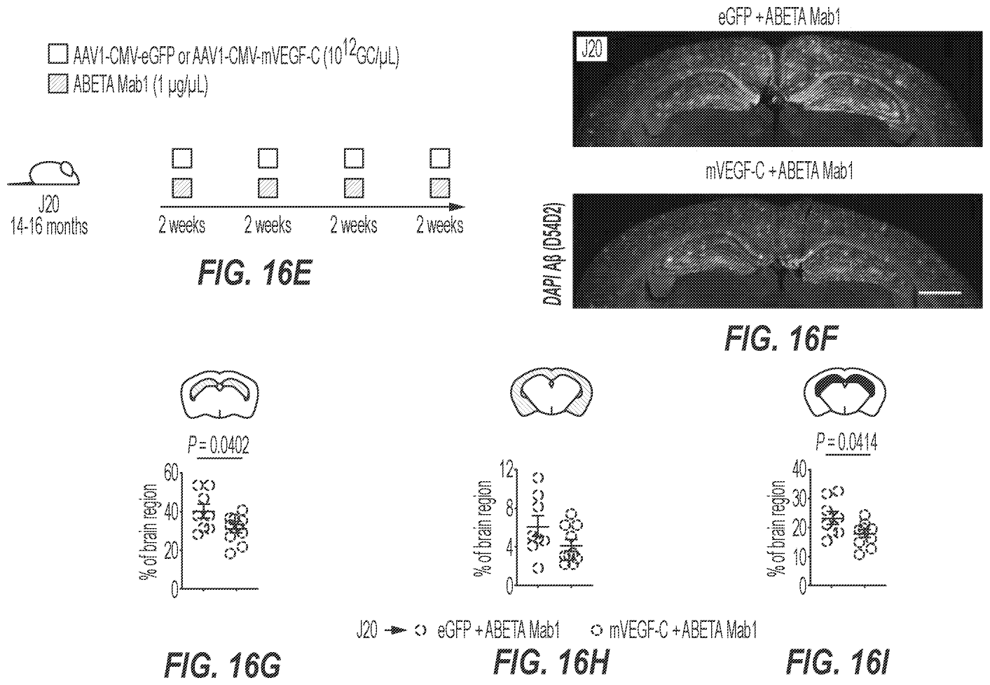

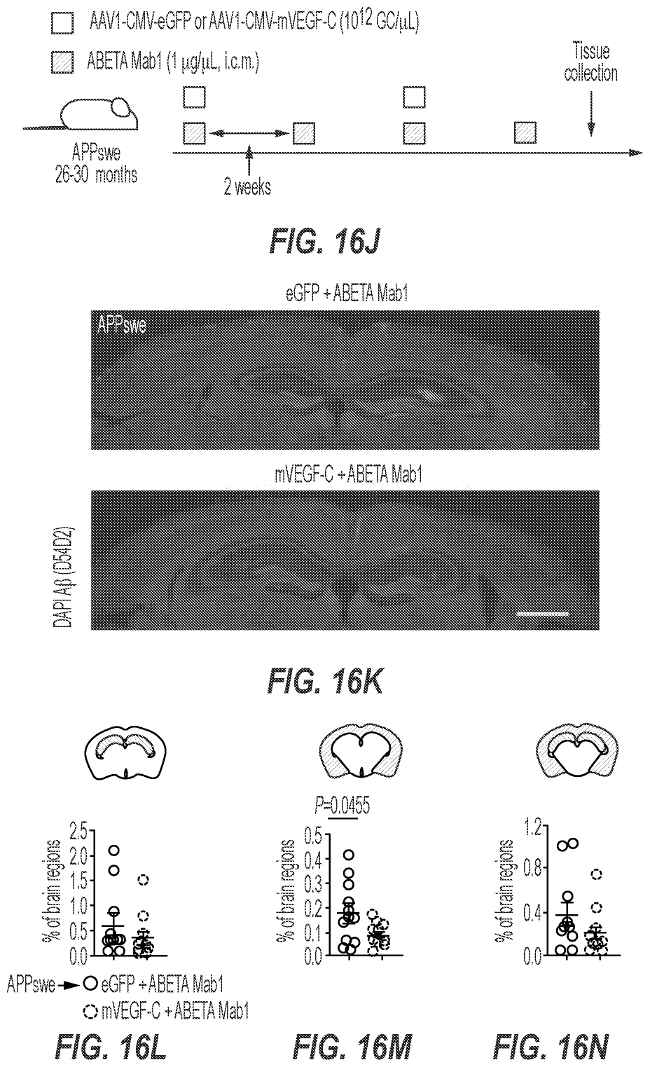

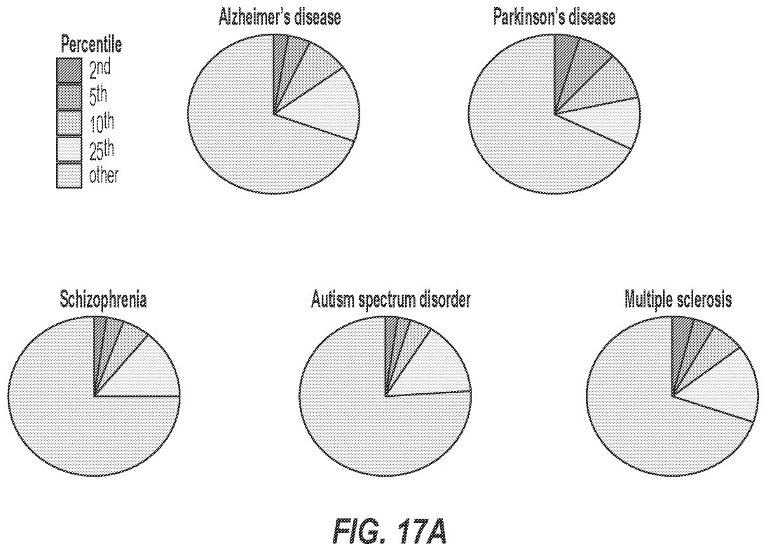

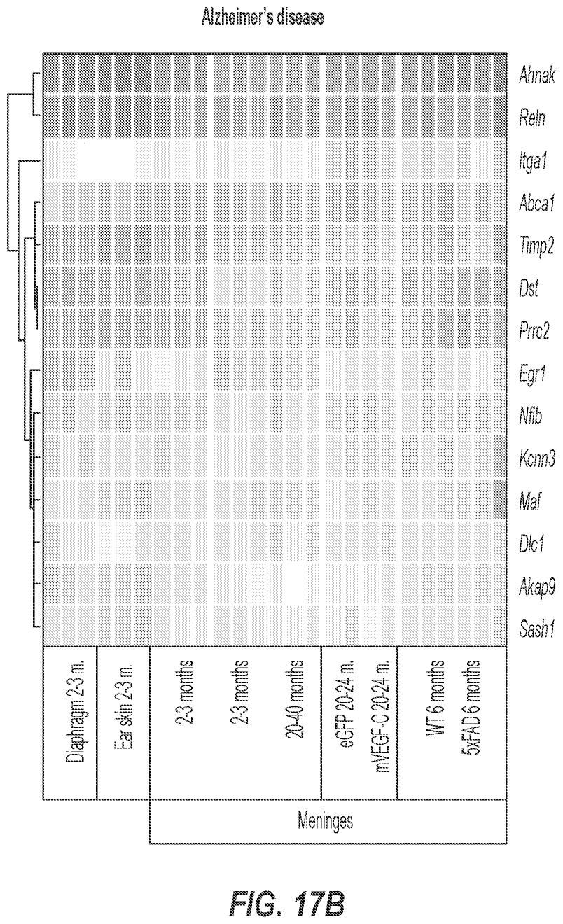

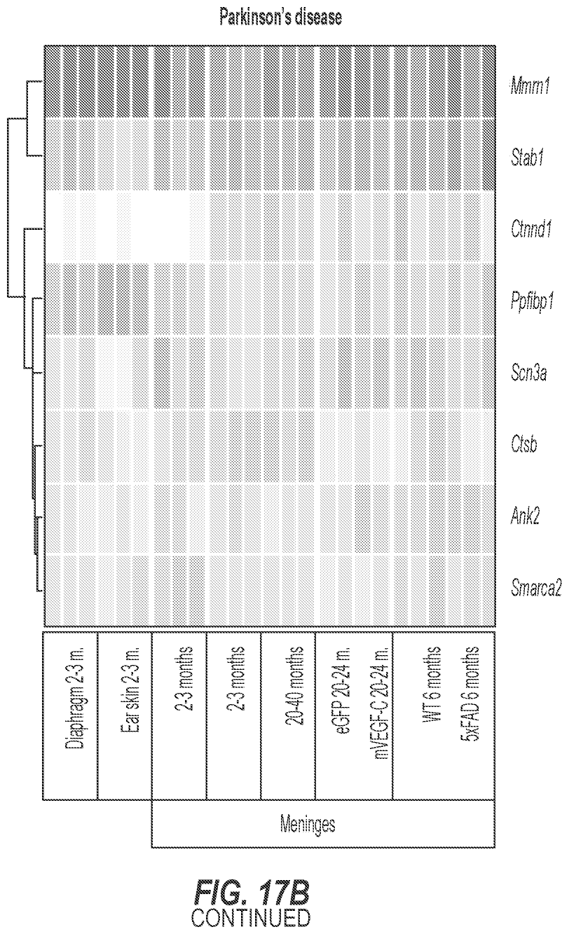

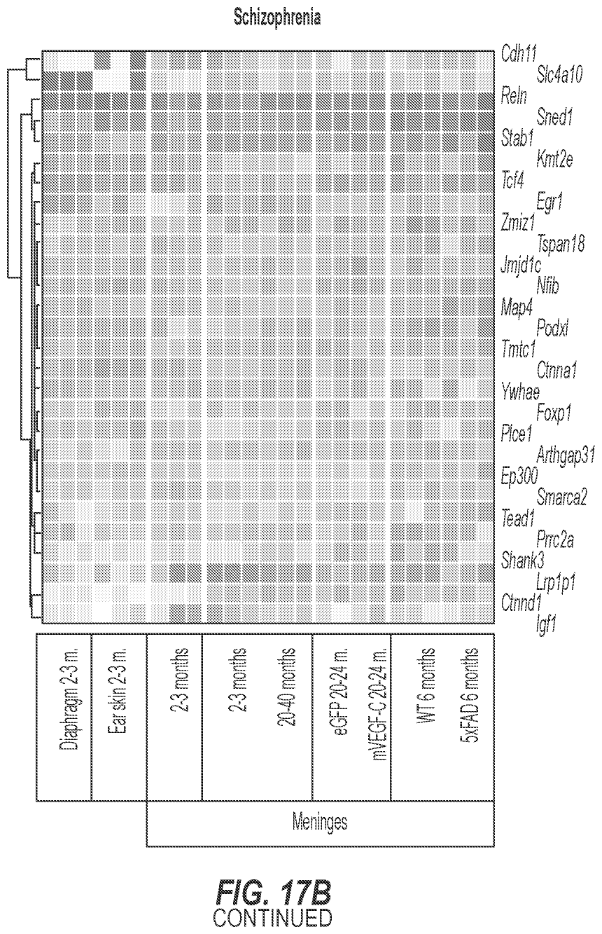

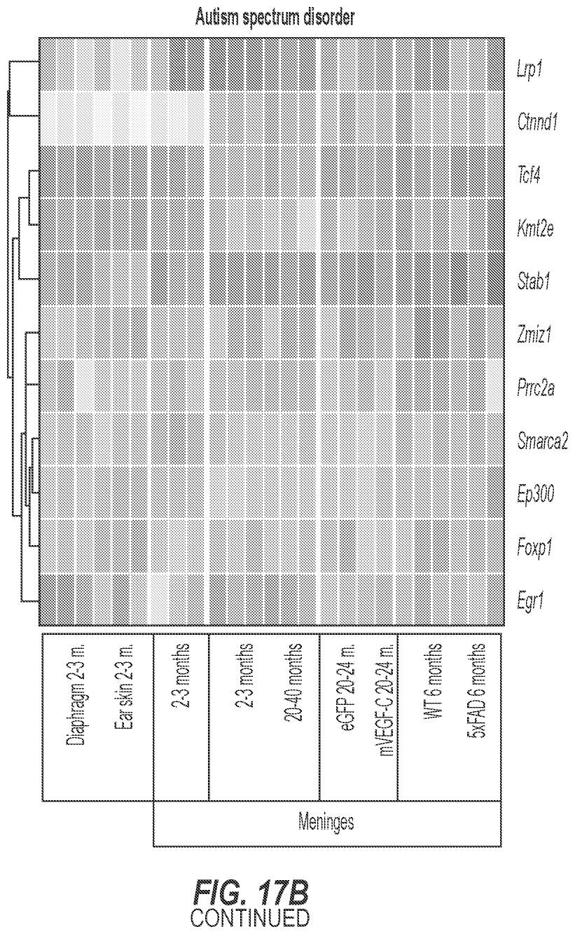

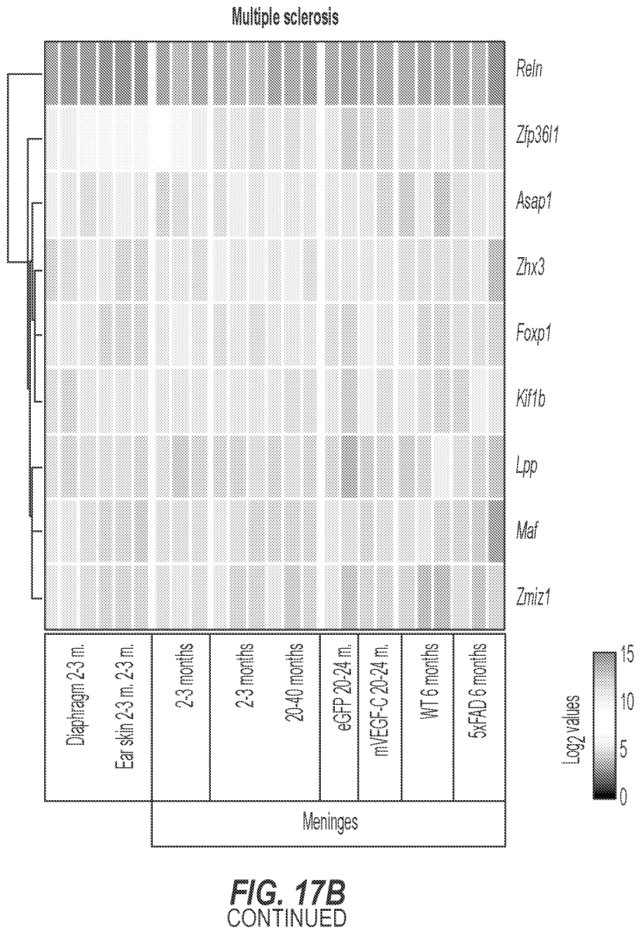

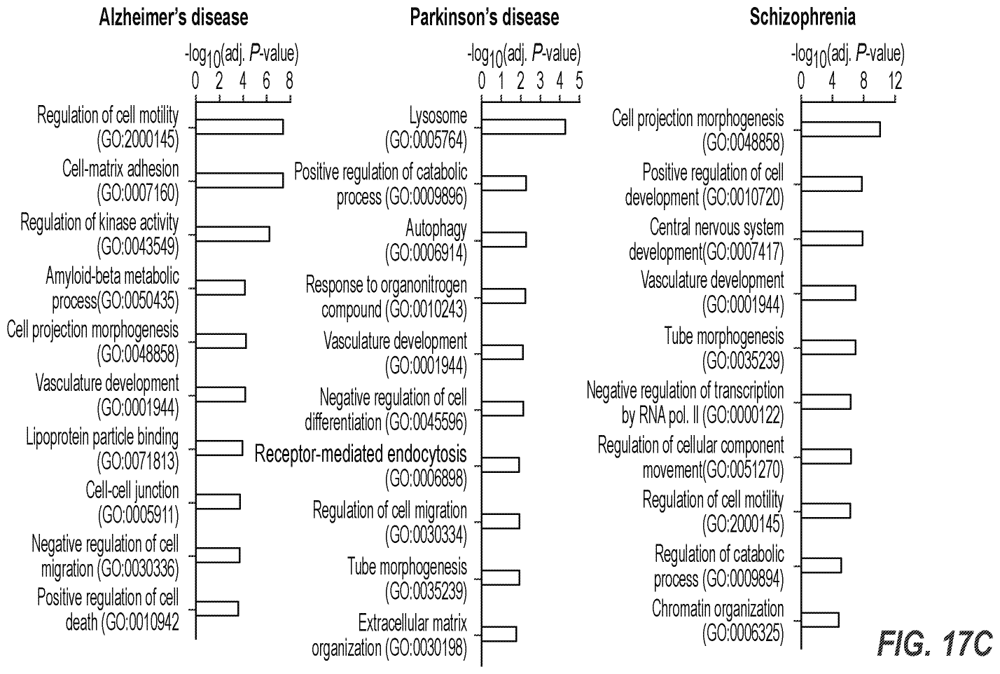

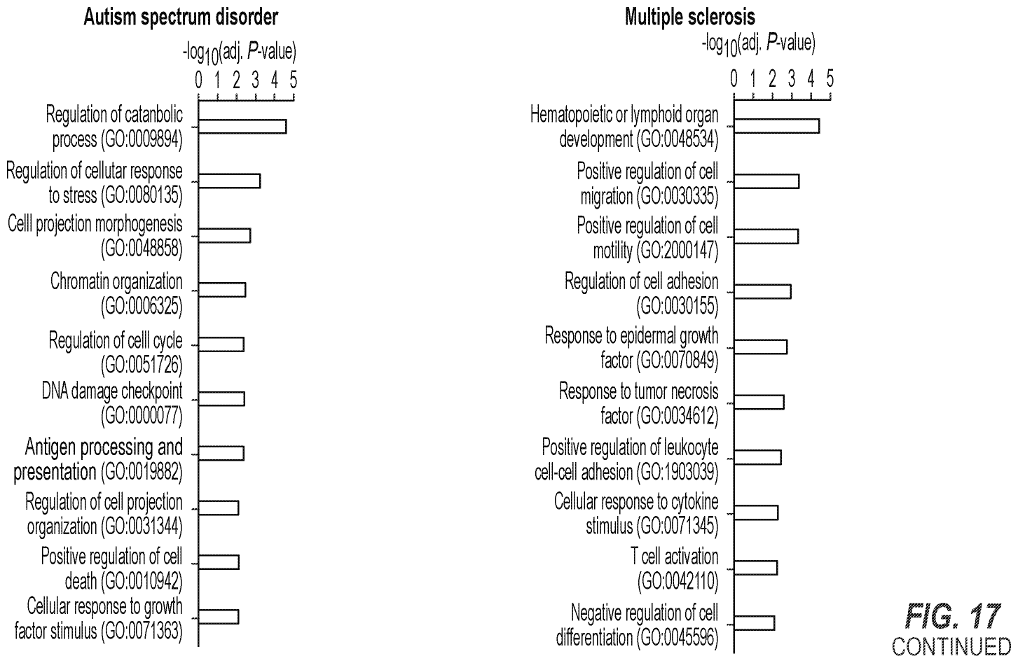

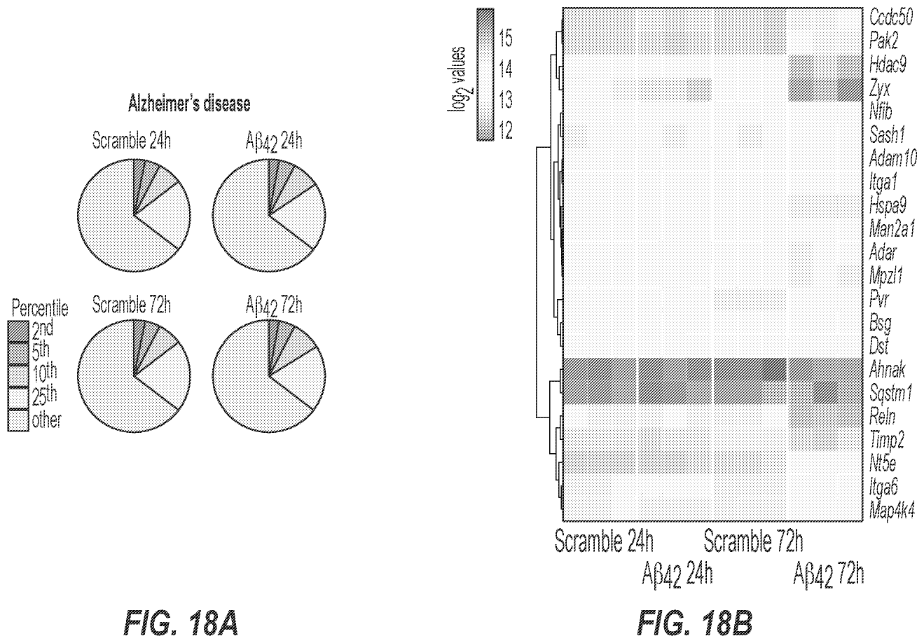

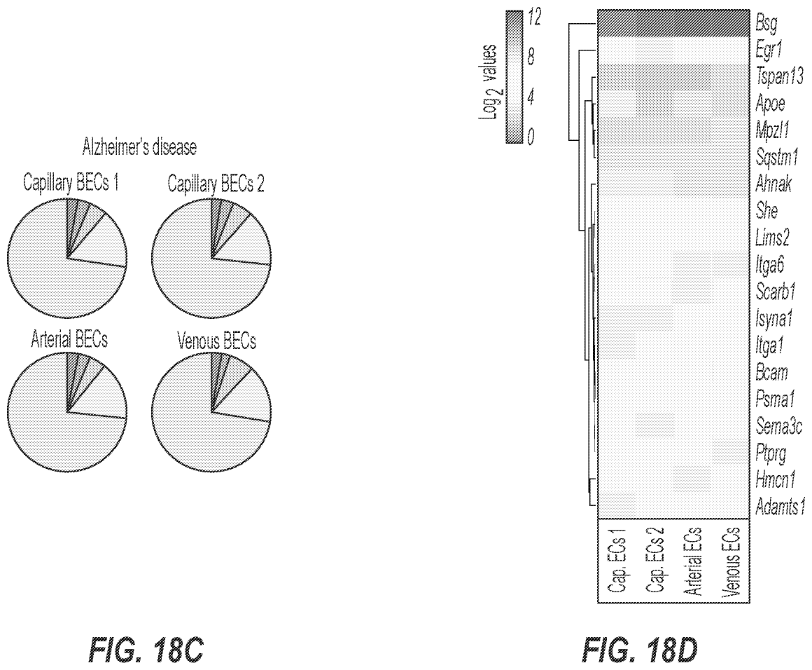

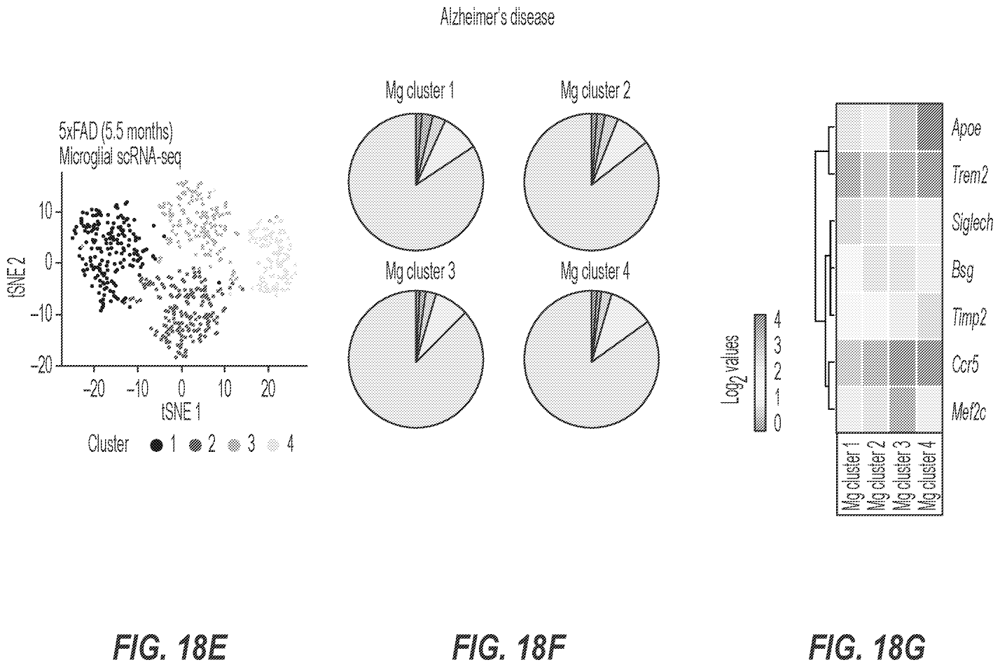

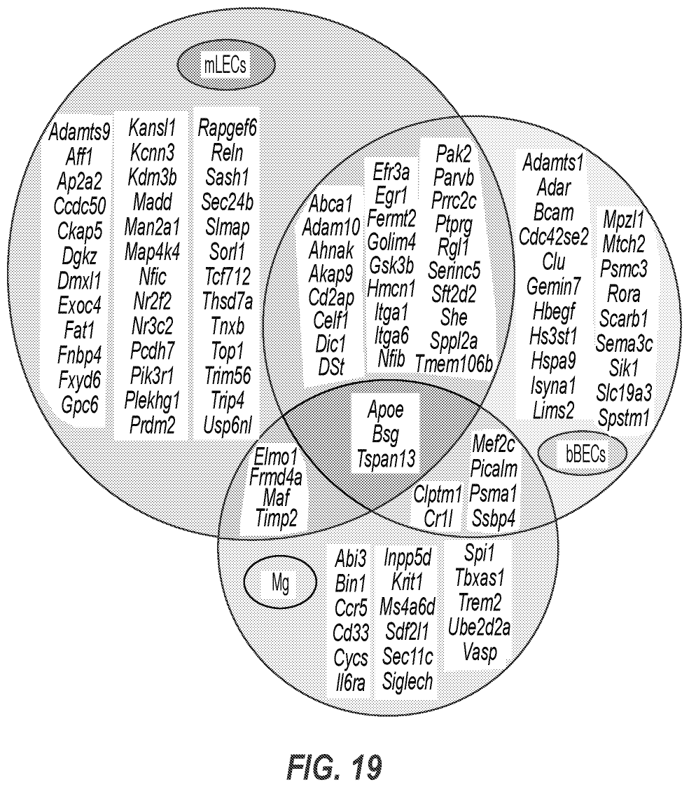

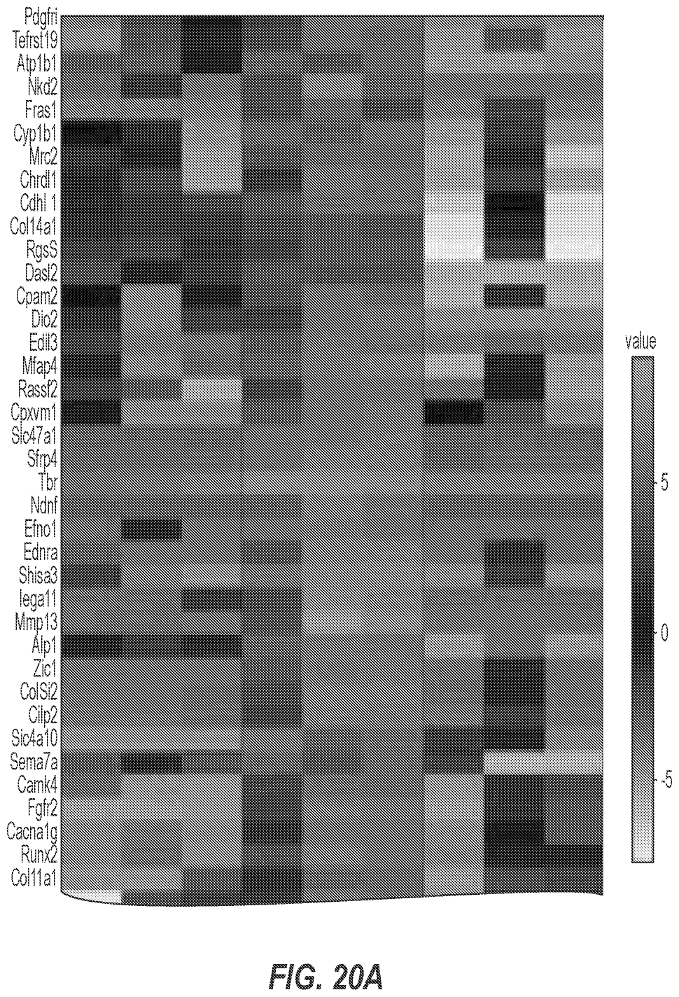

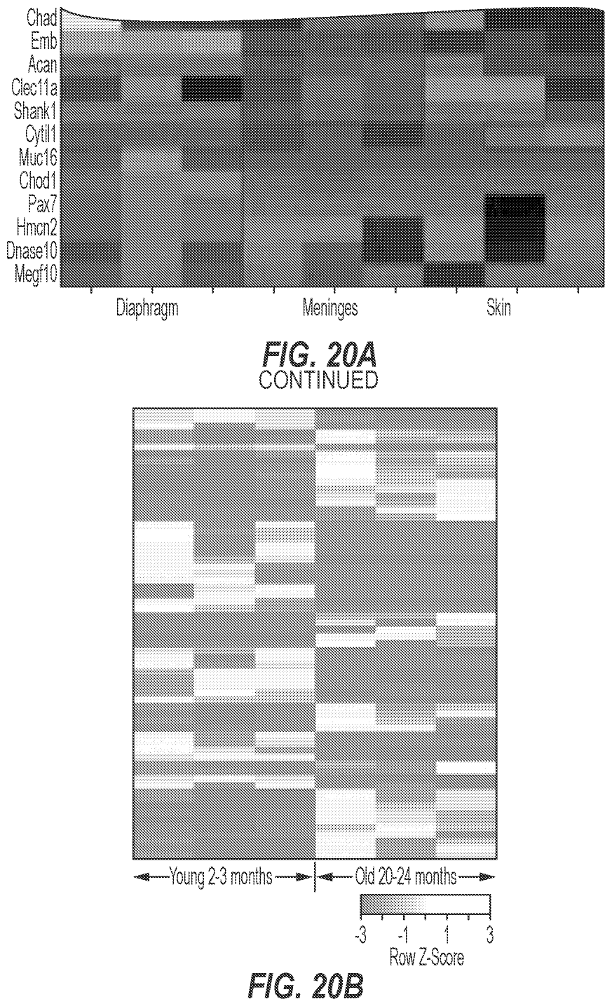

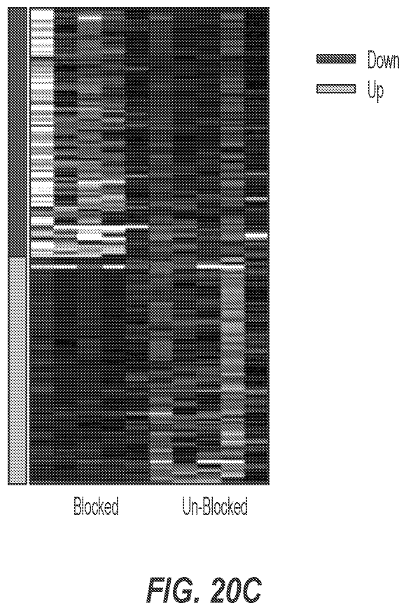

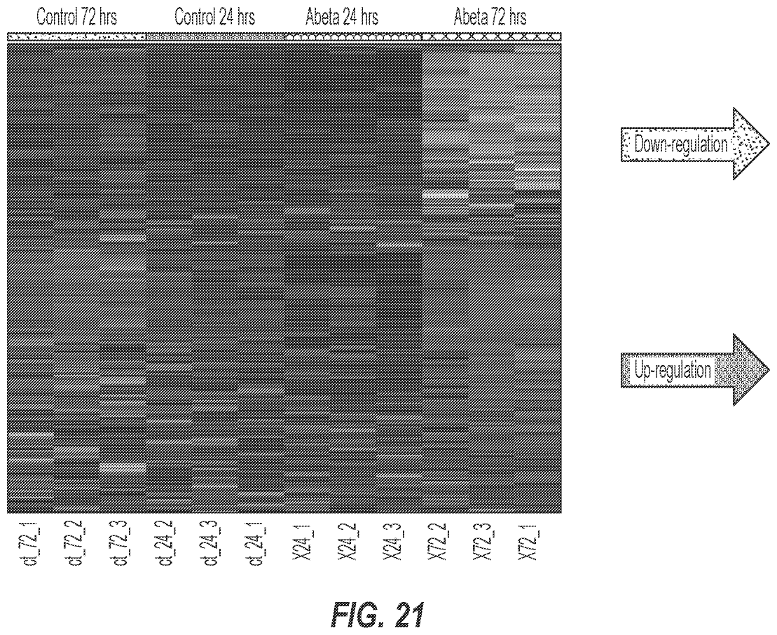

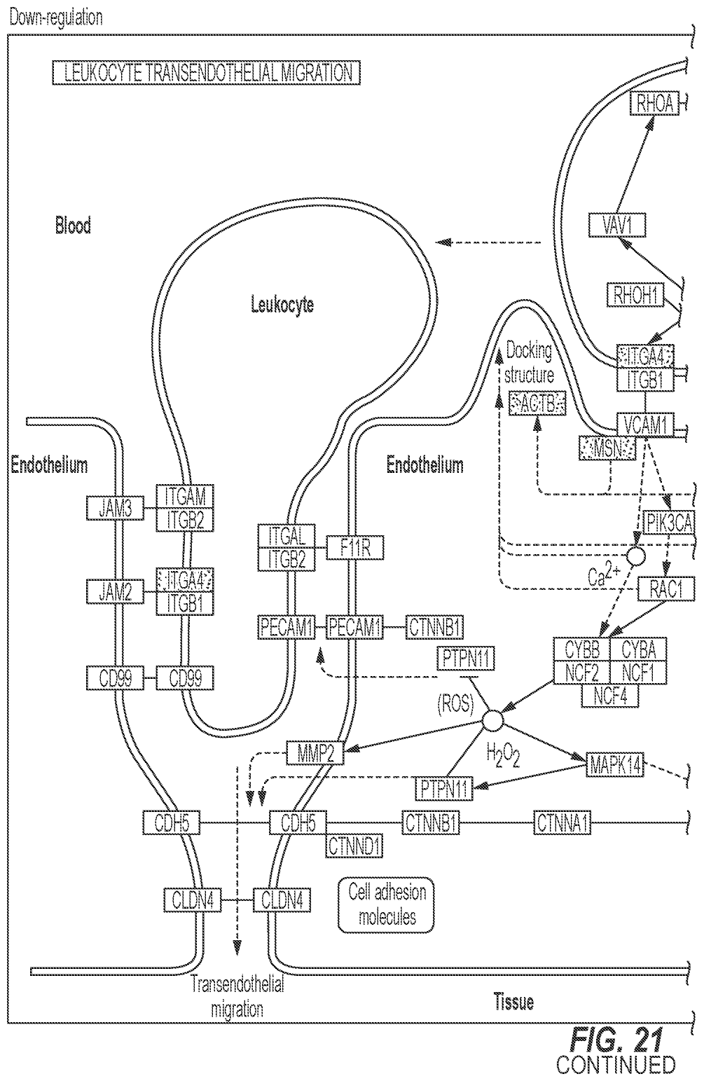

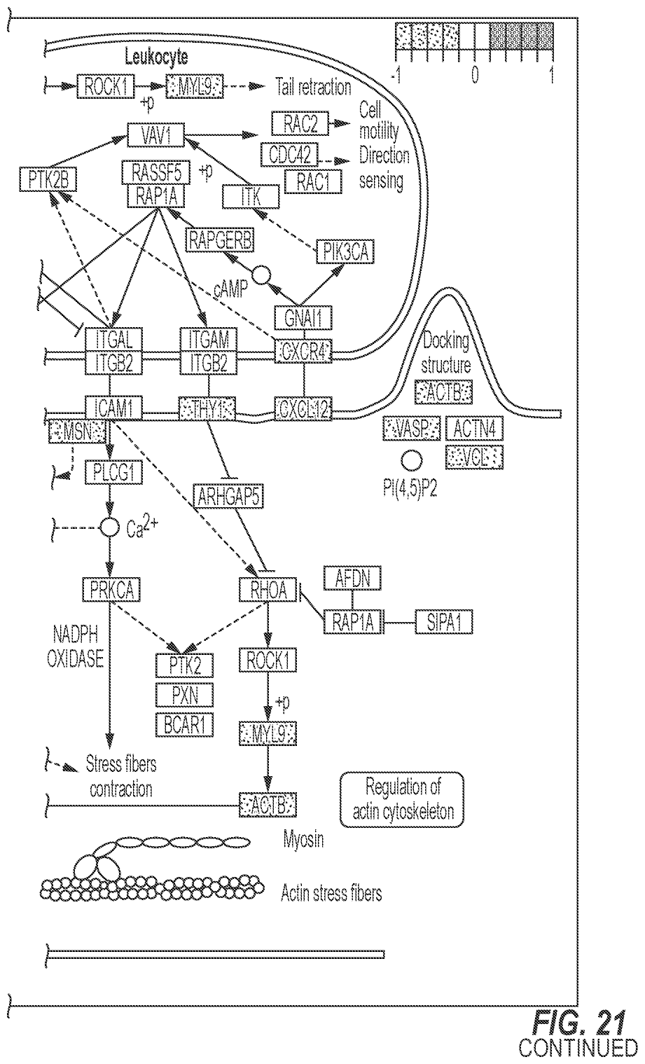

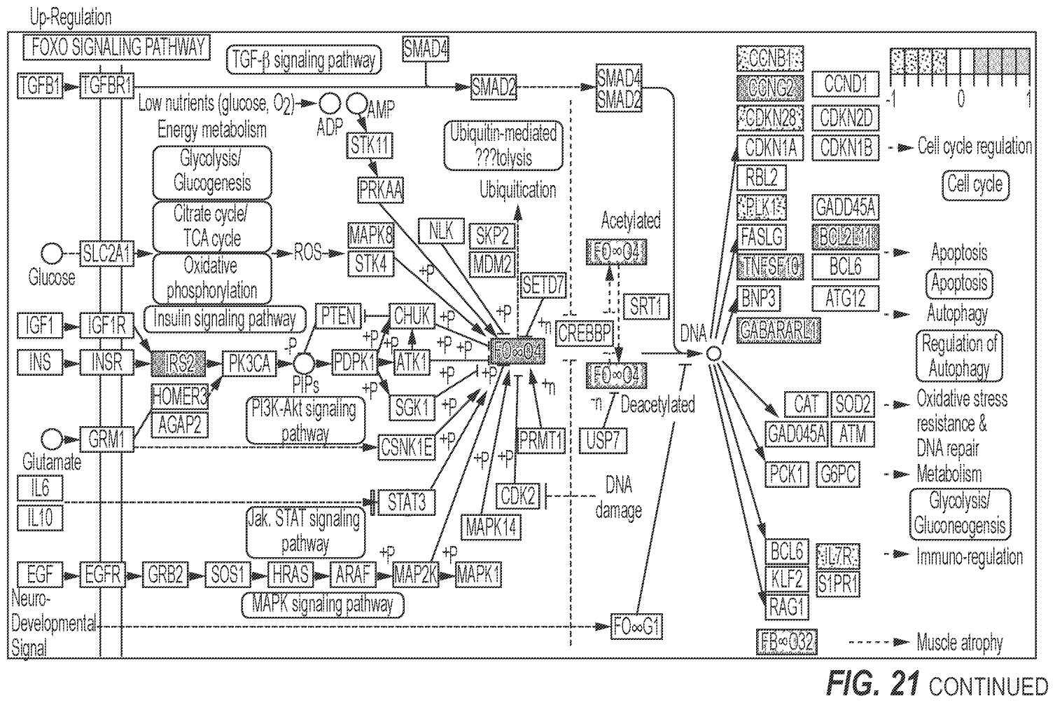

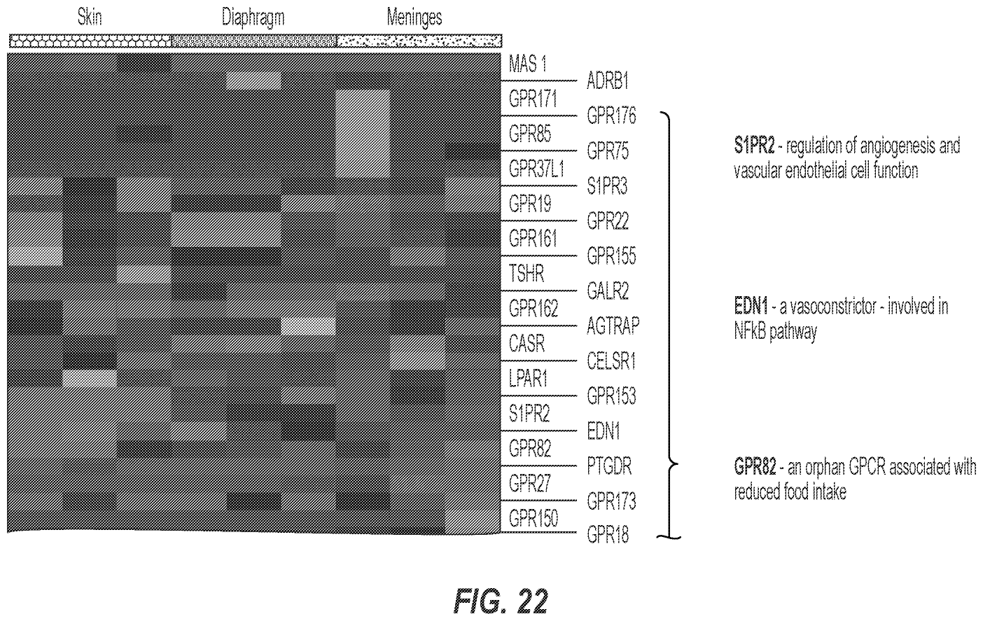

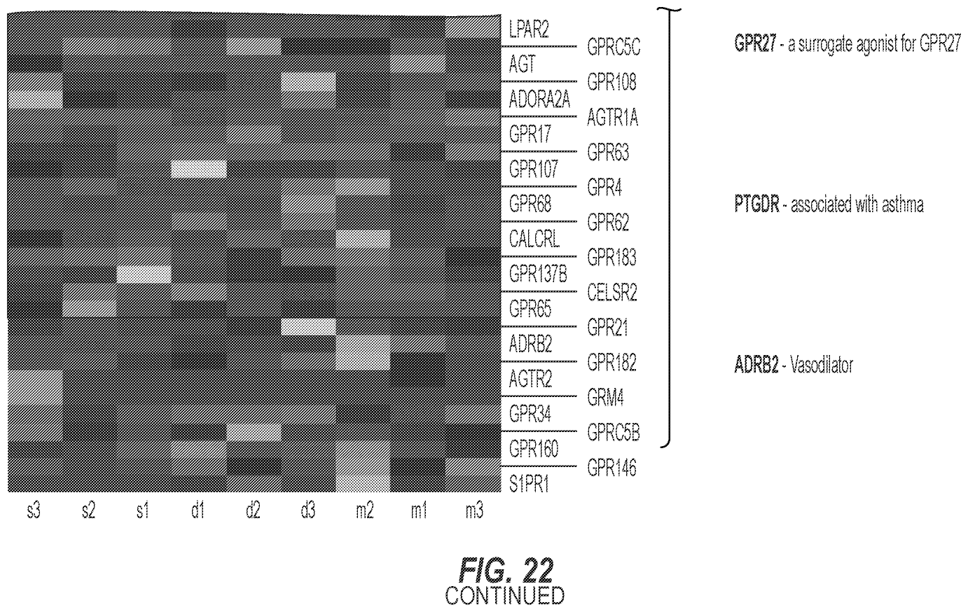

FIG. 13R is histograms showing the expression levels of H-2Kd in the fluorescence minus one (FMO) sample (grey) or in concatenated immune cell populations from the Vis. and Vis./photo. groups. Cells considered to be positive for H-2Kd are demarcated in the different histograms.

FIGS. 13S-13U are geometric mean fluorescence intensity (gMFI) values relative to H-2Kd in CD45^{int}CD11b⁺ (FIG. 13S), CD45high CD1b⁺ (FIG. 13T) and CD45^{high}CD11b^{neg} (FIG. 13U) cells. Results in FIGS. 130-13Q and FIGS. 13S-13U are presented as mean±s.e.m.; n=5 per group; two-tailed unpaired Student's T test.

FIGS. 14A-14I show that meningeal lymphatic vessel ablation precludes brain Aβ plaque clearance by ABETA Mab1 administered into the CSF.

FIG. 14A shows that adult 4-4.5 months-old male 5×FAD mice were injected (i.c.m.) with Visudyne (5 μL) plus photoconversion (Vis./photo.) or Visudyne without photoconversion (Vis.). One week later, 5 μL of ABETA Mab1 antibodies or the same volume of the control murine IgG (mIgG) antibodies were directly injected into the CSF (i.c.m.), both at 1 μg/L. Injections with antibodies were repeated two weeks later. Additional steps of meningeal lymphatic vessel ablation or control interventions were followed by two more i.c.m. injections with antibodies according to the scheme.

FIG. 14B shows representative images of meningeal whole mounts stained for CD31, LYVE-1 and Aβ (stained with the D54D2 antibody; scale bar, 2 mm).

FIG. 14C is a graph showing the coverage by Aβ as a percentage of the meningeal whole mount.

FIG. 14D shows representative images of brain sections from 5×FAD mice stained for Aβ (stained with the D54D2 antibody) and with DAPI (scale bar, 2 mm).

FIGS. 14E-14G are graphs showing number of Aβ plaques per mm² of brain section (FIG. 14E), average size of Aβ plaques (m²) (FIG. 14F) and total coverage of Aβ plaques (% of brain section) (FIG. 14G) in each group.

FIG. 14H is representative inset showing an example of a Prussian blue focus in a brain tissue section of a 5×FAD mouse (scale bar, 100 m).

FIG. 14I is a graph showing the quantifications of Prussian blue foci per brain section in each group.

Results in FIGS. 14C, 14E-14G and FIG. 14I are presented as mean s.e.m.; n=8 in Vis. plus mIgG, Vis. plus ABETA Mab1 and Vis./photo. plus mIgG, n=7 in Vis./photo. plus ABETA Mab1; two-way ANOVA with Holm-Sidak's multiple comparisons test; data are representative of 2 independent experiments.

FIGS. 15A-15R show combination therapy with mVEGF-C and anti-Abeta antibody (ABETA Mab1) induces meningeal lymphangiogenesis and boosts brain Aβ plaque clearance.

FIG. 15A shows that adult 5×FAD mice were injected with 5 μL (i.c.m.) of AAV1 expressing enhanced green fluorescent protein (eGFP) or murine VEGF-C (mVEGF-C), under the cytomegalovirus (CMV) promoter (each at 10¹² GC/μL), in combination with either mIgG2a or anti-Abeta antibody (each at 1 μg/μL) as indicated in the scheme.

FIG. 15B show representative images of brain sections stained for Aβ (stained with the D54D2 antibody) and with DAPI (scale bar, 2 mm).

FIG. 15C is graph showing coverage of AR as percentage of brain section in each group.

FIG. 15D shows representative images from the brain cortex stained for Aβ (stained with the Amilo-Glo), CD68 and IBA1 (scale bar, 50 m).

FIGS. 15E-15G are graphs showing the coverage by IBA1⁺ cells (% of field) (FIG. 15E), number of peri-AP plaque IBA1⁺ cells (FIG. 15F) and percentage of IBA1 occupied by CD68 (FIG. 15G) in each group.

Results in FIGS. 15C, 15E-15G are presented as mean s.e.m.; n=12 in mVEGF-C plus mIgG and n=13 in eGFP plus mIgG, eGFP plus ABETA Mab1 and mVEGF-C plus ABETA Mab1; two-way ANOVA with Holm-Sidak's multiple comparisons test; data result from 2 independent experiments.

FIG. 15H is representative stereomicroscopy images of lymphatic vessels stained for LYVE-1 around the transverse sinus (TS) at the dorsal brain meninges or around the sigmoid (SS) and petrosquamosal (PSS) sinuses at the basal brain meninges still attached to the skull cap (scale bars, 500 m).

FIGS. 15I-15J are graphs showing LYVE-1⁺ vessel total length (in mm) and branching points in the dorsal meninges (FIG. 15I) and total length (in mm) and branching points in the basal meninges (FIG. 15J). Results in are presented as mean±s.e.m.; n=6 in mIgG2a groups and n=7 in anti-Abeta antibody groups; Two-way ANOVA with Sidak's multiple comparisons test; data in FIGS. 15H-15J is representative of 2 independent experiments.

FIG. 15K is representative images of meningeal whole mounts stained for CD31 and LYVE-1 (scale bar, 1 mm; inset scale bar, 300 m).

FIGS. 15L and 15M are graphs showing b) coverage of CD31⁺ LYVE-1^{neg} vessels (% of meningeal whole mount) (FIG. 15L) and c) branching points and coverage of LYVE-1⁺ vessels (% of meningeal whole mount) (FIG. 15M). Results in FIGS. 15L and 15M are presented as mean±s.e.m.; n=7 in eGFP+mIgG2a and n=6 in eGFP+anti-Abeta antibody, mVEGF-C+mIgG2a and mVEGF-C+anti-Abeta antibody; Two-way ANOVA with Sidak's multiple comparisons test; data in FIGS. 15K-15M is representative of 2 independent experiments.

FIG. 15N is representative images of brain sections from 5×FAD mice stained for As and with DAPI (scale bar, 2 mm).

FIGS. 15O-15R are graphs showing the coverage of Aβ (% of brain region/section) in the hippocampus (FIG. 15O), cortex/striatum/amygdala (FIG. 15P), thalamus/hypothalamus (FIG. 15Q) and the whole brain section (FIG. 15R).

FIG. 15S is representative images from the brain cortex stained for Aβ, LAMP-1 and Fibrinogen (scale bar, 200 μm).

FIGS. 15T and 15U are graphs showing the coverage (% of field) by LAMP-1⁺ dystrophic neurites (FIG. 15T) and Fibrinogen (FIG. 15U) in cortical vasculature.

Results in FIGS. 15O-15R, 15T, 15U, and 15E-15G are presented as mean s.e.m.; n=12 in mVEGF-C+mIgG2a and n=13 in eGFP+mIgG2a, eGFP+anti-Abeta antibody and mVEGF-C+anti-Abeta antibody; Two-way ANOVA with Sidak's multiple comparisons test, data in FIGS. 15D-15G and 15N-15U results from 2 independent experiments.

FIGS. 16A-16N show viral-mediated expression of mVEGF-C induces transcriptomic changes in aged meningeal LECs and improves the efficacy of anti-Abeta antibody treatment (ABETA Mab1) in aged AD transgenic mice.

FIG. 16A shows that aged WT mice (20-24 months of age) were injected with 2 μL (i.c.m.) of AAV1 expressing eGFP or mVEGF-C, under the CMV promoter (each at 10¹³ GC/μL). One month later, mice were transcardially perfused, skull caps were collected, meninges harvested and LECs were sorted by FACS for bulk RNA-seq.

FIG. 16B is PCA plot showing segregation between eGFP and mVEGF-C meningeal LEC transcriptomes.

FIG. 16C is a heatmap of top 50 differentially expressed genes. Color scale bar represents expression values for each sample as standard deviations from the mean across each gene in FIG. 19C.

FIG. 16D is volcano plot showing the significantly down-regulated and up-regulated genes between meningeal LECs from the mVEGF-C and eGFP groups.

Data in FIGS. 16B-16D consists of n=2 per group; individual RNA samples result from LECs pooled from 10 meninges over 2 independent experiments; differentially expressed genes plotted in FIG. 16D were determined using a F-test with adjusted degrees of freedom based on weights calculated per gene with a zero-inflation model and Benjamini-Hochberg corrected P-values.

FIG. 16E shows that aged J20 mice (14-16 months-old) were injected with 5 μL (i.c.m.) of AAV1 expressing eGFP or mVEGF-C (each at 10¹² GC/μL) in combination with either mIgG2a or anti-Abeta antibody (each at 1 μg/μL) as indicated in the scheme.

FIG. 16F is representative images of brain sections from J20 mice stained for Aβ and with DAPI (scale bar, 1 mm).

FIGS. 16G-16I, are graphs showing coverage of Aβ (% of region) in the hippocampus (FIG. 16G), cortex/striatum/amygdala (FIG. 16H) and combined regions (FIG. 16I). Results are presented as mean±s.e.m.; n=8 in eGFP+anti-Abeta antibody and n=10 in mVEGF-C+anti-Abeta antibody; two-tailed unpaired Student's T test.

FIG. 16J shows aged APPswe mice (26-30 months-old) were injected with 5 μL (i.c.m.) of AAV1 expressing eGFP or mVEGF-C (each at 10¹² GC/μL) in combination with either mIgG2a or anti-Abeta antibody (each at 1 μg/μL) as indicated in the scheme.

FIG. 16K is representative images of brain sections from APPswe mice stained for Aβ and with DAPI (scale bar, 1 mm).

FIGS. 16L-16N are graphs showing coverage of Aβ (% of region) in the hippocampus (FIG. 16L), cortex/striatum/amygdala (FIG. 16M) and combined regions (FIG. 16N). Results in are presented as mean±s.e.m.; n=11 per group; two-tailed unpaired Student's T test.

FIGS. 17A-17C show genes associated with increased risk for Alzheimer's disease and other neurological disorders are highly expressed in mouse lymphatic endothelial cells.

FIG. 17A is pie charts showing the proportion of genes associated with higher risk for Alzheimer's disease, Parkinson's disease, Schizophrenia, Autism spectrum disorder and Multiple sclerosis, for which the average expression across all LEC RNA-seq datasets was in the top 2^{nd}, 5^{th} 10^{th}, or 25^{th} percentile out of all genes.

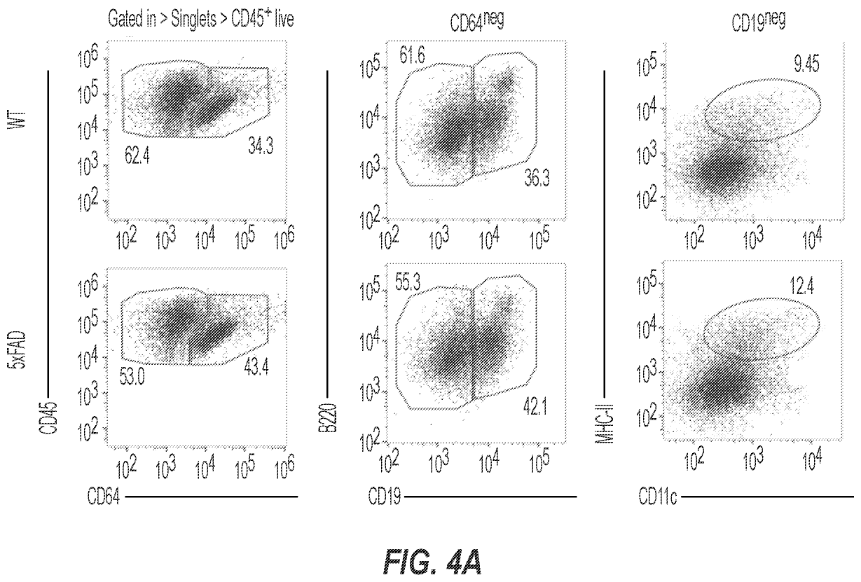

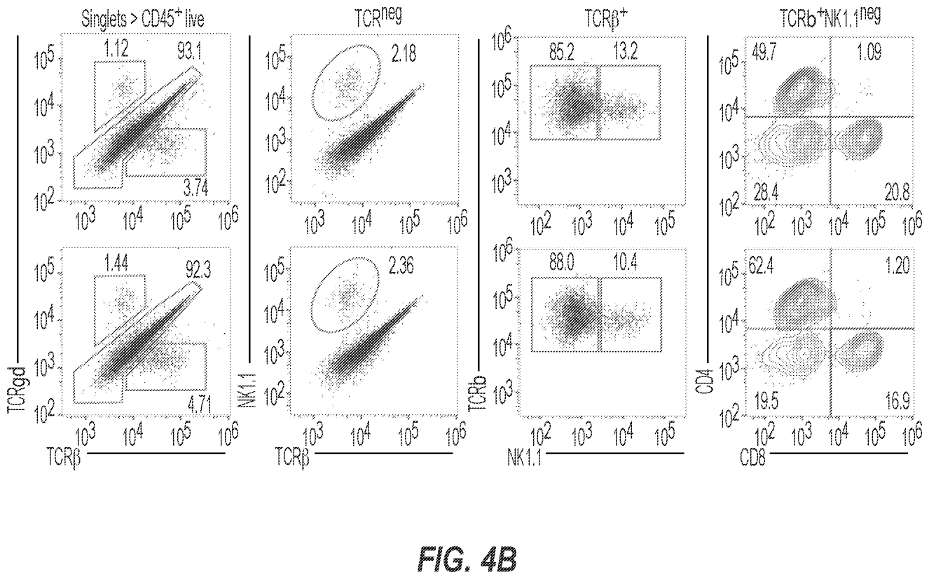

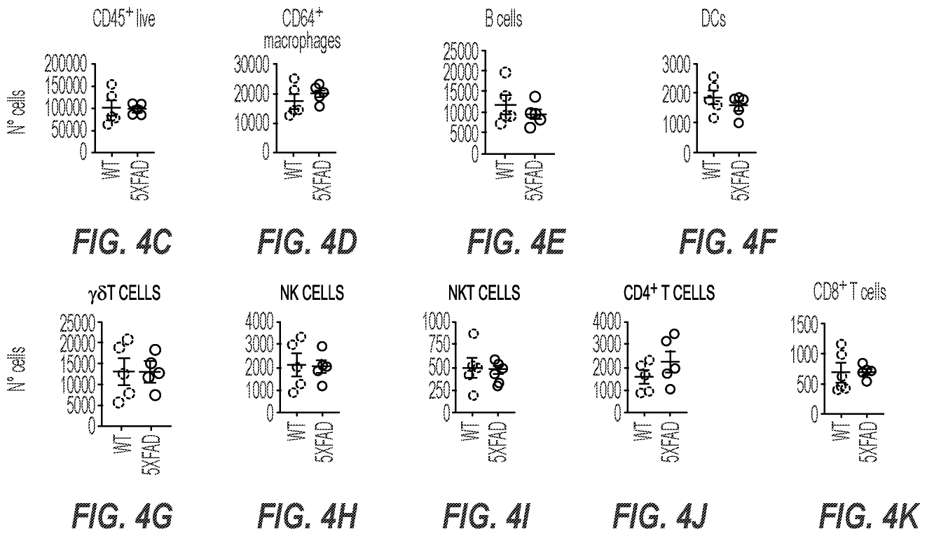

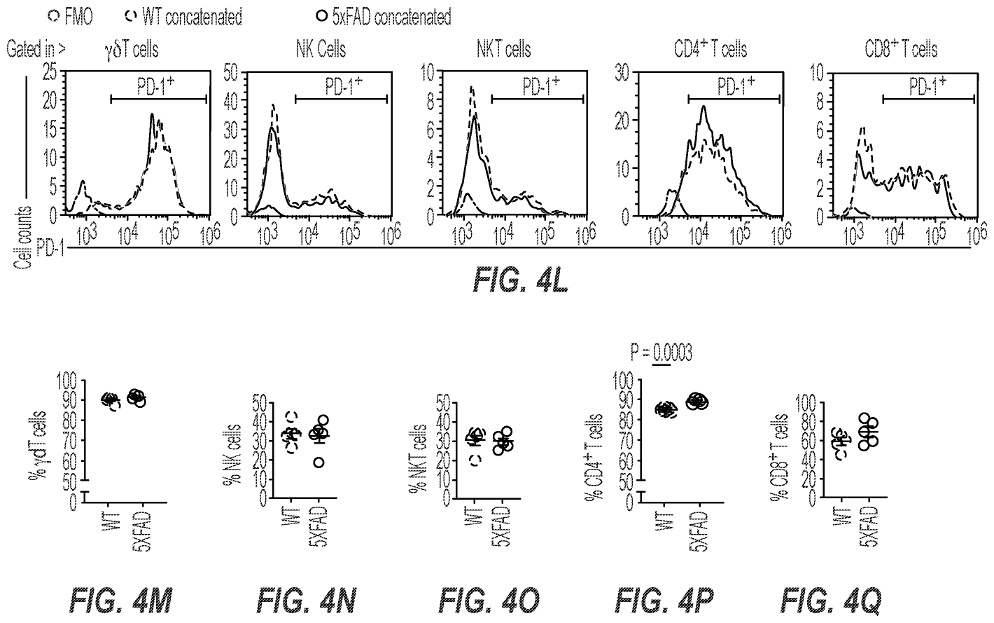

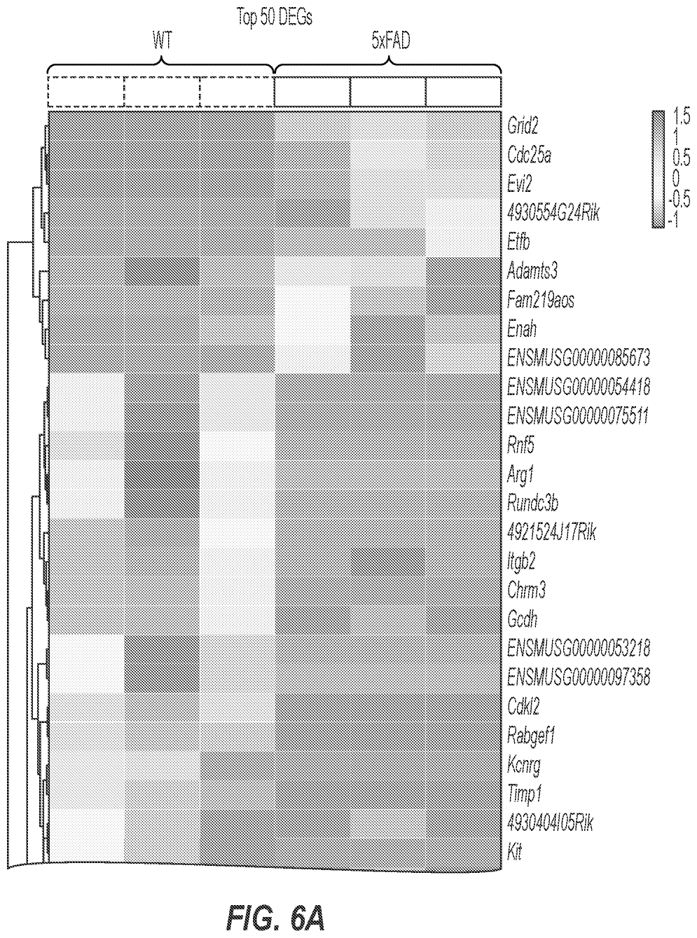

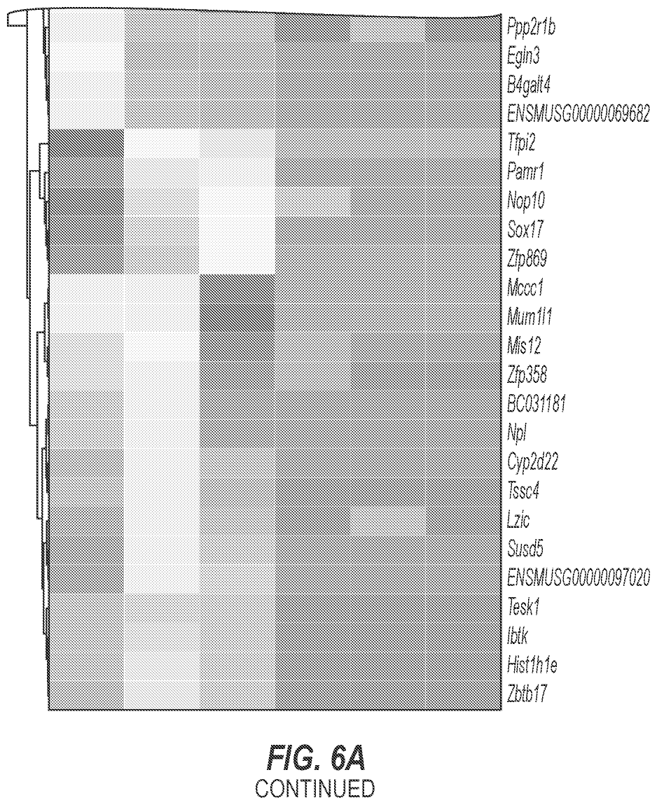

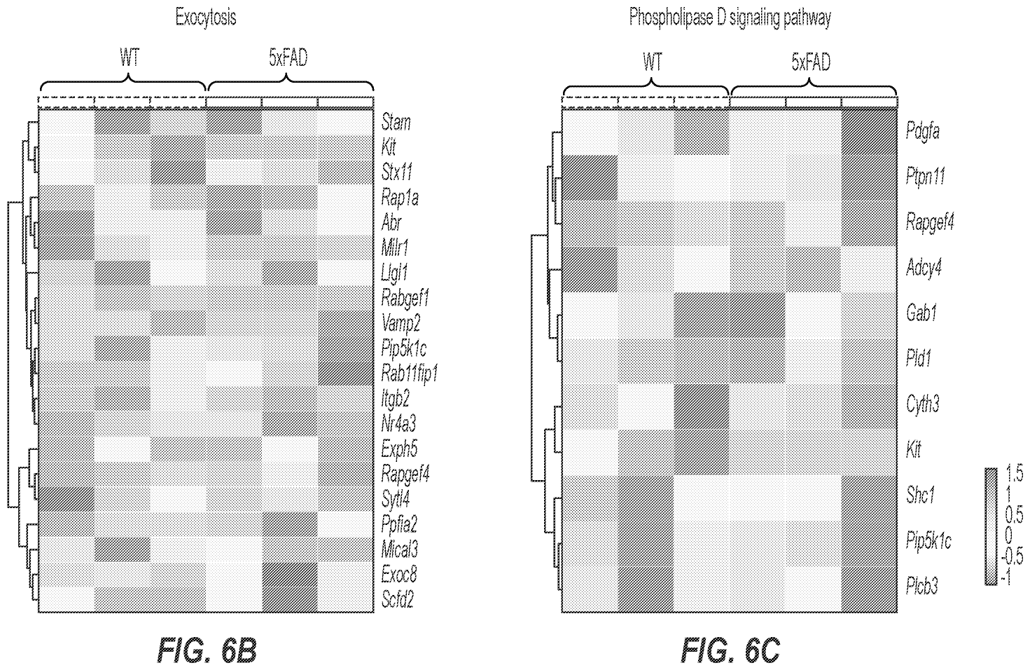

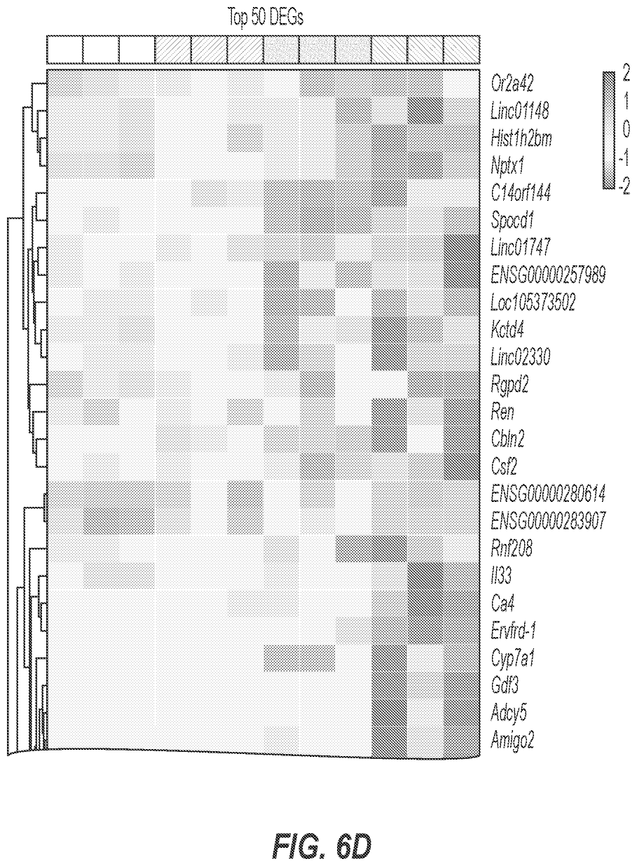

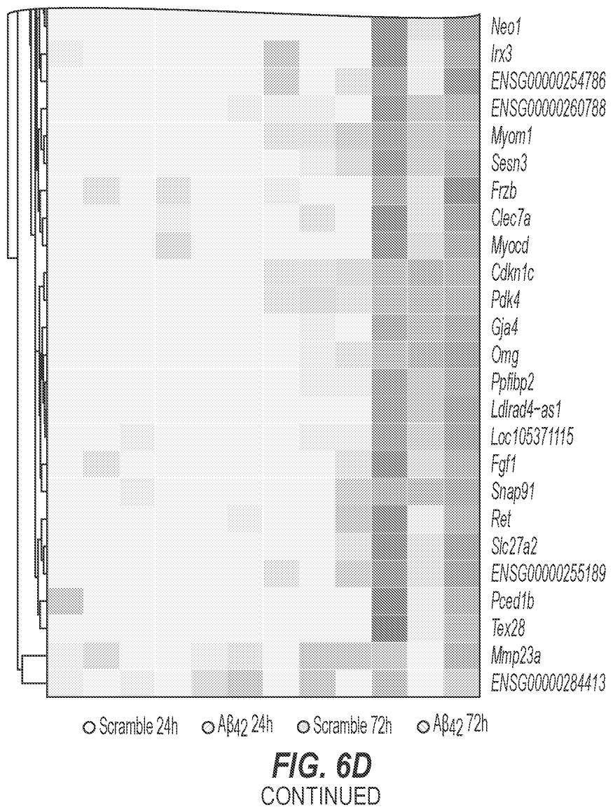

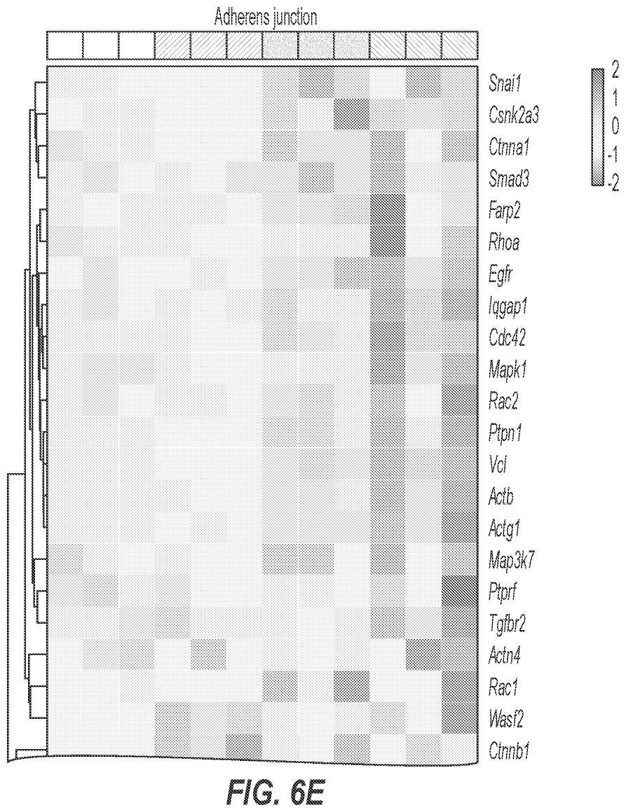

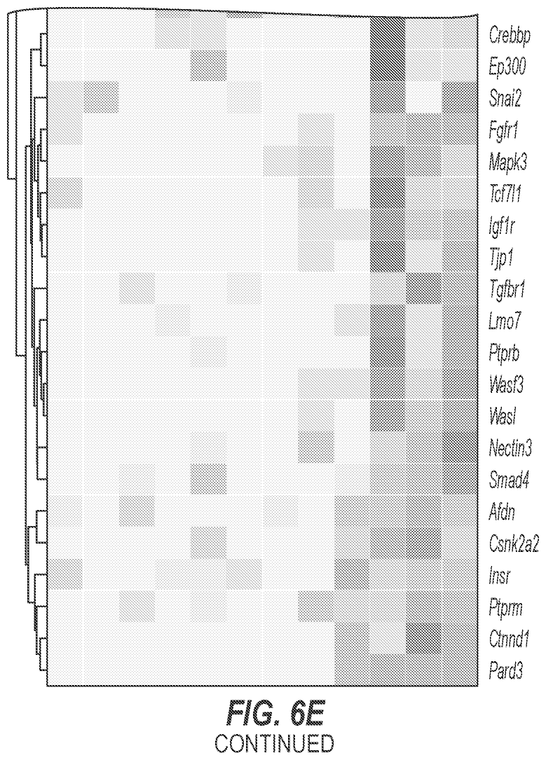

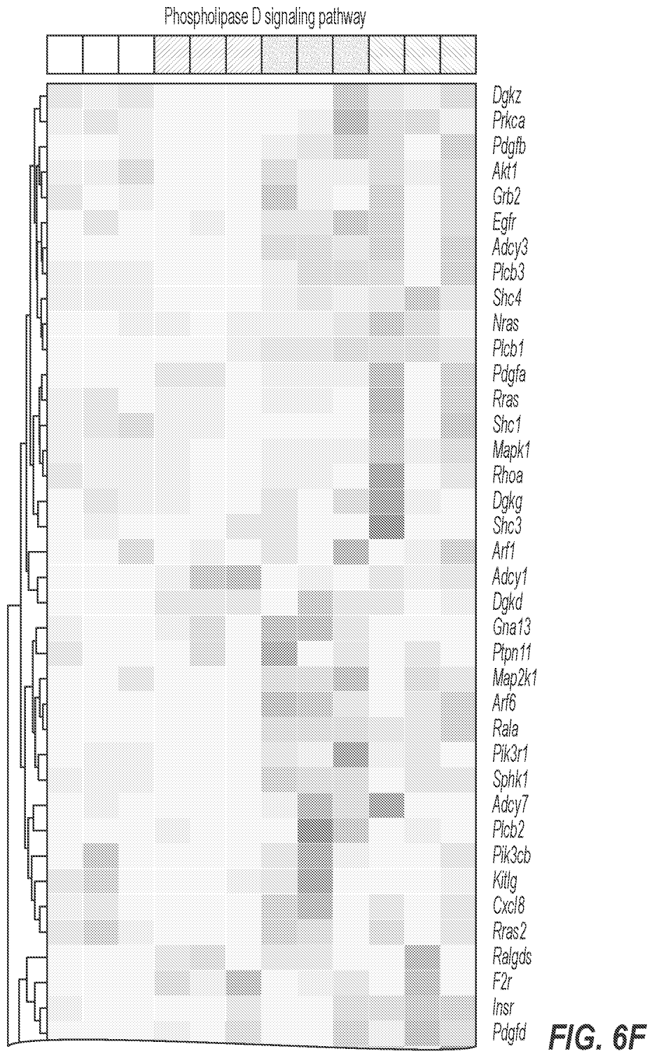

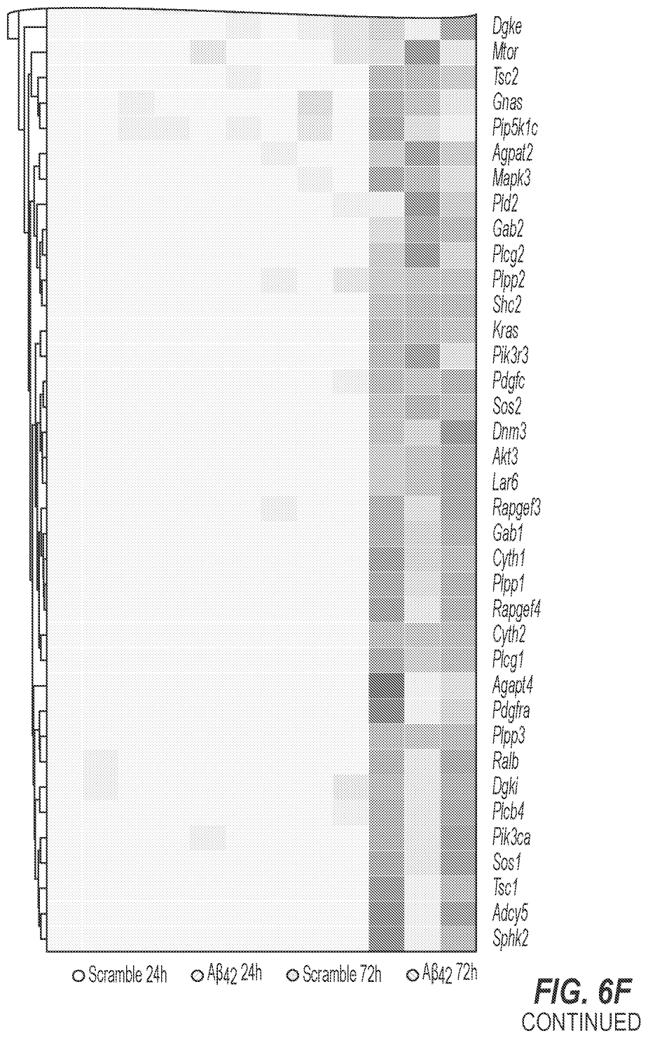

FIG. 17B is heatmaps showing the log 2-normalized expression values (depicted in color scale bar) for disease-associated genes whose average expression values fall within the top 2^{nd}, 5^{th}, percentile of genes expressed across RNA-seq datasets obtained from LECs isolated from diaphragm, ear skin and meninges of 2-3 months (m.) old mice (Louveau, A. et al. CNS lymphatic drainage and neuroinflammation are regulated by meningeal lymphatic vasculature. Nat Neurosci 21, 1380-1391, (2018)), from meninges of 2-3 or 20-24 months-old mice (Da Mesquita, S. et al. Functional aspects of meningeal lymphatics in ageing and Alzheimer's disease. Nature 560, 185-191, (2018)), from meninges of 20-24 months-old mice injected with AAV1-CMV-eGFP or AAV1-CMV-mVEGF-C-WPRE (one month after i.c.m. injection—see Example 10 for details and FIGS. 4A-4C) and from meninges of 6 months-old WT or 5×FAD mice (see FIGS. 6A-6C for more results and details).

FIG. 17C is gene sets obtained by functional enrichment of 25^{th} percentile disease-associated genes expressed across the different LEC RNA-seq datasets. Data in FIGS. 17A-17C consists of n=2 or 3 per group; individual RNA samples result from LECs pooled from 10 mice; in FIG. 17C, the Benjamini-Hochberg correction was used to adjust the associated P-values (adj. P-value <0.05) and the functional enrichment of differential expressed genes was determined with Fisher's exact test.

FIGS. 18A-18D show genes associated with increased risk for Alzheimer's disease are highly expressed in cultured human LECs and brain blood endothelial cells.

FIG. 18A is pie charts showing the proportion of genes associated with higher risk for Alzheimer's disease for which the average expression in cultured human LEC RNA-seq datasets was in the top 2^{nd}, 5^{th}, 10^{th}, or 25^{th} percentile out of all genes.

FIG. 18B is a heatmap showing the log 2-normalized expression values (depicted in color scale bar) for disease-associated genes whose average expression values fall within the top 2^{nd} percentile of genes expressed in the RNA-seq datasets obtained from cultured human LECs.

FIG. 18C is pie charts showing the proportion of genes associated with higher risk for Alzheimer's disease for which the average expression in brain capillary endothelial cells (ECs) 1, capillary ECs 2, arterial ECs and venous ECs (obtained from the scRNA-seq dataset published by Vanlandewijck et al. (Vanlandewijck, M. et al. A molecular atlas of cell types and zonation in the brain vasculature. Nature 554, 475-480, (2018))) was in the top 2^{nd}, 5^{th}, 10^{th}, or 25^{th} percentile out of all genes.

FIG. 18D is a heatmap showing the log 2-normalized expression values (depicted in color scale bar) for disease-associated genes whose average expression values fall within the top 2^{nd} percentile of genes expressed in capillary ECs 1, capillary ECs 2, arterial ECs and venous ECs.

FIG. 18E shows that the transcriptome of myeloid cells (live CD45⁺Ly6G⁻CD11b⁺ cells) sorted from the brain cortex of 5.5 month-old 5×FAD mice was analyzed by single-cell RNA-seq (scRNA-seq). The graph shows the unsupervised clustering and tSNE representation of four distinct clusters of microglia (Mg).

FIG. 18F depicts pie charts showing the proportion of Alzheimer's disease-associated gene single nucleotide polymorphisms, for which the average expression was in the top 2^{nd} 5^{th}, 10^{th}, or 25^{th} percentile out of all genes in each microglial cluster.

FIG. 18G is a heatmap showing the expression values for Alzheimer's disease-associated genes whose average expression values fall within the top 2^{nd} percentile of all genes expressed in each microglial cluster.

Data in FIGS. 18C and 18D resulted from the analysis of a scRNA-seq dataset published by Vanlandewijck et al⁷⁸; data in FIGS. 18E-18G resulted from the scRNA-seq analysis of 651 microglia; in FIGS. 18D and 18G, scale bars represent log 2-normalized expression values; see FIGS. 17A-17C and FIG. 19 for more details and data.

FIG. 19 is a Venn diagram showing overlap in Alzheimer's SNP associated genes in the top 10^{th} percentile for EC, LEC and microglia.

FIGS. 20A-20C depict heatmaps showing the study of the differences in lymphatic endothelial cell constitution.

FIG. 20A depicts heatmaps showing that meningeal lymphatic endothelial cells have signatures that distinguish them from diaphragm and skin lymphatics.

FIG. 20B depicts heatmaps showing the changes observed in meningeal lymphatics of young and old mice.

FIG. 20C depicts heatmaps showing the changes observed in gene expression level in hippocampal cells after blockage of meningeal lymphatics.

FIG. 21 depicts heatmaps and graphs showing Alzheimer's disease-specific pathways impacted in LEC cultures treated with Aβ in a temporal manner.

FIG. 22 depicts heatmaps showing the identification of differentially expressed genes (DEGs) uniquely expressed in the meningeal lymphatics.

### DETAILED DESCRIPTION

This invention is based upon, at least partially, the unexpected discovery that the treatment with flow modulators, e.g., VEGF-c, in combination with a neurological therapeutic agent, e.g., an amyloid-β antibody, can synergize to reduce protein aggregates, e.g., amyloid-β plaques, in the central nervous system, e.g., brain. Flow modulators can increase flow for example, by increasing the diameter of a meningeal lymphatic vessel of the subject, by increasing the quantity of meningeal lymphatic vessels of the subject, and/or by increasing drainage through meningeal lymphatic vessels of the subject. Thus, fluid flow in the central nervous system of the subject can be increased. Neurological therapeutic agents interact with a target, e.g., protein aggregate, to reduce the contribution of the target to the pathogenesis of a neurologic disease. Without wishing to be bound by any theory, the flow modulators may facilitate the removal of the “end product” of the interaction between neurological therapeutic agents and the target, e.g., the complex formed between a pathological protein and an antibody, by various mechanisms as described in detail herein, e.g., increasing the drainage of the “end product” to certain specific sites, e.g., deep cervical lymph nodes, thereby improving the treatment of neurodegenerative disease. Alternatively, or in addition, the flow modulator may facilitate access of the neurological therapeutic agent to its target. Accordingly, a treatment combining a flow modulator described herein with a therapeutic agent described herein may improve the extent of the desired effect on the pathology of the neurological disease, e.g., reduction of pathological protein aggregates, reduction of inflammation at the site of aggregation, etc., as compared to a treatment with the therapeutic agent alone or as compared to a treatment with the flow modulator alone. Provided herein are compositions and methods using one or more flow modulators, e.g., VEGF-c, in combination with a neurological therapeutic agents to increase the therapeutic effect over the single agent alone.

Traditionally, the central nervous system was viewed as being immune privileged, and was believed to lack a classical lymphatic drainage system. As described herein, a lymphatic system is present in meningeal spaces, and functions in draining macromolecules, immune cells, and debris from the central nervous system (CNS). Moreover, it has been discovered herein that combinations of agents can modulate drainage by the meningeal lymphatic drainage can affect certain diseases of the brain and central nervous system. In particular, as described in several embodiments herein, modulating lymphatic vessels to increase flow in accordance with some embodiments herein can synergize with neurological therapeutic agents to alleviate symptoms of neurological diseases, for example proteinopathies such as tauopathies and/or amyloidoses (e.g., AD), including cognitive symptoms, and accumulation of amyloid beta plaques. Accordingly, in some embodiments, methods, compositions, and uses for treating, preventing, inhibiting, or ameliorating symptoms of neurological diseases, for example proteinopathies such as tauopathies and/or amyloidoses (e.g., AD) are described. The neurological diseases can be associated with increased concentration and/or the accumulation of macromolecules, cells, and debris in the CNS (for example, AD, which is associated with the accumulation of amyloid beta plaques). The methods, compositions, and uses can increase drainage by lymphatic vessel, and thus increase flow in CSF and ISF. Several embodiments herein are particularly advantageous because they include one, several or all of the following benefits: (i) increased flow in the CNS; (ii) decreased accumulation of macromolecules, cells, or debris in the CNS (for example, decreased accumulation of amyloid beta); and (iii) maintenance of or improvement in motor and/or cognitive function (for example memory function) in a subject suffering from, suspected of having, and/or at risk for a neurological disease (such as dementia in a neurological disease such as AD).

It has been shown that meningeal lymphatic vessels mediate drainage in the CNS, and that impaired meningeal lymphatic function impacts brain homeostasis. See, e.g., PCT Pub. No. 2017/210343, which is incorporated by reference herein in its entirety. Characteristics of meningeal lymphatic vessels are described for example, in PCT Pub. No. 2017/210343 at Example 13. Immune cells such as T cells and dendritic cells accumulate in the meningeal lymphatic vessels (See, e.g., PCT Pub. No. 2017/210343 at Examples 14-21). Impairing meningeal vessels significantly decreases drainage into deep cervical lymph nodes, and impact immune cell size and coverage, and inhibits immune cell migration (PCT Pub. No. 2017/210343 at Examples 2 and 24-25).

Flow and Flow Modulators

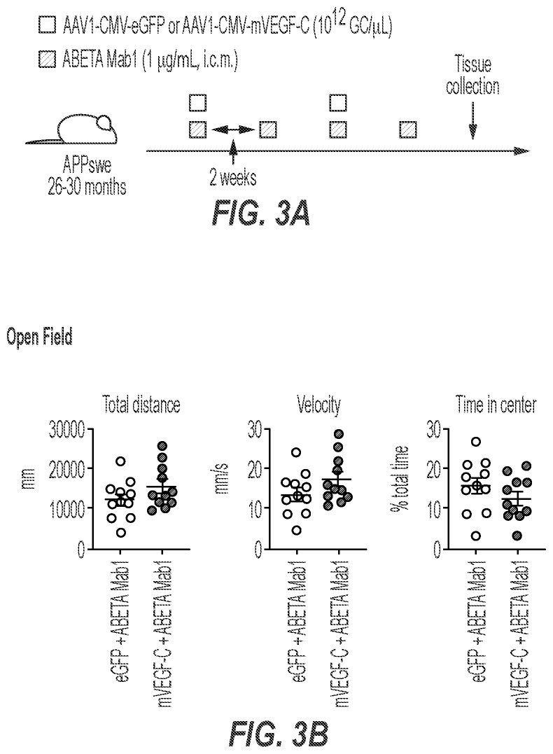

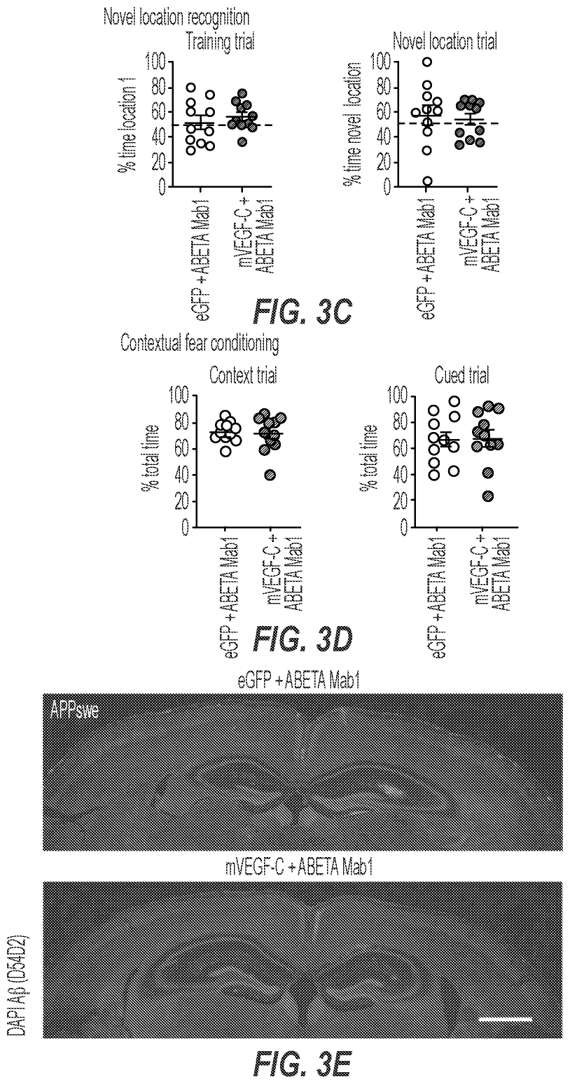

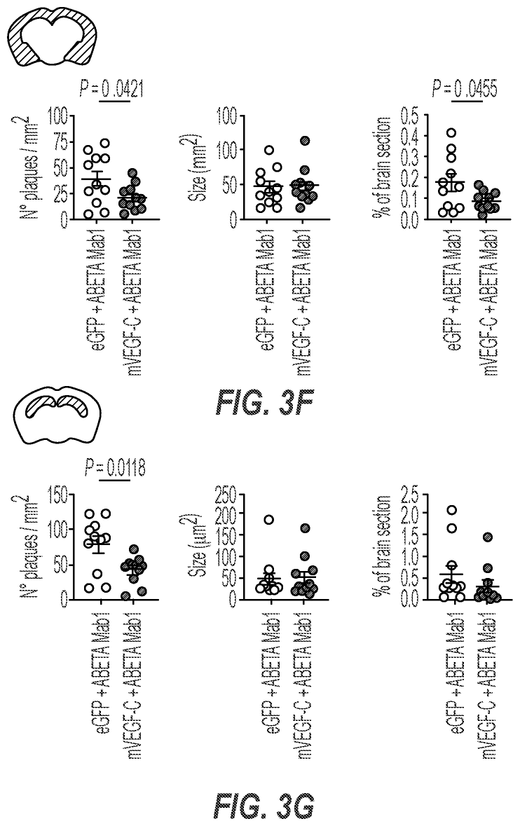

As used herein “flow” shall be given its ordinary meaning and shall also refer to a rate of perfusion through an area of the central nervous system of a subject. Flow in some embodiments, can be measured as a rate at which a label or tracer in CSF perfuses through a particular area of the central nervous system (see, e.g., FIGS. 3A-3J of WO2017/210343). As such, flow can be compared between two subjects or two sets of conditions by ascertaining how quickly an injected label or tracer perfuses throughout a particular area or volume of the brain and/or other portion of the CNS.

As used herein, “flow modulators” shall be given its ordinary meaning and shall also broadly refer to classes of compositions that can increase or decrease the passage of substances into and out of meningeal lymphatic vessels, and thus can modulate flow in CSF and ISF, and/or, can modulate immune cell migration within, into, and out of the meningeal lymphatic vessels.

It is shown that, increasing the passage and substances into and out of meningeal lymphatic vessels can increase flow in CSF and ISF (see Examples 4-6 and FIGS. 26-29 of WO2017/210343). Without being limited by theory, it is contemplated, according to several embodiments herein, that removal of macromolecules through meningeal lymphatic vessels can keep their concentrations low in the CSF, allowing a gradient to clear macromolecules from the parenchyma. As such, the higher the rate of drainage of molecules by meningeal lymphatic vessels, the higher the rate of flow of molecules in the CNS (e.g., in CSF and ISF). Furthermore, the higher the rate of fluid flow and drainage in the CNS, the higher the rate of clearance and/or the lower the concentration of cells, macromolecules, waste, and debris form the CNS. In some embodiments, flow modulators increase the diameter of meningeal lymphatic vessels, which increases drainage, resulting in increased flow in the CSF and ISF. In some embodiments, flow modulators increase the number of meningeal lymphatic vessels, thus increasing net drainage, resulting in increased flow in the CSF and ISF. Examples of suitable flow modulators for increasing flow (for example by increasing meningeal lymphatic vessel diameter) in accordance with various embodiments herein include, but are not limited to, VEGFR3 agonists, for example VEGF-c and VEGF-d, and Fibroblast Growth Factor 2 (FGF2), and functional fragments, variants, analogs, and mimetics of these molecules.

In methods, uses, or compositions of some embodiments, a flow modulator (e.g., VEGFR3 agonists, or FGF2) comprises or consists essentially of a polypeptide or protein that comprises a modification, for example a glycosylation, PEGylation, or the like.

In some embodiments, a composition or composition for use in accordance with methods and uses described herein comprises or consists essentially of one or more flow modulators (e.g., VEGFR3 agonists, or FGF2), one or more neurological therapeutic agents (e.g., amyloid beta antibody), and a pharmaceutically acceptable diluent or carrier. Examples of suitable pharmaceutically acceptable carriers and formulations are described in “Remington: The Science and Practice of Pharmacy” 22nd Revised Edition, Pharmaceutical Press, Philadelphia, 2012, which is hereby incorporated by reference in its entirety. In some embodiments, the composition comprises or consists essentially of a unit dose of a flow modulator effective for increasing flow of CNS fluids, increasing clearance of molecules in the CNS, or reducing a quantity of accumulated amyloid beta plaques in accordance with methods or uses as described herein. In some embodiments, the composition comprises, or consists essentially of a single unit dose of flow modulator effective for increasing flow, increasing clearance reducing accumulated amyloid beta plaques, reducing immune cell migration, or reducing inflammation. In some embodiments, the effective amount of flow modulator is about 0.00015 mg/kg to about 1.5 mg/kg (including any other amount or range contemplated as a therapeutically effective amount of a compound as disclosed herein), is less than about 1.5 mg/kg (including any other range contemplated as a therapeutically effective amount of a compound as disclosed herein), or is greater than 0.00015 mg/kg (including any other range contemplated as a therapeutically effective amount of a compound as disclosed herein).

VEGFR3 Agonists

VEGFR3, also known as FLT4, is a receptor tyrosine kinase, and its signaling pathway has been implicated in embryonic vascular development, and adult lymphangiogenesis. Upon binding of ligand, VEGFR3 dimerizes, and is activated through autophosphorylation. It is shown herein that VEGFR3 agonists are a class of flow modulators that increase the diameter of meningeal lymphatic vessels, and which increase drainage and the flow of CSF and ISF in accordance with some embodiments herein (see Examples 4-6, FIGS. 26, 27A-27D, 28A, and 28C of WO 2017/210343). As such, VEGFR3 agonists are suitable for methods, compositions, and uses for treating, ameliorating, reducing the symptoms of, or preventing neurological diseases associated with accumulation of molecules in the brain, for example proteinopathies as described herein (e.g., tauopathies and/or amyloidoses such as AD), in accordance with some embodiments herein. Accordingly, in some embodiments, such as methods or compositions for which increased drainage and flow are desired, a flow modulator comprises, consists of, or consists essentially of a VEGFR3 agonist.

An effective amount of VEGFR3 agonist in accordance with methods, compositions, and uses herein can be understood in terms of its ability to increase meningeal vessel diameter, by its ability to increase flow of CSF or ISF, or by its ability to treat, ameliorate, or prevent (by its ability to increase clearance of substances such as proteins from the CNS, for example amyloid beta), symptoms of a neurological disease such as proteinopathies as described herein (e.g., tauopathies and/or amyloidoses such as AD), for example quantities of beta-amyloid plaques or measurements of cognitive function. Accordingly, in compositions, methods, and uses of some embodiments, an effective amount of VEGFR3 agonist increases meningeal vessel diameter by at least about 2%, for example, at least about 2%, 3%, 4%, 5%, 6%, 7%, 8%, 9%, 10%, 15%, 20%, 25%, 30%, 35%, 40%, 45%, 50%, 55%, 60%, 65%, 70%, or 75%, including ranges between any two of the listed values. In compositions, methods, and uses of some embodiments, an effective amount of VEGFR3 agonist increases flow of the CSF or ISF by at least about 2%, for example, at least about 2%, 3%, 4%, 5%, 6%, 7%, 8%, 9%, 10%, 15%, 20%, 25%, 30%, 35%, 40%, 45%, 50%, 55%, 60%, 65%, 70%, or 75%, including ranges between any two of the listed values.

Example VEGFR3 agonists suitable for methods, uses, and compositions in accordance with some embodiments herein include the polypeptides VEGF-c and VEGF-d, the amino acid sequences of which are shown in Table 1, below, as well as variants and analogs of VEGF-c and/or VEGF-d. By way of example, VEGF-c, in accordance with some embodiments herein has been demonstrated to increase the diameters of meningeal lymphatic vessels, and to increase drainage, CSF and ISF flow, and clearance in the CNS. See Example 4 of WO2017/210343. In some embodiments, a VEGFR3 agonist comprises, consists of, or consists essentially of VEGF-c. In some embodiments, a VEGFR3 agonist comprises, consists of, or consists essentially of VEGF-d. In some embodiments, VEGF-c and VEGF-d together agonize VEGFR3, and can be provided in a single composition, or in separate compositions. In some embodiments, a VEGFR3 agonist comprises, consists of, or consists essentially of an analog, variant, or functional fragment, such as a mutant, ortholog, fragment, or truncation of VEGF-c or VEGF-d, for example a polypeptide comprising, or consisting essentially of an amino acid sequence having at least about 80% identity to SEQ ID NO: 1 or 2 or 3, for example at least about 80%, 85%, 90%, 95%, 97%, 98%, or 99% identity, including ranges between any two of the listed values.

As shown in Examples 5, 6, and 11 of WO2017/210343, exogenous nucleotides encoding a VEGFR3 agonist, such as VEGF-c, can also be suitable for methods, uses, and compositions in accordance with some embodiments herein. Accordingly, in some embodiments, a nucleotide encoding VEGF-c or VEGF-d as describe herein is expressed in a subject in order to administer the VEGFR3 agonist to a subject. For example, an exogenous vector such as a retroviral, lentiviral, adenoviral, or adeno-associated viral vector comprising or consisting essentially of a nucleic acid encoding a VEGFR agonist as described here can be inserted into a host nucleic acid of the subject (for example in the genome of a somatic cell of the subject). In some embodiments, the vector further comprises transcriptional machinery to facilitate the transcription of the nucleic acid encoding the VEGFR agonist, for example, a core promoter, transcriptional enhancer elements, insulator elements (to insulate from repressive chromatin environments), and the like.

TABLE 1A
Example VEGFR3 agonists

| | UniProt | |
|---|---|---|
| Agonist | Accession | SEQ ID NO: |
| VEGF-c | P49769 | SEQ ID NO: 1 |
| | | (MHLLGFFSVACSLLAAALLPGPREAPAAAAAFESGLDLSDAEPD |
| | | AGEATAYASKDLEEQLRSVSSVDELMTVLYPEYWKMYKCQLRKGG |
| | | WQHNREQANLNSRTEETIKFAAAHYNTEILKSIDNEWRKTQCMPR |
| | | EVCIDVGKEFGVATNTFFKPPCVSVYRCGGCCNSEGLQCMNTSTS |
| | | YLSKTLFEITVPLSQGPKPVTISFANHTSCRCMSKLDVYRQVHSI |
| | | IRRSLPATLPQCQAANKTCPTNYMWNNHICRCLAQEDFMFSSDAG |
| | | DDSTDGFHDICGPNKELDEETCQCVCRAGLRPASCGPHKELDRNS |
| | | CQCVCKNKLFPSQCGANREFDENTCQCVCKRTCPRNQPLNPGKCA |
| | | CECTESPQKCLLKGKKFHHQTCSCYRRPCTNRQKACEPGFSYSEE |
| | | VCRCVPSYWKRPQMS) |
| VEGF-d | O43915 | SEQ ID NO: 2 |
| | | (MYREWVVVNVFMMLYVQLVQGSSNEHGPVKRSSQSTLERSEQQI |
| | | RAASSLEELLRITHSEDWKLWRCRLRLKSFTSMDSRSASHRSTRF |
| | | AATFYDIETLKVIDEEWQRTQCSPRETCVEVASELGKSTNTFFKP |
| | | PCVNVFRCGGCCNEESLICMNTSTSYISKQLFEISVPLTSVPELV |
| | | PVKVANHTGCKCLPTAPRHPYSIIRRSIQIPEEDRCSHSKKLCPI |
| | | DMLWDSNKCKCVLQEENPLAGTEDHSHLQEPALCGPHMMFDEDRC |
| | | ECVCKTPCPKDLIQHPKNCSCFECKESLETCCQKHKLFHPDTCSC |
| | | EDRCPFHTRPCASGKTACAKHCRFPKEKRAAQGPHSRKNP) |
| VEGF-C156S | Q6FH59 | SEQ ID NO: 3 |
| | | (MHLLGFFSVACSLLAAALLPGPREAPAAAAAFESGLDLSDAEPD |
| | | AGEATAYASKDLEEQLRSVSSVDELMTVLYPEYWKMYKCQLRKGG |
| | | WQHNREQANLNSRTEETIKFAAAHYNTEILKSIDNEWRKTQCMPR |
| | | EVCIDVGKEFGVATNTFFKPPCVSVYRCGGCCNSEGLQCMNTSTS |
| | | YLSKTLFEITVPLSQGPKPVTISFANHTSCRCMSKLDVYRQVHSI |
| | | IRRSLPATLPQCQAANKTCPTNYMWNNHICRCLAQEDFMFSSDAG |
| | | DDSTDGFHDICGPNKELDEETCQCVCRAGLRPASCGPHKELDRNS |
| | | CQCVCKNKLFPSQCGANREFDENTCQCVCKRTCPRNQPLNPGKCA |
| | | YECTESPQKCLLKGKKFHHQTCSCYRRPCTNRQKACEPGFSYSEE |
| | | VCRCVPSYWKRPQMS) |

In methods or compositions of some embodiments, the VEGFR3 agonist comprises a modification, for example a glycosylation, PEGylation, or the like. In some embodiments, a composition for use in accordance with the methods described herein comprises the VEGFR3 agonist (e.g., VEGF-c and/or VEGF-d), and a pharmaceutically acceptable diluent or carrier.

FGF2

In some embodiments, the flow modulator comprises or consists essentially of Fibroblast Growth Factor 2 (FGF2). Without being limited by theory, it is contemplated that FGF2 can increase drainage (and flow) of CSF or ISF in meningeal lymphatic vessel, for example by increasing the diameter of meningeal lymphatic vessel. An example of a suitable FGF2 amino acid sequence in accordance with some embodiments is provided as Uniprot Accession No. P09038 (human FGF2) (SEQ ID NO: 4—

MVGVGGGDVEDVTPRPGGCQISGRGARGCNGIPGAAAWEAALPRRRPRRH
PSVNPRSRAAGSPRTRGRRTEERPSGSRLGDRGRGRALPGGRLGGRGRGR
APERVGGRGRGRGTAAPRAAPAARGSRPGPAGTMAAGSITTLPALPEDGG
SGAFPPGHFKDPKRLYCKNGGFFLRIHPDGRVDGVREKSDPHIKLQLQAE
ERGVVSIKGVCANRYLAMKEDGRLLASKCVTDECFFFERLESNNYNTYRS
RKYTSWYVALKRTGQYKLGSKTGPGQKAILFLPMSAKS).

Routes of Administration

Flow modulators (e.g., FGF2 or VEGFR3 agonists such as VEGF-c) and/or neurological therapeutic agents (e.g., amyloid beta antibodies) in accordance with methods, compositions for use, or uses of embodiments herein can be administered to a subject using any of a number of suitable routes of administration, provided that the route of administration administers the flow modulator to the CNS (such as the meningeal space) of a subject. It is noted that many compounds do not readily cross the blood-brain barrier, and as such, some routes of administration such as intravenous will not necessarily deliver the flow modulator and/or neurological therapeutic agent to the CNS (unless the flow modulator can readily cross the blood-brain barrier). By “administering to the CNS of a subject,” as used herein (including variations of this root term), it is not necessarily required that a flow modulator and/or neurological therapeutic agent be administered directly to the CNS (such as meningeal space), but rather, this term encompasses administering a flow modulator and/or neurological therapeutic agent directly and/or indirectly to the CNS. It is contemplated that administering the flow modulator and/or neurological therapeutic agent so that it is in fluid communication with the CNS (e.g., meningeal space) of the subject in accordance with some embodiments herein (typically by administering the flow modulator and/or neurological therapeutic agent on the “brain” side of the blood-brain barrier), the flow modulator and/or neurological therapeutic agent will be administered to the meningeal space. Accordingly, in some embodiments, the flow modulator and/or neurological therapeutic agent is not administered systemically. In some embodiments, the flow modulator and/or neurological therapeutic agent is not administered systemically, but rather is administered to a fluid, tissue, or organ in fluid communication with the CNS (such as the meningeal space), and on the brain side of the blood-brain barrier. In some embodiments, the flow modulator and/or neurological therapeutic agent is not administered systemically, but rather is administered to the CNS. In some embodiments, the flow modulator and/or neurological therapeutic agent is administered to the CNS, but is not administered to any organ or tissue outside of the CNS. In some embodiments, the flow modulator and/or neurological therapeutic agent is not administered to the blood. In some embodiments, the flow modulator and/or neurological therapeutic agent is not administered to a tumor, or to the vasculature of a tumor. It is contemplated that a flow modulator and neurological therapeutic agent can be administered together (in a single composition), or separately (e.g., in separate compositions, which can be administered to the same location at the same or different times, or can be administered to different locations at the same or different times). Accordingly, in some embodiments, a flow modulator and neurological therapeutic agent can be administered together (in a single composition). In some embodiments, a flow modulator and neurological therapeutic agent are administered in separate compositions. In some embodiments, a flow modulator and neurological therapeutic agent are administered in separate compositions to different sites of administration on a subject at the same time. In some embodiments, a flow modulator and neurological therapeutic agent are administered in separate compositions to different sites of administration on a subject at different times (for example, the flow modulator can be administered prior to the neurological therapeutic agent, or the flow modulator can be administered after the neurological therapeutic agent). In some embodiments, a flow modulator and neurological therapeutic agent are administered in separate compositions to the same site of administration on a subject at different times (for example, the flow modulator can be administered prior to the neurological therapeutic agent, or the flow modulator can be administered after the neurological therapeutic agent).

Neurological diseases or disorders include, but are not limited to proteinopathies, for example tauopathies and/or amyloidoses such as AD (for example familial AD and/or sporadic AD). Neurological diseases or disorders include, but are not limited to AD (for example familial AD and/or sporadic AD), dementia, age-related dementia, Parkinson's disease (PD), cerebral edema, amyotrophic lateral sclerosis (ALS), Pediatric Autoimmune Neuropsychiatric Disorders Associated with Streptococcal Infections (PANDAS), meningitis, hemorrhagic stroke, autism spectrum disorder (ASD), brain tumor, and epilepsy. In some embodiments, for any method or composition for use described herein, the neurodegenerative disease is selected from the group consisting of: AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CID), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items. In some embodiments, the neurological disease comprises, consists essentially of, or consists of a proteinopathy, for example AD (such as familial AD and/or sporadic AD), Down's syndrome, HCHWA-D, Familial Danish/British dementia, PD, DLB, LB variant of AD, MSA, FENIB, ALS, FTD, HD, Kennedy disease/SBMA, DRPLA; SCA type I, SCA2, SCA3 (Machado-Joseph disease), SCA6, SCA7, SCA17, CID (such as familial CID), Kuru, GSS, FFI, CBD, PSP, CAA, or a combination of two or more of any of the listed items. In some embodiments, the neurological disease or disorder is AD, dementia, or PD. In some embodiments, the neurological disease or disorder comprises, consists essentially of, or consists of a proteinopathy, for example a tauopathy and/or amyloidosis such as AD (for example familial AD and/or sporadic AD). In some embodiments, the neurological disease or disorder comprises, consists essentially of, or consists of AD (for example familial AD and/or sporadic AD). By “biologically compatible form suitable for administration in vivo” is meant a form of the flow modulator and/or neurological therapeutic agent to be administered in which any toxic effects are outweighed by the therapeutic effects of the flow modulator and/or neurological therapeutic agent. The term “subject” is intended to include living organisms in which a neurological disease or disorder can be identified, e.g., mammals. Examples of subjects include humans, dogs, cats, mice, rats, and transgenic species thereof.

Administration of a flow modulator (e.g., FGF2 or VEGFR3 agonist such as VEGF-c) and/or neurological therapeutic agent (e.g., amyloid beta antibody) as described herein can be in any pharmacological form including a therapeutically active amount of an agent alone or in combination with a pharmaceutically acceptable carrier.

Administration of a therapeutically active amount of the therapeutic composition (e.g., flow modulator and/or neurological therapeutic agent) of the present disclosure is defined as an amount effective, at dosages and for periods of time necessary, to achieve the desired result. For example, a therapeutically active amount of a flow modulator and/or neurological therapeutic agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation. The flow modulator (e.g., FGF2 or VEGFR3 agonist such as VEGF-c) and/or neurological therapeutic agent (e.g., amyloid beta antibody) of the disclosure described herein can be administered in a convenient manner such as by transcranial administration, intrathecal administration, intraventricular administration, and/or intraparenchymal administration by contact with cerebral spinal fluid (CSF) of the subject, administration by pumping into CSF of the subject, administration by implantation into the skull or brain, administration by contacting a thinned skull or skull portion of the subject with the agent, injection (subcutaneous, intravenous, etc.), oral administration, inhalation, transdermal application, or rectal administration. In some embodiments, the flow modulator and/or neurological therapeutic agent are administered to the subject (or formulated for administration) by a route selected from the group consisting of intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, or a combination of any of the listed routes. For example, the flow modulator and/or neurological therapeutic agent can be administered to the subject (or formulated for administration) by a route selected from the group consisting of intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, or a combination of any of the listed routes. The flow modulator and the neurological therapeutic agent may be administered by the same route, or by different routes. In some embodiments, the neurological therapeutic agent is administered by intravenous infusion, and the flow modulator is administered by any route of administration described herein. In some embodiments, the neurological therapeutic agent and the flow modulator are both administered by intravenous infusion. Depending on the route of administration, the flow modulator and/or neurological therapeutic agent can be coated in a material to protect the compound from the action of enzymes, acids and other natural conditions which may inactivate the compound. For example, for administration of flow modulator and/or neurological therapeutic agent, by other than parenteral administration, it may be desirable to coat the flow modulator and/or neurological therapeutic agent with, or co-administer the flow modulator and/or neurological therapeutic agent with, a material to prevent its inactivation. For example, the neurological therapeutic agent alone, or with the flow modulator can be administered via intravenous infusion. The intravenous infusion may be repeated, for example, once every 1 week, 2 weeks, 3 weeks, 4 weeks, month, or two months including ranges between any two of the listed values, for example, once every 1-2 weeks, 1-4 weeks, 1 week-1 month, 2-4 weeks, or 2 weeks-1 month. By way of example, the neurological therapeutic agent may be a monoclonal antibody specific for amyloid beta, for example bapineuzumab, gantenerumab, aducanumab, solanezumab, and/or crenezumab. In some embodiments, the monoclonal antibody specific for amyloid beta, such as bapineuzumab, gantenerumab, aducanumab, solanezumab, and/or crenezumab, is administered monthly via intravenous infusion.

In some embodiments, the flow modulator (e.g., FGF2 or VEGFR3 agonist such as VEGF-c) and/or neurological therapeutic agent (e.g., amyloid beta antibody) is administered nasally. For example, the flow modulator and/or neurological therapeutic agent can be provided in a nasal spray, or can be contacted directly with a nasal mucous membrane.

In some embodiments, the flow modulator (e.g., FGF2 or VEGFR3 agonist such as VEGF-c) and/or neurological therapeutic agent (e.g., amyloid beta antibody) is administered through contacting with CSF of the subject. For example, the flow modulator and/or neurological therapeutic agent can be directly injected into CSF of a patient (for example into a ventricle of the brain). Suitable apparatuses for injection can include a syringe, or a pump that is inserted or implanted in the subject and in fluid communication with CSF. In some embodiments, a composition comprising or consisting essentially of the flow modulator and/or neurological therapeutic agent, for example a slow-release gel, is implanted in a subject so that it is in fluid communication with CSF of the subject, and thus contacts the CSF.

In some embodiments, the flow modulator (e.g., FGF2 or VEGFR3 agonist such as VEGF-c) and/or neurological therapeutic agent (amyloid beta antibody) is administered transcranially. For example, a composition comprising or consisting essentially of the flow modulator and/or neurological therapeutic agent such as a gel can be placed on an outer portion of the subject's skull, and can pass through the subject's skull. In some embodiments, the flow modulator and/or neurological therapeutic agent is contacted with a thinned portion of the subject's skull to facilitate transcranial delivery.

In some embodiments, the flow modulator (e.g., FGF2 or VEGFR3 agonist such as VEGF-c) and/or neurological therapeutic agent (e.g., amyloid beta antibody) is administered by expressing a nucleic acid encoding the flow modulator and/or neurological therapeutic agent in the subject. A vector comprising or consisting essentially of the nucleic acid, for example a viral vector such as a retroviral vector, lentiviral vector, or adenoviral vector, or adeno-associated viral vector (AAV) can be administered to a subject as described herein, for example via injection or inhalation. In some embodiments, the nucleic acid is administered as an mRNA as described herein, for example as a chemically modified messenger RNA (mRNA). In some embodiments, expression of the nucleic acid is induced in the subject, for example via a drug or optical regulator of transcription.

In some embodiments, the flow modulator (e.g. the VEGFR3 antagonist such as VEGF-c or FGF2) and/or neurological therapeutic agent (e.g., amyloid beta antibody) is administered selectively to the meningeal space of the subject, or is for use in administration selectively to the meningeal space of the subject. As used herein administered “selectively” and variations of the root term indicate that the flow modulator is administered preferentially to the indicated target (e.g. meningeal space) compared to other tissues or organs on the same side of the blood brain barrier. As such, direct injection to meningeal spaces of the brain would represent “selective” administration, whereas administration to CSF in general via a spinal injection would not. In some embodiments, the flow modulator and/or neurological therapeutic agent is administered selectively to the meningeal space, and not to portions of the CNS outside of the meningeal space, nor to any tissues or organs outside of the CNS. In some embodiments, the flow modulator is administered selectively to the CNS, and not to tissue or organs outside of the CNS such as the peripheral nervous system, muscles, the gastrointestinal system, musculature, or vasculature.

For any of the routes of administration listed herein in accordance with methods, uses, and compositions herein, it is contemplated that a flow modulator (e.g., FGF2 or VEGFR3 agonist such as VEGF-c) and/or neurological therapeutic agent (e.g., amyloid beta antibody) can be administered in a single administration, or in two or more administrations, which can be separated by a period of time. For example, in some embodiments, the flow modulator and/or neurological therapeutic agent as described herein can be administered via a route of administration as described herein hourly, daily, every other day, every three days, every four days, every five days, every six days, weekly, biweekly, monthly, bimonthly, and the like. In some embodiments, the flow modulator and/or neurological therapeutic agent is administered in a single administration, but not in any additional administrations.

Some embodiments include methods of making a composition or medicament comprising or consisting essentially of a flow modulator (e.g., FGF2 or VEGFR3 agonist such as VEGF-c) and/or neurological therapeutic agent (e.g., amyloid beta antibody) as described herein suitable for administration according to a route of administration as described herein. For example, in some embodiments, a composition comprising or consisting essentially of a VEGFR3 agonist (such as VEGF-c) and an amyloid beta antibody is prepared for nasal administration, administration to the CSF, or transcranial administration. For example, in some embodiments, a composition comprising or consisting essentially of a VEGFR3 antagonist (such as VEGF-c) and amyloid beta antibody is prepared for nasal administration, administration by contacting with CSF, or transcranial administration.

Conventional methods, known to those of ordinary skill in the art of medicine, can be used to administer a pharmaceutical composition comprising a flow modulator (e.g. the VEGFR3 antagonist or FGF2) and/or neurological therapeutic agent (e.g, amyloid beta antibody) to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intraventricular, intraparenchymal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

In some examples, the pharmaceutical composition comprising the flow modulator (e.g. the VEGFR3 antagonist or FGF2) and/or neurological therapeutic agent (e.g., amyloid beta antibody) is administered intrathecally, intraventricularly, and/or intraparenchymally, e.g., via an injection into the spinal canal, or into the subarachnoid space.

In some embodiments, the flow modulator (e.g., FGF2 or VEGFR3 agonist such as VEGF-c) and/or neurological therapeutic agent (e.g., amyloid beta antibody) is administered intraventricularly, i.e., into the lateral ventricle of the brain. In some embodiments, the flow modulator and/or neurological therapeutic agent is administered intraparenchymally, i.e., into the brain parenchyma. In some embodiments, the flow modulator and/or neurological therapeutic agent is administered and delivered through an implanted catheter connected to a pump, which contains a reservoir of the composition and controls the rate of delivery. In some embodiments, the flow modulator and/or neurological therapeutic agent is released into the cerebrospinal fluid (CSF) of the cisterna magna. In any of these embodiments, the catheter can either be introduced between the first and second cervical vertebrae (C1-C2 interspace) or into the intracranial ventricles. In some embodiments, an Ommaya reservoir (consisting of a catheter in one lateral ventricle attached to a reservoir implanted under the scalp) is used as an intraventricular catheter system for the administration and delivery of the flow modulator and/or neurological therapeutic agent into the cerebrospinal fluid.

In some embodiments, the flow modulator (e.g., FGF2 or VEGFR3 agonist such as VEGF-c) and/or neurological therapeutic agent (e.g., amyloid beta antibody) is administered at the site of an amyloid beta plaque.

Injectable compositions (such as pharmaceutical compositions comprising a flow modulator and/or neurological therapeutic agent) of some embodiments may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble compositions comprising flow modulator (e.g., FGF2 or VEGFR3 agonist such as VEGF-c) and/or neurological therapeutic agent (e.g., amyloid beta antibody) can be administered by the drip method, whereby a pharmaceutical formulation containing the flow modulator and/or neurological therapeutic agent and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the flow modulator and/or neurological therapeutic agent, can be dissolved and administered in a pharmaceutical excipient such as Water-for-Injection, 0.9% saline, or 5% glucose solution. In some embodiments, the composition comprises VEGFR3 agonist and/or FGF2.

In one embodiment, a flow modulator (e.g., FGF2 or VEGFR3 agonist such as VEGF-c) and/or neurological therapeutic agent (e.g., amyloid beta antibody) is administered via site-specific or targeted local delivery techniques. Examples of site-specific or targeted local delivery techniques include various implantable depot sources of the flow modulator or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568. The flow modulator (e.g., FGF2 or VEGFR3 agonist such as VEGF-c) and/or neurological therapeutic agent (e.g., amyloid beta antibody) can be administered in a pharmaceutical composition as described herein.

Targeted delivery of therapeutic compositions (comprising a flow modulator and/or neurological therapeutic agent as described herein) containing an antisense polynucleotide, expression vector (viral or non-viral), or subgenomic polynucleotides, or mRNA is also contemplated within the disclosure. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., a polynucleotide encoding a flow modulator and/or neurological therapeutic agent as described herein) are administered in a range of about 100 ng to about 200 mg of DNA for local administration in a gene therapy protocol. In some embodiments, ranges of about 500 ng to about 50 mg, about 1 μg to about 2 mg, about 5 μg to about 500 μg, and about 20 μg to about 100 μg of DNA or more can also be used during a gene therapy protocol. In some embodiments, DNA is administered at a concentration of about 100 ng/ml to about 200 mg/ml.

The flow modulator (e.g., FGF2 and/or VEGFR3 agonists such as VEGF-c, described herein) and/or neurological therapeutic agent (e.g., amyloid beta antibody) can comprise, consist essentially of, or consist of one or more therapeutic polynucleotides and polypeptides, and can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide (for example, encoding a flow modulator and/or neurological therapeutic agent as described herein) and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). AAV vectors are particularly suitable for delivery of a payload into the central nervous system. Over 100 AAV serotypes have been identified that differ in the binding capacity of capsid proteins to specific cell surface receptors that can transduce different cell types and brain regions in the CNS. Non-limiting examples of AAV serotypes include AAV1, AAV2/1, AAVDJ, AAV8, AAVDJ8, AAV9, and AAVDJ9.

Promoters to drive expression in the brain can be constitutive, such as beta-actin, phosphoglycerate kinase 1 or CMV promoters, or tissue specific. Examples of tissue specific promoters which can be used to drive expression in brain tissues include the synapsin, Glial fibrillary acidic protein (GFAP), glutamic acid decarboxylase (GAD67), homeobox Dlx5/6, glutamate receptor 1 (GluR1), and preprotachykinin 1 (Tac1), and Musashi1 promoters. These promoters show diversity of transcriptional activity and cell-type specificity of expression. Accordingly, in some embodiments, a promoter is selected based on the desired expression in a cell type, tissue or brain region. Accordingly, in some embodiments, the expression of the nucleic acid of the interest, e.g., encoding a flow modulator and/or neurological therapeutic agent as described herein, is under control of a brain tissue specific promoter, including but not limited to, synapsin, GFAP, GAD67, homeobox Dlx5/6, GluR1, and Tac1, and Musashi1 promoters or others known in the art.

A large number of tumor-specific promoters have been employed in gene therapy approaches. For example, the hTERT promoter has been used to drive cancer-specific expression in a number different types of cancer tissues. Alpha-fetoprotein (AFP) and erb2 promoters have been used to target hepatic cancer and breast cancer, respectively. Several promoters, including carcinoembryonic antigen (CEA), cyclooxygenase-2 (COX-2), hTERT, and Urokinase-type plasminogen activator receptor (uPAR) have been used to direct suicide genes into colorectal carcinoma cells (Rama et al., Disease Markers (2015). Alternatively, a promoter active under hypoxic conditions can be used tumor specific expression in the tumor microenvironment, such as the HIF-1 promoter. Several transgenes have been successfully expressed under the control of a hypoxia-inducible promoter, e.g., p53 for induction of apoptosis, HSV thymidine kinase, bacterial nitroreductase, VEGF receptor 1-Ig, CD40 ligand, and IL-4 (see, e.g., Guo, Virus Adaptation and Treatment 2011:371-82 The impact of hypoxia on oncolytic virotherapy). Accordingly, in some embodiments, the expression of the nucleic acid of the interest, e.g., encoding a flow modulator (such as FGF2 and/or VEGFR3 agonists such as VEGF-c, described herein) and/or neurological therapeutic agent (e.g., amyloid beta antibody), is under control of a tumor specific promoter, including but not limited to, hTERT, AFP, erb2 CEA, COX-2, and uPAR promoters, or a hypoxia inducible promoter, including but not limited to HIF1.

Administration of DNA linked to killed adenovirus as described in Curiel, Hum. Gene Ther. (1992) 3:147 can also be employed.

In some embodiments, non-viral delivery vehicles and methods are employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

In some embodiments, the flow modulator (e.g., FGF2 or VEGFR3 agonist such as VEGF-c) and/or neurological therapeutic agent (e.g., amyloid beta antibody) is administered as an mRNA (e.g., a mRNA encoding the VEGFR3 agonist). In some embodiments, chemically modified messenger RNA (mRNA) is employed. Modified mRNA evades recognition by the innate immune system and is less immunostimulating than dsDNA or regular mRNA. Additionally, cytoplasmic delivery of mRNA circumvents the nuclear envelope, which can result in a higher expression level. Exemplary mRNA introduction methods are described in Rhoads et al. (Methods in Molecular Biology, vol. 1428, DOI 10.1007/978-1-4939-3625-01), and references therein.

In some embodiments, nucleic acids (e.g., comprising or encoding a flow modulator and/or neurological therapeutic agent) are delivered to a cell naked, i.e., free from complexing agents, for example, lipid agents and polymer agents, etc. In some embodiments, naked mRNA is delivered by injection (intradermal, intrathecal, intraventricular, intraparenchymal, etc).

The flow modulator and/or neurological therapeutic agent nucleic acids or polypeptides of compositions and methods of some embodiments may be formulated according to methods known in the art, and the formulations may further include, but are not limited to, cell penetration agents, a pharmaceutically acceptable carrier, delivery agents, a bioerodible or biocompatible polymers, solvents, and sustained-release delivery depots.

In some embodiments, the nucleic acid comprising or encoding the flow modulator and/or neurological therapeutic agent (e.g., RNAi agent, siRNA, ASO, LNA, or mRNA, or DNA) comprises, consists essentially of, or consists of modified nucleic acids or nucleobases. Example flow modulators that can be encoded include VEGFR3 agonist or antagonist, or FGF2. Example neurological therapeutic agents include amyloid beta antibodies. In some embodiments, the nucleic acid encoding the flow modulator and/or neurological therapeutic agent comprises, consists essentially of, or consists of an antisense oligonucleotide (ASO). By way of example, the ASO can hybridize to a complementary mRNA and mediate silencing of expression from the mRNA, such as by blocking ribosome binding to the mRNA and/or recruiting RNase H to mediate degradation of the mRNA. For example, the nucleic acid molecule can be a mimetic, can include a modified sugar backbone, one or more modified internucleoside linkages (e.g., one or more phosphorothioate and/or heteroatom internucleoside linkages), one or more modified bases, and the like. In some embodiments, the nucleic acid has a morpholino backbone structure. In some embodiments, the nucleic acid has one or more locked nucleic acids (LNAs). Suitable sugar substituent groups include methoxy (—O—CH3), aminopropoxy (—OCH2 CH2 CH2NH2), allyl (—CH2-CH═CH2), —O-allyl (—O—CH2-CH═CH2) and fluoro (F). 2′-sugar substituent groups may be in the arabino (up) position or ribo (down) position. In some embodiments, the nucleic acid has base modifications. Base modifications include synthetic and natural nucleobases. Suitable base modifications include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methylpseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In some embodiments, the flow modulator and/or neurological therapeutic agent can comprise naturally occurring and/or artificial nucleic acid, for example a mimetic, one or more modified internucleoside linkages, and/or one or more modified bases, such as base modifications as described herein.

In some embodiments, the nucleic acid (e.g., of the flow modulator and/or neurological therapeutic agent) is an mRNA which has at least one modification in one of the bases A, G, U and/or C. In some embodiments, the mRNA has at least one 5′ terminal cap is selected from the group consisting of Cap0, Cap 1, ARCA, inosine, N1-methyl-guanosine, 2′fluoro-guanosine, 7-deaza-guanosine, 8-oxo-guanosine, 2-amino-guanosine, LNA-guanosine and 2-azido-guanosine. In some embodiments the mRNA has a polyA or a polyT tail, for example, of 100-200 nucleotides. In some embodiments, the comprises a 5′ untranslated region and/or a 3′ untranslated region. In some examples, the UTR(s) are not derived from the native untranslated region corresponding to the polypeptide of interest, e.g., are beta-globin UTRs. In some embodiments, the sequence composition of the mRNA is altered by incorporating the most GC-rich codon for each amino acid. For example, expression of proteins from synthetic mRNA can be diminished if the mRNA contains rare codons or rate-limiting regulatory sequences. Redesign of the mRNA by using synonymous but more frequently used codons can increase the rate of translation and hence, translational yield (Gustafsson et al., Trends Biotechnol 22:346-353). In some embodiments, the nucleic acids of the disclosure are codon optimized, e.g., to optimize translation and reduce immunogenicity. Codon optimization tools known in the art employ algorithms for codon optimization, many of which take codon usage tables, codon adaptability, mRNA structure, and various cis-elements in transcription and translation into consideration. A non-limiting example of a useful platform is ptimumGene™ algorithm from GenScript.

Structural modifications to mRNA (such as flow modulator and/or neurological therapeutic agent mRNA) increase stability and translational efficiency can be divided into the various domains of mRNA: cap, UTRs, coding region, poly(A) tract, and 3′-end. mRNA is produced according to methods known in the art (see e.g., Rhoads (ed.), Synthetic mRNA: Production, Introduction Into Cells, and Physiological Consequences, Methods in Molecular Biology, vol. 1428, DOI 10.1007/978-1-4939-3625-0_1). mRNA is synthesized in vitro, e.g., using T7 polymerase-mediated transcription from a linearized DNA template containing an open reading frame, flanking 5′ and 3′ untranslated regions and a poly-A tail. A Cap structure, such as a Cap1 structure, can be enzymatically added to the 5′ end to produce the final mRNA. In some embodiments, the nucleobases bases are modified. For example, in some embodiments, uridine is completely substituted with N1-methylpseudouridine to reduce potential immunostimulatory activity and to improve protein expression relative to unmodified mRNA. Alternatively one or more of the modifications described supra can be used. After the mRNA is purified, the mRNA is diluted, frozen or prepared for administration. Suitable buffer solutions are known in the art. A non-limiting example of a suitable buffer is a solution containing 2.94 mg/mL sodium citrate dihydrate at pH 6.5 and 7.6 mg/mL sodium chloride (Gan et al., Nature Communications, volume 10, Article number: 871 (2019)).

In some embodiments, the nucleic acid (e.g., comprising or encoding a flow modulator and/or neurological therapeutic agent) includes a conjugate moiety (e.g., one that enhances the activity, cellular distribution or cellular uptake of the oligonucleotide). These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a nucleic acid.

The particular dosage regimen, i.e., dose, timing and repetition, used in the methods of some embodiments herein depend on the particular subject and that subject's medical history.

In some embodiments, more than one flow modulator and/or neurological therapeutic agent, or a combination of a flow modulator and/or neurological therapeutic agent and another suitable therapeutic agent, may be administered to a subject in need of the treatment. The flow modulator (e.g., FGF2 and/or VEGFR3 agonist such as VEGF-c) and/or neurological therapeutic agent (e.g., amyloid beta antibody) can also be used in conjunction with other agents that serve to enhance and/or complement the effectiveness of the flow modulator and/or neurological therapeutic agent. In some embodiments, the flow modulator and/or neurological therapeutic agent is administered.

Treatment efficacy for a target neurological disease/disorder, for example a proteinopathy (e.g., a tauopathy and/or amyloidosis such as AD), for example in the head, skull, meninges, central nervous system, and/or brain as described herein can be assessed by methods well-known in the art. The target neurological disease/disorder can comprise amyloid beta plaques.

Pharmaceutical Compositions

A flow modulator (e.g., FGF2 or VEGFR3 agonist such as VEGF-c) and/or neurological therapeutic agent (e.g., amyloid beta antibody), as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease, e.g., as described herein. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the flow modulator (e.g., FGF2 or VEGFR3 agonist such as VEGF-c) and/or neurological therapeutic agent (e.g., amyloid beta antibody) (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). The liposomes can be extruded through filters of defined pore size to yield liposomes with the desired diameter. Without being limited by theory, it is contemplated that minimizing lipid diameter can inhibit or avoid interaction between the liposome and circulating proteins, thus prolonging the circulation time of the liposome. Lipsomes for mRNA delivery reviewed, for example, in Reichmuth et al., Ther. Deliv. 7: 319-334 (2016), which is incorporated by reference in its entirety herein. In some embodiments, the liposome has a diameter smaller than the interior diameter of a meningeal lymphatic vessel, so that the liposome may travel through the meningeal lymphatics. In some embodiments, the liposome has a dimeter of less than 150 nm, for example, less than 140 nm, 130 nm, 120 nm, 110 nm, 100 nm, 90 nm, 80 nm, 70 nm, 60 nm, 50 nm, 40 nm, 30 nm, 20 nm, or 10 nm, including ranges between any two of the listed values. Without being limited by theory, it is contemplated that a target cell can endocytose of a liposome comprising mRNA, and following release of the mRNA from the lipsome, the mRNA can be available in the cytosol of the target cell. Without being limited by theory, it is contemplated that the inclusion of an amine group at or near the surface of the liposome can maintain a neutral or mildly cationic surface charge at physiological pH, so as to minimize non-specific protein interactions can facilitate release of the mRNA in the cytosol. In some embodiments, the liposomes are administered to a subject in vivo according to a route of administration as described herein, for example parenteral or intranasal.

In some embodiments, the flow modulator (e.g., FGF2 or VEGFR3 agonist such as VEGF-c) and/or neurological therapeutic agent (e.g., amyloid beta antibody) mRNA is contained in 30-[N—(N′,N′-dimethylaminoethane) carbamoyl](DC-Cholesterol)/dioleoylphosphatidylethanolamine (DOPE) liposomes. In some embodiments, the VEGFR3 agonist is encapsulated with DOTAP. In some embodiments, the VEGFR3 agonist is encapsulated in a cationic lipid preparation. RNA can also be protected against degradation by complexing with the polycationic protein protamine. Accordingly, in some embodiments, VEGFR3 agonist mRNA is complexed with protamine, for example, according to the curevac RNActive® platform. Without being limited by theory, complexing with protamine is contemplated to inhibit or limit immunogenicity of the composition comprising the mRNA.

Excipients suitable for flow modulators (e.g., FGF2 or VEGFR3 agonist such as VEGF-c) and/or neurological therapeutic agents (e.g., amyloid beta antibody) as described herein can include, without limitation, lipidoids, liposomes, lipid nanoparticles, polymers, lipoplexes, core-shell nanoparticles, peptides, proteins, carbohydrates, cells loaded with nucleic acids or polypeptides of the disclosure, hyaluronidase, nanoparticle mimics and combinations thereof. In some embodiments, nucleic acids can be delivered using a Gene Gun.

In some embodiments, nucleic acids, e.g., DNA, mRNA, siRNA, etc., may be formulated in lipidoids. Non-limiting examples of such lipidoids contain amino-alkyl-acrylate and -acrylamide materials and are known in the art (see e.g., Love et al, PNAS May 25, 2010 107 (21) 9915). C16-96, C14-110, and C12-200 are other examples of lipidoids, which can be prepared, complexed with nucleic acid, e.g., siRNA or mRNA, and delivered according to Love et al, PNAS May 25, 2010 107 (21) 9915.

In some embodiments, the nucleic acid of the disclosure is administered in stable nucleic acid lipid particle (SNALP) formulations.

In one embodiment, nucleic acids or polypeptides described herein may be formulated in lipid nanoparticles. The formulation may be influenced by parameters including, but not limited to, the selection of the cationic lipid component, the degree of cationic lipid saturation, the nature of the PEGylation, ratio of all components and biophysical parameters such as size (Semple et al. Nature Biotech. 2010 28:172-176). A non-limiting example of a cationic lipid that is suitable for formulation of nucleic acids, e.g., mRNA, 1,2-dilinoleyloxy-3-dimethylaminopropane (DLinDMA). This cationic lipid can be synthesized and used as a main component of lipid nanoparticles. An ethanol dilution process is used to produce small uniform lipid particles with a high RNA encapsulation efficiency (Geall et al., PNAS Sep. 4, 2012 109 (36) 14604-14609).

In one embodiment, nucleic acids or polypeptides described herein may be formulated in exosomes, microvesicles, and/or extracellular vesicles. The exosomes, microvesicles, and/or extracellular vesicles may be loaded with at least one VEGFR3 agonist or VEGFR3 antagonist and delivered to cells or tissues. Exosomes which can function as nucleic acid delivery vehicles are known in the art and are for example described in U.S. Pat. No. 9,629,929.

The flow modulator (e.g., FGF2 or VEGFR3 agonist such as VEGF-c) and/or neurological therapeutic agent (e.g., amyloid beta antibody), or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the polypeptide or nucleic acid of the disclosure, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid).

In some embodiments, the compositions described herein may include at least one polymer such as, but not limited to, polyethenes, polyethylene glycol (PEG), poly(1-lysine)(PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[a-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

A self-assembling polyplex nanomicelle composed of a polyethylene glycol-polyamino acid block copolymer was used to administer luciferase-expressing mRNA with nucleoside modification into the CNS by intrathecal injection into the cisterna magna of mice (Ushida et al., PLoS One 8, e56220(2013). Accordingly, in some embodiments, the nucleic acid, e.g., mRNA, of the disclosure is delivered intrathecally in a polyplex nanomicelle composed of a polyethylene glycol-polyamino acid block copolymer.

The pharmaceutical compositions of some embodiments herein are to be used for in vivo administration, and are sterile. Sterilization can be readily accomplished by, for example, filtration through sterile filtration membranes. Therapeutic flow modulator and/or neurological therapeutic agent compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an VEGFR3 agonist or VEGFR3 antagonist with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

The flow modulator (e.g., FGF2 or VEGFR3 agonist such as VEGF-c) and/or neurological therapeutic agent (e.g., amyloid beta antibody) may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of a flow modulator (e.g., FGF2 or VEGFR3 agonist such as VEGF-c) and/or neurological therapeutic agent (e.g., amyloid beta antibody) suitable for injectable use include sterile aqueous solutions (where water soluble) or dispersions and sterile powders for the extemporaneous preparation of sterile injectable solutions or dispersion. In all cases the composition will in some embodiments be sterile and must be fluid to the extent that the composition has easy syringeability (such as the composition easily passing from a container through an injection needle into a syringe prior to injection) and injectability (such as the composition easily passes from a syringe through an injection needle into an administration site of the subject)(See, e.g., Cilurzo et a. AAPS PharmSciTech. 12: 604-609 (2011) for a review of syringeability and injectability). It will in some embodiments be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a flow modulator (e.g., FGF2 or VEGFR3 agonist such as VEGF-c) and/or neurological therapeutic agent (e.g., amyloid beta antibody) of the disclosure in the required amount in an appropriate solvent with one or a combination of ingredients enumerated above, as required, followed by filtered sterilization. Generally, dispersions are prepared by incorporating the active flow modulator and/or neurological therapeutic agent compound into a sterile vehicle which contains a basic dispersion medium and the required other ingredients from those enumerated above. In the case of sterile powders for the preparation of sterile injectable solutions, and, in some embodiments methods of preparation are vacuum drying and freeze-drying which yields a powder of the flow modulator and/or neurological therapeutic agent plus any additional desired ingredient from a previously sterile-filtered solution thereof.

When the flow modulator (e.g., FGF2 or VEGFR3 agonist such as VEGF-c) and/or neurological therapeutic agent (e.g., amyloid beta antibody) is suitably protected, as described herein, the flow modulator and/or neurological therapeutic agent can be orally administered, for example, with an inert diluent or an assimilable edible carrier. As used herein “pharmaceutically acceptable carrier” has its ordinary meaning as understood in the art in view of the specification, and includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active flow modulator and/or neurological therapeutic agent, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, has its ordinary meaning as understood in the art in view of the specification, and includes physically discrete units suited as unitary dosages for the mammalian subjects (such as humans) to be treated; each unit containing a predetermined quantity of active compound (e.g., flow modulator and/or neurological therapeutic agent) calculated to produce the desired therapeutic effect in association with the required pharmaceutical carrier. The specification for the dosage unit forms of the disclosure are dictated by, and directly dependent on, (a) the unique characteristics of the active flow modulator and/or neurological therapeutic agent and the particular therapeutic effect to be achieved, and (b) the limitations inherent in the art of compounding such an active flow modulator and/or neurological therapeutic agent for the treatment of sensitivity in individuals. In some embodiments, a flow modulator and/or neurological therapeutic agent of the disclosure is an antibody. As defined herein, a therapeutically effective amount of antibody (e.g., an effective dosage) ranges from about 0.001 to 30 mg kg body weight, in some embodiments about 0.01 to 25 mg kg body weight, in some embodiments about 0.1 to 20 mg kg body weight, and in some embodiments about 1 to 10 mg/kg, 2 to 9 mg/kg, 3 to 8 mg kg, 4 to 7 mg/kg, or 5 to 6 mg kg, or a range defined by any two of the preceding values, body weight. The skilled artisan will appreciate that certain factors may influence the dosage required to effectively treat a subject, including but not limited to the severity of the disease or disorder, previous treatments, the general health and/or age of the subject, and other diseases present.

Moreover, treatment of a subject with a therapeutically effective amount of a flow modulator (e.g., FGF2 or VEGFR3 agonist such as VEGF-c) and/or neurological therapeutic agent (such as an amyloid beta antibody) can include a single treatment or, in some embodiments, can include a series of treatments. In some embodiments, a subject is treated with antibody (such as a neurological therapeutic agent comprising, consisting essentially of, or consisting of an amyloid beta antibody) in the range of between about 0.1 to 20 mg kg body weight, one time per week for between about 1 to 10 weeks, in some embodiments between 2 to 8 weeks, in some embodiments between about 3 to 7 weeks, and in some embodiments for about 4, 5, or 6 weeks, or a range defined by any two of the preceding values. It will also be appreciated that the effective dosage of antibody (e.g., amyloid beta antibody) used for treatment may increase or decrease over the course of a particular treatment. Changes in dosage may result from the results of diagnostic assays. In addition, an antibody of the disclosure can also be administered in combination therapy with, e.g., chemotherapeutic agents, hormones, antiangiogens, radiolabeled, compounds, or with surgery, cryotherapy, and/or radiotherapy. An antibody of the disclosure (e.g., amyloid beta antibody) can also be administered in conjunction with additional forms of therapy (such as one or more conventional therapies, which may include, for example, an antibody, peptide, a fusion protein and/or small molecule), either consecutively with, pre- or post- the additional therapy. For example, the antibody can be administered with a therapeutically effective dose of chemotherapeutic agent. In some embodiment, the antibody can be administered in conjunction with chemotherapy to enhance the activity and efficacy of the chemotherapeutic agent. The Physicians' Desk Reference (PDR) discloses dosages of chemotherapeutic agents that have been used in the treatment of various neurological disease and disorders. The dosing regimen and dosages of these aforementioned chemotherapeutic drugs that are therapeutically effective will depend on the particular neurological disease or disorder, being treated, the extent of the disease and other factors familiar to the physician of skill in the art and can be determined by the physician.

In addition, the flow modulators (e.g., FGF2 or VEGFR3 agonists such as VEGF-c) and/or neurological therapeutic agents (e.g., amyloid beta antibodies) of the disclosure described herein can be administered using nanoparticle-based composition and delivery methods well known to the skilled artisan. For example, nanoparticle-based delivery for improved nucleic acid (e.g., small RNAs) therapeutics are well known in the art (Expert Opinion on Biological Therapy 7: 1811-1822).

A flow modulator (e.g., FGF2 or VEGFR3 agonist such as VEGF-c) and/or neurological therapeutic agent (e.g., amyloid beta antibody) can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

Neurological Therapeutic Agents

The present disclosure provides therapy or treatment of neurodegenerative diseases using neurological therapeutic agents in combination with flow modulators. The neurological therapeutic agent (e.g., an amyloid beta antibody) surprisingly synergizes with the flow modulator (e.g., VEGFR3 agonist and/or FGF2) to improve the treatment of a subject. Without wishing to be bound by theory, flow modulators increase fluid flow in the central nervous system (e.g., brain) and as a result improve the delivery of the neurological therapeutic agents and/or drainage of the lymphatic vessel within the central nervous system (e.g., brain). In one non-limiting example, improved drainage allows the removal of pathological aggregates targeted by the therapeutic agent. Accordingly, without wishing to be bound by theory, a treatment combining a flow modulator described herein with a therapeutic agent described herein may improve the extent of the desired effect on the pathology of the neurological disease, e.g., reduction of pathological protein aggregates, reduction of inflammation at the site of aggregation, etc., as compared to a treatment with the therapeutic agent alone or as compared to a treatment with the flow modulator alone.

As used herein, a “neurological therapeutic agent” refers to an agent that treats, prevents, inhibits, ameliorates, or reduces the symptoms of one or more neurological diseases, for example proteinopathies as described herein (e.g., tauopathies and/or amyloidoses such as AD). In certain embodiments, the neurological therapeutic agent is selected from a group consisting of a small molecule, an oligopeptide, a polypeptide, an antibody, a nucleic acid, a recombinant virus, a vaccine, and a cell.

The neurological therapeutic agent may target a peptide or a protein that is involved in the pathogenesis of a neurologic disease. The exemplary target peptides or proteins include, but are not limited to Aβ (Amyloid-β peptide), synuclein, fibrin, tau, apolipoprotein E (Apoe), TDP43, prion protein, Huntingtin exon 1, ABri peptide, ADan peptide, fragments of immunoglobulin light chains, fragments of immunoglobulin heavy chains, full or N-terminal fragments of serum amyloid A protein (SAA), transthyretin (TTR), β₂-microglobulin, N-terminal fragments of apolipoprotein A-I (ApoAI), C-terminal extended apolipoprotein A-II (ApoAII), N-terminal fragments of apolipoprotein A-IV (ApoAIV), apolipoprotein C-II (ApoCII), apolipoprotein C-III (ApoAIII), fragments of gelsolin, lysozyme, fragments of fibrinogen α-chain, N-terminal truncated cystatin C, islet amyloid polypeptide (IAPP), calcitonin, atrial natriuretic factor (ANF), N-terminal fragments of prolactin (PRL), insulin, medin, lactotransferrin, odontogenic ameloblast-associated protein (ODAM), pulmonary surfactant-associated protein C (SP-C), leukocyte cell-derived chemotaxin-2 (LECT-2), galectin 7 (Gal-7), Comeodesmosin (CDSN), C-terminal fragments of kerato-epthelin (pih-h3), semenogelin-1 (SGI), proteins S100A8/A9, Enfuvirtide, GSK-3β, MARK, CDK5, tyrosine kinase Fyn, protein phosphatase 2A (PP2A), LRRK2, GBA, NF-κB p65 (see, Chiti et al., Protein Misfolding, Amyloid Formation, and Human Disease: A Summary of Progress Over the Last Decade, Annu. Rev. Biochem., 86:35.1-35.42 (2017)).

The neurological therapeutic agent may target a peptide or a protein specifically or non-specifically. As used herein, the terms “specific targeting” or “specifically targets” refer to an ability to discriminate between possible peptides or proteins in the environment in which the interaction between the neurological therapeutic agent and the target is to occur. In some embodiments, a neurological therapeutic agent that interacts, e.g., preferentially interacts, with one particular peptide or protein when other potential neurological therapeutic agents are present is said to “specifically target” the peptide or protein with which it interacts. In some embodiments, specific targeting is assessed by detecting or determining the degree of association between the neurological therapeutic agent and its target. In some embodiments, specific targeting is assessed by detecting or determining ability of the neurological therapeutic agent to compete with an alternative interaction between its target and another entity. In some embodiments, specific targeting is assessed by performing such detections or determinations across a range of concentrations. Exemplary specific targeting includes, but is not limited to, specific binding of an antibody to its target protein. Exemplary non-specific targeting includes, but is not limited to, the interaction between a small molecule and a class of proteins or peptides. For example, a tyrosine kinase inhibitor may non-specifically inhibit several protein tyrosine kinases. In some embodiments, a neurological therapeutic agent of the invention may modulate, e.g., increase or decrease, the activity (e.g., enzyme activity) of its target protein or proteins. In some embodiments, a neurological therapeutic agent may modulate, e.g., increase or decrease, the folding and/or aggregation a peptide or protein. For example, a neurological therapeutic agent may binds to a protein, e.g., amyloid-3 or tau protein, to reduce the misfolding and aggregation thereof.

A neurological therapeutic agent may modulate a target peptide or protein directly or indirectly. For example, by direct modulation, the neurological therapeutic agent may interact with the target peptide or protein directly, e.g., an antibody binds to the target peptide or protein. In some other examples, the neurological therapeutic agent may indirectly modulate the target peptide or protein by interacting with another peptide or protein, and modulate the target by interacting with the another peptide or protein. For example, a protein kinase inhibitor may interact with a protein kinase to indirectly modulate the phosphorylation status of the target peptide or protein.

A neurological therapeutic agent may also not target a peptide or protein that is involved in the genesis of a proteinopathy. For example, a neurological therapeutic agent of the invention may prevent or reduce the downstream event of the proteinopathy, such as neuroinflammation that is associated with a neurodegenerative disease.

In certain embodiments, the neurological therapeutic agent comprises a small molecule. The term “small molecule,” as used herein, refers to a low molecular weight (e.g., <900 daltons) organic compound. Exemplary small molecules that can be used as neurological therapeutic agents include, but are not limited to, Donepezil, Galantamine, Rivastigmine, Memantine, Lanabecestat, Atabecestat, Verubecestat, Elenbecestat, Semagacestat, Tarenflurbil, Brexipiprazole, AXS-05 (Axsome Therapeutics), AC-1204 (Accera), masitinib, amilomotide, guanfacine hydrochloride, octohydroaminoacridine succinate, lumateperone tosylate, AVP-786 (Avanir Pharmaceuticals), ALZT-OP1, AZD-1080 (AstraZeneca), ASN 120290 (Asceneuron SA), GV-971, CNP520 (Novartis), DNL104 (RIPK1 inhibitor), DNL747 (RIPK1 inhibitor), Namzaric, Namenda XR, Reminyl, tideglusib (NP031112, NP-12), saracatinib (AZD0530), NPT200-11, NPT088, NPT100-18A thiamet-G, methylene blue, LMTX (leuco-methylthionium bis (hydro-methanesulfonate (LM™) and leuco-methylthionium dihydrobromide (LMTB), SEL-141 (Selvita SA), TauRx Therapeutics), nilvadipine, AZPZs, AZP2006, RPEL, PE859, SLM, SLOH, IMS-088 (ImStar), derivatives of 2,4-thiazolidinedione, rhein-huprine hydrid, 1-benzylamino-2-hydroxyalkyl derivatives, apomorphine, carbenoxolone, trazodone, hexachlorophene, rifaximin, memantine hydrochloride, rotigotine ER, Duopa (AbbVie), donepezil hydrochloride, Madopar (Taiyo Pharma), droxidopa, pimavanserin tartrate, deutetrabenazine, zonisamide, cysteamine, galantamine hydrobromide, tetrabenazine, Stalevo (Novartis), ropinirole hydrochloride, Rytary (Amneal Pharmaceuticals), istradefylline, apomorphine hydrochloride, cerliponase alfa, amantadine hydrochloride, idebenone, bromocriptine mesylate, Neodopasol (Daiichi Sankyo Co. Ltd.), Ecalevo (Sandoz), Neodopaston (Daiichi Sankyo Co. Ltd.), rasagiline mesylate, benztropine mesylate, biperiden hydrochloride, cabergoline, carbidopa, cerebrolysin, diphenhydramine, diphenhydramine hydrochloride, entacapone, ergoloid mesylates, ianabecestat, ibudilast, levodopa, mazaticol, nicergoline, opicapone, orphenadrine, oxytocin, pergolide mesylate, piroheptine, pramipexole dihydrochloride, procyclidine hydrochloride, profenamine, rasagiline, risperidone, ropinirole hydrochloride, safinamide mesylate, scopolamine, scopolamine hydrobromide, selegiline hydrochloride, talipexole, taurine, tertomotide, tetrabenazine, tiapride, tolcapone, trihexyphenidyl, zonisamide, cycrimine, tacrine hydrochloride, lemborexant, ABBV-951 (AbbVie), acetylleucine, ASD-005 (Asdera), ASD-006 (Asdera), azeliragon, cromolyn in combination with ibuprofen, dexamethasone sodium phosphate, E-2027 (Eisai), F-627 (Generon (Shanghai))), omaveloxolone, RG-6042 (Chugai), RT-001 (Retrotope Inc), cyclobenzaprine hydrochloride, Trappsol Cyclo, tricaprilin, troriluzole hydrochloride, valproate sodium, vatiquinone, venglustat malate, verdiperstat, aplindore fumarate, betamethasone, dexmedetomidine, dextro epicatechin, laquinimod sodium, masupirdine, mesdopetam, montelukast sodium, neflamapimod, vafidemstat, alpha-dihydrotetrabenazine, Bisnorcymserine, dimethyl fumarate, lithium citrate, nabiximols, gemfibrozil in combination of tretinoin, davunetide, gemfibrozil, hydralazine hydrochloride, idalopirdine, lithium salicylate, E-2012 (Eisai), ELND-005, begacestat, FK-962 (Astellas), GSI-136 (Pfizer), S-8510 (Shionogi), TAK-065 (Takeda), ABT-099 (AbbVie), and tolfenamic acid.

A small molecule neurological therapeutic agent may exert the therapeutic effects through various mechanisms. For example, the small molecule neurological agent may reduce mitochondrial dysfunction and ROS production, protein oxidation, lipid peroxidation, nitrosative stress, protein aggregation, amyloidopathy, tauopathy, DNA damage, depletion of endogenous antioxidant enzymes, proteosomeal dysfunction, microglial activation, neuroflammation, and/or neuroepigenetic modification.

A small molecule neurological therapeutic agent may include p2-adrenergic agonists, c-Ab1 inhibitors, cholinesterase inhibitors, leucine-rich repeat kinase 2 inhibitors, glucocerebrosidase inhibitors, glycogen synthase kinase 3p inhibitors, N-acetylglucosaminidase inhibitors, O-GlcNAcase inhibitors, or anti-inflammatory compounds.

In some embodiments, the neurological therapeutic agent comprises a protein or peptide. Exemplary proteins or peptides include, but are not limited to, insulin and interferon gamma-1b. In certain embodiments, the peptide or protein is a chaperone protein or a co-chaperone that facilitates the proper folding of a target peptide or protein. Chaperone proteins assist the conformational folding or unfolding and the assembly or disassembly of other proteins. Exemplary chaperone proteins include, but are not limited to, heat shock proteins (e.g., Hsp104, Hsp90, Hsp70, Hsp27), αB-crystallin, clusterin, a2-macroglobulin, haptoglobin, human tetrameric transthyretin, proSAAS, protein 7B2, ERdj3/DNAJB11, GRP78/BiP, GRP94, GRP170, calnexin, calreticulin, and protein disulfide isomerase. Co-chaperones are proteins that assist chaperones in protein folding and other functions. Exemplary co-chaperones include, but are not limited to J-proteins, DnaJ, Hsp40, DNAJC5, auxilin, RME-8, and Aha1.

In some embodiments, the neurological therapeutic agent comprises a vaccine. As used herein, a vaccine is a composition that provides active acquired immunity to a particular disease, such as a neurodegenerative disease, e.g., Alzheimer's disease. A vaccine typically contains a protein or a peptide that may be disease specific (expressed exclusively by the diseased cell) or disease associated (expressed preferentially by the diseased cell). A vaccine can typically include an adjuvant. The vaccine stimulates the body's immune system to recognize the target and to eliminate or reduce the effect of the target. For example, a vaccine may be directed to amyloid-beta and stimulate the immune system to generate active acquired immunity, e.g., specific antibodies or T cells that recognize amyloid-beta and reduce or eliminate the formation of amyloid plaque. Vaccines can be prophylactic or therapeutic. A vaccine may also be a nucleic acid encoding a protein or a peptide or extract from diseased cells. Exemplary vaccines used in neurodegenerative diseases include, but are not limited to AN-1792. A vaccine may be a nucleic acid vaccine, e.g., DNA vaccine or RNA vaccine.

In certain embodiments, the neurological therapeutic agent is a nucleic acid or polynucleotide. The nucleic acids or polynucleotides of the invention may include deoxynucleotides, ribonucleotides, modified deoxynucleotides, modified ribonucleotides (e.g., chemical modifications, such as modifications that alter the backbone linkages, sugar molecules, and/or nucleic acid bases), and artificial nucleic acids. In some embodiments, the polynucleotide includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mPvNA, rPvNA, modified RNA, miRNA, gRNA, and siRNA or other RNA or DNA molecules.

In certain embodiments, the polynucleotide of the invention comprises a sequence that encodes a peptide or a protein, e.g., chaperone protein, antibody, or a protein or peptide used in a vaccine, disclosed herein. In some embodiment, the nucleic acid or polynucleotide is an inhibitory polynucleotide that inhibits the expression of a gene, e.g., the APP gene that encodes amyloid p protein, or the MAPT gene that encodes the Tau protein. The inhibitory polynucleotide may be RNAi, shRNA, siRNA, or antisense RNA.

The polynucleotides of the invention, e.g., protein coding nucleic acid, may be expressed from transcription units inserted into DNA or RNA vectors (see, e.g., Couture, A, et al., TIG. (1996), 12:5-10; WO 00/22113, WO 00/22114, and U.S. Pat. No. 6,054,299). In some embodiments, expression is sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of a polynucleotide encoding a neurological therapeutic agent comprising a nucleic acid molecule can be transcribed from a promoter in an expression vector. Expression vectors are generally DNA plasmids or viral vectors. Expression vectors compatible with eukaryotic cells, preferably those compatible with vertebrate cells, can be used to produce recombinant constructs for the expression of a neurological therapeutic agent as described herein. Delivery of the neurological therapeutic agent expressing vector can be systemic, such as by intravenous or intramuscular administration, by administration to target cells ex-planted from the patient followed by reintroduction into the patient, or by any other means that allows for introduction into a desired target cell, e.g., intracerebroventricularly (i.c.v.) or intra-cisterna magna (ICM).

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of a protein of the invention will generally require regulatory elements, e.g., promoters, enhancers, etc., to ensure the expression of the protein in target cells. Other aspects to consider for vectors and constructs are known in the art.

Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lenti viruses. In general, a suitable vector contains an origin of replication functional in at least one organism, a promoter sequence, convenient restriction endonuclease sites, and one or more selectable markers.

In some particular embodiments, the virus is adeno-associated viruses. AAV-mediated delivery of transgene is described elsewhere herein.

Expression of natural or synthetic nucleic acids is typically achieved by operably linking a nucleic acid encoding the gene of interest to a promoter, and incorporating the construct into an expression vector. The vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like.

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

The methods to deliver a polynucleotide or a nucleic acid to a cell are known in that art. The delivery of the nucleic acid neurological therapeutic agent of the invention to a cell e.g., a cell within a subject, such as a human subject (e.g., a subject in need thereof, such as a subject having a neurodegenerative disease, e.g., Alzheimer's disease) may be achieved in a number of different ways. For example, delivery may be performed by contacting a cell with a nucleic acid of the invention either in vitro, ex vivo, or in vivo. In vivo delivery may be performed directly by administering a composition comprising a peptide or protein neurological therapeutic agent to a subject. Alternatively, in vivo delivery may be performed indirectly by administering one or more vectors that encode and direct the expression of the neurological therapeutic agent. These alternatives are discussed further below.

In general, any method of delivery of a nucleic acid of the invention (in vitro, ex vivo, or in vivo) may be adapted for use with the nucleic acid of the invention (see e.g., Akhtar S. and Julian R L., (1992) Trends Cell. Biol. 2(5):139-144 and WO94/02595, which are incorporated herein by reference in their entireties). For in vivo delivery, factors to be considered for delivering a nucleic acid of the invention include, for example, biological stability of the neurological therapeutic agent, prevention of non-specific effects, and accumulation of the neurological therapeutic agent in the target tissue. The non-specific effects of a neurological therapeutic agent can be minimized by local administration, for example, by direct injection or implantation into a tissue or topically administering a composition comprising the neurological therapeutic agent. Local administration to a treatment site maximizes local concentration of the neurological therapeutic agent, limits the exposure of the neurological therapeutic agent to systemic tissues that can otherwise be harmed by the neurological therapeutic agent or that can degrade the neurological therapeutic agent, and permits a lower total dose of the neurological therapeutic agent to be administered.

For administering a nucleic acid of the invention systemically for the treatment of a disease, such as a neurodegenerative disease, the nucleic acid, e.g., a nucleic acid encoding an antibody specifically binding Tau protein, can be modified or alternatively delivered using a drug delivery system; both methods act to prevent the rapid degradation of the nucleic acid molecule by endo- and exo-nucleases in vivo. Modification of a nucleic acid molecule also permits targeting of the nucleic acid to a target tissue and avoidance of undesirable off-target effects. For example, a nucleic acid molecule of the invention may be modified by chemical conjugation to lipophilic groups such as cholesterol to enhance cellular uptake and prevent degradation.

Alternatively, a nucleic acid of the invention may be delivered using a drug delivery system such as a nanoparticle, a dendrimer, a polymer, a liposome, or a cationic delivery system. Positively charged cationic delivery systems facilitate binding of nucleic acid molecule (e.g., negatively charged molecule) and also enhance interactions at the negatively charged cell membrane to permit efficient uptake of a nucleic acid molecule by the cell. Cationic lipids, dendrimers, or polymers can either be bound to a nucleic acid, or induced to form a vesicle or micelle (see e.g., Kim SH. et al., (2008) Journal of Controlled Release 129(2):107-116) that encases the neurologic therapeutic agent. The formation of vesicles or micelles further prevents degradation of the neurological therapeutic agent when administered systemically. Methods for making and administering cationic complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of a nucleic acid of the invention include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a nucleic acid (e.g., DNA, or mRNA) forms a complex with cyclodextrin for systemic administration. Methods for administration and pharmaceutical compositions comprising cyclodextrins may be found in U.S. Pat. No. 7,427,605, the entire contents of which are incorporated herein by reference.

The nucleic acid of the invention may be incorporated into pharmaceutical compositions suitable for administration. Such compositions typically include one or more species of nucleic acid and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, subcutaneous, intraperitoneal or intramuscular injection, or intrathecal or intraventricular administration.

The route and site of administration may be chosen to enhance delivery or targeting of the nucleic acid of the invention to a particular location. For example, to target brain cells, intravenous, i.c.v, or ICM injection may be used.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added.

Compositions for administration may include sterile aqueous solutions which may also contain buffers, diluents, and other suitable additives. Formulations for may include sterile aqueous solutions which may also contain buffers, diluents, and other suitable additives. For intravenous use, the total concentration of solutes may be controlled to render the preparation isotonic.

In one embodiment, the administration of a nucleic acid of the invention is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider. The composition may be provided in measured doses or in a dispenser which delivers a metered dose. Selected modes of delivery are discussed in more detail below.

In certain embodiments, the neurological therapeutic agent comprises a cell. A variety of cells, e.g., immune cells (such as T cell specifically targeting amyloid plaque, B cell producing antibody specifically binding amyloid, T_{reg} cell reducing inflammatory reaction), may be used as a neurological therapeutic agent, including, fresh samples derived from subjects, primary cultured cells, immortalized cells, cell-lines, hybridomas, etc. The cells to be used as a neurological therapeutic agent may also include stem cells, such as embryonic stem cells, induced pluripotent stem cells, mobilized peripheral blood stem cells. The cells may be used for various therapeutic applications.

In some embodiments, the cell may be genetically engineered to express a neurological therapeutic agent, e.g., a protein, a peptide, an antibody, or an inhibitory RNA of the invention.

In some embodiments, the neurological therapeutic agent comprises an antibody. In certain embodiments, the antibody of the invention binds specifically to a peptide or a protein that forms pathological protein aggregate. Such a peptide or protein includes, but is not limited to amyloid precursor protein, amyloid beta, fibrin, tau, apolipoprotein E (Apoe), alpha-synuclein, TDP43, and huntingtin. For example, the neurological therapeutic agent can comprise, consist essentially of, or consist of an antibody selected from the group consisting of: bapineuzumab, gantenerumab, aducanumab, solanezumab, crenezumab, pepinemab, ozanezumab, lecanemab, ABT-099, AT-1501, BIIB054, and PRX002. For example, the neurological therapeutic agent can comprise, consist essentially of, or consist of an antibody that binds specifically to a protein associated with AD, for example an antibody (such as a human or humanized antibody) that binds specifically to amyloid beta, for example, bapineuzumab, gantenerumab, aducanumab, solanezumab, immunoglobulin, BAN2401, semorinemab, zagotenemab, and/or crenezumab. In the case of AD, example small molecules that inhibit aggregation include apomorphine and carbenoxolone. It will be appreciated that in methods, compositions, and uses as described herein, when a flow modulator and a neurological therapeutic agent are both administered to the subject, unless stated otherwise, the flow modulator will be understood to be a different molecule than the neurological therapeutic agent. In some embodiments, the neurological therapeutic agent comprises, consists essentially of, or consist of an antibody or binding fragment selected from the group consisting of bapineuzumab, gantenerumab, aducanumab, solanezumab, and crenezumab.

In some embodiments, if the target protein is Tau protein, exemplary antibodies include, but are not limited to, ABBV-8E12 (AbbVie), Gosuranemab (BIIB092, IPN007, Bristol-Myers Squibb), PHF1, MC1, DA31, 4E6G7, 6B2G12, TOMA, PHF6, PHF13, HJ9.3, HJ9.4, HJ8.5, 43D, 77E9, AT8, MAb86, pS404 mAb IgG2, pS409-tau, Armanezumab, PHF1, Ta9, Ta4, Ta1505, and DC8E8 (see, Jadhav et al., A walk through tau therapeutic strategies, Acta Neuropathologica Communications, 7:22, 1-31 (2019)). In some other embodiments, if the target protein is Tau protein, a vaccine (peptide) may be used as the neurological therapeutic agent. Exemplary vaccines include, but are not limited to, Tau 379-408, Tau 417-426, Tau 393-408, Tau 379-408, Tau 195-213, Tau 207-220, Tau 224-238, Tau aa 395-406, Human paired helical filaments (PHF's) Tau 229-237, Tau 199-208, Tau 209-217, Tau 294-305, and Tau 379-408 (see, Jadhav et al, supra).

In certain embodiments, if the target protein is fibrin, exemplary antibodies include, but are not limited to, 5B8 as described in Ryu et al., Fibrin-targeting immunotherapy protects against neuroinflmmation and neurodegeneration, Nature Immunology 19, 1212-1223 (2018).

In some embodiments, if the target protein is apolipoprotein E (Apoe), exemplary antibodies include, but are not limited to HAE4 as described in Liao et al., Targeting of nonlipidated, aggregated apoE with antibodies inhibits amyloid accumulation, J. of Clin. Invest., 128(5): 2144-2155.

In some embodiments, for example, if the proteinopathy is Huntington's disease, the antibody may be an antibody that binds specifically to semaphorin-4D, for example pepinemab, or an antibody that binds specifically to huntingtin, for example, the antibodies disclosed in US20170166631. In some embodiments, for example, if the proteinopathy is ALS, the antibody binds specifically to neurite outgrowth inhibitor A (e.g., ozanezumab) or to CD40L (e.g., AT-1501 (Anelixis)). In some embodiments, for example, if the proteinopathy is Parkinson's disease, the neurological therapeutic agent may be an antibody binds specifically to alpha-synuclein. Exemplary anti-alpha-synuclein antibodies include, but are not limited to, BIIB054 (Biogen), PRX002/RG7935 (Roche), prasinezumab (Roche), PD-1601 (AbbVie), 1H7, 5C1, A1-A6, 9E4, 274, NbSyn87*PEST, NAC32, NAC1, AC14, VH14*PEST, syn303, AB1, Human single-chain Fv D10, D5, syn-01, syn-O2, syn-04, mAb47, syn-10H, syn-F1, syn-F2, LS4-2G12 (see, Wang, et al., Progress of immunotherapy of anti-α-synuclein in Parkinson's disease, Biomedicine & Pharmacotherapy, 115: 108843 (2019)). In some embodiments, for example, if the proteinopathy is Parkinson's disease, the antibody may be cinpanemab, ABBV-0805 (AbbVie). In some embodiments, the neurological therapeutic agent comprises, consists essentially of, or consists of an antibody or binding fragment selected from the group consisting of bapineuzumab, gantenerumab, aducanumab, solanezumab, and crenezumab, pepinemab, ozanezumab, AT-1501, BIIB054, and PRX002.

In certain embodiments, for example, if the target protein is TDP43, exemplary antibodies include, but are not limited to, the antibodies disclosed in U.S. Pat. No. 10/202,443, U.S. Pat. No. 8,940,872, or Patent Publication Nos. WO2018218352, WO2019134981, (incorporated herein by reference), antibody 3B12A (disclosed in Scientific Reports, 8:6030 (2018), DOI:10.1038/s41598-018-24463-3).

In some embodiments, the antibody of the present invention includes bispecific antibodies of multispecific antibodies. Bispecific antibodies or multispecific antibodies include recognize more two or more epitopes. The two or more epitopes may be located on a same protein or on different proteins. Exemplary bispecific or multispecific antibodies include, but are not limited to bispecific monoclonal antibodies to inhibit BACE1 and MAPT for Alzheimer's Disease developed by Denali Therapeutic Inc.

The base structure of an antibody is a tetramer, which includes two heavy chains and two light chains. Each chain comprises a constant region, and a variable region. Generally, the variable region is responsible for binding specificity of the antibody. In a typical antibody, each variable region comprises three complementarity determining regions (CDRs) flanked by four framework regions. As such, a typical antibody variable region has six CDRs (three heavy chain CDRs, three light chain CDRs), some or all of which are generally involved in binding interactions by the antibody. The CDRs can be numbered according to an art-recognized method, for example the methodology of Kabat (Kabat, et al. in “Sequences of Proteins of Immunological Interest” Public Health Service, NIH, Washington D.C. (1987)), Chothia (Chothia and Lesk, J. Mol. Biol., 196, 901-917 (1987).), AbM (Martin et al., Proc. Natl. Acad. Sci. USA, 86:9268-9272 (1989)), contact definition (MacCallum et al., J. Mol. Biol., 262: 732-745 (1996)), or IMGT (Lefranc, “Unique database numbering system for immunogenetic analysis”, Immunology Today, 18: 509 (1997), LIGM:194). An antibody that “specifically” binds (or binds “specifically”) to an antigen (for example amyloid beta) has its ordinary and customary meaning as would be understood by one of ordinary skill in the art in view of this disclosure. It refers to the antibody preferentially binding to the antigen compared to one or more other substances. By way of example, an antibody that specifically binds to amyloid beta of some embodiments binds to amyloid beta with a numerically lower dissociation constant (K_{D}) than to other substances present in the CNS. In some embodiments, an antibody that specifically binds to amyloid beta binds with a K_{D} that is less than or equal to 1000 nM, 500 nM, 200 nM, 100 nM, 75 nM, 50 nM, 25 nM, 20 nM, 15 nM, 10 nM, 5 nM, 2 nM, 1 nM, 500 pM, 100 pM, 75 pM, 50 pM, 25 pM, 10 pm, or 5 pM, including ranges between any two of the listed values. A K_{D} for a particular antigen (for example, amyloid beta) can be determined, for example, by surface plasmon resonance, for example using a BIACORE apparatus.

A neurological therapeutic agent as in accordance with methods, compositions, and uses of embodiments herein can comprise, consist essentially of, or consist of any of a number of antibodies. Example monoclonal amyloid beta antibodies that can be used as a neurological therapeutic agents in accordance with embodiments herein include bapineuzumab, gantenerumab, aducanumab, solanezumab, and crenezumab. Solanezumab and crenezumab bind to a helix-beta coil epitope in the midsection of amyloid beta, while bapineuzumab, gantenerumab, and aducanumab bind to the N-terminal region of amyloid beta. In some embodiments, a neurological therapeutic agent comprises, consists essentially of, or consists of an antibody selected from the group consisting of bapineuzumab, gantenerumab, aducanumab, solanezumab, and crenezumab, or an antigen binding fragment of any of the listed antibodies. In some embodiments, a neurological therapeutic agent comprises, consists essentially of apomorphine or carbenoxolone. In some embodiments, a neurological therapeutic agent comprises, consists essentially of, or consists of an antibody selected from the group consisting of bapineuzumab, gantenerumab, aducanumab, solanezumab, and crenezumab, or an antigen binding fragment of any of the listed antibodies, or apomorphine or carbenoxolone. In some embodiments, a neurological therapeutic agent comprises, consists essentially of, or consists of an antibody or antigen binding fragment that comprises a heavy chain variable region and a light chain variable region of any one of bapineuzumab, gantenerumab, aducanumab, solanezumab, or crenezumab. In some embodiments, a neurological therapeutic agent comprises, consists essentially of, or consists of an antibody or antigen binding fragment that comprises a heavy chain variable region and a light chain variable region that are, respectively, at least 80%, 85%, 90%, 95%, 96%, 97%, 98%, or 99% identical to a heavy chain variable region and light chain variable of bapineuzumab, gantenerumab, aducanumab, solanezumab, or crenezumab. In some embodiments, a neurological therapeutic agent comprises, consists essentially of, or consists of an antibody or antigen binding fragment that comprises a light chain variable region that is at least 80%, 85%, 90%, 95%, 96%, 97%, 98%, or 99% identical to a light chain variable region of bapineuzumab, gantenerumab, aducanumab, solanezumab, or crenezumab. The antibody or antigen binding fragment can further comprise a heavy chain variable region of the noted antibody (bapineuzumab, gantenerumab, aducanumab, solanezumab, or crenezumab). In some embodiments, a neurological therapeutic agent comprises, consists essentially of, or consists of an antibody or antigen binding fragment that comprises a HCDR1, a HCDR2, and a HCDR3 of a heavy chain variable region, and a LCDR1, a LCDR2, and a LCDR3 of a light chain variable region of any one of bapineuzumab, gantenerumab, aducanumab, solanezumab, or crenezumab (that is, the antibody or antigen binding fragment comprises the noted six CDR's of any one of the listed antibodies). In some embodiments, the neurological therapeutic agent comprises, consists essentially of, or consist of an antibody selected from the group consisting of bapineuzumab, gantenerumab, aducanumab, solanezumab, and crenezumab.

In some embodiments, the neurological therapeutic agent comprises a chimeric antigen receptor T-cell (CAR-T cell) that binds specifically to a protein associated with the proteinopathy of the patient. In some embodiments, for example if the neurological disease or disorder comprises a tauopathy and/or an amyloidosis such as AD, the neurological therapeutic agent comprises a chimeric antigen receptor T-cell (CAR-T cell) that binds specifically to an amyloid beta protein. For example, the CAR-T cell can comprise a chimeric antigen receptor comprising a heavy chain variable region and a light chain variable region of any of the antibodies described herein. For example, the CAR-T cell can comprise a chimeric antigen receptor comprising a HCDR1, HCDR2, HCDR3, LCDR1, LCDR2, and LCDR3 of any of the antibodies described herein.

A number of approaches are available for producing suitable antibodies that specifically bind to a target peptide or protein, e.g., amyloid beta, α-synuclein, fibrin, tau, apolipoprotein E (Apoe), or TDP43, in accordance with methods and uses of embodiments herein. For example, in some embodiments, a host organism is immunized with an antigen comprising, consisting essentially of, or consisting of an amyloid beta, for example amyloid precursor protein (APP) or a fragment thereof. By way of example, a sequence of APP is available as Uniprot accession no. P56199 (SEQ ID NO: 5

MLPGLALLLLAAWTARALEVPTDGNAGLLAEPQIAMFCGRLNMHMNVQNG
KWDSDPSGTKTCIDTKEGILQYCQEVYPELQITNVVEANQPVTIQNWCKR
GRKQCKTHPHFVIPYRCLVGEFVSDALLVPDKCKFLHQERMDVCETHLHW
HTVAKETCSEKSTNLHDYGMLLPCGIDKFRGVEFVCCPLAEESDNVDSAD
AEEDDSDVWWGGADTDYADGSEDKVVEVAEEEEVAEVEEEEADDDEDDED
GDEVEEEAEEPYEEATERTTSIATTTTTTTESVEEVVREVCSEQAETGPC
RAMISRWYFDVTEGKCAPFFYGGCGGNRNNFDTEEYCMAVCGSAMSQSLL
KTTQEPLARDPVKLPTTAASTPDAVDKYLETPGDENEHAHFQKAKERLEA
KHRERMSQVMREWEEAERQAKNLPKADKKAVIQHFQEKVESLEQEAANER
QQLVETHMARVEAMLNDRRRLALENYITALQAVPPRPRHVFNMLKKYVRA
EQKDRQHTLKHFEHVRMVDPKKAAQIRSQVMTHLRVIYERMNQSLSLLYN
VPAVAEEIQDEVDELLQKEQNYSDDVLANMISEPRISYGNDALMPSLTET
KTTVELLPVNGEFSLDDLQPWHSFGADSVPANTENEVEPVDARPAADRGL
TTRPGSGLTNIKTEEISEVKMDAEFRHDSGYEVHHQKLVFFAEDVGSNKG
AIIGLMVGGVVIATVIVITLVMLKKKQYTSIHHGVVEVDAAVTPEERHLS
KMQQNGYENPTYKFFEQMQN).

By way of example, a polypeptide comprising, consisting essentially of, or consisting of the amino acid sequence of SEQ ID NO: 5 sequence can be used to immunize a host in order to produce antibodies that bind specifically to amyloid beta in accordance with some embodiments. The host organism can be a non-human mammal such as a mouse, rat, guinea pig, rabbit, donkey, goat, or sheep. Isolated antibody-producing cells can be obtained from the host organism, and the cells (or antibody-encoding nucleic acids thereof) can be screened for antibodies that binds specifically to amyloid beta. In some embodiments, antibody-producing cells are immortalized using hybridoma technology, and the resultant hybridomas are screened for antibodies that bind specifically to amyloid beta. In some embodiments, antibody-encoding nucleic acids are isolated from antibody-producing cells, and screened for antibodies that bind specifically to amyloid beta. An example protocol for screening human B cell nucleic acids is described in Huse et al., Science 246:1275-1281 (1989), which is hereby incorporated by reference in its entirety. In some embodiments, nucleic acids of interest are identified using phage display technology (See, e.g., Dower et al., WO 91/17271 and McCafferty et al., WO 92/01047, each of which is hereby incorporated by reference in its entirety). Phage display technology can also be used to mutagenize variable regions (or portions thereof such as CDRs) of antibodies previously shown to have affinity for amyloid beta. Variant antibodies can then be screened by phage display for antibodies having desired affinity to amyloid beta.

In some embodiments, the antibody that specifically binds to amyloid beta is formatted as an antigen binding fragment (which may be referred to herein simply as a “binding fragment”). Example antigen binding fragments suitable for methods and uses of some embodiments can comprise, consist essentially of, or consist of a construct selected from the group consisting of Fab, Fab′, Fab′-SH, F(ab′)₂, and Fv fragments; Fd fragment; minimal recognition units consisting of the amino acid residues that mimic the hypervariable region of an antibody (e.g., an isolated complementarity determining region (CDR) such as a CDR3 peptide), or a constrained FR3-CDR3-FR4 peptide; minibodies; diabodies; and single-chain fragments such as single-chain Fv (scFv) molecules. Bispecific or multispecific antibodies or antigen binding fragments are also contemplated in accordance with methods and uses of some embodiments. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. Unless stated otherwise, wherever an “antibody” is mentioned herein, a binding fragment of that antibody is also contemplated. In some embodiments, the amyloid beta antibody is chimeric, human, or humanized. In some embodiments, the amyloid beta antibody is human, or humanized.

In some embodiments, for example if human monoclonal antibodies are of interest, the host comprises genetic modifications to produce or facilitate the production of human immunoglobulins. For example, XenoMouse™ mice were engineered with fragments of the human heavy chain locus and kappa light chain locus, respectively, which contained core variable and constant region sequences (described in detail Green et al. Nature Genetics 7:13-21 (1994), which is hereby incorporated by reference in its entirety). For example, mice have been engineered to produce antibodies comprising a human variable regions and mouse constant regions. The human heavy chain and light chain variable regions can then be reformatted onto a human constant region to provide a fully human antibody (described in detail in U.S. Pat. No. 6,787,637, which is hereby incorporated by reference in its entirety), For example, in a “minilocus” approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal such as a mouse (See, e.g., U.S. Pat. No. 5,545,807, which is hereby incorporated by reference in its entirety). Another approach, includes reconstituting SCID mice with human lymphatic cells, e.g., B and/or T cells. The mice are then immunized with an antigen and can generate an immune response against the antigen (See, e.g., U.S. Pat. No. 5,476,996, which is hereby incorporated by reference in its entirety).

In some embodiments, a host monoclonal antibody is formatted as a chimeric antibody or is humanized, so that the antibody comprises at least some human sequences. By way of example, By way of example, an approach for producing humanized antibodies can comprise CDR grafting. For example, an antigen can be delivered to a non-human host (for example a mouse), so that the host produces antibody against the antigen. In some embodiments, monoclonal antibody is generated using hybridoma technology. In some embodiments, V gene utilization in a single antibody producing cell of the host is determined. The CDR's of the host antibody can be grafted onto a human framework. The V genes utilized in the non-human antibody can be compared to a database of human V genes, and the human V genes with the highest homology can be selected, and incorporated into a human variable region framework. See, e.g., Queen, U.S. Pat. No. 5,585,089, which is hereby incorporated by reference in its entirety.

Isolated oligonucleotides encoding an antibody of interest can be expressed in an expression system, such as a cellular expression system or a cell-free system in order to produce an antibody that binds specifically to amyloid beta in accordance with methods and uses of embodiments herein. Exemplary cellular expression systems include yeast (e.g., mammalian cells such as CHO cells or BHK cells, E. coli, insect cells, Saccharomyces, Pichia) transformed with recombinant yeast expression vectors containing the nucleotide sequences encoding antibodies; insect cell systems infected with recombinant virus expression vectors (e.g., baculovirus) containing sequences encoding antibodies; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing nucleotide sequences encoding antibodies; mammalian cell systems (e.g., COS, CHO, BHK, 293, 3T3) harboring recombinant expression constructs containing promoters derived from the genome of mammalian cells (e.g., metallothionein promoter) or from mammalian viruses.

In certain embodiments, a neurological therapeutic agent of the invention may be used in combination with one or more different neurological therapeutic agents of the invention. For example, a small molecule neurological therapeutic agent may be used in combination with an antibody therapeutic agent. Different neurological therapeutic agents may be formulated in a same pharmaceutical composition or in different pharmaceutical compositions. Different neurological therapeutic agents may be administered concurrently or sequentially.

Neurological Diseases

Methods, uses, and compositions in accordance with some embodiments herein can be useful for diagnosing, treating, preventing, inhibiting, ameliorating, or reducing the symptoms of one or more neurological diseases, or compositions for use in these methods. In some embodiments, the neurological disease is a proteinopathy. In some embodiments, the neurological disease comprises a proteinopathy as described herein (e.g., a tauopathy and/or amyloidosis such as AD). Such diseases can occur in subjects, for example humans, as well as non-human animals, such as non-human mammals, and non-human primates in some embodiments.

In some embodiments, a neurological disease such as a neurodegenerative, neurodevelopmental, neuroinflammatory, or neuropsychiatric disease associated with accumulation of macromolecules, cells, and debris in the CNS is treated, prevented, inhibited, or reduced by methods, uses, or compositions that increase flow, drainage, and/or clearance in meningeal lymphatic vessels. In some embodiments, neurodegenerative, neurodevelopmental, neuroinflammatory, or neuropsychiatric diseases associated with accumulation of macromolecules, cells, and debris in the CNS are treated, prevented, inhibited, or reduced by methods, uses, or compositions that counteract the effects (e.g., changes in the hippocampal transcriptome) of decreased flow with or without restoring flow. In some embodiments, neurological diseases associated with accumulation of macromolecules, cells, and debris in the CNS are treated, prevented, inhibited, or reduced. Examples of neurological diseases include proteinopathies, for example tauopathies and/or amyloidosis such as AD (e.g., familial AD and/or sporadic AD). Examples of neurological diseases include AD (such as familial AD and/or sporadic AD), dementia, age-related dementia, PD, cerebral edema, ALS, PANDAS, meningitis, hemorrhagic stroke, ASD, epilepsy, Down's syndrome, HCHWA-D, Familial Danish/British dementia, DLB, LB variant of AD, MSA, FENIB, FTD, HD, Kennedy disease/SBMA, DRPLA; SCA type I, SCA2, SCA3 (Machado-Joseph disease), SCA6, SCA7, SCA17, CJD (such as familial CJD), Kuru, GSS, FFI, CBD, PSP, CAA, AIDS-related dementia complex, or a combination of two or more of the listed items. By way of example, neurological diseases can include AD, dementia, age-related dementia, PD, cerebral edema, ALS, PANDAS, meningitis, hemorrhagic stroke, ASD, and epilepsy. In some embodiments, the neurological disease comprises, consists essentially of, or consists of a proteinopathy, for example AD (such as familial AD and/or sporadic AD), Down's syndrome, HCHWA-D, Familial Danish/British dementia, PD, DLB, LB variant of AD, MSA, FENIB, ALS, FTD, HD, Kennedy disease/SBMA, DRPLA; SCA type I, SCA2, SCA3 (Machado-Joseph disease), SCA6, SCA7, SCA17, CJD (such as familial CJD), Kuru, GSS, FFI, CBD, PSP, CAA, AIDS-related dementia complex, or a combination of two or more of any of the listed items. In some embodiments, neurological diseases associated with amyloid beta for example an amyloidosis such as AD (e.g., familial AD and/or sporadic AD) in the CNS are treated, prevented, inhibited, or reduced by methods, uses, or compositions that counteract the effects of decreased flow with or without restoring flow.

In some embodiments, the neurological disease, for example a proteinopathy (such as a tauopathy and/or an amyloidosis, e.g., AD) can be prevented, treated, or ameliorated prophylactically. Accordingly, a subject having one or more risk factors for the neurological disease can be determined to be in need of receiving a method, use, or composition described herein. For example, a subject may have accumulated amyloid beta plaques in their CNS, and may benefit from increased flow, increased drainage, increased clearance and/or reduction of amyloid beta plaques, even if they do not yet have an neurological disease diagnosis based on cognitive symptoms.

A number of risk factors for AD are suitable as risk factors in accordance with methods, compositions, and uses of some embodiments herein, for example familial AD, a genetic marker for AD, or a symptom of AD such as early dementia. The foremost risk factor for sporadic AD is age. However, increased risk of this form of AD has also been attributed to diverse genetic abnormalities. One of them is diploidy for apolipoprotein-Eε4 (Apo-Eε4), widely viewed as a major genetic risk factor promoting both early onset of amyloid beta aggregation and defective amyloid beta clearance from the brain (Deane et al., 2008; Zlokovic, 2013). Other genetic variants that significantly increase the risk for sporadic AD are Apo-J (or clusterin), phosphatidylinositol-binding clathrin assembly protein (PICALM), complement receptor 1 (CR1), CD33 or Siglec-3, and triggering receptor expressed on myeloid cells 2 (TREM2). All of these proteins, interestingly, have been implicated in different mechanisms of amyloid beta removal from the brain (Bertram et al., 2008; Guerreiro et al., 2013; Harold et al., 2009; Lambert et al., 2009, 2013; Naj et al., 2011). In some embodiments, the risk factor for AD is selected from the group consisting of at least one of the following: diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), age, or a symptom of dementia.

Methods of Identifying a Subject Having Enhanced Risk of Developing Neurological Diseases

In one aspect, the invention is based upon, at least in part, the surprising discovery that a subject has degeneration of lymphatic vasculature in the central nervous system of the subject prior to the onset of the neurological disease. The term “degeneration of lymphatic vasculature,” as used herein, refers to the reduction or loss or lymphatic vessel coverage (in area) in the central nervous system. The reduced coverage may be cause by the reduced length, the diameter, and/or branching point of lymphatic vessels. In certain embodiments, the degeneration of lymphatic vessel occurs at the superior sagittal sinus, dural confluence of sinuses, the transverse (TS), sigmoid (SS), or petrosquamosal (PSS) sinuses.

Accordingly, in some embodiments, the invention provides a method of identifying a subject that has an enhanced risk of developing neurological disease prior to the onset of the neurological disease. The term “enhanced risk,” as used herein, refers to a higher probability to develop certain neurological diseases, e.g., Alzheimer's disease, as compared to a reference probability (reference risk). The reference risk is the probability of developing such a neurological disease in general population. The method includes detecting the degeneration of lymphatic vasculature in the central nervous system of the subject. Any methods that can be used to detect the degeneration of the lymphatic vasculature in the central nervous system are encompassed in this invention. In certain embodiments, the detection method is a non-invasive detection method that visualizes the lymphatic vasculature of the central nervous system. Exemplary non-invasive detection method includes magnetic resonance imaging as described in Abstinta et al., Human and nonhuman primate meninges harbor lymphatic vessels that can be visualized noninvasively by MRI, eLife 2017: 6: e29738, DOI: 10.7554, incorporated herein by reference. To use MRI to visualize the lymphatic vessels in the central nervous system, a magnetic dye is administered to the subject. The magnetic dye has molecules that are small enough to leak out of blood vessels in the dura into lymphatic vessels, but too big to pass through the blood-brain barrier and enter other parts of the brain. By adjusting the parameters of the MRI, the lymphatic vessels of the central nervous system can be specifically visualized.

In certain embodiments, the lymphatic vasculature can be visualized using in vivo fluorescence imaging method. Exemplary fluorescence imaging in human was described in Piper et al., Toward whole-body fluorescence imaging in humans, PLoS One, 2013; 8(12): e83749, incorporated herein by reference.

The degeneration of lymphatic vasculature may be reflected in the decrease of lymphatic coverage by about 10%, about 20%, about 30%, about 40%, about 50%, about 60%, about 70%, about 80%, about 90%, about 99%, or between about 10% to about 99%.

In one aspect, this invention is based upon, at least in part, that the gene expression profile in lymphatic endothelia cells or immune cells in the central nervous system is altered in a subject that has an enhanced risk of developing a neurological disease, e.g., Alzheimer's disease. In certain embodiments, the alteration in the gene expression profile is the change of gene expression level of one or more genes in Tables 2-29.

Accordingly, in some embodiments, the present invention provides a method of identifying a subject that has an enhanced risk of developing a neurological disease, e.g., Alzheimer's disease, prior to the onset of the neurological disease. The method includes detecting the alteration of gene expression level in one or more genes in Table 2-29 in lymphatic endothelial cells (LECs) or immune cells in central nervous system. The immune cells may be from the meninges or brain cortices. The gene expression level is “altered,” e.g., increased or depressed as compared to a reference gene expression level. The reference gene expression level may be the gene expression level of a healthy subject who is known to have the reference risk of developing a neurologic disease, e.g., Alzheimer's disease, or the average gene expression level in a general population.

LECs or immune cells from central nervous system may be obtained from biopsy from deep cervical lymph nodes. Single cell RNA sequence may be then performed on the cells to determine the alteration in the gene expression level.

In some embodiments, the expression level of certain genes may be reduced by about 10%, about 20%, about 30%, about 40%, about 50%, about 60%, about 70%, about 80%, about 90%, about 99%, or between about 10% to about 99%. In some other embodiments, the expression level of certain genes may be increased by about 10%, about 20%, about 30%, about 40%, about 50%, about 60%, about 70%, about 80%, about 90%, about 99%, between about 10% to about 99%, about one fold, about two fold, about 4 fold, about 8 fold, about 16 fold, about 32 fold, about 50 fold, about 100 fold, or about one to about 100 fold, or more than about 100 fold.

In one aspect, this invention is based upon, at least in part, that a subject with enhanced risk of developing a neurological disease, e.g., Alzheimer's disease, has increased number of immune cell in the central nervous system.

Accordingly, in some embodiments, the present invention provides a method of identifying a subject that has an enhanced risk of developing a neurological disease, e.g., Alzheimer's disease, prior to the onset of the neurological disease. The method includes detecting the increase in the number of immune cells in the central nervous system. The number of immune cells increases if more immune cells are identified in the central nervous system as compared to a reference number of immune cells. The reference number of immune cells is a number of immune cells in the central nervous system of a healthy subject who is known to have a reference risk or the number of immune cells in the central nervous system in a general population.

In certain embodiments, the immune cells are CD45^{high} microglia or recruited lymphocytes. In some embodiments, the immune cells are CD45^{int} microglia or recruited lymphocytes that express H-2KD. In some other embodiments, the immune cells are selected from the group consisting of B cells, CD4⁺ T cells, CD8⁺ T cells, and type 3 innate lymphoid cells (ILC3s).

In some embodiments, the number of immune cells in a subject's central nervous system may be determined by in vivo fluorescence imaging. For example, an antibody specific to a cell surface protein may be conjugated to a fluorescence entity. The antibody-fluorescence entity complex may be administered to a subject. The fluorescence density may reflect the number of the immune cells.

In some other embodiments, the number of immune cells may be increased by about 10%, about 20%, about 30%, about 40%, about 50%, about 60%, about 70%, about 80%, about 90%, about 99%, between about 10% to about 99%, about one fold, about two fold, about 4 fold, about 8 fold, about 16 fold, about 32 fold, about 50 fold, about 100 fold, or about one to about 100 fold, or more than about 100 fold.

In some aspect, the present invention is based upon, at least in part, the discovery that certain genes containing neurological disease-associated Single Nucleotide Polymorphism (SNP) are highly expressed in lymphatic endothelial cells. Accordingly, the present invention provides a method of identifying a subject that has an enhanced risk of developing a neurological disease. The method includes detecting one or more single nucleotide polymorphisms as listed in Table 2-29.

In one aspect, the subject is a human subject. The human subject may be about 20 years old, about 30 years old, about 40 years old, about 50 years old, about 60 years old, about 70 years old, about 80 years old, about 90 years old, about 100 years old, or any age between about 20 and about 100 years old. In some embodiments, the human subject is previously known to have an enhanced risk of developing a neurologic disease, e.g., Alzheimer's disease. Such an enhanced risk may be evaluated by investigating the family history of the subject or by genetic screening.

Methods of Reducing Risk of Developing Neurological Disease

In one aspect, the present invention is based upon, at least in part, the surprising discovery that administration of a neurological therapeutic agent, e.g., an antibody against Aβ, prior to the onset of a neurological disease, e.g., Alzheimer's disease, can reduce the risk of developing such neurological disease. Accordingly, the present invention provides a method of reducing the risk, or delaying the onset of a neurological disease. The method includes administering to a subject an effective amount of a neurological therapeutic agent according to the present invention. In some embodiment, the subject is identified to have an enhanced risk of developing the neurological disease according to any method disclosed herein.

In certain embodiments, the method of reducing the risk, or delaying the onset of a neurological disease further include administering to the subject an effective amount of flow modulator according to the present invention.

Methods, Compositions, or Uses for Increasing Flow

Some embodiments include methods of, compositions for use, or uses for increasing flow in fluid in the central nervous system of a subject, or compositions for use in these methods. It noted that in some embodiments, the components of any of the noted compositions can be provided separately as “product combinations” in which the components are provided in two or more precursor compositions, which can either be combined to form the final composition (e.g., mix a flow modulator with a neurological disease therapeutic agent to arrive at a final composition comprising the flow modulator neurological disease therapeutic agent), or used in conjunction to achieve an effect similar to the single composition (e.g., administer a flow modulator and neurological disease therapeutic agent to a subject simultaneously or sequentially). Some embodiments include a composition or product combination comprising a flow modulator (e.g., VEGFR3 agonist and/or FGF), and a neurological disease therapeutic agent. The neurological disease therapeutic agent can be different from the flow modulator. The composition can be for medical use, for example, for use in treating, preventing, or ameliorating the symptoms of a neurological disease, for example a proteinopathy as described herein (e.g., a tauopathy and/or amyloidosis such as AD). The methods or uses can include determining whether the subject is in need of increased fluid flow in the central nervous system. If the subject is in need of increased fluid flow, the method or use can include administering an effective amount of flow modulator (such as a VEGFR3 agonist and/or FGF2) to a meningeal space of the subject and administering a neurological therapeutic agent to the subject (for example, to the CNS, such as the meningeal space). The flow modulator (e.g., VEGFR3 agonist and/or FGF2) and neurological therapeutic agent can be administered in the same composition, or in separate compositions as described herein. Without being limited by theory, the amount of flow modulator (e.g., VEGFR3 agonist and/or FGF2) can increase flow for example, by increasing the diameter of a meningeal lymphatic vessel of the subject, by increasing the quantity of meningeal lymphatic vessels of the subject, and/or by increasing drainage through meningeal lymphatic vessels of the subject. Thus, fluid flow in the central nervous system of the subject can be increased. Further, the neurological therapeutic agent can treat, inhibit, ameliorate, reduce the symptoms of, reduce the likelihood of, or prevent the neurological disease. In some embodiments, the neurological therapeutic agent (e.g., amyloid beta antibody) synergizes with the flow modulator (e.g., VEGFR3 agonist and/or FGF2). The synergy can comprise greater clearance of protein deposits (e.g., amyloid deposits) than either the neurological therapeutic agent or flow modulator on its own, for example at least 5%, 10%, 15%, 20%, 25%, 30%, 40%, 57%, 60%, 70%, 80%, or 90% less amyloid plaque density, and/or at least 5%, 10%, 15%, 20%, 25%, 30%, 40%, 57%, 60%, 70%, 80%, 90% lower amyloid plaque size compared to either the neurological therapeutic agent or flow modulator alone. In some embodiments, the synergy comprises enhancement of memory, and/or delay in the onset or progression of dementia. In some embodiments, the fluid comprises cerebral spinal fluid (CSF), interstitial fluid (ISF), or both. In some embodiments, the VEGFR3 agonist comprises, consists essentially of, or consists of VEGF-c or VEGF-d or an analog, variant, or fragment thereof. It is also contemplated that for compositions and methods and uses in some embodiments herein, FGF2 can be substituted for the indicated VEGFR3 agonist in order to increase flow, or can be used in addition to a VEGFR3 agonist in order to increase flow.

Such methods of, compositions for, or use for increasing fluid flow in the CNS can be useful for treating, preventing, or ameliorating the symptoms of neurological diseases associated with the increased concentration and/or accumulation of molecules, cells. or debris in the CNS (e.g., protein deposits such as amyloid deposits), for example in a neurological disease, for example a proteinopathy such as a tauopathy and/or amyloidosis (e.g., AD). Accordingly, in some embodiments, a subject can be determined to be in need of increased fluid flow by determining whether the subject has a neurological disease, or is at risk of developing a neurological disease. The disease can be associated with the increased concentrations and/or accumulation of molecules or cells or debris in the CNS, for example a proteinopathy such as a tauopathy and/or amyloidosis (e.g., AD). In some embodiments, the subject can be determined to be at risk for the disease, for example through having familial occurrence of the disease, by having one or more genetic or protein or metabolite markers associated with the disease, through advanced age, or by exhibiting symptoms of the disease, for example early dementia in the case of AD. As used herein, “advanced age” refers to an age characterized by a decrease in memory function, decrease in CSF production, substantial increases in neuronal senescence, and in the context of some embodiments, can include at least 65 years of age in a human, for example, at least 60, 65, 70, 75, 80, or 85, including ranges between any of these values. In some embodiments, determining whether the subject is in need of increased fluid flow comprises determining the subject to have a neurological disease, for example a proteinopathy such as a tauopathy and/or amyloidosis (e.g., AD). In some embodiments, determining whether the subject is in need of increased fluid flow comprises determining the subject to have a risk factor for the neurological disease associated with the increased concentration and/or accumulation of molecules or macromolecules or cells or debris in the CNS as described herein. In some embodiments, determining whether the subject is in need of increased fluid flow comprises determining the subject to have a risk factor, and also determining the subject to have the disease itself. In some embodiments, the neurological disease is Alzheimer's disease, and the risk factor is a risk factor for Alzheimer's disease as described herein. In some embodiments, the flow modulator (e.g., VEGFR3 agonist and/or FGF2) is administered to the subject after determining that the subject has a risk factor for the neurological disease (even if the subject does not necessarily have the disease itself), for example for prophylactic treatment or prevention. In some embodiments, the flow modulator (e.g., VEGFR3 agonist and/or FGF2) is administered to the subject after determining that the subject has the neurological disease.

Without being limited by theory, it is contemplated, according to several embodiments herein, that systemic administration is not required for the flow modulator (e.g., VEGFR3 agonist and/or FGF2) to effectively modulate meningeal lymphatic vessel size and drainage, or flow, and/or for the combination of the neurological therapeutic agent and flow modulator to inhibit, treat, reduce the likelihood of, delay the onset of, prevent, or ameliorate symptoms of the neurological disease. Accordingly, in some embodiments, the flow modulator (e.g., VEGFR3 agonist and/or FGF2), and/or the neurological therapeutic agent is administered selectively to the meningeal space of the subject. In the method, use, or composition of some embodiments, the flow modulator (e.g., VEGFR3 agonist and/or FGF2) is administered to the meningeal space, and the neurological therapeutic agent is administered to the subject. The neurological therapeutic agent may be administered to the meningeal space, or to a different location, for example, subcutaneously, intravenously, parenterally, orally, by inhalation, transdermally, or by rectal administration. In the method, use, or composition of some embodiments, the flow modulator (e.g., VEGFR3 agonist and/or FGF2), and/or the neurological therapeutic agent is administered to the meningeal space, but is not administered outside the CNS. In the method, use, or composition of some embodiments, the flow modulator (e.g., VEGFR3 agonist and/or FGF2), and/or the neurological therapeutic agent is administered to the meningeal space, but is not administered to the blood. In the method, use, or composition of some embodiments, the flow modulator (e.g., VEGFR3 agonist and/or FGF2), and/or the neurological therapeutic agent is administered to the subject by a route selected from the group consisting of at least one of the following: nasal administration, transcranial administration, contact with cerebral spinal fluid (CSF) of the subject, pumping into CSF of the subject, implantation into the skull or brain, contacting a thinned skull or skull portion of the subject with the VEGFR3 agonist and/or FGF2 and/or the neurological therapeutic agent, or expression in the subject of a nucleic acid encoding the VEGFR3 agonist and/or FGF2 and/or the neurological therapeutic agent, or a combination of any of the listed routes. In some embodiments, it is the VEGFR3 agonist that is administered. In the method, use, or composition of some embodiments, the VEGFR3 agonist is selected from the group consisting of at least one of the following: VEGF-c, VEGF-d, or an analog, variant, or functional fragment thereof. In the method, use, or composition of some embodiments, the neurological therapeutic agent comprises, consists essentially of, or consists of an amyloid beta antibody. In the method, use, or composition of some embodiments, the neurological therapeutic agent comprises, consists essentially of, or consists of an amyloid beta antibody.

In the method, use, or composition of some embodiments, the administration of the flow modulator (VEGFR3 agonist such as VEGF-c, and/or FGF2) and the neurological therapeutic agent (e.g., amyloid beta antibody) results in an increase in CNS fluid flow, meningeal lymphatic vessel diameter, meningeal lymphatic vessel number, meningeal lymphatic vessel drainage, or amelioration of symptoms of a neurological disease. For example, in some embodiments, the administration of the flow modulator (e.g., VEGFR3 agonist and/or FGF2) and the neurological therapeutic agent increases diameter of the meningeal lymphatic vessel is increased by at least about 5%, for example at least about 5%, 10%, 15%, 20%, 25%, 30%, 35%, 40%, 45% or 50%, including ranges between any two of the listed values. In some embodiments, an average diameter of a population of meningeal lymphatic vessels of the subject is increased by a value noted herein. In some embodiments, the administration of the flow modulator (e.g., VEGFR3 agonist and/or FGF2) and the neurological therapeutic agent increases fluid flow in the central nervous system of the subject, comprising increasing a rate of perfusion of fluid throughout an area of the subject's brain. In some embodiments, for example if the subject has AD, the administration of the flow modulator (VEGFR3 agonist such as VEGF-c, and/or FGF2), and neurological therapeutic agent (e.g., amyloid beta antibody) increases the ISF flow and reduces the quantity and/or average size of amyloid beta plaques in the subject's CNS. For example, the quantity of accumulated amyloid beta plaques can be reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values. For example, the average size of accumulated amyloid beta plaques can be reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values. It is shown herein that some brains of humans with AD have structures resembling amyloid beta plaques in the meninges. Accordingly, in some embodiments, at least some of the accumulated amyloid beta plaques are in the meninges of the subject's brain. In some embodiments, administering the combination of the flow modulator (VEGFR3 agonist such as VEGF-c, and/or FGF2), and the neurological therapeutic agent (e.g., amyloid beta antibody) increases clearance of soluble molecules in the brain of the subject. Clearance of soluble molecules can be ascertained, for example, by monitoring the retention of a particular compound, molecule, or label over an area of the brain over a particular period of time. In some embodiments, administering the combination of the FGF2 or VEFR3 agonist (e.g., VEGF-c) and the neurological therapeutic agent (e.g., amyloid beta antibody) increases clearance of soluble molecules in the brain of the subject by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values.

Methods, Compositions, and Uses for Reducing Protein Aggregates

Some embodiments include methods, compositions for use, and uses for reducing a quantity of protein aggregates, such as amyloid beta, fibrin, tau, or alpha-synuclein aggregates. In one embodiment, the protein aggregate comprises amyloid beta plaques. Accordingly, the present invention provides compositions and methods for reducing accumulated amyloid beta plaques, or decreasing the rate of accumulation of amyloid beta plaques, in a subject having a neurological disease or a risk factor for such a disease, or compositions for use in such methods.

The methods or uses can include determining the subject to have the neurological disease or the risk factor. The methods or uses can include administering a flow modulator (e.g., VEGFR3 agonist and/or FGF2) to a meningeal space of the subject, so that fluid flow (e.g., flow of ISF, CSF, or both) in the central nervous system of the subject is increased, and further administering and a neurological therapeutic agent to the subject (the neurological therapeutic agent can be administered to a meningeal space or to a different location). Through increased fluid flow, the quantity of accumulated amyloid beta plaques in the subject can be reduced, or the rate of accumulation can be reduced. By way of example, the VEGFR3 agonist can comprise (or consist essentially of, or consist of) VEGF-c, and the neurological therapeutic agent can comprise (or consist essentially of, or consist of) an amyloid beta antibody. In some embodiments, at least some of the accumulated amyloid beta plaques are in the meninges of the subject's brain. In some embodiments, the quantity of accumulated amyloid beta plaques, the average size of the accumulated amyloid beta plaques, and/or the rate of accumulation, is reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values. In some embodiments, the flow modulator (e.g., VEGFR3 agonist and/or FGF2) and/or the neurological therapeutic agent is administered selectively to the meningeal space. In some embodiments, the flow modulator (e.g., VEGFR3 agonist and/or FGF2) and/or the neurological therapeutic agent is administered to the CNS, but not outside the CNS. In some embodiments, the flow modulator (e.g., VEGFR3 agonist and/or FGF2) and/or the neurological therapeutic agent is administered to the CNS, but not blood. In some embodiments, the VEGFR3 agonist is selected from the group consisting of at least one of the following: VEGF-c, VEGF-d, or an analog, variant, or functional fragment thereof. In some embodiments, the neurological therapeutic agent comprises, consists essentially of, or consists of an amyloid beta antibody.

In some embodiments, administering the flow modulator (e.g., VEGFR3 agonist and/or FGF2) and/or the neurological therapeutic agent increases the diameter of a meningeal lymphatic vessel of the subject's brain by at least 2%, for example at least 2%, 3%, 4%, 5%, 6%, 7%, 8%, 9%, 10%, 15%, 20%, 25%, 30%, 35%, 40%, 45%, or 50%, including ranges between any two of the listed values, thus increasing flow in ISF. As noted herein, increased fluid flow in the central nervous system of the subject comprises an increased rate of perfusion of fluid throughout an area of the subject's brain. For example, increased fluid flow in the central nervous system of the subject can comprise an increased rate of perfusion out of the subject's central nervous system.

In some embodiments, the subject is known to have the neurological disease, for example AD (such as familial AD and/or sporadic AD), Down's syndrome, HCHWA-D, Familial Danish/British dementia, PD, DLB, LB variant of AD, MSA, FENIB, ALS, FTD, HD, Kennedy disease/SBMA, DRPLA; SCA type I, SCA2, SCA3 (Machado-Joseph disease), SCA6, SCA7, SCA17, CID (such as familial CID), Kuru, GSS, FFI, CBD, PSP, CAA, or a combination of two or more of any of the listed items. By way of example, the neurological disease can comprise a proteinopathy. In some embodiments, the method further includes determining that the subject has the neurological disease. In some embodiments, for example if the method or use is prophylactic, the method comprises determining whether the subject has the risk factor for the neurological disease, even if the subject does not necessarily have a diagnosis for the disease itself. For example, risk factors for AD that are useful in accordance with methods, compositions, and uses of some embodiments herein include diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), familial AD, advanced age, or a symptom of dementia.

In certain embodiments, the present invention provides methods of reducing extracellular protein aggregates, e.g., amyloid plaque, or protein aggregates released by a cell, e.g., a neuron. For example, a neurological therapeutic agent, such as an antibody or an anti-aggregation small molecule compound, may binds or interacts with an amyloid plaque to reduce the protein aggregation.

In some embodiments, the present invention provides methods of reducing intracellular protein aggregates, e.g., a huntingtin aggregate within a cell. Through increased fluid flow by a flow modulator of the invention, e.g., VEGF-c, a neurological therapeutic agent may be delivered into a cell to reduce the formation of the protein aggregate. For example, a small inhibitory RNA may be delivered to a cell to reduce the expression of a protein, e.g., amyloid-beta, thereby reducing the formation of amyloid aggregate in the cell.

Methods, Compositions, and Uses of Increasing Clearance of Molecules from the CNS

Some embodiments include a method, use, or composition for use in increasing clearance of molecules (such as proteins, e.g., amyloid beta) from the central nervous system of a subject. The method or use can comprise administering a composition comprising, consisting of, or consisting essentially of a flow modulator (e.g., VEGFR3 agonist and/or FGF2) to a meningeal space of the subject, in which fluid flow in the central nervous system of the subject is increased, and administering a neurological therapeutic agent (e.g., amyloid beta antibody) to the subject. Thus, the method or use can increase the clearance of molecules from the CNS of the subject. The neurological therapeutic agent may be administered to the meningeal space, or to a different location in the subject. Increased clearance of molecules from the CNS of the subject can comprise an increased rate of movement of molecules from the CSF to deep cervical lymph nodes, and thus can be ascertained by monitoring the rate of movement of molecules and/or labels in the CNS to deep cervical lymph nodes. In some embodiments, the flow modulator (e.g., VEGFR3 agonist and/or FGF2) and/or the neurological therapeutic agent is administered selectively to the meningeal space. In some embodiments, a composition comprising, consisting of, or consisting essentially of the flow modulator (e.g., VEGR3 agonist and/or FGF2) and/or the neurological therapeutic agent is administered to the CNS, but not outside the CNS. In some embodiments, the flow modulator (e.g., VEGFR3 agonist and/or FGF2) is administered to the CNS, but not blood. By way of example, the VEGFR3 agonist can be selected from the group consisting of one or more of the following: VEGF-c, VEGF-d, or an analog, variant, or functional fragment thereof.

Without being limited by theory, it is contemplated that, according to several embodiments herein, increasing flow by increasing the diameter of, increasing drainage by, and/or increasing the quantity of meningeal lymphatic vessels as described herein can increase clearance of molecules from the CNS of the subject, and thus reduces the concentration and/or accumulation of the molecules in the CNS and brain in accordance with some embodiments herein. Accordingly, in some embodiments, increasing clearance of molecules in the CNS reduces concentration and/or accumulation of the molecules in the CNS and brain. For example, if amyloid beta plaques are present in the CNS of the subject, increasing clearance can reduce amyloid beta plaques, or decrease the rate of their accumulation. Without being limited by theory, it is contemplated that by clearing soluble amyloid beta from the CNS, a gradient will favor solubilization of amyloid beta plaques, so that fluids in the CNS continue to flow and the CNS continues to be cleared, amyloid beta plaques can diminish, or the rate of increase can be reduced. Thus, decreases of amyloid beta plaques can represent a decrease in an etiology of a disease caused by amyloid beta plaques, and, more generally can indicate an increase in fluid flow in the CNS, for example via drainage by meningeal lymphatic vessels. In some embodiments, a quantity of accumulated amyloid beta plaques in the central nervous system, or the rate of accumulation thereof, is reduced by at least 2%, for example at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values, following administration of the flow modulator (e.g., VEGFR3 agonist and/or FGF2) and the neurological therapeutic agent (e.g., amyloid beta antibody). In some embodiments, amyloid beta plaques are cleared from meningeal portions of the central nervous system of the subject. In some embodiments, increased fluid flow in the central nervous system of the subject comprises an increased rate of perfusion of fluid throughout an area of the subject's brain. In some embodiments, increased fluid flow in the central nervous system of the subject comprises an increased rate of perfusion out of the subject's central nervous system.

As discussed herein, methods, uses, and compositions for increasing clearance of molecules from the CNS can be useful in treating, preventing, or ameliorating symptoms of neurological diseases, for example diseases associated with accumulation of macromolecules, cells, or debris in the CNS. Accordingly, in some embodiments, the method or use further includes determining the subject to have such a neurological disease, or a risk factor for such a neurological disease. Example neurological diseases include AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items. In some embodiments, the neurological disease comprises, consists essentially of, or consists of a proteinopathy, for example a tauopathy or an amyloidosis such as AD (e.g., familial AD and/or sporadic AD).

In some embodiments, for any of the methods, compositions, or uses for increasing flow, increasing clearance, increasing drainage, increasing meningeal lymphatic diameter, and/or reducing amyloid beta plaques noted herein a FGF2 or a VEGFR3 agonist as described herein, and a neurological therapeutic agent can be administered. In some embodiments, the VEGFR3 agonist is selected from the group consisting of one or more of the following: VEGF-c, VEGF-d, or an analog, variant or functional fragment of either of these, and the neurological therapeutic agent comprises, consists essentially of, or consists of an amyloid beta antibody. In some embodiments, the VEGFR3 agonist and/or FGF2 and/or the neurological therapeutic agent (e.g., amyloid beta antibody) is administered selectively to the meningeal space of the subject. In some embodiments, the VEGFR3 agonist and/or FGF2 and/or the neurological therapeutic agent (e.g., amyloid beta antibody) is administered to the subject by a route selected from the group consisting of at least one of the following: nasal administration, transcranial administration, contact cerebral spinal fluid (CSF) of the subject, pumping into CSF of the subject, implantation into the skull or brain, contacting a thinned skull or skull portion of the subject with the VEGFR3 agonist and/or FGF2 and the neurological therapeutic agent (e.g., amyloid beta antibody), or expression in the subject of a nucleic acid encoding the VEGFR3 agonist and/or FGF2, or a combination of any of the listed routes. In some embodiments, the VEGFR3 agonist and/or FGF2 is administered to the subject after determining the subject to have the risk factor for the neurological disease. In some embodiments, the VEGFR3 agonist and/or FGF2 is administered to the subject after determining the subject to have the neurological disease. The VEGFR3 agonist and/or FGF2 and the neurological therapeutic agent (e.g., amyloid beta antibody) can be administered in an effective amount to treat, inhibit, ameliorate symptoms of, delay the onset of, reduce the likelihood of, prevent the neurological disease.

Methods, Compositions, and Uses of Increasing Clearance of Cells from the CNS

In some aspect, the present invention is based upon, at least in part, the discovery that the number of immune cells increase in the central nervous system of a subject that has an enhance risk of developing a neurological disease, e.g., Alzheimer's disease. Accordingly, in some embodiments, the present invention provides a method of reducing the number of immune cells in the central nervous system of a subject.

Some embodiments of the disclosure include a method, use, or composition for use in increasing clearance of cells (such as immune cells) from the central nervous system of a subject. The method or use can comprise administering a composition comprising, consisting of, or consisting essentially of a flow modulator (e.g., VEGFR3 agonist and/or FGF2) to a meningeal space of the subject, in which fluid flow in the central nervous system of the subject is increased, and administering a neurological therapeutic agent to the subject.

Thus, the method or use can increase the clearance of cells from the CNS of the subject. The neurological therapeutic agent may be administered to the meningeal space, or to a different location in the subject. Increased clearance of cells from the CNS of the subject can comprise an increased rate of movement of cells from the CSF to deep cervical lymph nodes, and thus can be ascertained by monitoring the rate of movement of cells and/or labels in the CNS to deep cervical lymph nodes. In some embodiments, the flow modulator (e.g., VEGFR3 agonist and/or FGF2) and/or the neurological therapeutic agent is administered selectively to the meningeal space. In some embodiments, a composition comprising, consisting of, or consisting essentially of the flow modulator (e.g., VEGR3 agonist and/or FGF2) and/or the neurological therapeutic agent is administered to the CNS, but not outside the CNS. In some embodiments, the flow modulator (e.g., VEGFR3 agonist and/or FGF2) is administered to the CNS, but not blood. By way of example, the VEGFR3 agonist can be selected from the group consisting of one or more of the following: VEGF-c, VEGF-d, or an analog, variant, or functional fragment thereof.

According to several embodiments herein, increasing flow by increasing the diameter of, increasing drainage by, and/or increasing the quantity of meningeal lymphatic vessels as described herein can increase clearance of cells, e.g., from the CNS of the subject, and thus reduces the concentration and/or accumulation of the cells, e.g., immune cells, in the CNS and brain in accordance with some embodiments herein. Accordingly, in some embodiments, increasing clearance of cells, e.g., immune cells, in the CNS reduces concentration and/or accumulation of the cells, e.g., immune cells, in the CNS and brain. Accordingly, the flow modulators (e.g., VEGF-c) of the invention synergize with the neurological therapeutic agents of the invention to reduce the number of cells that contribute to the pathogenesis of a neurodegenerative disease, e.g., Alzheimer's disease, resulting in a greater anti-inflammatory effect than either flow modulator or therapeutic agent alone.

Immune cells may contribute to the pathogenesis of a neurodegenerative disease through chronic inflammation. For example, immune cells such as T cells and B cells may contribute to chronic inflammation through secretion of proinflammatory cytokines. Without being limited by theory, it is contemplated that by clearing immune cells from the CNS, the neuroinflammation associated with neurodegenerative diseases, such as Alzheimer's disease, may be reduced and ameliorate the symptoms of the disease. Thus, decreases of immune cells can represent a decrease in an etiology of a disease caused by chronic inflammation, and, more generally can indicate an increase in fluid flow in the CNS, for example via drainage by meningeal lymphatic vessels. In some embodiments, a quantity of immune cells in the central nervous system, or the rate of accumulation thereof, is reduced by at least 2%, for example at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values, following administration of the flow modulator (e.g., VEGFR3 agonist and/or FGF2) and the neurological therapeutic agent. In some embodiments, amyloid beta plaques are cleared from meningeal portions of the central nervous system of the subject. In some embodiments, increased fluid flow in the central nervous system of the subject comprises an increased rate of perfusion of fluid throughout an area of the subject's brain. In some embodiments, increased fluid flow in the central nervous system of the subject comprises an increased rate of perfusion out of the subject's central nervous system.

As discussed herein, methods, uses, and compositions for increasing clearance of cells, e.g., immune cells, from the CNS can be useful in treating, preventing, or ameliorating symptoms of neurological diseases, for example diseases associated with accumulation of macromolecules, cells, or debris in the CNS. Accordingly, in some embodiments, the method or use further includes determining the subject to have such a neurological disease, or a risk factor for such a neurological disease. Example neurological diseases include AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items. In some embodiments, the neurological disease comprises, consists essentially of, or consists of a proteinopathy, for example a tauopathy or an amyloidosis such as AD (e.g., familial AD and/or sporadic AD).

In some embodiments, for any of the methods, compositions, or uses for increasing flow, increasing clearance, increasing drainage, increasing meningeal lymphatic diameter, and/or reducing immune cells noted herein a FGF2 or a VEGFR3 agonist as described herein, and a neurological therapeutic agent can be administered. In some embodiments, the VEGFR3 agonist is selected from the group consisting of one or more of the following: VEGF-c, VEGF-d, or an analog, variant or functional fragment of either of these, and the neurological therapeutic agent comprises, consists essentially of, or consists of an amyloid beta antibody. In some embodiments, the VEGFR3 agonist and/or FGF2 and/or the neurological therapeutic agent is administered selectively to the meningeal space of the subject. In some embodiments, the VEGFR3 agonist and/or FGF2 and/or the neurological therapeutic agent is administered to the subject by a route selected from the group consisting of at least one of the following: nasal administration, transcranial administration, contact cerebral spinal fluid (CSF) of the subject, pumping into CSF of the subject, implantation into the skull or brain, contacting a thinned skull or skull portion of the subject with the VEGFR3 agonist and/or FGF2 and the neurological therapeutic agent, or expression in the subject of a nucleic acid encoding the VEGFR3 agonist and/or FGF2, or a combination of any of the listed routes. In some embodiments, the VEGFR3 agonist and/or FGF2 is administered to the subject after determining the subject to have the risk factor for the neurological disease. In some embodiments, the VEGFR3 agonist and/or FGF2 is administered to the subject after determining the subject to have the neurological disease. The VEGFR3 agonist and/or FGF2 and the neurological therapeutic agent can be administered in an effective amount to treat, inhibit, ameliorate symptoms of, delay the onset of, reduce the likelihood of, prevent the neurological disease.

### Additional Embodiments

All technical and scientific terms used herein have the meaning as would be understood by one of ordinary skill in the art to which this subject matter belongs, in view of this disclosure.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable sub-combination. All combinations of the embodiments pertaining to the subject matter herein are specifically contemplated and are disclosed herein just as if each and every combination was individually and explicitly disclosed. In addition, all sub-combinations of the various embodiments and elements thereof are also specifically contemplated and are disclosed herein just as if each and every such sub-combination was individually and explicitly disclosed herein.

Some aspects provide methods of treating a neurological disease (such as AD) in a subject comprising administering to the subject a therapeutically effective amount of a neurological therapeutic agent and a flow modulator that modulates one or more of a) drainage of the meningeal lymphatic vessel(s); b) diameter of the meningeal lymphatic vessel(s); c) lymphangiogenesis of the meningeal lymphatic vessel(s); d) contractility of the meningeal lymphatic vessel(s); and/or e) permeability of the meningeal lymphatic vessel(s). The present disclosure also provides methods of treating AD in a subject by administering to the subject a neurological therapeutic agent and a flow modulator that increases drainage of the meningeal lymphatic vessel(s), increases the diameter of the meningeal lymphatic vessel(s), causes lymphangiogenesis of the meningeal lymphatic vessel(s), modulates contractility of the meningeal lymphatic vessel(s) to increase drainage, and/or modulates the permeability of the meningeal lymphatic vessel(s) to increase drainage. The present disclosure also provides methods of treating a neurological disease such as AD described herein in a subject by administering to the subject a neurological therapeutic agent and a flow modulator that increases drainage of the meningeal lymphatic vessel(s), increases the diameter of the meningeal lymphatic vessel(s), causes lymphangiogenesis of the meningeal lymphatic vessel(s), modulates contractility of the meningeal lymphatic vessel(s) to increase drainage, and/or modulates the permeability of the meningeal lymphatic vessel(s) to increase drainage.

Below are non-limiting examples of some embodiments herein:

### EXAMPLES

### Example 1: Adult 5×FAD Meninges and Brain

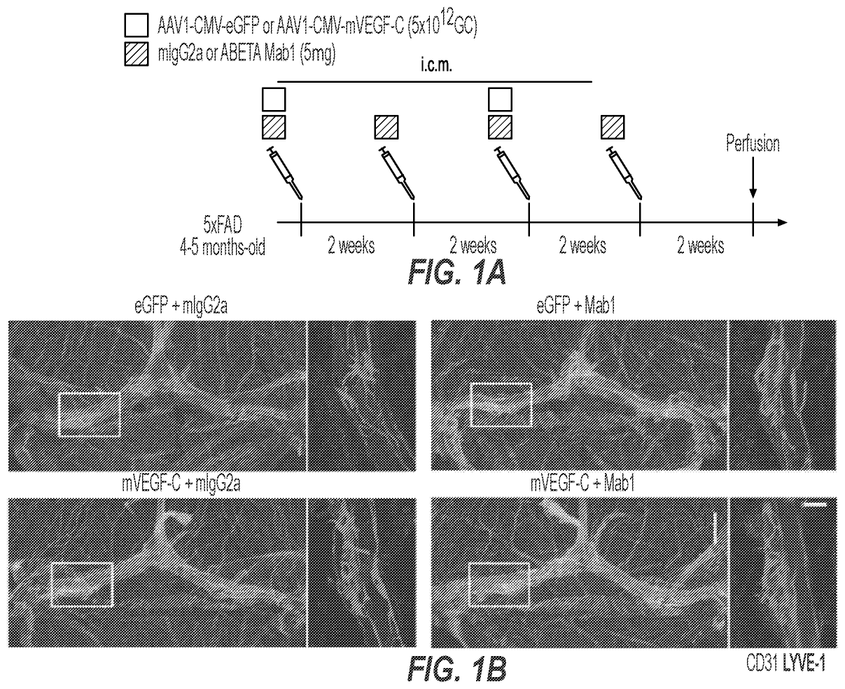

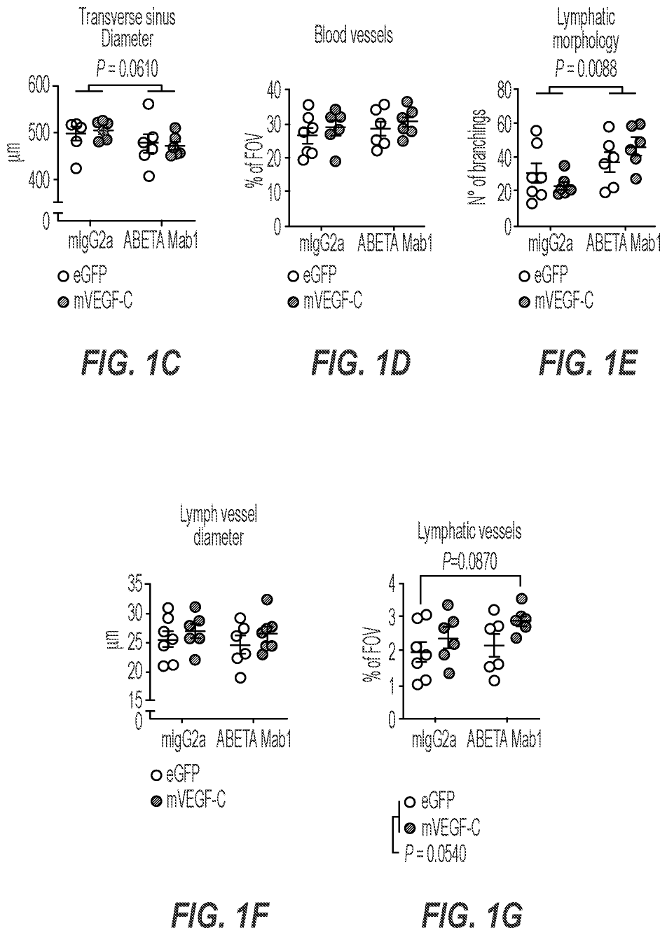

Adult 5×FAD mice were treated with a combination of VEGF-c and an antibody that binds amyloid beta (ABETA Mab1) administered into the cisterna magna (i.c.m). After administration of AAV1 vector expressing VEGF-c or eGFP and the antibody according to schematic shown in FIG. 1A, brain and meningeal morphology was observed. Representative images of the meningeal whole-mounts of 5×FAD mice treated with different combinations of mIgG2a or monoclonal anti-amyloid beta antibody (“ABETA Ab”) with AAV1-CMV-eGFP or AAV1-CMV-mVEGF-C are shown in FIG. 1B. Meninges were stained for LYVE-1 and CD31; scale bar, 1 mm; inset, 300 m (FIG. 1B) Measurements of transverse sinus diameter, coverage by LYVE-1negCD31⁺ blood vessels, total number of lymphatic branches, transverse sinus lymphatic vessel diameter and coverage by LYVE-1+ lymphatic vessels (See FIGS. 1C-1G). Results in FIGS. 1C-1G are presented as mean±s.e.m.; n=7 in eGFP+mIgG2a, n=6 in eGFP+ABETA Ab, in mVEGF-C+mIgG2a and in mVEGF-C+ABETA Ab; Two-way ANOVA with Sidak's multiple comparison test. In FIGS. 1D and 1G, the units for the Y-axis are percentage of field of view (“% FOV”). It can be seen that the flow modulator VEGF-c increased lymphatic diameter, and synergized with the amyloid beta antibody to enhance lymphatic branching (FIGS. 1C and 1E). These experiments show that a flow modulator and neurological therapeutic agent in accordance with some embodiments herein can synergize to enhance lymphatic branching in the CNS of a model of AD comprising amyloid beta plaques.

### Example 2: Adult 5×FAD Brain

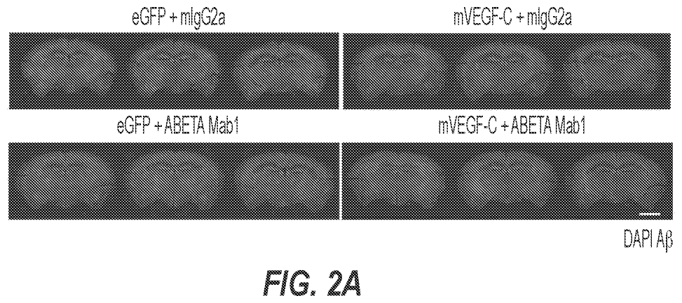

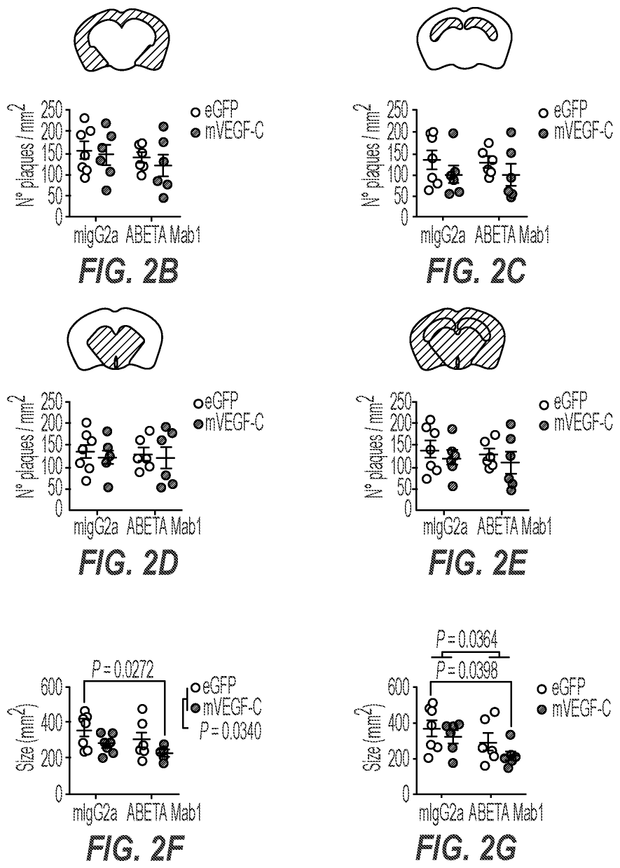

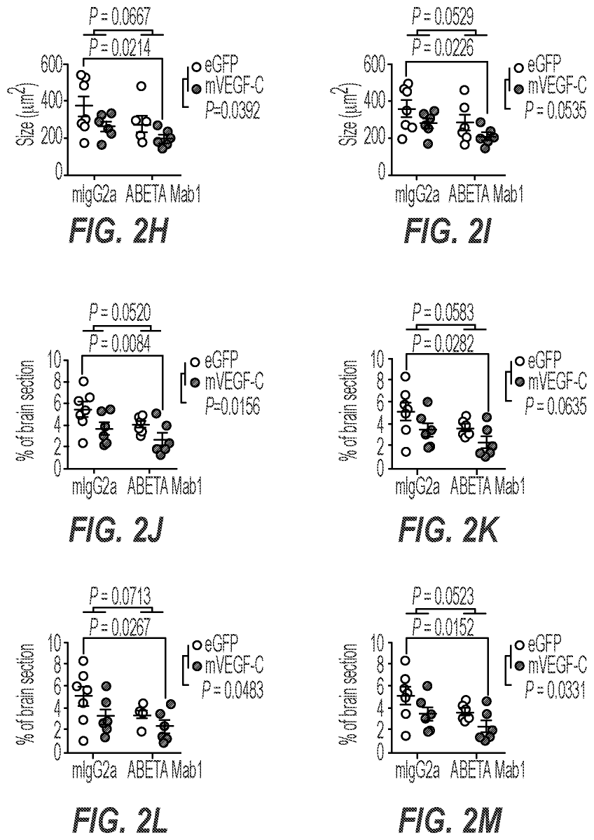

Adult 5×FAD mice were treated with a combination of VEGF-c and an antibody that binds amyloid beta. Representative images of the brain sections of 5×FAD mice treated with different combinations of mIgG2a or ABETA Ab with AAV1-CMV-eGFP or AAV1-CMV-mVEGF-C. Representative images of the brain sections of these mice are shown in FIG. 2A. Brain sections were stained for Aβ) and with DAPI; scale bar, 2 mm. b-m, Plaque density (number of plaques per mm²), average size (m²) and coverage (% of brain section) in particular brain regions (cortex and amygdala; hippocampus; thalamus and hypothalamus) or in the whole brain section. Results in FIGS. 2B-2M are presented as mean±s.e.m.; n=7 in eGFP+mIgG2a, n=6 in eGFP+ABETA Ab, in mVEGF-C+mIgG2a and in mVEGF-C+ABETA Ab; Two-way ANOVA with Sidak's multiple comparison test. Moreover, amyloid plaque size and brain coverage were reduced in several regions of mice receiving combination treatment of AAV1 expressing VEGF-c and antibody, particularly when, compared to mice receiving control mIgG2a only (see FIGS. 2F-2M). This experiment shows that a flow modulator and neurological therapeutic agent in a model of AD comprising amyloid beta plaques in accordance with some embodiments herein can synergize to reduce amyloid beta plaque size, density, and coverage (as percent of brain region).

### Example 3: Old APPswe Behavior and Brain

Age of APPswe mice and treatment regimen of anti-amyloid beta antibody with AA1 vector expressing VEGF-c or eGFB via i.c.m are shown in FIG. 3A. Results of behavioral tests (open field (OF), novel location recognition (NLR) and contextual fear conditioning (CFC)) are shown in FIGS. 3B-3D. Administration of VEGF-c treatment reduced the number of plaques in the cortex and amygdala (FIG. 3F) or in the hippocampus (FIG. 3G) Total distance, velocity and time in center of the arena (% of total time) in the OF test are shown in FIG. 3B. Time investigating one of the object location (% of total time investigating objects) in the training trial and time investigating the novel object location (% of total time investigating) in the NLR test are shown in FIG. 3C. Time freezing (% of total time) in the context trial and in cued trial of the CFC are shown in FIG. 3D. Representative images of the brain sections of APPswe mice treated with anti-Abeta antibody and with AAV1-CMV-eGFP or AAV1-CMV-mVEGF-C are shown in FIG. 3E. Brain sections were stained for Aβ and with DAPI; scale bar, 1 mm. FIGS. 3F-3G show plaque density (number of plaques per mm²), average size (m²) and coverage (% of brain section) in the cortex and amygdala or in the hippocampus. Results in FIGS. 3B-3D, 3F, and 3G are presented as mean s.e.m.; n=11 per group; Two-tailed unpaired Student's T test. This experiment is another example that demonstrates that a flow modulator and neurological therapeutic agent can synergize to reduce amyloid beta plaque burden in another model of AD (APPswe mice).

### Example 4: Alzheimer's Disease and Introduction

Alzheimer's disease (AD) is the most prevalent form of dementia. A growing body of evidence points to passive immunotherapy as a promising therapeutic strategy to slow AD progression. Administration of monoclonal antibodies against amyloid beta (Aβ) has been shown to reduce brain senile plaque load both in AD transgenic mouse models and in AD patients. While dysfunctional meningeal lymphatic vasculature plays an important role in Aβ accumulation, it was previously unknown if or how altered brain drainage mediated by meningeal lymphatics affects immunotherapy in AD. Based on the studies in adult and aged AD transgenic mice, it was demonstrated herein early alterations, induced in part by Aβ, in meningeal lymphatic endothelial cells, and that manipulation of lymphatic vessel drainage at the dorsal brain meninges affects clearance of Aβ by monoclonal antibodies. It is further shown herein that genes associated with increased risk for AD and other neurological disorders are highly expressed in the lymphatic vasculature, suggesting that more meaningful clinical results might be achieved by stratifying patients based on their meningeal lymphatic function. Overall, this new evidence strongly supports the notion that enhancement of meningeal lymphatic drainage could provide an important adjuvant to current monoclonal antibody-based passive immunotherapies in AD.

The prevalence of AD and other dementias is expected to increase owing to better health care and higher life expectancy. Increased accumulation and aggregation of Aβ, the main constituent of senile plaques in the brain parenchyma, is one of the key pathological hallmarks of AD (Benilova, I., Karran, E. & De Strooper, B. The toxic Abeta oligomer and Alzheimer's disease: an emperor in need of clothes. Nat Neurosci 15, 349-357, (2012); Bateman, R. J. et al. Clinical and biomarker changes in dominantly inherited Alzheimer's disease. N Engl J Med 367, 795-804, (2012)). Pathological accumulation of Aβ in the brain results, in part, from the age-related progressive impairment of cleansing mechanisms (Mawuenyega, K. G. et al. Decreased clearance of CNS beta-amyloid in Alzheimer's disease. Science 330, 1774, (2010); Tarasoff-Conway, J. M. et al. Clearance systems in the brain—implications for Alzheimer disease. Nat Rev Neurol 12, 248, (2016)), including the meningeal lymphatic vasculature (Da Mesquita, S. et al. Functional aspects of meningeal lymphatics in ageing and Alzheimer's disease. Nature 560, 185-191, (2018); Da Mesquita, S., Fu, Z. & Kipnis, J. The Meningeal Lymphatic System: A New Player in Neurophysiology. Neuron 100, 375-388, (2018)). Passive immunotherapy, using monoclonal antibodies against Aβ, is among the most promising of the therapeutic strategies aimed at enhancing the clearance of toxic Aβ species from the brain (Bacskai, B. J. et al. Imaging of amyloid-beta deposits in brains of living mice permits direct observation of clearance of plaques with immunotherapy. Nat Med 7, 369-372, (2001); Bard, F. et al. Peripherally administered antibodies against amyloid beta-peptide enter the central nervous system and reduce pathology in a mouse model of Alzheimer disease. Nat Med 6, 916-919, (2000); Sevigny, J. et al. Addendum: The antibody aducanumab reduces Abeta plaques in Alzheimer's disease. Nature 546, 564, (2017)). Two clinical trials of the anti-Aβ monoclonal antibody Aducanumab, EMERGE and ENGAGE, have recently yielded somewhat contradictory results, since cognitive decline was significantly reduced in patients receiving the highest dose (10 mg/kg) in the EMERGE cohort but not in the ENGAGE cohort (Howard, R. & Liu, K. Y. Questions EMERGE as Biogen claims aducanumab turnaround. Nat Rev Neurol, (2019)). This controversy, along with the meager clinical improvement observed in patients with mild cognitive impairment and AD who were enlisted in trials involving other monoclonal antibodies (Salloway, S. et al. Two phase 3 trials of bapineuzumab in mild-to-moderate Alzheimer's disease. N Engl J Med 370, 322-333, (2014); Logovinsky, V. et al. Safety and tolerability of BAN2401—a clinical study in Alzheimer's disease with a protofibril selective Abeta antibody. Alzheimers Res Ther 8, 14, (2016)), highlights the need for a better understanding of possible factors that might influence the efficacy of anti-Aβ immunotherapy in AD.

It has been previously shown that induction of meningeal lymphatic dysfunction exacerbates brain and meningeal Aβ pathology in different transgenic mouse models of familial AD (Da Mesquita, S. et al. Functional aspects of meningeal lymphatics in ageing and Alzheimer's disease. Nature 560, 185-191, (2018)). Accordingly, it was hypothesized that altered meningeal lymphatic function would affect brain fluid drainage and recirculation, thereby changing the availability of monoclonal antibodies administered to target and clear brain Aβ deposits. This hypothesis led to further investigate changes in the meningeal lymphatic vasculature at different ages in transgenic mouse models of AD, as well as the potential therapeutic implications of modulating this brain-draining meningeal lymphatic system.

### Example 5: Meningeal Lymphatics Become Impaired in 5×FAD Mice

It was previously showed that young-adult (˜3 month-old) 5×FAD mice and age-matched wild-type (WT) littermates present no meningeal lymphatic dysfunction as assessed morphologically and functionally (Da Mesquita, S. et al. Functional aspects of meningeal lymphatics in ageing and Alzheimer's disease. Nature 560, 185-191, (2018)). Likewise, no major changes in the meningeal immune response are detectable at this young age (FIGS. 4A-4Q).

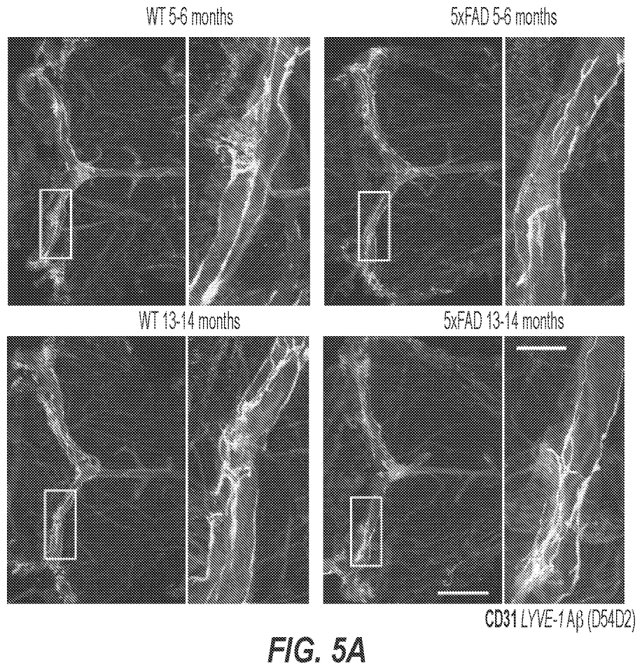

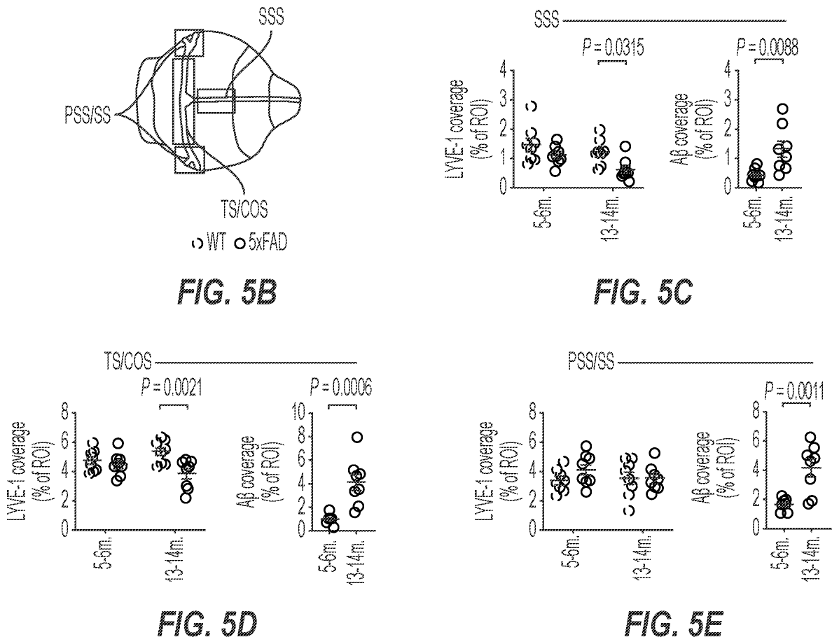

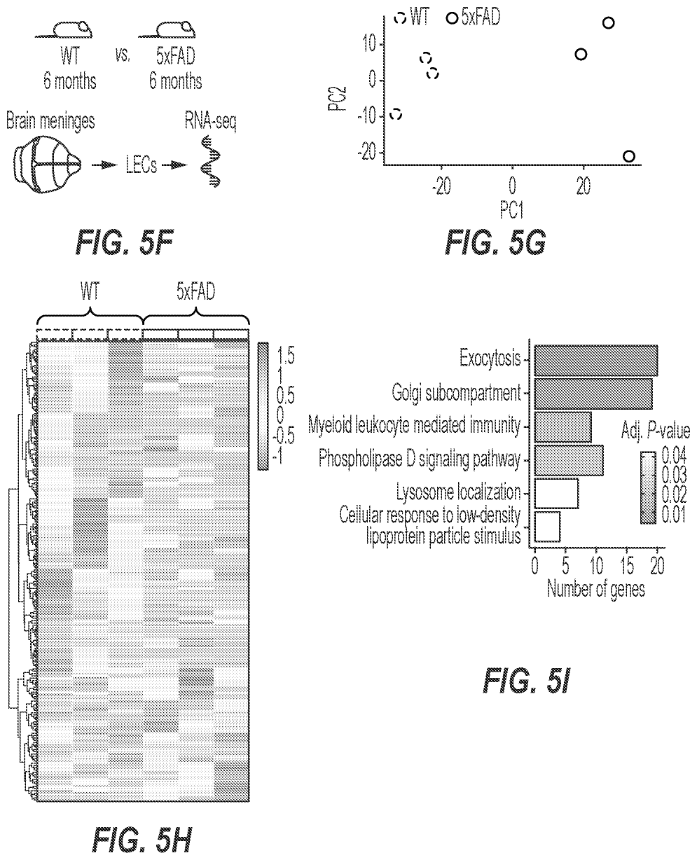

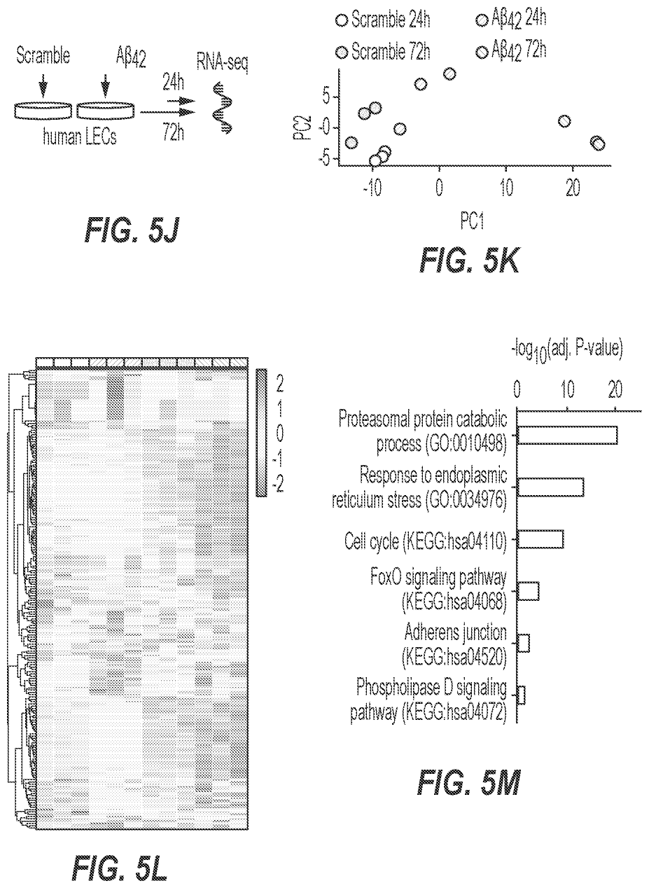

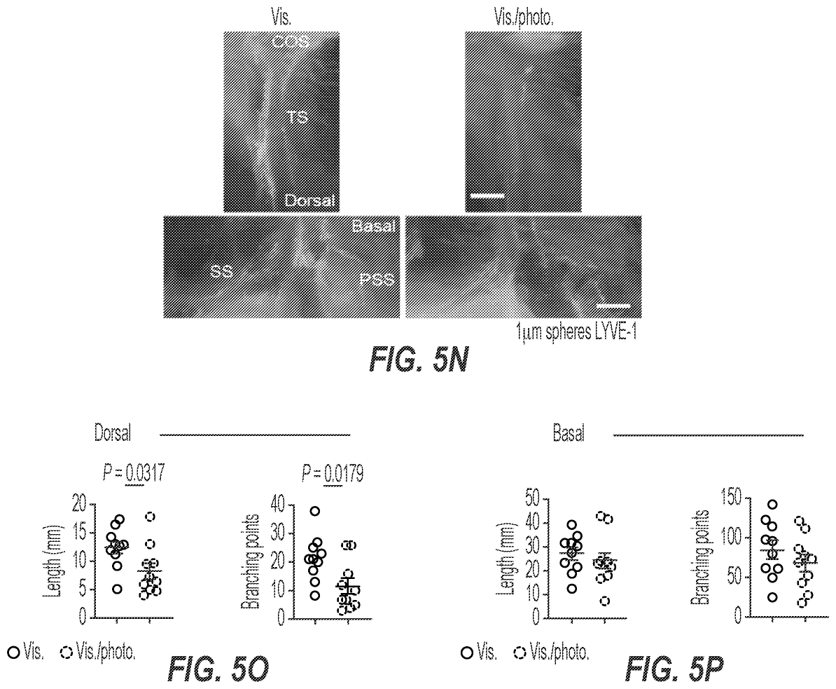

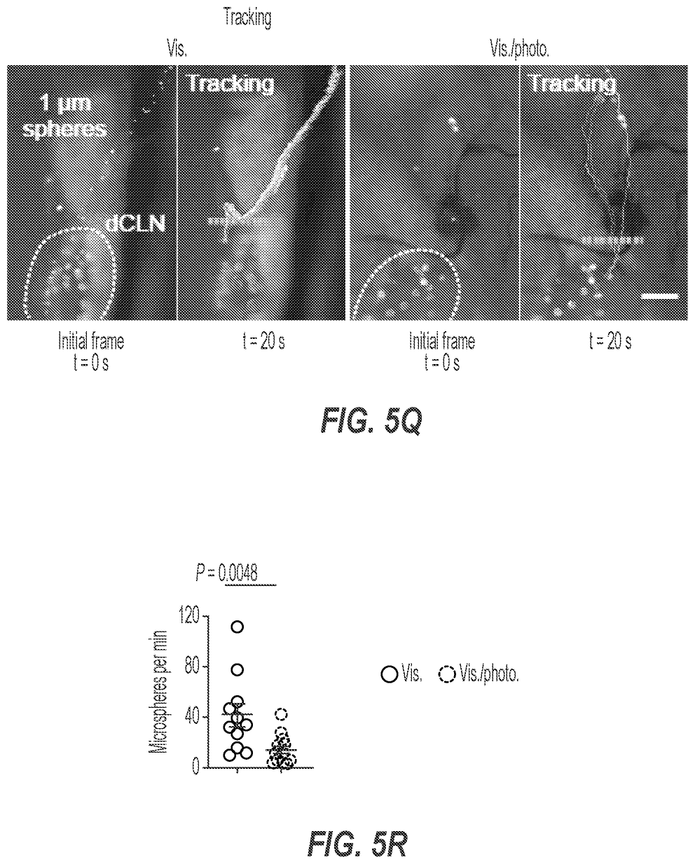

It was next sought to investigate whether changes in meningeal lymphatic vasculature and immunity would emerge with aging in these AD transgenic mice. This study began by comparing meningeal lymphatic vessel coverage between WT and 5×FAD mice (age-matched littermates) at 5-6 and 13-14 months of age (FIG. 5A-5E). Although no changes were observed at 5-6 months, a significant decrease in lymphatic vessel coverage along the superior sagittal sinus (SSS), transverse sinus (TS) and the confluence of sinuses (COS) was observed at 13-14 months in the meninges of 5×FAD mice (FIGS. 5C-5D). No changes in meningeal lymphatic coverage around the petrosquamosal (PSS) and sigmoid (SS) sinuses were observed between the two groups (FIG. 5E). The deterioration of the lymphatic vasculature at the dorsal meninges observed in 13-14 months-old 5×FAD mice was accompanied by a significant increase in Aβ deposition throughout all the analyzed regions of the meningeal whole mount (FIGS. 5C-5E). Interestingly, extensive meningeal Aβ deposition along the blood and lymphatic vasculature (FIG. 5A) was more evident at anatomical locations previously shown to be ‘hot spots’ for CSF access to the lymphatic vasculature at the dorsal meninges ensheathing the brain (Louveau, A. et al. CNS lymphatic drainage and neuroinflammation are regulated by meningeal lymphatic vasculature. Nat Neurosci 21, 1380-1391, (2018).

Next, meningeal lymphatic endothelial cells (LECs) were isolated from WT and 5×FAD mice at the age of 6 months to analyze their transcriptomes by bulk RNA sequencing (RNA-seq; FIG. 5F and FIGS. 6A-6C). Tables 2-7 summarize the results as shown in FIGS. 6A-6C. It was found that at this relatively young age, LECs taken from the 5×FAD mice already showed significant changes in the expression of genes involved in the regulation of Golgi apparatus and exocytosis, phospholipase D signaling, and response to low-density lipoprotein (FIGS. 5G-5I and FIGS. 6B and 6C). Tables 8 and 9 summarize the results as shown in FIG. 5H.

To determine whether the increased levels of Aβ alone could account for the observed changes in the LEC transcriptome, human LECs were incubated with 100 nM human Aβ₁₋₄₂ (a monomeric/dimeric preparation) or control scrambled human Aβ₁₋₄₂ peptide for 24 or 72 h (FIG. 5J). Changes in gene expression in these human LECs were indeed observed upon their incubation with Aβ₁₋₄₂ for 24 h and were even more pronounced at 72 h (FIGS. 5K and 5L). Tables 10 and 11 summarizes the results as shown in FIG. 5L. Within the significantly altered functional pathways, genes detected involved in Forkhead box O signaling, in maintenance of adherens junctions between LECs and, as before, in phospholipase D signaling (FIG. 5M and FIGS. 6D-6F). Tables 12-17 summarize the results as shown in FIGS. 6D-6F.

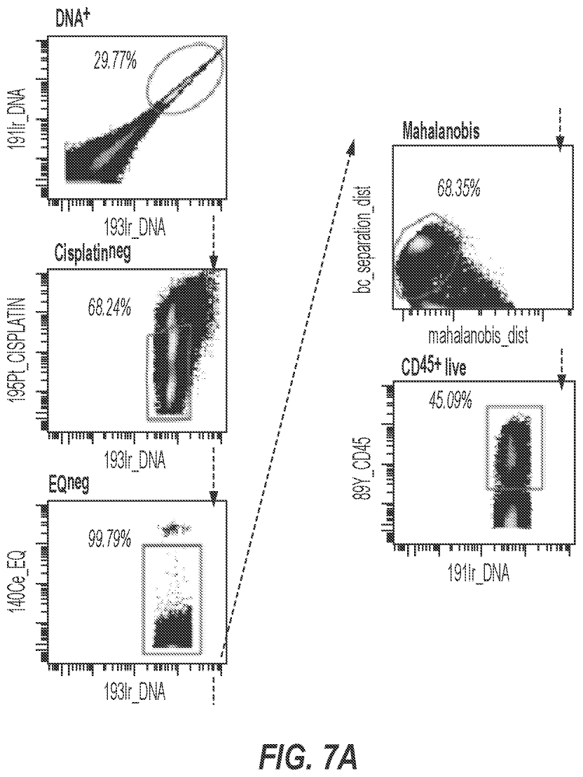

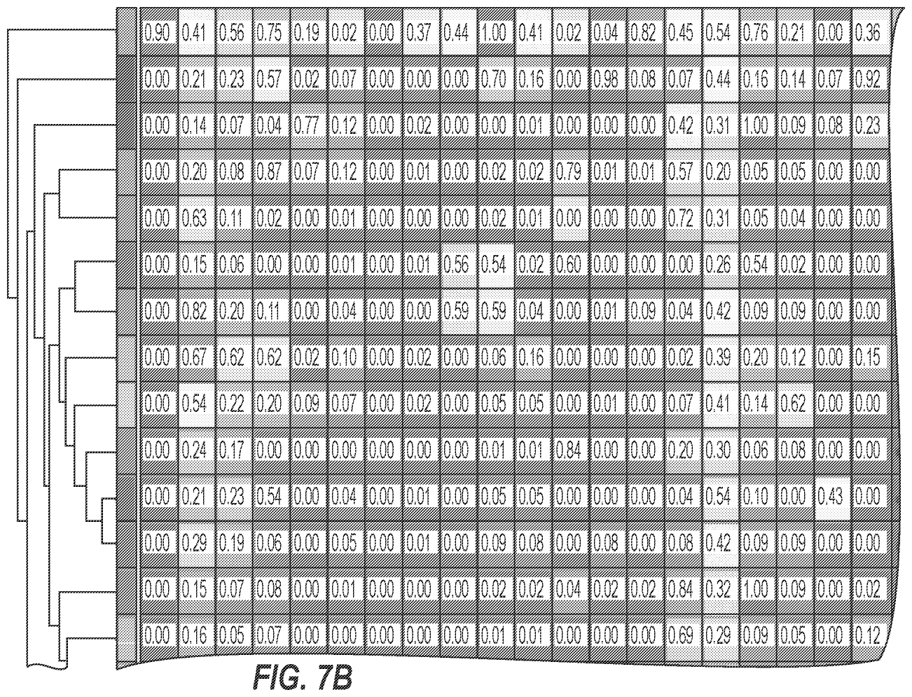

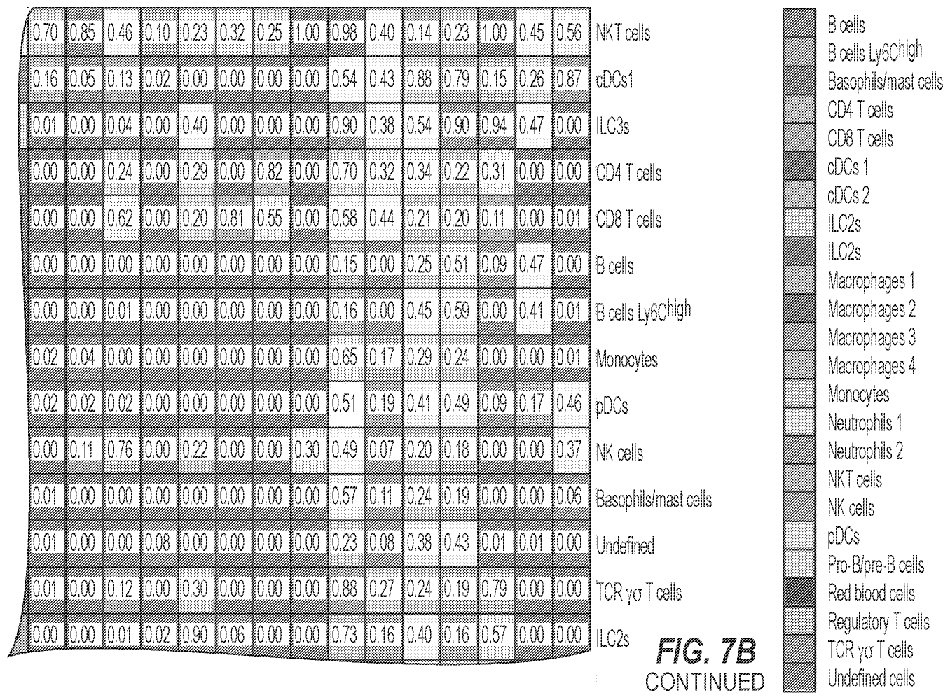

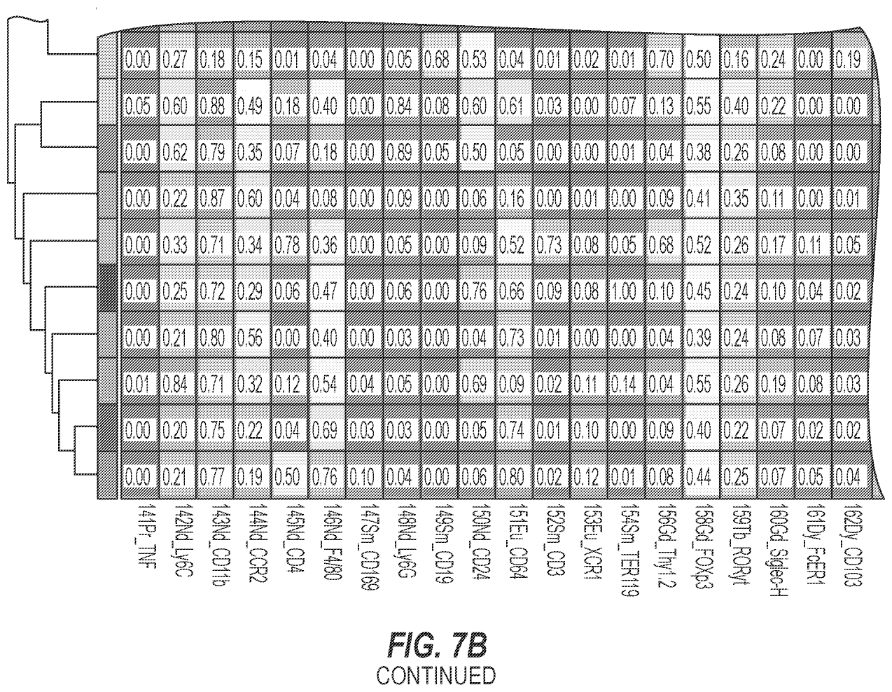

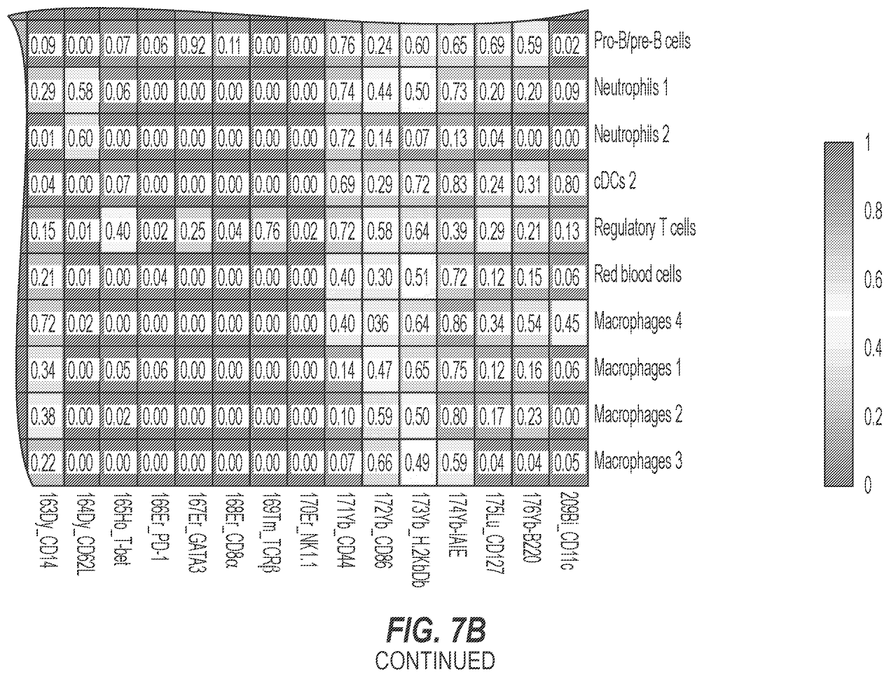

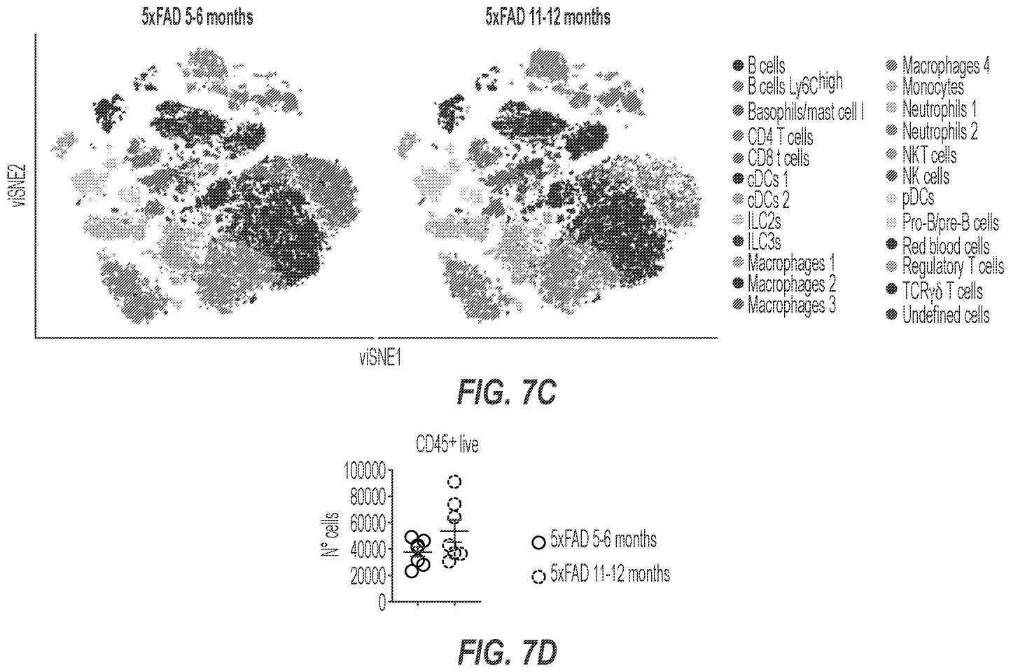

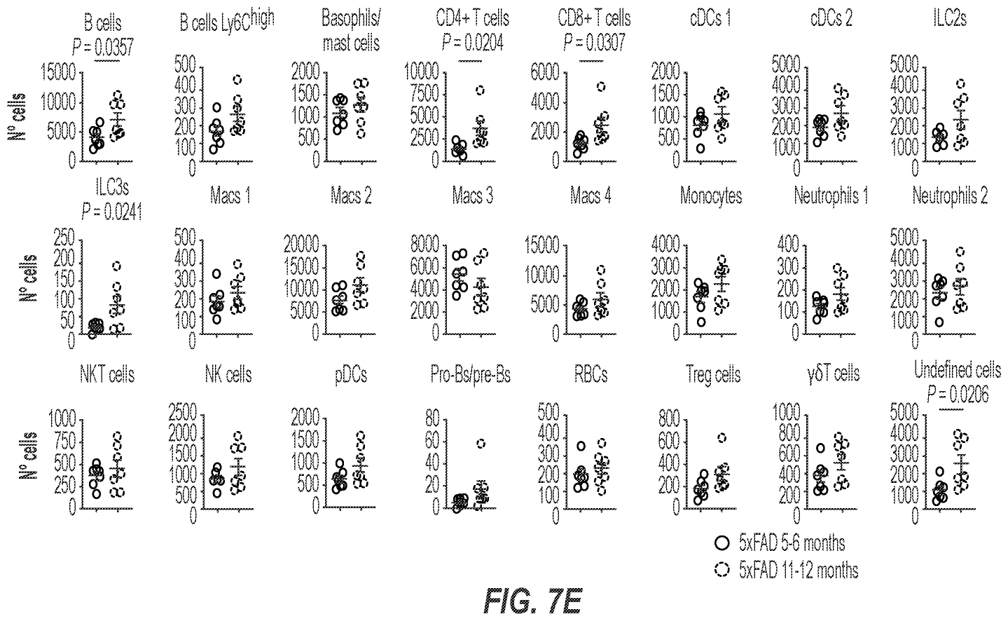

Aberrant activity of phospholipase D1 and D2 and altered levels of low-density lipoprotein have been previously implicated in AD. These results suggest that progressive meningeal Aβ deposition might disrupt the interaction of these molecules with the meningeal LECs in 5×FAD mice and underly the deterioration of lymphatic vasculature. On the assumption that this marked accumulation and deposition of Aβ could have an impact not only on the meningeal lymphatic vasculature but also on the local immune response, it was sought to characterize the meningeal immunity at 5-6 and at 11-12 months. Mass cytometric analysis of different leukocyte populations in the meninges of 5×FAD mice (FIGS. 7A-7D) revealed a significant increase in the numbers of B cells, CD4⁺ T cells and CD8⁺ T cells in middle-aged (11-12 months-old) mice relative to their numbers at 5-6 months (FIG. 7E). Table 18 summarizes the results as shown in FIG. 7B. The degeneration of lymphatic vasculature observed in middle-aged 5×FAD mice was corroborated by an accumulation of adaptive immune cells in the meninges, which—as shown in a previous study (Louveau, A. et al. CNS lymphatic drainage and neuroinflammation are regulated by meningeal lymphatic vasculature. Nat Neurosci 21, 1380-1391, (2018))—can be indicative of impaired meningeal lymphatic drainage.

### Example 6: Meningeal Lymphatic Drainage Affects Anti-Aβ Immunotherapy

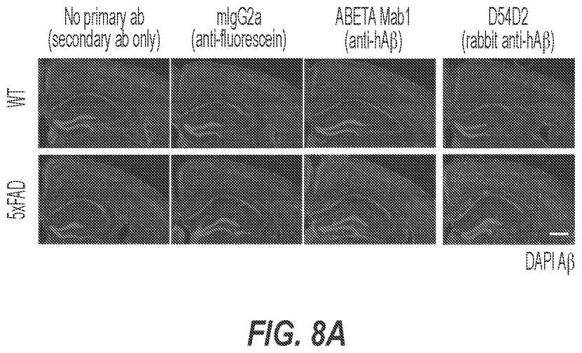

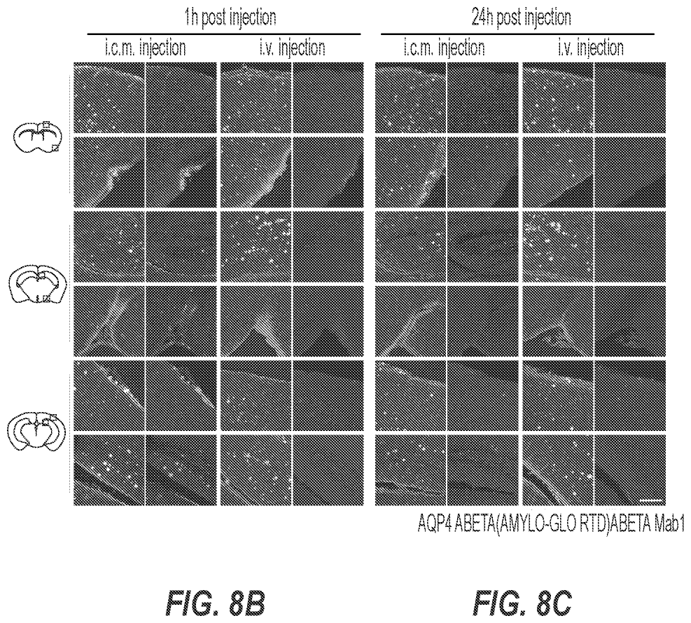

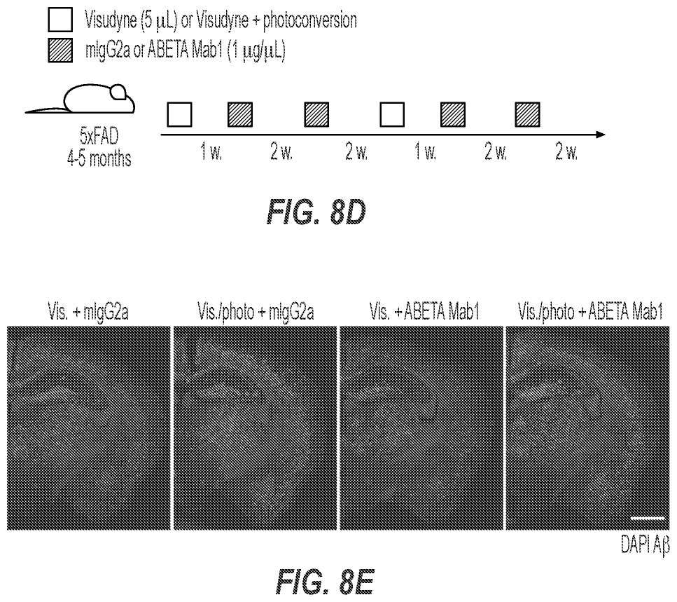

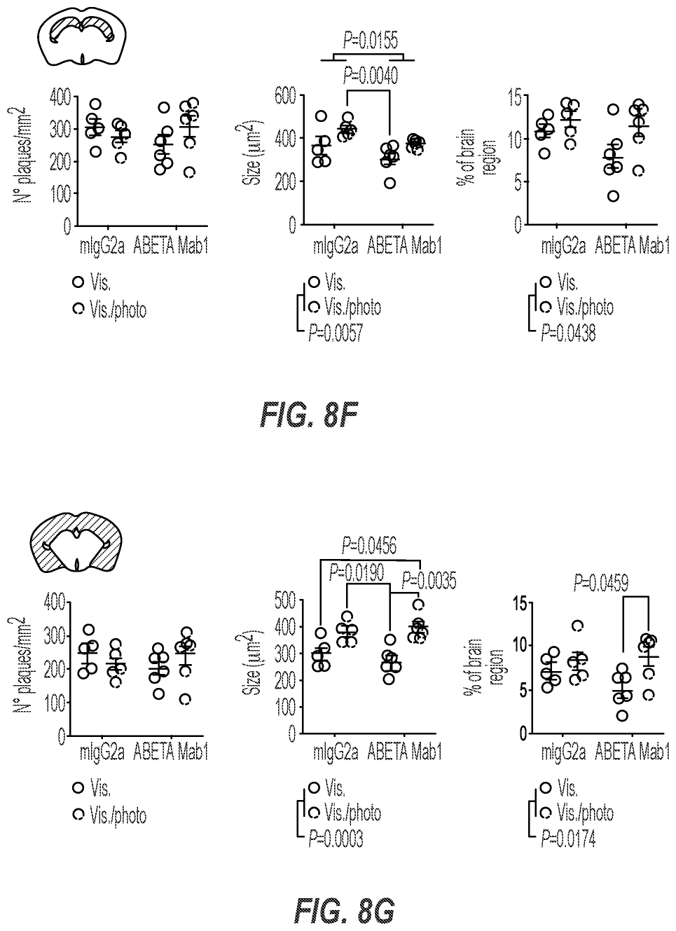

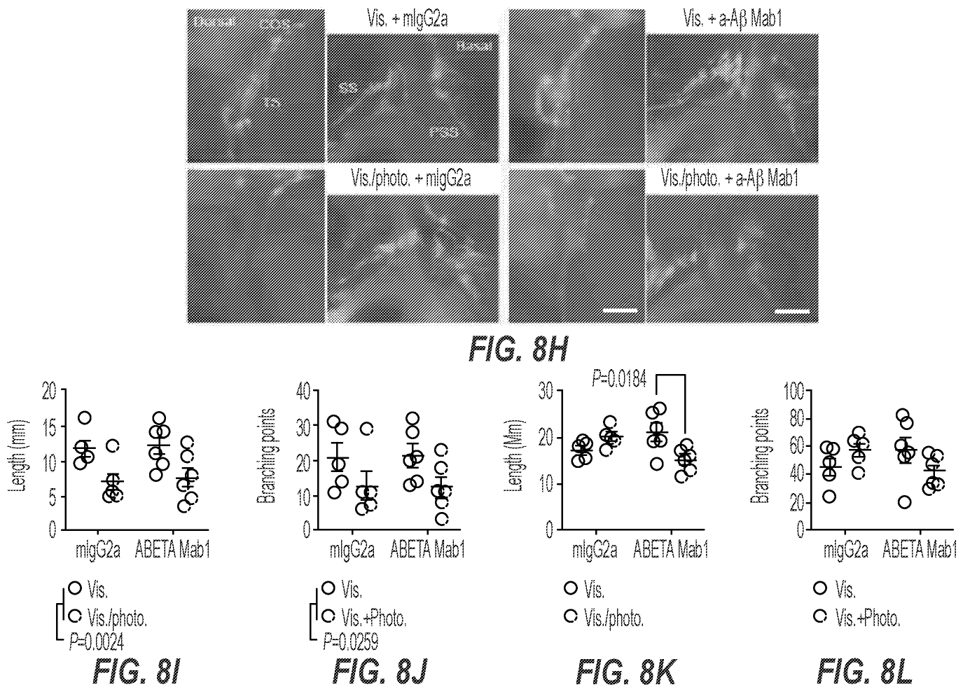

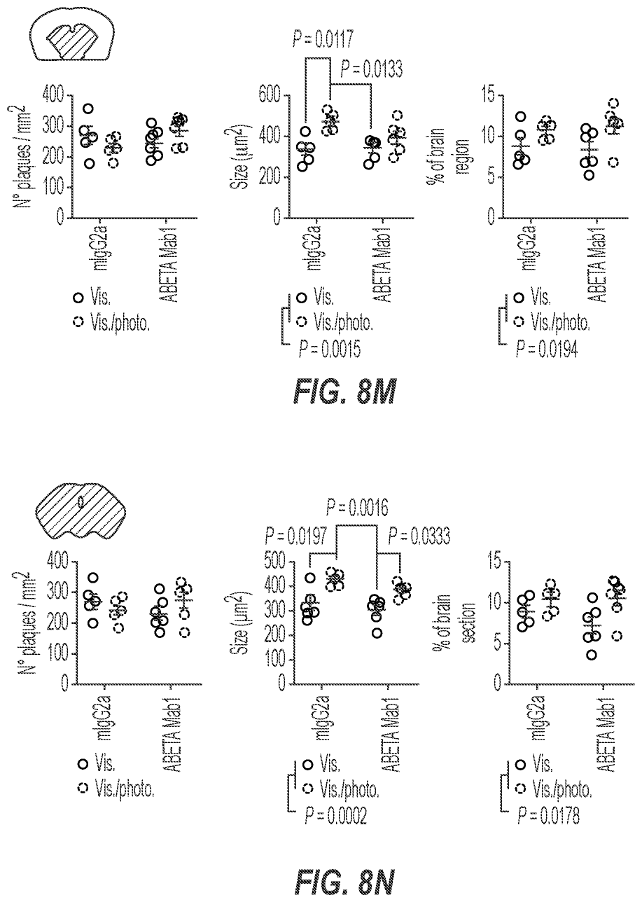

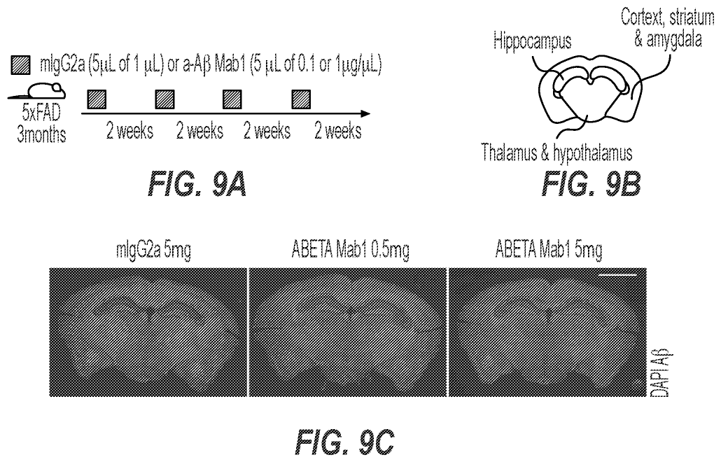

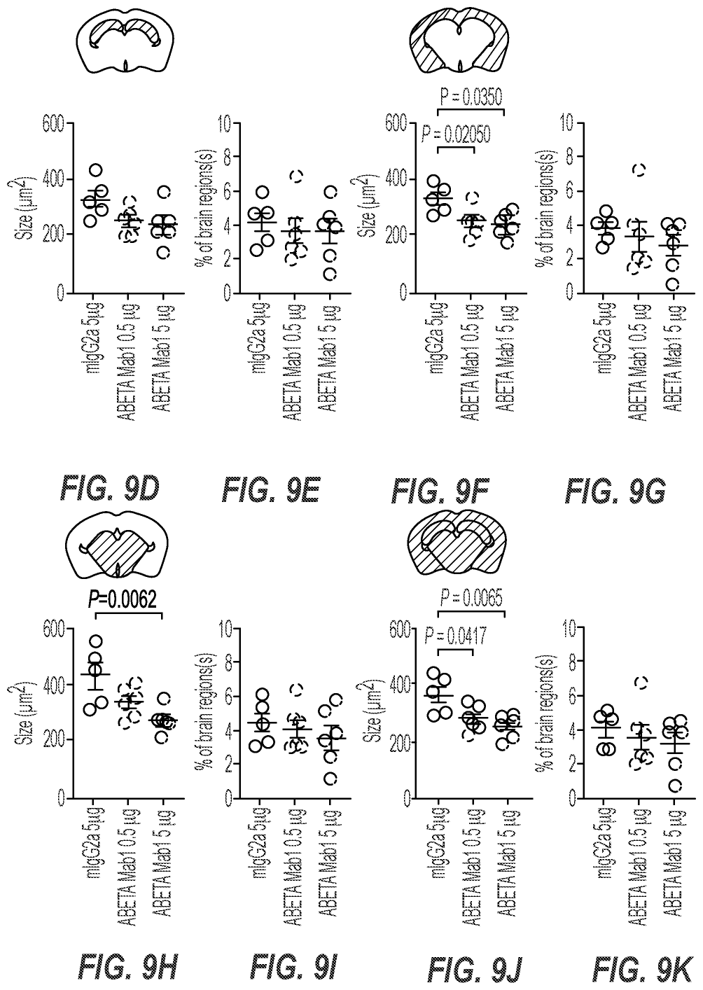

In this study, incubation of brain sections from WT or 5×FAD mice confirmed that anti-Abeta antibody specifically recognizes human Aβ deposited in the brains of 5×FAD mice, but not of WT mice (FIG. 8A). Murine IgG2a isotype antibody (mIgG2a, clone 4-4-20e), used here as a control, did not recognize Aβ plaques in brain sections from 5×FAD mice (FIG. 8A). To determine the efficient route for targeting of brain Aβ plaques, anti-Abeta antibody was injected into 5-month-old 5×FAD mice, either via the CSF (5 μL at 1 μg/μL) by intra-cisterna magna (i.c.m.) infusion or intravenously (i.v., 100 μL at 1 μg/L). Both at 1 h and at 24 h after anti-Abeta antibody injection, the recognition of Aβ plaques was more pronounced when anti-Abeta antibody was administered into the CSF (FIG. 8B) than via the i.v. route (FIG. 8C). The results, in agreement with a recent report (Plog, B. A. et al. Transcranial optical imaging reveals a pathway for optimizing the delivery of immunotherapeutics to the brain. JCI Insight 3, (2018)), suggest that bypassing the tight blood-brain barrier in adult 5×FAD mice, via direct delivery into the CSF, allows a better access of anti-Aβ antibodies to parenchymal Aβ aggregates. To determine whether delivery of anti-Abeta antibody into the CSF can in fact promote clearance of brain Aβ plaques, anti-Abeta antibody (0.5 or 5 μg) or mIgG2a (5 μg) was injected directly into the cisterna magna of 3-month-old 5×FAD mice every 2 weeks for 2 months (FIG. 9A). This regimen resulted in a significant reduction, across different brain regions, of Aβ plaque size in the anti-Abeta antibody-injected groups, with a stronger effect obtained for the higher dose of 5 μg (FIGS. 9B-9K).

Based on recent experimental evidence for an impaired perivascular CSF influx (via the glymphatic pathway) in mouse models of meningeal lymphatic dysfunction (Da Mesquita, S. et al. Functional aspects of meningeal lymphatics in ageing and Alzheimer's disease. Nature 560, 185-191, (2018); Zou, W. et al. Blocking meningeal lymphatic drainage aggravates Parkinson's disease-like pathology in mice overexpressing mutated alpha-synuclein. Transl Neurodegener 8, 7, (2019); Wang, L. et al. Deep cervical lymph node ligation aggravates AD-like pathology of APP/PS1 mice. Brain Pathol 29, 176-192, (2019)), it was postulated that exacerbation of meningeal lymphatic dysfunction in 5×FAD mice would dampen the clearance of Aβ plaques owing to reduced access of anti-Abeta antibody to the brain parenchyma. To test this, induced meningeal lymphatic dysfunction was introduced in WT or 5×FAD mice using a previously described method of lymphatic vessel photoablation (Da Mesquita, S. et al. Functional aspects of meningeal lymphatics in ageing and Alzheimer's disease. Nature 560, 185-191, (2018); Louveau, A. et al. CNS lymphatic drainage and neuroinflammation are regulated by meningeal lymphatic vasculature. Nat Neurosci 21, 1380-1391, (2018), achieved by injecting Visudyne into the CSF and five consecutive transcranial photoconversion steps of the drug by a 689-nm-wavelength nonthermal red light. Analysis of meningeal lymphatic morphology in WT mice 1 week later revealed efficient ablation of the lymphatic vasculature lining the transverse sinus (in the dorsal meninges, FIGS. 5N and 5O), but no significant changes in the continuing lymphatic vessels present around the SS and PSS (in the basal meninges, FIGS. 5N and 5P). The fluorescent microsphere drainage from the CSF into the dCLNs in WT mice was also measured using in-vivo stereomicroscopic imaging of the collecting lymphatic vessel afferent to the dCLN (FIG. 5Q). This revealed a significant reduction in CSF drainage 1 week after photoablation of the lymphatic vasculature at the dorsal meninges (FIG. 5R). These results reinforced previous findings emphasizing the important contribution of initial lymphatics present at the dorsal brain meninges (Da Mesquita, S. et al. Functional aspects of meningeal lymphatics in ageing and Alzheimer's disease. Nature 560, 185-191, (2018); Louveau, A. et al. CNS lymphatic drainage and neuroinflammation are regulated by meningeal lymphatic vasculature. Nat Neurosci 21, 1380-1391, (2018); Louveau, A. et al. Structural and functional features of central nervous system lymphatic vessels. Nature 523, 337-341, (2015)), namely along the transverse sinus, for drainage of CSF components into the dCLNs, and call into question a more recent assertion specifying a key role for the lymphatics in the basal region of the meninges (Ahn, J. H. et al. Meningeal lymphatic vessels at the skull base drain cerebrospinal fluid. Nature 572, 62-66, (2019)).

To test the effect of decreased meningeal lymphatic drainage on anti-Abeta antibody-mediated Aβ plaque clearance, meningeal lymphatic vessel ablation was induced in 4-5 months-old 5×FAD mice, the mice were allowed to recover for 1 week, and then anti-Abeta antibody (ABETA Mab1) was administered into the CSF. By the end of the anti-Abeta antibody treatment regimen (described in FIG. 8D), mice with intact meningeal lymphatic vasculature (FIGS. 8H-8L) presented significantly less Aβ plaque load than mice with ablated meningeal lymphatics across different brain regions, especially in the cortex (FIGS. 8D-8G and FIGS. 8M and 8N).

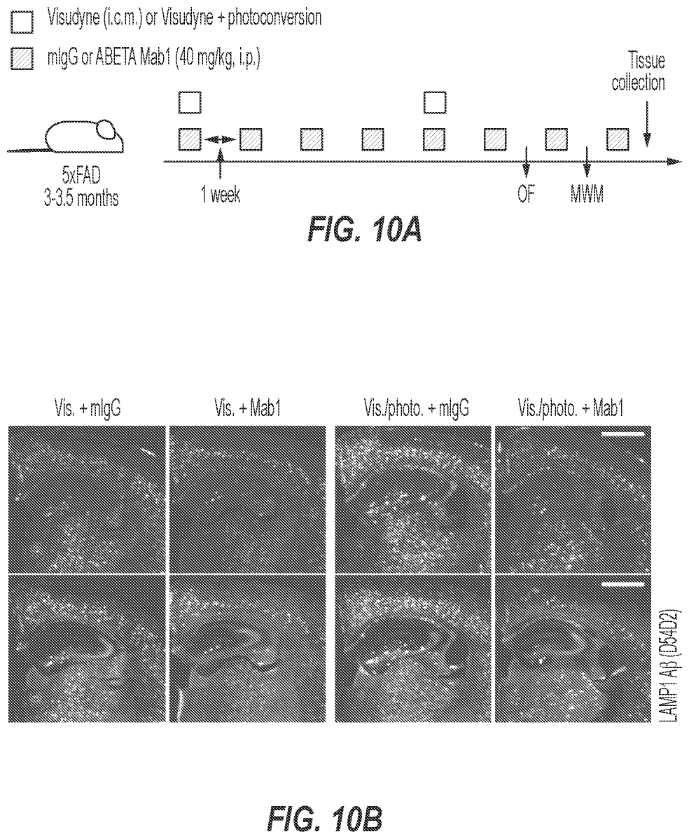

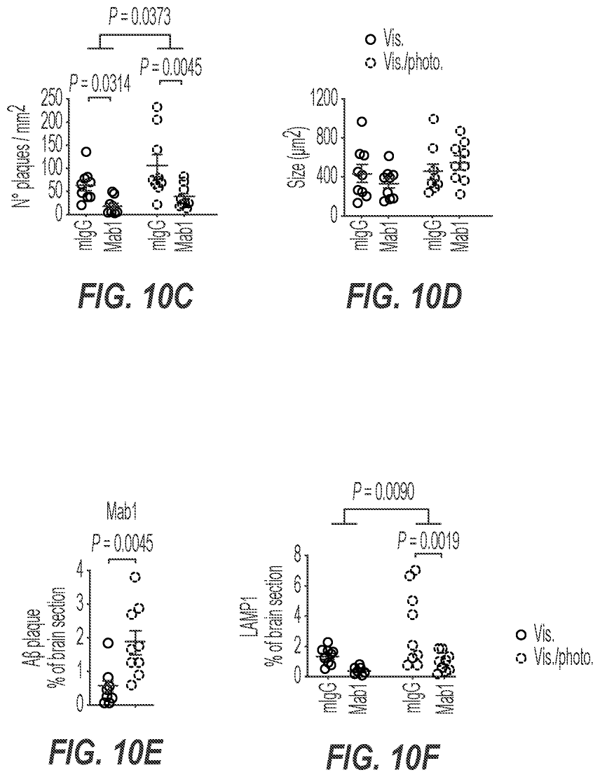

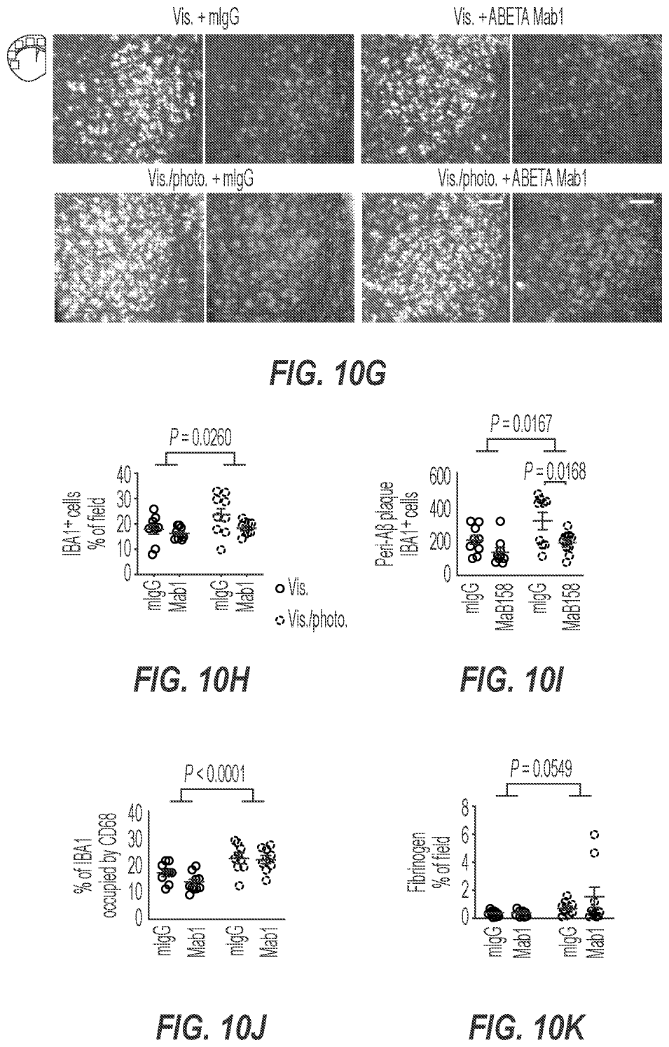

In another set of experiment 5×FAD mice of 3-3.5 months-old were injected (i.p.) with anti-Abeta antibody (ABETA Mab1) or mIgG (each at 40 mg/kg) following the exact same treatment regimen (FIG. 10A). Treatment with ABETA Mab1 proved to be efficient in reducing the density of Aβ plaques in the brain, regardless of the degree of meningeal lymphatic drainage (FIGS. 10B, 10C). ABETA Mab1 did not affect the average size of Aβ plaques (FIG. 10D). However, 5×FAD mice with dysfunctional meningeal lymphatics (Vis./photo. groups) that received ABETA Mab1 antibody showed significantly higher brain Aβ plaque coverage, when compared to their controls with intact meningeal lymphatic vasculature (Vis. groups; FIG. and 10E). Similar outcomes were observed in both cohorts in terms of brain coverage by LAMP1⁺ dystrophic neurites, which were significantly increased in 5×FAD mice with dysfunctional meningeal lymphatics (FIGS. 10B and 10F). Accordingly, measurements of IBA1⁺ cell coverage, number of peri-Aβ plaque IBA1⁺ cells, levels of CD68 on IBA1l cells and fibrinogen coverage (per field of view) revealed an aberrant activation of myeloid cells and increased fibrinogen levels in the brain of mice with reduced meningeal lymphatic drainage (FIGS. 10G-10K).

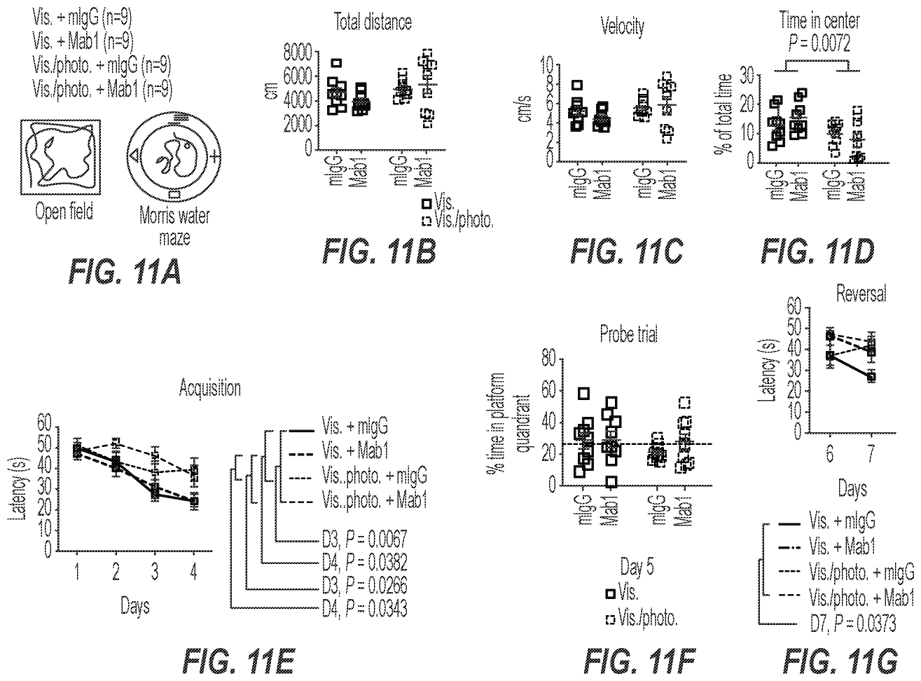

Noteworthy, the heightened neuroinflammatory response observed in 5×FAD mice with ablated meningeal lymphatic vessels was concurrent with behavioral deficits (FIGS. 11A-11G). Mice with impaired meningeal lymphatics spent less time in the center of the open field arena (FIGS. 11A-11D) and took more time to find the submerged platform in the acquisition of the Morris water maze test (FIGS. 11E-11G), when compared to their control counterparts. Prolonged treatment with the monoclonal antibody ABETA Mab1 was not able to improve the performance of 5×FAD mice in the open field or the Morris water maze (FIGS. 11A-11G). Altogether, this data is indicative of a deleterious effect of reduced meningeal lymphatic drainage on brain fibrinogen levels and neuroinflammation in 5×FAD mice, which, in accordance with previous studies, have a great impact on brain function and translate into heightened anxious-like behavior and accelerated cognitive decline.

Based on recent experimental evidence for an impaired perivascular CSF influx (via the glymphatic pathway) in mouse models of meningeal lymphatic dysfunction, it was postulated that exacerbation of meningeal lymphatic dysfunction in 5×FAD mice would dampen the clearance of Aβ plaques, owing to reduced access of monoclonal antibodies to the brain parenchyma. To test this, meningeal lymphatic vessel ablation was induced in 4-4.5 months-old 5×FAD mice, the mice were allowed to recover for 1 week, and then ABETA Mab1 or the same amount of control mIgG (a total of 5 μg each) were administered into the CSF via intra-cisterna magna injection (this regimen was repeated twice, as described in FIG. 14A). Prolonged ablation of lymphatic vasculature in the dorsal region of the meninges of 5×FAD mice led to significantly higher Aβ burden in the meninges and brain parenchyma and abrogated Aβ plaque clearance mediated by ABETA Mab1 delivered into the CSF (FIGS. 14B-14G). Moreover, meningeal lymphatic ablation resulted in increased number of ferric iron deposits (depicted by Prussian blue staining) in the brains of 5×FAD mice, a feature that was not affected by ABETA Mab1 treatment (FIGS. 14H and 14I).

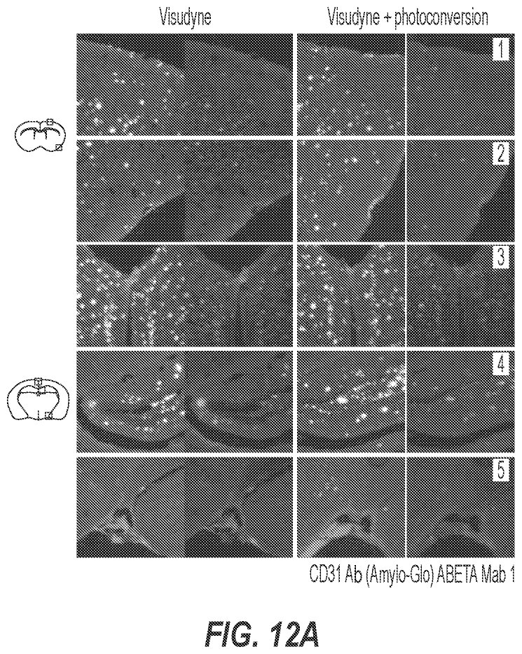

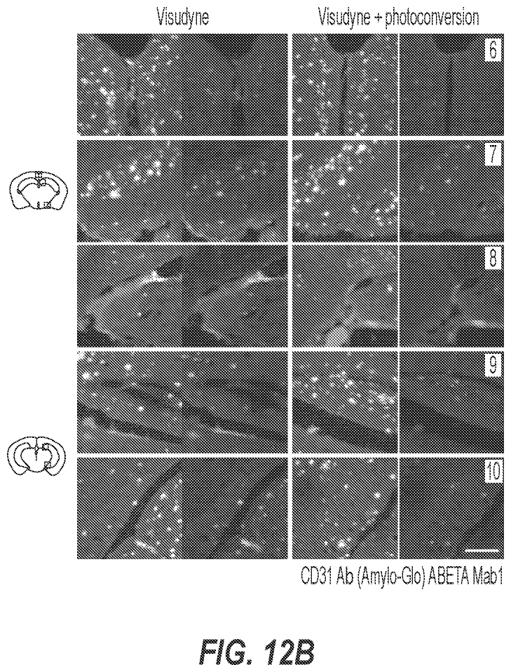

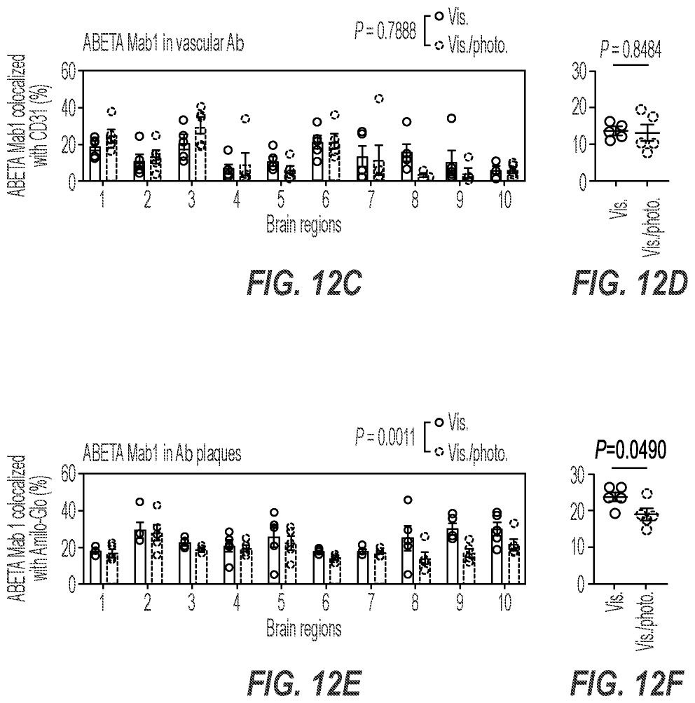

In an attempt to explain the reduced efficacy of anti-Abeta antibody observed in the mice with impaired meningeal lymphatic drainage, 1 hour after introducing antibodies into the CSF, assays were performed to measure the amounts of anti-Abeta antibody (ABETA Mab1) in the brain that were colocalized with CD31 vessels or with Aβ aggregates (FIGS. 12A-12F). Interestingly, although there were no differences in the amount of anti-Abeta antibody in the brain vasculature (FIGS. 12C and 12D), significantly less anti-Abeta antibody was found to be colocalized with brain parenchymal Aβ aggregates in the 5×FAD mice with ablated meningeal lymphatic vasculature (FIGS. 12E and 12F). These findings suggested that impairment of meningeal lymphatic drainage in 5×FAD mice leads to decreased perivascular influx of anti-Abeta antibody from the CSF into the brain, reduced access of anti-Abeta antibody to brain parenchymal Aβ plaques, and less clearance of Aβ plaques by anti-Abeta antibody.

Brain myeloid phagocytes, namely microglia and recruited macrophages, were shown to be closely involved in Fc receptor-mediated clearance of parenchymal Aβ aggregates upon injection of monoclonal antibodies against Aβ (Bard, F. et al. Peripherally administered antibodies against amyloid beta-peptide enter the central nervous system and reduce pathology in a mouse model of Alzheimer disease. Nat Med 6, 916-919, (2000); Wilcock, D. M. et al. Microglial activation facilitates Abeta plaque removal following intracranial anti-Abeta antibody administration. Neurobiol Dis 15, 11-20, (2004); Koenigsknecht-Talboo, J. et al. Rapid microglial response around amyloid pathology after systemic anti-Abeta antibody administration in PDAPP mice. J Neurosci 28, 14156-14164, (2008)). Thus, it was hypothesized that altered microglial function in mice with impaired meningeal lymphatics was contributing to decreased clearance of Aβ by anti-Abeta antibody. Single-cell RNA-seq was performed on live myeloid CD45⁺Ly6G^{neg}CD11b⁺ cells sorted from the brain cortices of 5×FAD mice with intact or ablated meningeal lymphatics (FIG. 13A). After in-silico removal of undesired cell contaminants, shared nearest neighbor clustering and t-distributed stochastic neighbor embedding, four clusters of microglia were identified (FIG. 13B). Meningeal lymphatic ablation in the 5×FAD mice did not affect the frequencies of homeostatic microglia (clusters 1 and 2) or of microglia displaying the two-step disease-associated signature, namely the Triggering receptor expressed on myeloid cells 2 (Trem2)-independent (cluster 3) or the Trem2-dependent (cluster 4) gene expression profiles (Keren-Shaul, H. et al. A Unique Microglia Type Associated with Restricting Development of Alzheimer's Disease. Cell 169, 1276-1290 e1217, (2017)) (FIGS. 13C and 13D). Table 19 summarizes the results as shown in FIG. 13D. However, analysis of the genes that are differentially expressed in the two groups (intact or ablated meningeal lymphatics) revealed a downregulation of hexosaminidase subunit beta (Hexb) and an upregulation of apolipoprotein E (Apoe) across all microglial clusters, and these changes were significant when all cells were pooled by group (FIGS. 13E-13H). Although no changes were observed in the expression of genes encoding for Fc receptor proteins in microglia, the decreased efficacy of anti-Abeta antibody treatment in 5×FAD mice with impaired meningeal lymphatic vessels might be associated with differential expressions of Hexb and Apoe and with a significantly higher expression of the major histocompatibility complex II genes H2-Aa, H2-Ab1 and Cd74, and the major histocompatibility complex I gene H2-D1 (FIGS. 13I-13L), all important participants in the regulation of microglial function and response (Keren-Shaul, H. et al. A Unique Microglia Type Associated with Restricting Development of Alzheimer's Disease. Cell 169, 1276-1290 e1217, (2017); Krasemann, S. et al. The TREM2-APOE Pathway Drives the Transcriptional Phenotype of Dysfunctional Microglia in Neurodegenerative Diseases. Immunity 47, 566-581 e569, (2017)). Concomitantly, it was observed that there was a significant increase in cortical CD45^{high} cells (recruited leukocytes and/or activated microglia, FIGS. 13M-13Q) and in the levels of H-2Kd (encoded by the H2-D1 gene) in both CD45^{int}CD11b⁺ (microglia) and CD45high CD11b^{neg} (recruited lymphoid cells) cells in mice with meningeal lymphatic dysfunction (FIGS. 13R-13U). These observations corroborate the single-cell RNA-seq data from microglia, and also point to a possible role of H-2Kd-expressing microglia and recruited leukocytes (coming from the blood) to the worsen Aβ plaque load observed in mice with impaired meningeal lymphatic drainage.

### Example 7: Enhancing Meningeal Lymphatics Modulates Anti-Aβ Immunotherapy

Most monoclonal anti-Aβ antibodies tested in clinical trials have failed to significantly prevent cognitive decline in AD patients, possibly owing to serious side effects such as deleterious activation of blood vasculature and microglia (Ryu, J. K. et al. Fibrin-targeting immunotherapy protects against neuroinflammation and neurodegeneration. Nat Immunol 19, 1212-1223, (2018); Merlini, M. et al. Fibrinogen Induces Microglia-Mediated Spine Elimination and Cognitive Impairment in an Alzheimer's Disease Model. Neuron 101, 1099-1108 e1096, (2019); Hong, S. et al. Complement and microglia mediate early synapse loss in Alzheimer mouse models. Science 352, 712-716, (2016)) or microhemorrhages in the brain and meninges (Salloway, S. et al. Two phase 3 trials of bapineuzumab in mild-to-moderate Alzheimer's disease. N Engl J Med 370, 322-333, (2014); Sperling, R. et al. Amyloid-related imaging abnormalities in patients with Alzheimer's disease treated with bapineuzumab: a retrospective analysis. Lancet Neurol 11, 241-249, (2012); Pfeifer, M. et al. Cerebral hemorrhage after passive anti-Abeta immunotherapy. Science 298, 1379, (2002)). It was postulated that improving meningeal lymphatic drainage would improve the efficacy of anti-Abeta antibody and potentially reduce immunotherapy-associated side effects. To explore this possibility, a combination therapy was tested. Passive anti-Abeta antibody immunotherapy was combined with adeno-associated virus 1 (AAV1)-mediated expression of murine vascular endothelial growth factor-C (mVEGF-C, FIG. 15A). Increased VEGF-C signaling through VEGFR3 expressed by meningeal LECs was previously shown by research groups to improve meningeal lymphatic function and CSF drainage (Da Mesquita, S. et al. Functional aspects of meningeal lymphatics in ageing and Alzheimer's disease. Nature 560, 185-191, (2018); Louveau, A. et al. Structural and functional features of central nervous system lymphatic vessels. Nature 523, 337-341, (2015); Antila, S. et al. Development and plasticity of meningeal lymphatic vessels. J Exp Med 214, 3645-3667, (2017)). Introduction of mVEGF-C-expressing virus together with anti-Abeta antibody into the CSF of 5×FAD mice (following the regimen described in FIG. 15A) had a synergistic effect in terms of Aβ plaque clearance from the brain (FIGS. 15B and 15C). The reductions in brain Aβ load and microgliosis were accompanied by significant expansion of lymphatic vasculature around the transverse sinus at the dorsal meninges when compared to that seen in the groups treated with anti-Abeta antibody (ABETA Mab1) and the control AAV1 (expressing enhanced green fluorescent protein, eGFP) or with mIgG2a and the AAV1s (FIGS. 15H-15I and FIGS. 15K-15M). There were no significant differences between the groups in terms of length or complexity of lymphatic vasculature in the basal region of the meninges (FIGS. 15H and 15J), or the blood vessel coverage in meningeal whole mounts (FIGS. 15K and 15L).

In mice treated with mVEGF-C and anti-Abeta antibody (ABETA Mab1), measurements of brain Aβ plaque load, neurite dystrophy (assessed by the levels of lysosomal-associated membrane protein 1), vascular fibrinogen deposition, and myeloid (IBA1⁺) cell response at the end point of the treatment regimen (2 weeks after the last injection of mIgG2a or anti-Abeta antibody) revealed a significant reduction of Aβ plaque coverage throughout the entire forebrain (FIGS. 15N-15R), less cortical vascular fibrinogen (FIGS. 15S-15U), fewer IBA1⁺ cells clustering around Aβ deposits, and decreased amounts of CD68 in IBA1⁺ cells (FIGS. 15D-15G). Groups that received anti-Abeta antibody presented significantly less dystrophic neurites, regardless of the treatment with mVEGF-C (FIGS. 15S and 15T). In sum, combination therapy with mVEGF-C and anti-Abeta antibody revealed a close connection between restoration of meningeal lymphatic morphology and improved Aβ clearance by anti-Abeta antibody, with less vascular fibrinogen deposition and myeloid cell activation (which have been linked to worse disease outcome in AD transgenic mice (Ryu, J. K. et al. Fibrin-targeting immunotherapy protects against neuroinflammation and neurodegeneration. Nat Immunol 19, 1212-1223, (2018); Merlini, M. et al. Fibrinogen Induces Microglia-Mediated Spine Elimination and Cognitive Impairment in an Alzheimer's Disease Model. Neuron 101, 1099-1108 e1096, (2019); Hong, S. et al. Complement and microglia mediate early synapse loss in Alzheimer mouse models. Science 352, 712-716, (2016))).

Previously published data showed improved CSF perivascular influx (glymphatic function) into the brains of aged mice upon VEGF-C-induced enhancement of meningeal lymphatic drainage (Da Mesquita, S. et al. Functional aspects of meningeal lymphatics in ageing and Alzheimer's disease. Nature 560, 185-191, (2018)). Assays were performed to assess the meningeal LEC transcriptomic signature associated with improved meningeal lymphatic function following mVEGF-C treatment in 20-24 months-old mice (FIGS. 16A-16D). Tables 20 and 21 summarize the results as shown in FIG. 16C.

These results led us to further explore the effects of enhancing meningeal lymphatic drainage on Aβ clearance by monoclonal antibodies and brain tissue homeostasis in aged AD transgenic mice. Direct injection into the CSF of aged J20 mice (14-16 months-old, FIGS. 16E-16I) and of APPswe mice (26-30 months-old, FIGS. 16J-16N) with mVEGF-C-expressing antibody and anti-Abeta (ABETA Mab1) resulted in brain Aβ clearance when compared to that in mice treated with the control eGFP-expressing virus.

Specifically, J20 mice were treated with a combination of VEGF-c and ABETA Mab1 antibody that binds amyloid beta. Representative images of the brain sections of J20 mice treated with ABETA Mab1 and with the antibody and either AAV1-CMV-eGFP or AAV1-CMV-mVEGF-C are shown in FIG. 16F. Brain sections were stained for Aβ and with DAPI. FIGS. 16G-16I show amyloid beta plaque coverage (% of brain section) in the hippocampus, cortex and amygdala, or in the hippocampus, cortex and amygdala combined. Amyloid beta plaque coverage in the hippocampus, cortex and amygdala were lessened in mice that received AAV1-vector expressing VEGF-c (see FIGS. 16F-16I). This experiment shows that a flow modulator and neurological therapeutic agent in an additional model of AD (J20 mice) comprising amyloid beta plaques in accordance with some embodiments herein can synergize to reduce amyloid beta plaque coverage (as percent of brain region).

### Example 8: Disease-Associated Genes are Highly Expressed by Lymphatic Endothelial Cells

To further explore the relationship between changes in the lymphatic system and AD, it was hypothesized that higher risk for AD, owing to disease-associated single nucleotide polymorphisms (SNPs), could be directly linked to altered function of the lymphatic vasculature. During the last decade, genome-wide association studies have revealed numerous genes containing SNPs that affect the risk for AD (Guerreiro, R. et al. TREM2 variants in Alzheimer's disease. N Engl J Med 368, 117-127, (2013); Naj, A. C. et al. Common variants at MS4A4/MS4A6E, CD2AP, CD33 and EPHA1 are associated with late-onset Alzheimer's disease. Nat Genet 43, 436-441, (2011)), as well as for other neurological disorders (Buniello, A. et al. The NHGRI-EBI GWAS Catalog of published genome-wide association studies, targeted arrays and summary statistics 2019. Nucleic Acids Res 47, D1005-D1012, (2019)). Interestingly, many of the genes associated with increased risk for Alzheimer's, Parkinson's, schizophrenia, autism and multiple sclerosis are highly expressed in LECs (FIG. 17A). This is true not only for LECs isolated from the meninges, but also from different peripheral tissues (diaphragm and ear skin), and regardless of the age of the mice (2-3, 6, or 20-24 months), genotype (WT or 5×FAD) or viral-mediated expression of eGFP or mVEGF-C (FIG. 17B). Similar disease-associated gene expression was also observed in cultured human LECs and, remarkably, in capillary, arterial and venous endothelial cells of the brain vasculature (FIGS. 18A-18D), emphasizing the importance of lymphatic vasculature alongside brain blood vasculature in AD pathophysiology. Table 27 summarizes the results as shown in FIG. 18B. Table 28 summarizes the results as shown in FIG. 18D. Functional pathway analysis using disease-associated genes expressed in LECs revealed changes in vascular development (GO:0001944), cell projection (GO:0048858) and tube morphogenesis (GO:0030198), negative regulation of cell differentiation (GO:0045596) and migration (GO:0030334) and positive regulation of cell death (GO:0010942) across two or more diseases (FIG. 17C). Regarding AD-related pathways, changes were also observed in cell-matrix adhesion (GO:0007160), cell-cell junction (GO:0005911), amyloid beta metabolic process (GO:0050435) and lipoprotein particle binding (GO:0071813), which stress out once again that altered gene expression in LECs might lead to structural and morphological changes in the lymphatic vasculature and potentially impact on lymphatic vessel integrity, LEC survival and Aβ metabolism (FIG. 17C). Interestingly, meningeal LECs and brain BECs presented a higher proportion of highly expressed AD-associated genes (FIGS. 17A, 17B, 17C and FIGS. 18C, 18D), when compared to microglia from the 5×FAD cortex (FIGS. 18E-18G). In fact, within the 10^{th} percentile AD-associated genes, 39 genes were uniquely expressed by meningeal LECs, which was more than the 17 and 20 genes uniquely expressed by BECs and microglia, respectively (FIG. 19). Of note, Apoe, which is intrinsically linked to altered risk for late onset AD, was one of the genes found at the intersection between all three cell types (FIG. 19).

To identify target genes that are essential for normal function, gene expression profiles under various conditions were evaluated to study the difference in lymphatic endothelial cell constitution. As shown in FIG. 24A, meningeal lymphatic endothelial cells have signatures that distinguish them from diaphragm and skin lymphatics. Gene expression profiles were also evaluated in meningeal lymphatics of young and old mice. Changes in genes associated with immune system, growth factor, and extracellular matrix pathways in meningeal lymphatics were observed (FIG. 24B).

The change of the meningeal lymphatics affects the gene expression profile in brain tissue. As shown in FIG. 24C, changes in gene expression were observed in hippocampal cells after blockage of meningeal lymphatics.

The impact of Aβ on meningeal lymphatics was observed in LEC culture. The gene expression profile of key AD-specific pathways was impacted in LEC cultures treated with Aβ in a temporal manner (FIG. 25).

The differentially expressed genes that is uniquely expressed in the meningeal lymphatics were identified as potentially targets for drugs for the treatment of AD (FIG. 26).

### Example 9: Discussion

It has been previously shown that induction of meningeal lymphatic dysfunction in young-adult 5×FAD mice results in worsened meningeal amyloid angiopathy (Da Mesquita, S. et al. Functional aspects of meningeal lymphatics in ageing and Alzheimer's disease. Nature 560, 185-191, (2018)). In this disclosure, data obtained from the studies suggest that a buildup of aggregation-prone Aβ species around meningeal lymphatic vessels could induce changes in LEC transcriptome that are associated with an accelerated loss of lymphatic coverage and accumulation of adaptive immune cells in the meninges. The efficacy of passive immunotherapy with anti-Abeta antibody is greatly reduced in 5×FAD mice with defective meningeal lymphatic drainage. Most importantly, either ablating meningeal lymphatic vessels or enhancing their function by mVEGF-C in mouse models of familial AD led to significant transcriptional changes in microglial and brain BECs. These data suggests that it might be possible to devise strategies to therapeutically target both microglia and the brain blood vasculature, two important players in AD pathophysiology, by modulating the function of the lymphatic vasculature at the brain borders. These findings also underscore the importance of early diagnosis and therapeutic intervention in AD patients, preferentially at a stage when the meningeal lymphatic system is still operational. The advanced stage of disease (or simply the advanced age) at which antibody-based therapies are administered might explain their marginal beneficial effects and/or potential deleterious side effects which, as these results suggest, could be attributable, at least in part, to a compromised meningeal lymphatic function.

Altogether, results of combination therapy with mVEGF-C, either prophylactic to prevent meningeal lymphatic dysfunction in 4-5 months-old 5×FAD mice, or therapeutic to augment meningeal lymphatic drainage in aged J20 and APPswe mice (both of which develop less brain Aβ pathology and at a later age), show synergistically improved clearance of Aβ by anti-Abeta antibody. These results also seem to indicate that administering anti-Aβ antibody together with mVEGF-C directly into the CSF of aged mice, in detriment of a peripheral route of administration that has to rely on transport mechanisms across the blood-brain barrier, might improve Aβ plaque clearance from the brain. The data support a combination of immunotherapies that target brain Aβ (or potentially other disease-related proteins such as APOE (Liao, F. et al. Targeting of nonlipidated, aggregated apoE with antibodies inhibits amyloid accumulation. J Clin Invest 128, 2144-2155, (2018)), Tau (Yanamandra, K. et al. Anti-tau antibodies that block tau aggregate seeding in vitro markedly decrease pathology and improve cognition in vivo. Neuron 80, 402-414, (2013)) or fibrin (Ryu, J. K. et al. Fibrin-targeting immunotherapy protects against neuroinflammation and neurodegeneration. Nat Immunol 19, 1212-1223, (2018)) with therapeutic strategies aimed at improving meningeal lymphatic function, in order to maximize clearance of these pathological proteinaceous species from the brain in AD. Ultimately, therapeutic targeting of meningeal lymphatic vasculature might be of relevance for other neurodegenerative disorders characterized by protein misfolding and accumulation, such as Huntington's or Parkinson's diseases, where administration of antisense oligonucleotides (Kordasiewicz, H. B. et al. Sustained therapeutic reversal of Huntington's disease by transient repression of huntingtin synthesis. Neuron 74, 1031-1044, (2012)) or of monoclonal antibodies against α-synuclein (Weihofen, A. et al. Development of an aggregate-selective, human-derived alpha-synuclein antibody BIIB054 that ameliorates disease phenotypes in Parkinson's disease models. Neurobiol Dis 124, 276-288, (2019)) into the CSF are also being considered as promising therapeutic strategies.

### Example 10: Materials and Methods

The following materials and methods were used in the studies described in Examples 16-18.

Mouse strains and housing. Adult (2-3 months-old) male C57BL/6J wild type (WT) mice were purchased from the Jackson Laboratory (JAX stock #000664, Bar Harbor, Me., USA). Aged (20-24 months-old) WT mice were provided by the National Institutes of Health/National Institute on Aging (Bethesda, Md., USA). All mice were maintained in the animal facility for habituation for at least 1 week prior to the start of the experiment. Male hemizygous B6.Cg-Tg(APPSwFlLon,PSEN1*M146L*L286V)6799Vas/Mmjax (5×FAD, JAX stock #008730), B6.Cg-Tg(PDGFB-APPSwInd)20Lms/2Mmjax (J20, JAX stock #006293) and B6.Cg-Tg(APP695)3Dbo (APPswe, JAX stock #005866) were purchased from the Jackson Laboratory and bred in-house on a C57BL/6J background. In-house bred male or female transgene carriers and non-carrier (WT) littermates were used at different ages. The genotype and age of mice from different strains are indicated in figure schemes or legends throughout the manuscript. Male mice were used in the different experiments, unless stated otherwise. Mice of all strains were housed in an environment with controlled temperature and humidity and on a 12-hour light/dark cycle (lights on at 7:00). All mice were fed with regular rodent's chow and sterilized tap water ad libitum. All experiments were approved by the Institutional Animal Care and Use Committee of the University of Virginia.

Treatments with anti-Aβ monoclonal antibodies. ABETA Mab1 and respective control IgG2a (mIgG) antibodies were manufactured by Absolute Antibody Ltd., Oxford Centre for Innovation, United Kingdom. Anti-Aβ monoclonal antibodies and respective mIgG controls were administered via intraperitoneal (i.p.) injection, at a dose of 40 mg/kg. Antibody dosages are also specified in the main text, illustrated in schemes and in each figure legend. Alternatively, ABETA Mab1 and mIgG antibodies were injected directly into the CSF, following the methodology described in the next section.

Intra-cisterna magna and intravenous injections. Mice were anaesthetized by intraperitoneal (i.p.) injection of a mixed solution of ketamine (100 mg/Kg) and xylazine (10 mg/Kg) in saline. The skin of the neck was shaved and cleaned with iodine and 70% ethanol, ophthalmic solution placed on the eyes to prevent drying and the mouse's head was secured in a stereotaxic frame. After making a small (4-5 mm) skin incision, the muscle layers were retracted and the atlantooccipital membrane of the cisterna magna was exposed. Using a Hamilton syringe (coupled to a 33-gauge needle), the volume of the desired solution was injected into the CSF-filled cisterna magna compartment at a rate of ˜2.5 μL/min. After injecting, the syringe was left in place for at least 2 min to prevent back-flow of CSF. The neck skin was then sutured, the mice were allowed to recover in supine position on a heating pad until fully awake and subcutaneously injected with ketoprofen (2 mg/Kg). This method of intra-cisterna magna (i.c.m.) injection was used to administer 5 μL of either Visudyne® (verteporfin for injection, Valeant Ophtalmics), adeno associated viral vectors (AAV1-CMV-mVEGF-C-WPRE and AAV1-CMV-eGFP at 10¹² genome copies per mL, purchased from Vector BioLabs, Philadelphia) or different antibody solutions of murine Abeta MAb or IgG2a (manufactured by Absolute Antibody Ltd., Oxford Centre for Innovation, United Kingdom). Alternatively, antibodies were also injected into the tail vein of mice (i.v.). Antibody dosages/titers are specified in the main text and in each figure legend.

Meningeal lymphatic vessel ablation. Selective ablation of the meningeal lymphatic vessels was achieved by injection of Visudyne (Vis.) and consecutive transcranial photoconversion (photo.) steps following previously described methodology and regimens (Da Mesquita, S. et al. Functional aspects of meningeal lymphatics in ageing and Alzheimer's disease. Nature 560, 185-191, (2018); Louveau, A. et al. CNS lymphatic drainage and neuroinflammation are regulated by meningeal lymphatic vasculature. Nat Neurosci 21, 1380-1391, (2018)). Visudyne was reconstituted following manufacturer instructions, aliquoted and kept at −20° C. until further used. Immediately upon being thawed, Visudyne was injected into the CSF (i.c.m.) and, 15 min later, an incision was performed in the skin to expose the skull bone and photoconvert the drug by pointing a 689-nm wavelength nonthermal red light (Coherent Opal Photoactivator, Lumenis) on 5 different spots above the intact skull (close to the injection site, above the superior sagittal sinus close to the rostral rhinal vein, above the confluence of sinuses and above each transverse sinus). Each spot was irradiated with a light dose of 50 J/cm² at an intensity of 600 mW/cm² for a total of 83 s. Controls were injected with the same volume of Visudyne only, without the photoconversion step. The scalp skin was then sutured, the mice were allowed to recover on a heating pad until fully awake and subcutaneously injected with ketoprofen (2 mg/Kg).

In vivo measurement of CSF outflow into dCLNs. Upon i.c.m. injection of 5 μL of a suspension of 0.5 pm yellow-green fluorescent (505/515 nm) microspheres (FluoSpheres™ carboxylate-modified microspheres, Thermo Fisher Scientific) diluted in artificial CSF (1:1 v/v,) following the procedure described previously, the syringe was left in place for 10 min to prevent backflow and then the mouse was prepared for live imaging of microsphere drainage from the CSF into the dCLNs using a stereomicroscope (M205 FA, Leica Microsystems). The mouse was positioned supine with the head held in position with a length of suture behind the upper incisors and the upper limbs held in place with medical tape. Incisions were made from the center of the clavicle, anterior to the top of the salivary gland and lateral approximately 1 cm. The further preparation was performed on the right side, however in some instances moved to the left side when anatomical variation prevented imaging. The salivary gland was carefully separated at its lateral extent and gently retracted medially. The omohyoid and sternomastoid muscles were retracted laterally, exposing the dCLN. Imaging began approximately 15 minutes after i.c.m. injection. Images were acquired at 25-30 frames per second for a total of 60 seconds. After imaging, mice were euthanized by injection of Euthasol (10% v/v in saline). Fluorescent microsphere drainage was analyzed in FIJI software by drawing a line demarcating the draining lymphatic vessel afferent to the dCLN and manually counting the beads passing the line by a blinded experimenter. Mice were discarded from the analysis due to prior complications during the surgical procedure (e.g. hemorrhages) or due to failure in detecting microspheres draining into the dCLN during image acquisition. In representative images, microspheres were tracked using TrackMate (Tinevez, J. Y. et al. TrackMate: An open and extensible platform for single-particle tracking. Methods 115, 80-90, (2017)) to show the comulative tracks over a 20 see interval.

Open field test. Mice were habituated to the behavior room, including the background white noise, for at least 30 min prior to starting the test. Individual mice were then placed into the open field arena (made of opaque white plastic material, 35 cm×35 cm) by a blinded experimenter and allowed to explore it for 15 min. Total distance (in cm), velocity (in mm per second) and % time spent in the center (22 cm×22 cm area) were quantified using video tracking software (TopScan, CleverSys, Inc.) and analyzed in Microsoft Excel and Prism 8.3.4 (GraphPad Software, Inc.).

Morris water maze test. Mice were habituated to the behavior room, including the background white noise, for at least 30 min before starting the test. The MWM test consisted of 4 days of acquisition, 1 day of probe trial and 2 days of reversal. In the acquisition, mice performed four trials per day, for 4 consecutive days, to find a hidden 10-cm diameter platform located 1 cm below the water surface in a pool 1 m in diameter. Tap water was made opaque with nontoxic white paint (Prang ready-to-use washable tempera paint) and the water temperature was kept at 25±1° C. by addition of warm water. A dim light source was placed within the testing room and only distal visual cues were available above each quadrant of the swimming pool to aid in the spatial navigation and location of the submerged platform. The latency to platform, i.e., the time required by the mouse to find and climb onto the platform, was recorded for up to 60 seconds. Each mouse was allowed to remain on the platform for ˜15 seconds and was then moved from the maze to its home cage. If the mouse did not find the platform within 60 seconds, it was manually placed on the platform and returned to its home cage after ˜15 seconds. The inter-trial interval for each mouse was of at least 5 min. On day 5, the platform was removed from the pool, and each mouse was tested in a probe trial for 60 seconds. On days 1 and 2 of the reversal, without changing the position of the visual cues, the platform was placed in the quadrant opposite to the original acquisition quadrant and the mouse was retrained for four trials per day. All MWM testing was performed between 10 a.m. and 6 p.m., during the lights-on phase, by a blinded experimenter. During the acquisition, probe and reversal, data were recorded using the EthoVision automated tracking system (Noldus Information Technology). The mean latency (in seconds) of the four trials for each day of test and the % of time in the platform quadrant during the probe trial were calculated in Excel and statistically analyzed in Prism 8.3.4.

Tissue collection and processing. Mice were given a lethal dose of anesthetics by intraperitoneal (i.p.) injection of Euthasol (10% v/v in saline) and transcardially perfused with ice cold phosphate buffer saline (PBS, pH 7.4) with heparin (10 U/mL). After stripping the skin and muscle from the bone, the entire head was collected and drop fixed in 4% paraformaldehyde (PFA) for 24 hours at 4° C. After removal of the mandibles and nasal bone, the top of the skull (skull cap) was removed with fine surgical curved scissors (Fine Science Tools) by cutting clockwise, beginning and ending inferior to the right post-tympanic hook and kept in PBS 0.02% azide at 4° C. until further use. Fixed meninges (dura mater and arachnoid) were carefully dissected from the skullcaps with Dumont #5 and #7 fine forceps (Fine Science Tools) and kept in PBS 0.02% azide at 4° C. until further use. Alternatively, the skull was cut sagitally, along the median plane, and after removing the brain, the skull pieces with the attached meningeal layers were kept in PBS 0.02% azide at 4° C. until further use. The brains were kept in 4% PFA for additional 24 hours (48 hours in total). Fixed brains were washed with PBS, cryoprotected with 30% sucrose and frozen in Tissue-Plus® O.C.T. compound (Thermo Fisher Scientific). Fixed and frozen brains were sliced (50 μm thick sections) with a cryostat (Leica) and kept in PBS 0.02% azide at 4° C.

Immunohistochemistry, imaging and quantifications. The following steps were generally applied for free floating brain sections and meningeal whole mounts. When appropriate, prior to immunofluorescent staining, brain sections were stained for amyloid deposits with the Amylo-Glo® RTD™ reagent (Biosensis, Fine Bioscience Tools, South Australia), following manufacturer instructions. For immunofluorescence staining, tissue was rinsed in PBS and incubated with PBS 0.5% Triton X-100 (Thermo Fisher Scientific, PBS-T) for 30 min, followed by PBS-T containing 0.5% of normal serum (either goat or chicken) or 0.5% bovine serum albumin (BSA) for 30 min at room temperature (RT). This blocking step was followed by incubation with appropriate dilutions of primary antibodies: rat anti-LYVE-1-eFluor660 or anti-LYVE-1-Alexa Fluor®488 (eBioscience, clone ALY7, 1:200), Armenian hamster anti-CD31 (Millipore Sigma, MAB1398Z, clone 2H8, 1:200), rabbit anti-AQP4 (Millipore Sigma, A5971, 1:200), goat anti-IBA1 (Abcam, ab5076, polyclonal, 1:200), rat anti-CD68 (BioLegend, 137002, clone FA-11, 1:100), rat anti-LAMP-1 (Abcam, ab25245, clone 1D4B, 1:300), rabbit anti-Fibrinogen (Dako, A0080, polyclonal, 1:200), anti-Aβ_{1-37/42} (Cell Signaling, 8243S, clone D54D2, 1:400) in PBS-T containing 0.5% BSA overnight at 4° C. Meningeal whole mounts or brain sections were then washed 3 times for 10 min at RT in PBS-T followed by incubation with the appropriate rat, chicken, goat or donkey eFluor570 or Alexa Fluor® 488, 594, or 647 conjugated anti-rat, -goat, -rabbit, -mouse or -Armenian hamster IgG antibodies (Thermo Fisher Scientific, 1:500) for 1 hour at RT in PBS-T. After an incubation for 10 min with 1:5000 DAPI in PBS, the tissue was washed 3 times for 5 min with PBS, left to dry at RT (10-20 minutes) and mounted with Shandon™ Aqua-Mount (Thermo Fisher Scientific) and glass coverslips. To stain lymphatic vasculature in the intact skull cap meninges, the same skull hemisphere was incubated in PBS-T 0.5% BSA for 2 hours and then with anti-LYVE-1 eFluor 660 (1:100) in PBS-T 0.5% BSA for 48 hours. Skull caps were then washed 3 times for 1 hour with PBS-T and left washing in PBS-T overnight at 4° C. Skull caps were washed once with PBS kept in PBS at 4° C. Preparations were stored at 4° C. for no more than 1 week until images were acquired. A stereomicroscope (M205 FA, Leica Microsystems) was used to image the meningeal lymphatic vessels within the skull caps. A widefield microscope (DMI6000 B with Adaptive Focus Control, Leica Microsystems) was used for images of Aβ deposits in brain sections and a confocal microscope (FV1200 Laser Scanning Confocal Microscope, Olympus) to acquire all the other images. Upon acquisition, images were opened in the FIJI software for quantification. The ROI (region of interest) manager, Simple Neurite Tracer and Cell Counter plugins were used to measure total lymphatic vessel length and branching points in a particular region of the meningeal whole mount. The Threshold and Measure plugins were used to measure the coverage (as % of ROI or as area in μm²) by Aβ in the brain (in delineated hippocampus, cortex/striatum/amygdala, thalamus/hypothalamus, or whole brain section; plotted values resulted from the average of 3 representative sections per sample) and meninges, as well as LAMP-1, Fibrinogen and IBA1 in images of the brain cortex (plotted values resulted from the average of 4 representative images taken from 2 brain sections per sample). The Analyze Particles plugin (Size, 4-infinity μm²; Circularity, 0.05-1) was used to measure the number of Aβ plaques per mm² of brain region/section and average size of the plaques (μm²). The Cell Counter plugin was also used to quantify the number of peri-AP plaque IBA1⁺ cells (cell body within 10 μm of plaque). The Threshold and Image Calculator plugins were used to determine the % of colocalization between the signals of anti-Abeta antibody and CD31, anti-Abeta antibody and Aβ (Amylo-Glo RTD), or IBA1 and CD68 in brain images acquired using the confocal microscope. All measurements were performed by a blinded experimenter, Microsoft Excel was used to calculate average values in each experiment and statistical analysis performed using Prism 7.0a (GraphPad Software, Inc.).

Flow cytometry. Mice were injected with Euthasol (i.p.) and were transcardially perfused with ice cold PBS with heparin. The brains were collected into ice-cold RPMI 1640 (Gibco), and the cortices were dissected after removing hippocampus and remnants of choroid plexus and pia matter. Individual meninges were immediately dissected from the mouse's skull cap in ice-cold RPMI 1640. The tissues were digested for 20 min at 37° C. with 1 mg/mL of Collagenase VIII, 1 mg/mL of Collagenase D and 50 U/mL of DNAse I (all from Sigma Aldrich) in RPMI 1640. The same volume of RPMI with 5% FBS (Atlas Biologicals) and 10 mM EDTA (Thermo Fisher Scientific) was added to the digested tissue, which was then filtered through a 70 μm cell strainer (Fisher Scientific). The cell pellets were washed, resuspended in ice-cold fluorescence-activated cell sorting (FACS) buffer (pH 7.4; 0.1 M PBS; 1 mM EDTA and 1% BSA), preincubated for 10 min at 4° C. with Fc-receptor blocking solution (rat anti-mouse CD16/32, clone 93, BioLegend, 1:200 in FACS) and stained for extracellular markers with the following antibodies (all at 1:200 in FACS): anti-TCRy6-FITC (11-5811-82, eBioscience), anti-CD45-BB515 (564590, BD Bioscience), anti-NK1.1-PE (553165, BD Bioscience), anti-B220-PE (553090, BD Bioscience), anti-CD4-PerCP-Cy5.5 (550954, BD Bioscience), anti-CD64-PerCP-Cy5.5 (139308, BioLegend), anti-CD8α-PE-Cy7 (552877, BD Bioscience), anti-CD11c-PE-Cy7 (558079, BD Bioscience), anti-PD-1-APC (135210, BioLegend), anti-CD45-A700 (560510, BD Bioscience), anti-CD19-A700 (557958, BD Bioscience), anti-MHC-II-eFluor450 (48-5321-82, eBioscience) and anti-TCRβ-BV510 (563221, BD Bioscience). Cell viability was determined by using the Zombie NIR™ or Zombie AQUA™ Viability Kits following the manufacturer's instructions (BioLegend). After an incubation period of 25 min at 4° C., cells were washed with FACS buffer and fluorescence data was collected with a Gallios™ Flow Cytometer (Beckman Coulter, Inc.). Data was analyzed using FlowJo™ 10 software (Tree Star, Inc.). Briefly, singlets were gated using the height, area and the pulse width of the forward and side scatters and then viable leukocytes were selected as CD45⁺Zombie NIR^{neg} or Zombie AQUA^{neg} (CD45⁺ live). Cells were then gated for the appropriate cell type markers. An aliquot from the unstained single cell suspensions was incubated with ViaStain™ AOPI Staining Solution (CS2-0106, Nexcelom Bioscience) to provide accurate counts for each sample using Cellometer Auto 2000 (Nexcelom Bioscience). Data processing was done with Excel and statistical analysis performed using Prism 7.0a (GraphPad Software, Inc.).

Mass cytometry and high-dimensional data analysis. Prior to the start of the experiment, metal isotope-labeled antibodies were purchased from Fluidigm or conjugated in-house with Maxpar (MP) antibody conjugation kits (Fluidigm) following the manufacturer's protocol. Mice were euthanized and transcardially perfused, skull caps were collected, and brain meninges were harvested and digested to obtain a final single cell suspension following the same methodology described in the flow cytometry section. Cell suspensions resulting from each brain meninges were transferred in a 96-well plate and washed with MP PBS. An aliquot from the unstained single cell suspensions was incubated with ViaStain™ AOPI Staining Solution to provide accurate counts for each sample using Cellometer Auto 2000. Unless stated otherwise, all washes and incubations were performed with Maxpar Cell Staining Buffer (MP CSB). Individual samples were incubated with 50 μL of 2.5 μM cisplatin (Fluidigm) in MP PBS for 5 min at RT, followed by two washes, and preincubated with Fc-receptor blocking solution (rat anti-mouse CD16/32 in MP PBS supplemented with 0.5% BSA) for 15 min at 4° C. Cells were then stained for fixation-sensitive surface markers for 30 min at 4° C. with: anti-Ly6C-Nd-142 (clone, REF), anti-CD169-Sm-147 (clone H1.2F3, Maxpar Ready, conjugated in-house), anti-XCR1-Eu-153 (clone ZET, BioLegend, conjugated in-house), anti-Siglec-H-Gd-160 (clone, REF), anti-FcER1-Dy-161 (clone MAR-1, Maxpar Ready, conjugated in-house), anti-PD-1-Er-166 (clone, REF), anti-H-2Kb/db-Yb-173 (clone 28-8-6, BioLegend, conjugated in-house), anti-CCR2-FITC (clone, REF) and anti-Thy1.2-PE (clone 30-H12, eBioscience, conjugated in-house). After washing twice, cells were fixed in 1.6% PFA in MP PBS for 10 min at RT. Individual samples were barcoded using six palladium metal isotopes according to the manufacturer's instructions (Cell-ID 20-plex Pd barcoding kit, Fluidigm) to reduce tube-to-tube variability. All individual samples were combined in the same tube and subsequently stained as multiplexed samples by incubating for 30 min at RT with the following antibodies: anti-CD45-Yb-89 (clone 30-F11, Fluidigm 3089005B), anti-CD11b-Nd-143 (clone M1/70, Fluidigm 3143015B), anti-FITC-Nd-144 (clone FIT22, Fluidigm 3144006B), anti-CD4-Nd-145 (clone RM4-5, Fluidigm 3145002B), anti-F4/80-Nd-146 (clone BM8, Fluidigm 3146008B), anti-Ly6G-Nd-148 (clone 1A8, Maxpar Ready, conjugated in-house), anti-CD19-Sm-149 (clone 6D5, Fluidigm 3149002B), anti-CD24-Nd-150 (clone M1/69, Fluidigm 3150009B), anti-CD64-Eu-151 (clone X54-5/7.1, Fluidigm 3151012B), anti-CD3e-Sm-152 (clone 145-2C11, Fluidigm 3152004B), anti-Ter119-Sm-154 (clone Ter119, Fluidigm 3154005B), anti-PE-Gd-156 (clone PE001, Fluidigm 3156005B), anti-CD103-Dy-162 (clone FIB504, Fluidigm 3162026B), anti-CD14-Dy-163 (clone, REF), anti-CD62L-Dy-164 (clone MEL-14, Fluidigm 3164003B), anti-CD8α-Er-168 (clone SK1, Fluidigm 3168002B), anti-TCRβ-Tm-169 (clone H57597, Fluidigm 3169002B), anti-NK1.1-Er-170 (clone, REF), anti-CD44-Yb-171 (clone IM7, Fluidigm 3171003B), anti-CD86-Yb-172 (clone GL1, Fluidigm 3172016B), anti-I-A/I-E-Yb-174 (clone M5/114.15.2, Fluidigm 3174003B), anti-CD127-Lu-175 (clone A7R34, Fluidigm 3175006B), anti-B220-Yb-176 (clone RA3-682, Fluidigm 3176002B) and anti-CD11c-Bi-209 (clone N418, Fluidigm 3209005B). The fixed cells were washed and stained for intracellular antigens in MP Nuclear Antigen Staining Buffer Set (Fluidigm) for additional 30 min at RT with: anti-TNF-Pr-141 (clone MP6-XT22, Fluidigm 3141013B), anti-Foxp3-Gd-158 (clone FJK-16s, Fluidigm 3158003A) anti-RORgt-Tb-159 (clone B2D, Fluidigm 3159019B), anti-T-bet-Ho-165 (clone 4B10, Maxpar Ready, conjugated in-house) and anti-GATA3-Er-167 (clone TWAJ, Fluidigm 3167007A). Samples were washed twice in Nuclear Antigen Staining Buffer and incubated in 125 nM Ir-191/193 DNA intercalator solution (Cell-ID Intercalator-Ir in Maxpar Fix/Perm buffer, Fluidigm) overnight at 4° C. Before acquisition on a Helios Mass Cytometer (available at the University of Virginia Flow Cytometry Core Facility), samples were washed twice with MP Fix/Perm buffer and twice with MP water. Raw data was normalized for detector sensitivity by adding five element beads to the sample and processed as described previously (Finck, R. et al. Normalization of mass cytometry data with bead standards. Cytometry A 83, 483-494, (2013)). Samples were debarcoded using the Zunder Lab single-cell debarcoder in MATLAB and files uploaded in Cytobank. Raw data was manually gated to exclude debris, doublets, dead cells, normalization beads (191/1931r_DNA⁺, 195Pt_Cisplatin^{neg}, 140Ce_EQbeads^{neg}), correct for Mahalanobis distance and to select CD45⁺ cell events. Individual normalized and gatedfcs files containing single live CD45⁺ events were exported from Cytobank, read into R as a flowset using the flowCore package (R package version 1.48.1) and subjected to arcsinh transformation (cofactor=5). Clustering was performed using the default settings for Rphenograph (Cytofkit package for version 3.5 of Bioconductor) (Levine, J. H. et al. Data-Driven Phenotypic Dissection of AML Reveals Progenitor-like Cells that Correlate with Prognosis. Cell 162, 184-197, (2015)) and all markers in the panel with the exception of CD45. For the generation of heatmaps showing median marker expression, the median quantile scaled expression value among cells from each cluster was visualized. Initial Rphenograph nodes depicting the median marker expression values within each cluster were then examined and clusters were merged to reflect biologically meaningful populations. A subset of cells was selected for t-distributed stochastic neighbor embedding (tSNE) visualization by randomly sampling an equal number of cells from each replicate, totaling 12,000 cells from each condition. Frequencies were calculated as the number of live CD45⁺ cells from each sample belonging to each cluster divided by the total number of live CD45⁺ cells in that sample. Frequencies were then used to calculate the number of cells in each cluster for each group.

Sorting of mouse meningeal LECs and RNA isolation. This procedure was performed as described in previous publications (Da Mesquita, S. et al. Functional aspects of meningeal lymphatics in ageing and Alzheimer's disease. Nature 560, 185-191, (2018); Louveau, A. et al. CNS lymphatic drainage and neuroinflammation are regulated by meningeal lymphatic vasculature. Nat Neurosci 21, 1380-1391, (2018)). Adult WT or 5×FAD mice (6 months of age) or old mice (20-24 months of age, one month upon injection with different AAV1 vectors), were euthanized by i.p. injection of Euthasol and transcardially perfused with ice cold PBS with heparin. To obtain a suspension of meningeal lymphatic endothelial cells (LECs), skull caps were quickly collected and meninges (dura mater and arachnoid) were dissected using Dumont #5 and #7 fine forceps in complete media composed of DMEM (Gibco) with 2% FBS (Atlas Biologicals), 1% L-glutamine (Gibco), 1% penicillin/streptomycin (Gibco), 1% sodium pyruvate (Gibco), 1% non-essential amino-acids (Gibco) and 1.5% Hepes buffer (Gibco). Individual brain meninges were then incubated with 1 mL of DMEM with 1 mg/mL of Collagenase VIII (Sigma-Aldrich) and 35 U/mL of DNAse I (Sigma-Aldrich) for 15 min at 37° C. Cell suspensions from 10 individual meninges were then pooled into a single tube after filtration through a 70 μm nylon mesh cell strainer. Suspensions of meningeal LECs were pelleted, resuspended in ice-cold FACS buffer containing DAPI (1:1000, Thermo Fisher Scientific), anti-CD45-BB515 (1:200, clone 30-F11, BD Biosciences), anti-CD31-Alexa Fluor® 647 (1:200, clone 390, BD Biosciences) and anti-Podoplanin-PE (1:200, clone 8.1.1, eBioscience) and incubated for 15 min at 4° C. Cells were then washed and resuspended in ice-cold FACS buffer. Briefly, singlets were gated using the pulse width of the side scatter and forward scatter. Cells negative for DAPI were selected for being viable cells. The LECs were then gated as CD45-CD31⁺Podoplanin⁺ and sorted into a 96-well plate containing 100 μL of RNA extraction lysis buffer using the Influx™ Cell Sorter (BD Biosciences) that is available at the University of Virginia Flow Cytometry Core Facility. Total RNA isolation was immediately performed following the manufacturer's instructions (Arcturus PicoPure RNA Isolation Kit, Thermo Fisher Scientific). RNA samples were stored at −80° C. until further use.

Hippocampus dissection and RNA isolation. This procedure was performed as previously described, with minor modifications. The whole hippocampus was macrodissected from the right brain hemisphere in ice-cold advanced DMEM/F12 (Gibco, 12634010) using Dumont #5 and #7 fine forceps and immediately snap-frozen in dry ice and stored at −80° C. until further use. After defrosting on ice, samples were mechanically dissociated in the appropriate volume of extraction buffer from the RNA isolation kit (RNeasy mini kit, 74106, QIAGEN). Total RNA from each sample was isolated and purified using the kit components according to the manufacturer's instructions.

Cultures of human LECs. Adult human dermal lymphatic microvascular endothelial cells (CC-2810, Lonza) were cultured in Endothelial cell growth medium (EBM™-2 basal medium, CC-3156, Lonza) supplemented with recommended growth medium supplement SingleQuots™ (EGM™-2MV BulletKit, CC-4147, Lonza) as described previously (Harris, A. R., Perez, M. J. & Munson, J. M. Docetaxel facilitates lymphatic-tumor crosstalk to promote lymphangiogenesis and cancer progression. BMC Cancer 18, 718, (2018)). Briefly, cells were plated at a density of 5000 cells/cm² in 12-well plates. Medium was replaced every 3 days until cells reached 60-70% confluency. At this point the cells were incubated with complete EBM-2 plus scrambled human amyloid beta 1-42 peptides (scramble, AS-25383, Anaspec) or monomeric/dimeric human amyloid beta 1-42 peptides (Aβ₄₂, AS-20276, Anaspec) at a final concentration of 100 nM. The kinetics of monomeric/dimeric Aβ₄₂ aggregation in vitro was previously described upon analysis of Aβ₄₂ species collected from culture supernatants at 24 and 72h (Da Mesquita, S. et al. Lipocalin 2 modulates the cellular response to amyloid beta. Cell Death Differ 21, 1588-1599, (2014)). At the time points of 24 and 72 hours, the medium was removed, cells were washed once with PBS and total RNA was isolated from the cells according to the manufacturer's instructions (RNeasy mini kit, cat. no. 74106, Qiagen). Each independent sample resulted from total RNA pooled from 3 well replicates. RNA samples were stored at −80° C. until further use.

Bulk RNA sequencing. The Illumina TruSeq Stranded Total RNA Library Prep Kit was used for cDNA library preparation from total RNA samples isolated from cultured human dermal lymphatic microvascular endothelial cells. Sample quality control was performed on an Agilent 4200 TapeStation Instrument, using the Agilent D1000 kit, and on the Qubit Fluorometer (Thermo Fisher Scientific). For RNA sequencing (RNA-seq), libraries were loaded on to a NextSeq 500 (Illumina) using Illumina NextSeq High Output (150 cycle, #FC-404-2002) and Mid Output (150 cycle, #FC-404-2001) cartridges. Processing of total RNA extracted from mouse meningeal LECs (including linear RNA amplification and cDNA library generation) and RNA-seq was performed by HudsonAlpha Genomic Services Laboratory (Huntsville, Ala.). For mouse RNA sequencing experiments, the fastq files were downloaded using HudsonAlpha's provided software andfastq files were merged by lane. The mergedfastq files were then chastity filtered to remove low quality bases, trimmed using the trimmomatic software, and mapped to the UCSC mm10 genome using Salmon (Harrow, J. et al. GENCODE: the reference human genome annotation for The ENCODE Project. Genome Res 22, 1760-1774, (2012); Patro, R., Duggal, G., Love, M. I., Irizarry, R. A. & Kingsford, C. Salmon provides fast and bias-aware quantification of transcript expression. Nat Methods 14, 417-419, (2017)). For human RNA sequencing experiments, the rawfastq files were merged by lane, filtered, trimmed, and mapped to the UCSC hg38 genome using Salmon. The resulting transcript abundances were read into R and summarized using tximport (Soneson, C., Love, M. I. & Robinson, M. D. Differential analyses for RNA-seq: transcript-level estimates improve gene-level inferences. F1000Res 4, 1521, (2015)). Ensembl identifiers were mapped to gene symbols where possible and kept as Ensembl ID's where no mapping was identified. Additionally, counts for Ensembl ID's mapping to the same gene symbol were summed. Genes were filtered to remove those with fewer than five counts in at least two samples as well as those with high intra-sample standard deviations. The remaining counts were analyzed using DESeq2 for normalization, differential expression, and principal component analysis (PCA) (Love, M. I., Huber, W. & Anders, S. Moderated estimation of fold change and dispersion for RNA-seq data with DESeq2. Genome Biol 15, 550, (2014)). The Benjamini-Hochberg correction was used to adjust the associated P-values for differentially expressed genes and those with adjusted P-values below 0.05 were considered to be significant. Significantly differentially expressed gene sets were used as input for functional enrichment of GeneOntology (GO) terms as well as Kyoto Encyclopedia of Genes and Genomes (KEGG) pathways using the ClusterProfiler Bioconductor package (Yu, G., Wang, L. G., Han, Y. & He, Q. Y. clusterProfiler: an R package for comparing biological themes among gene clusters. OMICS 16, 284-287, (2012); Yu, G., Wang, L. G., Yan, G. R. & He, Q. Y. DOSE: an R/Bioconductor package for disease ontology semantic and enrichment analysis. Bioinformatics 31, 608-609, (2015)). Highly variable genes were defined as those with the largest variance across samples using the log transformed expression values. Heatmaps of differentially expressed genes, highly variable genes, and gene sets enriched for specific pathways were visualized using the R package pheatmap and expression values for each sample were represented as standard deviations from the mean across each gene. Code is available in the GitHub repository.

Neurological disease-associated gene expression in mouse LECs. Summary statistics were downloaded from the NHGRI-EBI GWAS Catalog for the following experimental factor ontologies: EFO_0000249, EFO 0003756, EFO_0003885, EFO_0002508, and EFO_0000692 on Oct. 24, 2019 (Buniello, A. et al. The NHGRI-EBI GWAS Catalog of published genome-wide association studies, targeted arrays and summary statistics 2019. Nucleic Acids Res 47, D1005-D1012, (2019)). For each disease the unique set of reported genes were used for comparison with the LEC bulk RNA-seq datasets and HGNC symbols were mapped to their MGI counterparts using the bioMart database (Durinck, S., Spellman, P. T., Birney, E. & Huber, W. Mapping identifiers for the integration of genomic datasets with the R/Bioconductor package biomaRt. Nat Protoc 4, 1184-1191, (2009); Durinck, S. et al. BioMart and Bioconductor: a powerful link between biological databases and microarray data analysis. Bioinformatics 21, 3439-3440, (2005)). Normalized counts from each of the LEC datasets were used to calculate average expression of each gene across samples. SNP reported genes were determined to be in the top percentiles based on their average expression. RNA-seq data of LECs from diaphragm or from ear skin were included in the heatmap visualizations for reference but were not used for calculation of the average expression. Heatmaps were visualized using the log 2 transformed expression values and the pheatmap package. Genes falling within the top 25^{th} percentile of highly expressed genes across the LEC datasets were used as gene sets for functional enrichment of GO terms and KEGG pathways using Fischer's exact test as implemented in the ClusterProfiler package (Yu, G., Wang, L. G., Han, Y. & He, Q. Y. clusterProfiler: an R package for comparing biological themes among gene clusters. OMICS 16, 284-287, (2012); Yu, G., Wang, L. G., Yan, G. R. & He, Q. Y. DOSE: an R/Bioconductor package for disease ontology semantic and enrichment analysis. Bioinformatics 31, 608-609, (2015)). Additionally, the normalized count matrix from GEO study GSE98816 (Vanlandewijck, M. et al. A molecular atlas of cell types and zonation in the brain vasculature. Nature 554, 475-480, (2018)) was downloaded and cells labelled as Endothelial Cells (EC) were extracted for further analysis in Seurat. The normalized counts were scaled, principal components analysis was applied using the top 2000 highly variable genes, and the top nine significant principal components were used for shared nearest neighbor clustering and tSNE. Five out of the seven identified clusters matched a similar transcriptional profile to the arterial, capillary, and venous endothelial cells identified by Vanlandewijck et al. (Vanlandewijck, M. et al. A molecular atlas of cell types and zonation in the brain vasculature. Nature 554, 475-480, (2018)) and were subset and reclustered. This analysis resulted in 4 clusters (capillary ECs 1, capillary ECs 2, arterial ECs and venous ECs). The average normalized expression of each gene was calculated within each cluster and the percentage of disease-associated genes falling within each quantile was determined based on average expression of the gene within the cluster. The union of the sets of disease-associated genes falling within the top 2^{nd} percentile of highly expressed genes for each cluster was visualized in the heatmap using log 2 average normalized expression values.

Brain myeloid cell sorting and single-cell RNA sequencing. 5×FAD mice were injected (i.c.m.) with Visudyne alone (3 mice) or with Visudyne followed by transcranial photoconversion steps (3 mice) following previously described methodology. The same procedures were repeated three weeks later. Six weeks after the initial interventions, mice were euthanized by i.p. injection of Euthasol and transcardially perfused with ice cold PBS with heparin. The skulls were collected, and the brain was carefully extracted into ice-cold HBSS medium (14025, Thermo Fisher Scientific). Under an articulated stereomicroscope, pia and choroid plexus tissue contaminants were carefully discarded and the brain cortices were collected by macrodissection. Brain tissue was passed through a 5 mL pipette tip (10 times), and digested for 30 min at 37° C. in HBSS with 1 mg/mL of Collagenase VIII, 1 mg/mL of Collagenase D, 50 U/mL of DNAse I and 25 μg/mL Actinomycin D to inhibit transcription mediated by all RNA polymerases (all from Sigma Aldrich). The same volume of DMEM/F12 (Gibco) with 5% FBS and 10 mM EDTA was added to the digested tissue, which was then passed through a 1 mL pipette tip (10 times) and filtered through a 70 μm cell strainer. The cell pellets were washed, resuspended in ice-cold FACS buffer, preincubated for 10 min at 4° C. with Fc-receptor blocking solution (rat anti-mouse CD16/32, clone 93, BioLegend, 1:200 in FACS) and stained for extracellular markers with the following antibodies (all at 1:200 in FACS): anti-CD11b PerCP-Cy5.5 (550993, BD Biosciences), anti-CD45 A700 (560510, BD Biosciences) and anti-Ly6G BV421 (562737, BD Biosciences). After an incubation period of 25 min at 4° C., cells were washed and resuspended in FACS buffer with SYTOX™ Green Nucleic Acid Stain following the manufacturer's instructions (Thermo Fisher Scientific) to determine the cell viability. Cells gated on singlets (based on height, area and the pulse width of the forward and side scatters) and SYTOX^{neg}Ly6G^{neg}C.D45⁺CD11b⁺ were sorted using the Influx™ Cell Sorter at the University of Virginia Flow Cytometry Core Facility. Single myeloid cells from samples pertaining to the same group were pooled into the same 1.5 mL tubes containing 0.04% non-acetylated BSA in PBS and diluted to 1000 cells per L estimated from counting on a hemocytometer and Trypan blue (Sigma Aldrich) staining. The sorted myeloid cells (˜2000 per sample) were loaded onto a Chromium™ Single Cell A Chip (PN-120236, 10X Genomics) placed in a 10X™ Chip Holder and run on a 10X™ platform Chromium™ Controller to generate cDNAs carrying cell- and transcript-specific barcodes. Sequencing libraries were constructed using the Chromium™ Single Cell 3′ Library & Gel Bead Kit v2 (PN-120237, 10X Genomics). Libraries were sequenced on the Illumina NextSeq using paired-end sequencing, with 100,000 reads targeted per cell. Binary base call (BCL) files were converted tofastq format using the Illumina bcl2fastq2 software. Fastq files were then mapped to the mm10 transcriptome using the Cellranger 3.0.2 pipeline, specifically the count function. The resulting gene by count matrices were then read into R and filtered to remove cells with fewer than 500 unique molecular counts, fewer than 400 unique genes, or greater than fifteen percent mitochondrial gene expression. Additionally, genes were filtered to retain only those which were expressed more than five cells. The remaining cells were then normalized using the scran normalization package (McCarthy, D. J., Campbell, K. R., Lun, A. T. & Wills, Q. F. Scater: pre-processing, quality control, normalization and visualization of single-cell RNA-seq data in R. Bioinformatics 33, 1179-1186, (2017)). The resulting normalized counts were then transformed from log 2 scale to the natural log scale for compatibility with Seurat. After further analysis of the dataset, small populations of potentially contaminating oligodendrocyte precursor cells, oligodendrocytes, neurons and astrocytes were identified and removed based on the expression levels of Cspg4, Mbp, Cam2ka, and Gfap genes, respectively. Additionally, after an initial clustering, one population of cells was removed based on cluster markers. The remaining 649 cells (409 in the 5×FAD Vis. group and 247 in the 5×FAd Vis./photo. group) were re-scaled in Seurat and the effects of sequencing depth per cell, number of unique features, and percentage of mitochondrial genes were regressed out. Highly variable genes were determined using the variance stabilizing transformation and the top 2000 most variable genes were used as input for PCA. The significance of the first twenty principal components was evaluated using the Jackstraw test. Based on these results, the percentage of variance explained by each component, and the number of cells in the dataset, the first five principal components were used for clustering and tSNE analysis. Cluster membership was determined using shared nearest neighbor graph embedding and optimization of the Louvain algorithm as implemented in the Seurat FindNeighbors and FindClusters functions with a resolution of 0.6. All differentially expressed genes were calculated using the Wilcoxon Rank Sum test as implemented in Seurat. For cluster markers, the FindAllMarkers function was used to test genes showing a minimum log fold change of 0.1 in each cluster versus all other clusters and expressed in a minimum of 30% of cells in the cluster of interest. For differentially expressed genes between groups, the FindMarkers function was used to test all genes expressed in at least 10% of cells within each group. P-values were adjusted using the Bonferroni correction method and genes were considered to be significantly differentially expressed if the adjusted P-values <0.05 (Butler, A., Hoffman, P., Smibert, P., Papalexi, E. & Satija, R. Integrating single-cell transcriptomic data across different conditions, technologies, and species. Nat Biotechnol 36, 411-420, (2018)).

Whole brain hemisphere blood vascular and myeloid cell sorting. Adult 5×FAD or aged APPswe mice were euthanized by i.p. injection of Euthasol and transcardially perfused with ice cold PBS with heparin. The skulls were collected, and the whole right brain hemisphere was carefully dissected into ice-cold advanced DMEM/F12 (Gibco, 12634010). Brain tissue was mechanically dissociated with sterile sharp scissors and digested for 45 min at 37° C. in advanced DMEM/F12 with 1 mg/mL of Collagenase VIII, 50 U/mL of DNAse I and 1% FBS. Every 15 min, the tissue suspensions were sequentially passed through a 10 mL serological pipette (10 times), followed by two passages through a 5 mL serological pipette (10 times each). Cellular suspensions were filtered through a 70 μm cell strainer, thoroughly mixed with an equal volume of 22% BSA in PBS and centrifuged at 1,000×g for 10 min at RT (without break), to remove the myelin. After discarding the upper layer and supernatant containing the myelin and cell debris, the cell pellets were washed with advanced DMEM/F12 supplemented with 10% FBS, resuspended in ice-cold FACS buffer, preincubated for 10 min at 4° C. with Fc-receptor blocking solution (anti-CD16/32, 101302, BioLegend, 1:200 in ice-cold FACS buffer) and stained for extracellular markers with the following antibodies (at 1:200 in ice-cold FACS buffer, unless stated otherwise): anti-CD13-FITC (at 1:50, 558744, BD Biosciences), anti-Ly6G-PE (127608, BioLegend), anti-CD11b PerCP-Cy5.5 (550993, BD Biosciences), anti-CD31-APC (17-0311-80, eBioscience) and anti-CD45-APC-Cy7 (103116, BioLegend). DAPI (0.1 μg/mL) was also added to access cell viability. After an incubation period of 25 min at 4° C., cells were washed and resuspended in ice-cold FACS buffer. Cells were gated on DAPI-singlets (based on height, area and the pulse width of the forward and side scatters), CD45⁺CD11b⁺Ly6G⁻ (myeloid cells), CD45⁻CD11b⁻CD13⁺CD31⁻ (mural cells) and CD45⁻CD11b⁻CD31⁺ (blood endothelial cells) and sorted using the Influx™ Cell Sorter at the University of Virginia Flow Cytometry Core Facility. The aforementioned enriched single cell populations pertaining to the same group were sorted into the same 1.5 mL tube previously coated (overnight) with 0.1% ultrapure non-acetylated BSA (Thermo Fisher Scientific, AM2616) in PBS and containing 500 μL of ice-cold advanced DMEM/F12. Single cells were centrifuged at 350×g for 5 min at 4° C. and resuspended in 0.10% ultrapure non-acetylated BSA in PBS at 1,000 cells per L estimated from counting on a hemocytometer and Trypan blue (MilliporeSigma) staining. Single-cell suspensions were kept on ice until further use.

Brain cortical myeloid cell sorting. 5×FAD mice (5.5 months-old) with intact or ablated meningeal lymphatics were euthanized by i.p. injection of Euthasol and transcardially perfused with ice cold PBS with heparin. The skulls were collected, and the brain was carefully extracted into ice-cold HBSS medium (14025, Thermo Fisher Scientific). Under an articulated stereo microscope, pia and choroid plexus tissue contaminants were carefully discarded and the brain cortices were collected by macrodissection. Brain tissue was passed through a 5 mL pipette tip (10 times) and digested for 30 min at 37° C. in HBSS with 1 mg/mL of Collagenase VIII, 1 mg/mL of Collagenase D, 50 U/mL of DNAse I and 25 μg/mL Actinomycin D to inhibit transcription mediated by all RNA polymerases (all from MilliporeSigma). The same volume of DMEM/F12 (Gibco) with 5% FBS and 10 mM EDTA was added to the digested tissue, which was then passed through a 1 mL pipette tip (10 times) and filtered through a 70 μm cell strainer. The cell pellets were washed, resuspended in ice-cold FACS buffer, preincubated for 10 min at 4° C. with Fe-receptor blocking solution (anti-CD16/32, 1:200 in FACS buffer) and stained for extracellular markers with the following antibodies (all at 1:200 in FACS buffer): anti-CD11b-PerCP-Cy5.5 (550993, BD Biosciences), anti-CD45-A700 (560510, BD Biosciences) and anti-Ly6G-BV421 (562737, BD Biosciences). After an incubation period of 25 min at 4° C., cells were washed and resuspended in FACS buffer with SYTOX™ Green Nucleic Acid Stain following the manufacturer's instructions (Thermo Fisher Scientific) to determine the cell viability. Cells were gated on singlets (based on height, area and the pulse width of the forward and side scatters) and SYTOX-Ly6G-CD45⁺CD11b⁺ (myeloid cells) and sorted using the Influx™ Cell Sorter at the University of Virginia Flow Cytometry Core Facility. Single myeloid cells from samples pertaining to the same group were sorted into the same 1.5 mL tubes previously coated with and containing 500 μL of ice-cold 0.1% ultrapure non-acetylated BSA in PBS. Single cells were centrifuged at 350×g for 5 min at 4° C. and resuspended in the same buffer to 1,000 cells per L estimated from counting on a hemocytometer and Trypan blue (MilliporeSigma) staining. Single-cell suspensions were kept on ice until further use.

Murine single-cell RNA sequencing. For the experiments involving sorted blood vascular and myeloid cells from whole brain hemispheres, ˜10,000 cells per sample were loaded onto a Chromium™ Single Cell A Chip (PN-120236, 10× Genomics) placed in a 10x™ Chip Holder and run on a 10×™ platform Chromium™ Controller to generate cDNAs carrying cell- and transcript-specific barcodes. For the experiment involving sorted myeloid cells only from the brain cortex, ˜2,000 cells per sample were loaded onto the Chromium™ Single Cell A Chip and run on a 10x™ platform Chromium™ Controller to generate the cDNAs. Sequencing libraries were constructed using the Chromium™ Single Cell 3′ Library & Gel Bead Kit v2 (PN-120237, 10× Genomics). After a quality control (QC) step performed on an Illumina MiSeqNano system, libraries were sequenced on the Illumina NextSeq platform using paired-end sequencing, with 100,000 reads targeted per cell. All murine single-cell RNA-seq data were generated in the Genome Analysis and Technology Core of the University of Virginia (RRID:SCR_018883). Binary base call (bcl) files were converted tofastq format using the Illumina bcl2fastq2 software. Fastq files were then mapped to the mm10 transcriptome using the Cellranger 3.0.2 pipeline, specifically the count function. The resulting gene by count matrices were then read into R and filtered to remove low quality cells based on unique molecular counts, unique genes, and percent mitochondrial gene expression. Additionally, genes were filtered to retain only those which were expressed more than five cells. The remaining cells were then normalized, and log transformed using the scran normalization package. The resulting normalized counts were then transformed from log₂ scale to the natural log scale for compatibility with Seurat v3 and the effects of sequencing depth per cell, number of unique features, and percentage of mitochondrial genes were regressed out. Highly variable genes were determined using the variance stabilizing transformation and the top 2,000 most variable genes were used as input for PCA. Principal components were selected based on an elbow plot. Alternatively, for the cortical myeloid single-cell dataset, the significance of the first twenty principal components was evaluated using the Jackstraw test. Based on these results, on the percentage of variance explained by each component and the number of cells in the dataset, the first five principal components were used for clustering and t-Stochastic Neighbor Embedding (tSNE) analysis. Cluster membership was determined using shared nearest neighbor graph embedding and optimization of the Louvain algorithm as implemented in the Seurat FindNeighbors and FindClusters functions with a resolution of 0.6. All differentially expressed genes were calculated using the Wilcoxon Rank Sum test as implemented in Seurat. For cluster markers, the FindAllMarkers function was used to test genes showing a minimum log fold change of 0.25 (for the whole brain vascular and microglia single-cell experiments) or 0.1 (for the cortical microglia single-cell experiment) in each cluster versus all other clusters and expressed in a minimum of 30% of cells in the cluster of interest. In all datasets, cluster annotation was performed manually based on the expression of canonical markers and clusters collapsed based on common cell types. A final number of 7286 cells, including 2625 microglia, 1958 capillary blood endothelial cells (BECs), 1412 venous BECs and 545 arterial BECs was obtained in the whole brain vascular and myeloid single-cell dataset obtained using adult 5×FAD mice. A final number of 7739 cells, including 2345 microglia, 2934 capillary BECs, 602 venous BECs and 766 arterial BECs, was obtained in the whole brain vascular and myeloid single-cell dataset obtained using aged APPswe mice. In the cortical myeloid single-cell dataset obtained using 5×FAD mice, small populations of potentially contaminating oligodendrocyte precursor cells, oligodendrocytes, neurons and astrocytes were identified and removed based on the expression levels of Cspg4, Mbp, Cam2ka, and Gfap genes, respectively. Additionally, after an initial clustering, one population of cells was removed based on cluster markers, leading to a final number of 649 microglia. For analysis of differentially expressed genes between groups in the whole brain BECs and microglia datasets, each cluster was filtered to include genes that had at least 5 transcripts in at least 5 cells, then the top 2,000 highly variable genes were determined and included for further analysis using the SingleCellExperiment modelGeneVar and getTopHVGs functions. After filtering, observational weights for each gene were calculated using the ZINB-WaVE zinbFit and zinbwave functions. These were then included in the edgeR model, which was created with the glmFit function, by using the glmWeightedF function. Results were then filtered using as threshold a Benjamini-Hochberg adjusted P-value <0.05 (statistically significant). Volcano plots were made with the EnhancedVolcano package. Genes matching this significance threshold were divided on the basis of up- or down-regulation and used as input for GO Analysis using the ClusterProfiler package. For analysis of differentially expressed genes between groups in the cortical microglia dataset, the FindMarkers function was used to test all genes expressed in at least 10% of cells within each group. P-values were adjusted using the Bonferroni correction method and genes were considered to be significantly differentially expressed if the adjusted P-values <0.05.

Neurological disease-associated gene expression. Summary statistics were downloaded from the NHGRI-EBI GWAS Catalog for the following experimental factor ontologies: EFO_0000249, EFO 0003756, EFO_0003885, EFO_0002508, and EFO_0000692 on Oct. 24, 2019. For each disease the unique set of reported genes were used for comparison with the LECs' bulk RNA-seq datasets and HGNC symbols were mapped to their MGI counterparts using the bioMart database. Normalized counts from each of the LECs' datasets were used to calculate average expression of each gene across samples. Reported disease-associate genes were determined to be in the top percentiles based on their average expression. RNA-seq data of LECs from diaphragm or from ear skin were included in the heatmap visualizations for reference but were not used for calculation of the average expression. Heatmaps were visualized using the log₂ transformed expression values and the pheatmap package. Genes falling within the top 25^{th} percentile of highly expressed genes across the LECs' datasets were used as gene sets for functional enrichment of GO terms and KEGG pathways using Fischer's exact test as implemented in the ClusterProfiler package. Additionally, the normalized count matrix from GEO study GSE98816 was downloaded and cells labelled as BECs were extracted for further analysis in Seurat. The normalized counts were scaled, principal components analysis was applied using the top 2,000 highly variable genes, and the top nine significant principal components were used for shared nearest neighbor clustering and tSNE. Five out of the seven identified clusters matched a similar transcriptional profile to the arterial, capillary, and venous BECs identified by Vanlandewijck et al and were subset and re-clustered. This analysis resulted in 4 clusters of brain BECs: capillary 1, capillary 2, arterial and venous. The average normalized expression of each gene was calculated within each cluster and the percentage of disease-associated genes falling within each quantile was determined based on average expression of the gene within the cluster. The union of the sets of disease-associated genes falling within the top 2^{nd} percentile of highly expressed genes for each cluster was visualized in the heatmap using log₂ average normalized expression values. Finally, a search was performed for the AD-associated genes in the cortical microglia single-cell RNA-seq dataset. The average normalized expression of each gene was calculated within each microglial cluster (clusters 1-4) and the percentage of AD-associated genes falling within each quantile was determined based on average expression of the gene within the cluster. The union of the sets of AD-associated genes falling within the top 2^{nd} percentile of highly expressed genes for each cluster was visualized in a heatmap using log₂ average normalized expression values. For the venn diagram comparing the AD-associated genes expressed in the top 10^{th} percentile of meningeal LECs, brain BECs (including all 4 clusters) and microglia (including all 4 clusters), the gene set was determined by ranking genes for that cell type based on average expression across all cells in the dataset rather than by cluster.

Statistical analysis and reproducibility. Sample sizes were chosen on the basis of standard power calculations (with α=0.05 and power of 0.8) performed for similar experiments that were previously published. In general, statistical methods were not used to re-calculate or predetermine sample sizes. Animals from different cages, but within the same experimental group, were selected to assure randomization. Experimenters were blinded to the identity of experimental groups from the time of euthanasia until the end of data collection and analysis for all the independent experiments. The Kolmogorov-Smirnov test was used to assess the distribution of the data. Variance was similar within independent groups of the same experiment and between groups from independent experiments. The ROUT test was used to identify and discard potential outliers (outliers are indicated in the source data files). Data in graphs was always presented as mean±standard error mean (s.e.m.). Two-group comparisons were made using two-tailed unpaired Student's T test. For comparisons of multiple factors (for example, lymphatic vessel ablation vs antibody treatment), two-way ANOVA with Holm-Sidak's multiple comparisons test was used. Repeated measures two-way ANOVA with Tukey's multiple comparisons test was used to analyze data acquired during the acquisition and reversal of the Morris water maze test. Statistical analysis in experiments involving mouse models, tissues and cells was performed in Prism version 8.3.4 (GraphPad Software, Inc.) or in R software (version 3.5.0). Statistical tests used for group or cluster comparisons in bulk, single-cell or single-nuclei RNA-seq experiment analysis are specified in the respective methods' sections.

Code and data availability. New RNA-seq data sets have been deposited online in the Gene Expression Omnibus (GEO database) under the accession number GSE141917. Previously published RNA-seq data sets can be found under the accession numbers GSE99743 and GSE104181. Code used to analyze the RNA-seq data is available online under GNU General Public license v3.0 at Github under Kipnis Lab/DaMesquita-2019. Custom code used and datasets generated and/or analyzed during the current study are also available from the corresponding authors upon reasonable request.

### Example 11: Treatment with VEGF-c and Aducanumab

A human subject is identified as suffering from Alzheimer's disease. A pharmaceutical composition comprising human VEGF-c and aducanumab is administered monthly to the subject via intravenous infusion. The intravenous administration is repeated monthly for eight months. Amelioration of behavior symptoms of Alzheimer's disease, including memory loss is observed. Reduction in quantity of density of amyloid beta plaques in the brain of the subject is observed by in vivo magnetic resonance imaging.

In some embodiments, the method, use, or composition comprises various steps or features that are present as single steps or features (as opposed to multiple steps or features). For example, in one embodiment, the method includes a single administration of a flow modulator, or the composition comprises or consists essentially of a flow modulator for single use. The flow modulator may be present in a single dosage unit effective for increasing flow. A composition or use may comprise a single dosage unit of a flow modulator effective for increasing flow as described herein. Multiple features or components are provided in alternate embodiments. In some embodiments, the method, composition, or use comprises one or more means for flow modulation. In some embodiments, the means comprises a flow modulator.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. For each method of described herein, relevant compositions for use in the method are expressly contemplated, uses of compositions in the method, and, as applicable, methods of making a medicament for use in the method are also expressly contemplated. For example, for methods of increasing flow that comprise a flow modulator, flow modulators for use in the corresponding method are also contemplated, as are uses of a flow modulator in increasing flow according to the method, as are methods of making a medicament comprising the flow modulator for use in increasing flow.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. For example, “about 5”, shall include the number 5. For example, the term “about” can indicate that the number differs from the reference number by less than 9%, 8%, 7%, 6%, 5%, 4%, 3%, 2%, or 1%. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

### APPENDIX: TABLES 2-29

TABLE 2
4921524J17Rik
4930404I05Rik
4930554G24Rik
Adamts3
Argi
B4galt4
BC031181
Cdc25a
Cdkl2
Chrm3
Cyp2d22
Egln3
Enah
Etfb
Evi2
Fam219aos
Gcdh
Grid2
Histlhle
Ibtk
Itgb2
Kcnrg
Kit
Lzic
Mcccl
Mis12
Mum111
Nop10
Npl
Pamr1
Ppp2r1b
Rabgef1
Rnf5
Rundc3b
Sox17
Susd5
Teski
Tfpi2
Timp1
Tssc4
Zbtb17
Zfp358
Zfp869
ENSMUSG00000053218
ENSMUSG00000054418
ENSMUSG00000069682
ENSMUSG00000075511
ENSMUSG00000085673
ENSMUSG00000097020
ENSMUSG00000097358

TABLE 3
4921524J17Rik
4930404105Rik
4930554G24Rik
Adamts3
Arg1
B4galt4
BC031181
Cdc25a
Cdkl2
Chrm3
Cyp2d22
Egln3
Enah
Etfb
Evi2
Fam219aos
Gcdh
Grid2
Histlhle
Ibtk
Itgb2
Kcnrg
Kit
Lzic
Mccc1
Misl2
Mum111
Nop10
Npl
Pamr1
Ppp2rlb
Rabgef1
Rnf5
Rundc3b
Sox17
Susd5
Tesk1
Tfpi2
Timp1
Tssc4
Zbtbl7
Zfp358
Zfp869
ENSMUSG00000053218
ENSMUSG00000054418
ENSMUSG00000069682
ENSMUSG00000075511
ENSMUSG00000085673
ENSMUSG00000097020
ENSMUSG00000097358

TABLE 4
Abr
Exoc8
Exph5
Itgb2
Kit
Llg11
Mical3
Milr1
Nr4a3
Pip5klc
Ppfia2
Rab11fip1
Rabgef1
Rap1a
Rapgef4
Scfd2
Stam
Stx1
Syt14
Vamp2

TABLE 5
Abr
Exoc8
Exph5
Itgb2
Kit
Llg11
Mical3
Milr1
Nr4a3
Pip5klc
Ppfia2
Rabi ifip1
Rabgefl
Rap1a
Rapgef4
Scfd2
Stam
Stx11
Syt14
Vamp2

TABLE 6
Adcy4
Cyth3
Gabl
Kit
Pdgfa
Pip5klc
Plcb3
Pld1
Ptpn11
Rapgef4
Shc1

TABLE 7
Adcy4
Cyth3
Gabi
Kit
Pdgfa
Pip5klc
Plcb3
Pld1
Ptpn1
Rapgef4
Shc1

TABLE 8
150001 lB03Rik
1700051A21Rik
2310033P09Rik
4921524J17Rik
4930404I05Rik
4930554G24Rik
Acad11
Adamts3
Amer1
Arg1
Arrdc1
AU022754
B4galt4
Banf1
BC031181
BC107364
C1qtnf7
Casp8
Cd8a
Cdc25a
Cdc25c
Cdc3711
Cdk12
Cdr21
Cfp
Chrm3
Clvs1
Cnot9
Col6a6
Cxxc5
Cyba
Cyp2d22
Cyth3
Dach1
Dmwd
Dnah10
Dnajc17
Dsn1
Efhd1
Egln3
Enah
Ephx1
Etfb
Evi2
Fam219aos
Fbx16
Fbxo44
Ftcd
Gcdh
Gm20939
Gpr34
Gpr75
Grid2
Gtf2f2
Gtf2h4
H2-B1
Hectd2os
Hemgn
Histlhle
Hmgn3
Hps6
Htrld
Ibtk
ldh3g
Itgb2
ltih2
Jazfl
Kcna4
Kcnq2
Kcnrg
Kdmlb
Kit
Klhdc7a
Lncenc1
Lzic
Mapk9
Mcccl
Milrl
Mir466k
Mis12
Mlf2
Mnatl
Mrps33
Mum111
Myo15
Myomi
Nagpa
Naip6
Nol41
Nop10
Np1
Nr4a2
Nr4a3
Nup54
Osbp111
Pa11d
Pamr1
Pcdhl7
Pdgfa
Phfl4
Pip5klc
Pparg
Ppfia2
Ppp2rlb
Ptgs2os2
Pwp2
Rabep2
Rabgef1
Ranbp9
Rap1a
Rapgef4
Ras112
Ren1
Rgs8
Riox2
Rnf24
Rnf5
Rrm1
Rte11
Rtp4
Rundc3b
Saraf
Ska2
Slc24a1
Slc25a48
Slc2a12
Snhg12
Snrnp40
Socs5
Sox17
Sp2
Sprtn
Stk17b
Stx11
Stx6
Suox
Susd5
Tesk1
Tfpi2
Timp1
Tlel
Tmem68
Tns2
Trabd
Trmt1
Tssc4
Uba5
Ulk1
Vcam1
Vps9d1
Wdr90
Wtip
Xylt1
Zbp1
Zbtbl7
Zfp358
Zfp869
ENSMUSG00000000948
ENSMUSG00000022187
ENSMUSG00000041449
ENSMUSG00000044867
ENSMUSG00000053218
ENSMUSG00000054418
ENSMUSG00000059229
ENSMUSG00000064582
ENSMUSG00000066170
ENSMUSG00000066538
ENSMUSG00000067292
ENSMUSG00000069682
ENSMUSG00000075511
ENSMUSG00000082432
ENSMUSG00000082965
ENSMUSG00000084843
ENSMUSG00000085087
ENSMUSG00000085279
ENSMUSG00000085391
ENSMUSG00000085673
ENSMUSG00000086146
ENSMUSG00000086166
ENSMUSG00000086172
ENSMUSG00000086625
ENSMUSG00000086736
ENSMUSG00000087083
ENSMUSG00000089146
ENSMUSG00000089793
ENSMUSG00000089883
ENSMUSG00000090722
ENSMUSG00000092090
ENSMUSG00000092395
ENSMUSG00000092811
ENSMUSG00000093190
ENSMUSG00000093261
ENSMUSG00000093499
ENSMUSG00000093650
ENSMUSG00000096528
ENSMUSG00000097020
ENSMUSG00000097088
ENSMUSG00000097358
ENSMUSG00000097429
ENSMUSG00000097770

TABLE 9
150001 lB03Rik
1700051A21Rik
2310033P09Rik
4921524J17Rik
4930404I05Rik
4930554G24Rik
Acad11
Adamts3
Amer1
Arg1
Arrdc1
AU022754
B4galt4
Banf1
BC031181
BC107364
Clqtnf7
Casp8
Cd8a
Cdc25a
Cdc25c
Cdc3711
Cdkl2
Cdr21
Cfp
Chrm3
Clvs1
Cnot9
Col6a6
Cxxc5
Cyba
Cyp2d22
Cyth3
Dach1
Dmwd
Dnah10
Dnajcl7
Dsn1
Efhd1
Egln3
Enah
Ephx1
Etfb
Evi2
Fam219aos
Fbx116
Fbxo44
Ftcd
Gcdh
Gm20939
Gpr34
Gpr75
Grid2
Gtff
Gtf2h4
H2-B1
Hectd2os
Hemgn
Hist1h1e
Hmgn3
Hps6
Htr1d
Ibtk
ldh3g
Itgb2
ltih2
Jazf1
Kcna4
Kcnq2
Kcnrg
Kdm1b
Kit
Klhdc7a
Lncenc1
Lzic
Mapk9
Mccc1
Milr1
Mir466k
Mis12
Mlf2
Mnatl
Mrps33
Mum111
Myo15
Myomi
Xylt1
Zbp1
Zbtbl7
Zfp358
Zfp869
Nagpa
Naip6
Nol41
Nop10
Np1
Nr4a2
Nr4a3
Nup54
Osbp111
Palld
Pamrl
Pcdh17
Pdgfa
Phfl4
Pip5k1c
Pparg
Ppfia2
Ppp2r1b
Ptgs2os2
Pwp2
Rabep2
Rabgef1
Ranbp9
Rap1a
Rapgef4
Ras112
Ren1
Rgs8
Riox2
Rnf24
Rnf5
Rrm1
Rte1
Rtp4
Rundc3b
Saraf
Ska2
Slc24a1
Slc25a48
Slc2al2
Snhgl2
Snrnp40
Socs5
Sox17
Sp2
Sprtn
Stk17b
Stx11
Stx6
Suox
Susd5
Tesk1
Tfpi2
Timp1
Tle1
Tmem68
Tns2
Trabd
Trmt1
Tssc4
Uba5
Ulk1
Vcam1
Vps9d1
Wdr90
Wtip
ENSMUSG00000000948
ENSMUSG00000022187
ENSMUSG00000041449
ENSMUSG00000044867
ENSMUSG00000053218
ENSMUSG00000054418
ENSMUSG00000059229
ENSMUSG00000064582
ENSMUSG00000066170
ENSMUSG00000066538
ENSMUSG00000067292
ENSMUSG00000069682
ENSMUSG00000075511
ENSMUSG00000082432
ENSMUSG00000082965
ENSMUSG00000084843
ENSMUSG00000085087
ENSMUSG00000085279
ENSMUSG00000085391
ENSMUSG00000085673
ENSMUSG00000086146
ENSMUSG00000086166
ENSMUSG00000086172
ENSMUSG00000086625
ENSMUSG00000086736
ENSMUSG00000087083
ENSMUSG00000089146
ENSMUSG00000089793
ENSMUSG00000089883
ENSMUSG00000090722
ENSMUSG00000092090
ENSMUSG00000092395
ENSMUSG00000092811
ENSMUSG00000093190
ENSMUSG00000093261
ENSMUSG00000093499
ENSMUSG00000093650
ENSMUSG00000096528
ENSMUSG00000097020
ENSMUSG00000097088
ENSMUSG00000097358
ENSMUSG00000097429
ENSMUSG00000097770

TABLE 10
ADAM12
ADAMTS9-AS2
ADCY5
ADGRE4P
ADRA2A
AMIGO2
APLN
AR
AREG
ARHGAP20
ARNT2
B4GALNT3
BMPER
C14orfl144
CA4
CBLN2
CCIN
CCL14
CD302
CD79B
CDH2
CDKNIC
CLEC7A
CSF2
CXCL1
CXCL5
CYP7A1
DIRAS2
DM1-AS
DNAJC5G
DNASE2B
E2F8
EGFR
ELMOD1
ENPP3
EPB41L3
ERVFRD-1
ETV4
FAM13C
FAM95B1
FBLN2
FERMT1
FGF1
FREM3
FRZB
FSTL4
GAL
GDF3
GJA4
GPC6
GPR146
GPR3
GUCY1A1
HCRTR1
HIST1H1B
HIST1H1D
HIST1H2AJ
HIST1H2BM
HIST1H3B
HIST1H3G
HMGA2
HSPA2
IL33
IRX3
IRX5
ITGB1-DT
ITGB4
JPH2
KCTD4
KLHL14
KLHL31
LDLRAD4-AS1
LINC00475
LINC01148
LINC01303
LINC01747
LINC02330
LOC100996643
LOC101928377
LOC102724488
LOC105371115
LOC105373502
LOC283194
LYPD6
MATN2
MBOAT1
MCM10
METTL7A
MMP10
MMP23A
MPP4
MTIA
MTIE
MYOCD
MYOM1
MYPN
NEO1
NEXMIF
NPTX1
NXPH3
OMG
OPNILW
OR2A42
PARD6G
PCDH19
PCDH7
PCDHA9
PCED1B
PDK4
PENK
PLAUR
PLCE1-AS1
PLEK2
PLEKHN1
PNMA2
POSTN
PPFIBP2
PRSS3
REN
RET
RGPD2
RNF208
RRM2
RSPH10B2
RTN4RL1
SALL2
SELP
SEMA3C
SESN3
SFTA1P
SHC4
SLC13A3
SLC16A12
SLC27A2
SLC38A4
SLC40A1
SLC5A3
SLC6A15
SLC6A2
SNAP91
SNTB1
SOX5
SPOCD1
STUM
STXBP6
SYN2
SYT13
TACSTD2
TC2N
TEAD3
TENM2
TEX28
TGFB2-AS1
TMEM178A
TMTC1
TRIBI
TRIM29
TRIM54
TSHZ2
TTC9B
TUBA IB
UBE2C
UBE2F-SCLY
UNC5D
USP43
VTRNAl-3
VWA5B2
WNT11
YPEL4
ENSG00000069712
ENSG00000176134
ENSG00000206532
ENSG00000225840
ENSG00000227158
ENSG00000229953
ENSG00000230869
ENSG00000235072
ENSG00000241280
ENSG00000254786
ENSG00000254966
ENSG00000255189
ENSG00000256566
ENSG00000257989
ENSG00000258780
ENSG00000258943
ENSG00000259712
ENSG00000260788
ENSG00000263426
ENSG00000267577
ENSG00000270607
ENSG00000271204
ENSG00000275400
ENSG00000277734
ENSG00000280614
ENSG00000280800
ENSG00000281181
ENSG00000281383
ENSG00000281881
ENSG00000283907
ENSG00000284413

TABLE 11
ADAM12
ADAMTS9-AS2
ADCY5
ADGRE4P
ADRA2A
AMIG02
APLN
AR
AREG
ARHGAP20
ARNT2
B4GALNT3
BMPER
C14orfl44
CA4
CBLN2
CCIN
CCL14
CD302
CD79B
CDH2
CDKNIC
CLEC7A
CSF2
CXCL1
CXCL5
CYP7A1
DIRAS2
DMI-AS
DNAJC5G
DNASE2B
E2F8
EGFR
ELMODI
ENPP3
EPB41L3
ERVFRD-1
ETV4
FAM13C
FAM95B1
FBLN2
FERMT1
FGF1
FREM3
FRZB
FSTL4
GAL
GDF3
GJA4
GPC6
GPR146
GPR3
GUCY1A1
HCRTR1
HIST1H1B
HIST1H1D
HIST1H2AJ
HIST1H2BM
HIST1H3B
HIST1H3G
HMGA2
MYOM1
MYPN
NEO1
NEXMIF
NPTX1
NXPH3
OMG
OPNILW
OR2A42
PARD6G
PCDH19
PCDH7
PCDHA9
PCED1B
PDK4
PENK
PLAUR
PLCE1-AS1
PLEK2
PLEKHN1
PNMA2
POSTN
PPFIBP2
PRSS3
REN
RET
RGPD2
RNF208
RRM2
RSPH10B2
RTN4RL1
SALL2
SELP
HSPA2
IL33
IRX3
IRX5
ITGB1-DT
ITGB4
JPH2
KCTD4
KLHL14
KLHL31
LDLRAD4-AS1
LINC00475
LINC01148
LINC01303
LINC01747
LINC02330
LOC100996643
LOC101928377
LOC102724488
LOC105371115
LOC105373502
LOC283194
LYPD6
MATN2
MBOATI
MCM10
METTL7A
MMP10
MMP23A
MPP4
MTIA
MTIE
MYOCD
SEMA3C
SESN3
SFTA1P
SHC4
SLC13A3
SLC16A12
SLC27A2
SLC38A4
SLC40A1
SLC5A3
SLC6A15
SLC6A2
SNAP91
SNTB1
SOX5
SPOCD1
STUM
STXBP6
SYN2
SYT13
TACSTD2
TC2N
TEAD3
TENM2
TEX28
TGFB2-AS1
TMEM178A
TMTC1
TRIBI
TRIM29
TRIM54
TSHZ2
TTC9B
UBE2C
UBE2F-SCLY
UNC5D
USP43
VTRNAl-3
VWA5B2
WNT11
YPEL4
ENSG00000069712
ENSG00000176134
ENSG00000206532
ENSG00000225840
ENSG00000227158
ENSG00000229953
ENSG00000230869
ENSG00000235072
ENSG00000241280
ENSG00000254786
ENSG00000254966
ENSG00000255189
ENSG00000256566
ENSG00000257989
ENSG00000258780
ENSG00000258943
ENSG00000259712
ENSG00000260788
ENSG00000263426
ENSG00000267577
ENSG00000270607
ENSG00000271204
ENSG00000275400
ENSG00000277734
ENSG00000280614
ENSG00000280800
ENSG00000281181
ENSG00000281383
ENSG00000281881
ENSG00000283907

TABLE 12
ADCY5
AMIG02
C14orfl44
CA4
CBLN2
CDKNIC
CLEC7A
CSF2
CYP7A1
ERVFRD-1
FGF1
FRZB
GDF3
GJA4
HIST1H2BM
IL33
IRX3
KCTD4
LDLRAD4-AS1
LINC01148
LINC01747
LINC02330
LOC105371115
LOC105373502
MMP23A
MYOCD
MYOMI
NEOl
NPTX1
OMG
OR2A42
PCED1B
PDK4
PPFIBP2
REN
RET
RGPD2
RNF208
SESN3
SLC27A2
SNAP91
SPOCD1
TEX28
ENSG00000254786
ENSG00000255189
ENSG00000257989
ENSG00000260788
ENSG00000280614
ENSG00000283907
ENSG00000284413

TABLE 13
ADCY5
AMIGO2
C14orf144
CA4
CBLN2
CDKNIC
CLEC7A
CSF2
CYP7A1
ERVFRD-1
FGF1
FRZB
GDF3
GJA4
HIST1H2BM
IL33
IRX3
KCTD4
LDLRAD4-AS1
LINC01148
LINC01747
LINC02330
LOC105371115
LOC105373502
MMP23A
MYOCD
MYOM1
NEO1
NPTX1
OMG
OR2A42
PCED1B
PDK4
PPFIBP2
REN
RET
RGPD2
RNF208
SESN3
SLC27A2
SNAP91
SPOCD1
TEX28
ENSG00000254786
ENSG00000255189
ENSG00000257989
ENSG00000260788
ENSG00000280614
ENSG00000283907
ENSG00000284413

TABLE 14
ACTB
ACTG1
ACTN4
AFDN
CDC42
CREBBP
CSNK2A2
CSNK2A3
CTNNA1
CTNNB1
CTNND1
EGFR
EP300
FARP2
FGFR1
IGF1R
INSR
IQGAP1
LMO7
MAP3K7
MAPK1
MAPK3
NECTIN3
PARD3
PTPN1
PTPRB
PTPRF
PTPRM
RAC1
RAC2
RHOA
SMAD3
SMAD4
SNAI1
SNAI2
TCF7L1
TGFBR1
TGFBR2
TJP1
VCL
WASF2
WASF3
WASL

TABLE 15
ACTB
ACTG1
ACTN4
AFDN
CDC42
CREBBP
CSNK2A2
CSNK2A3
CTNNA1
CTNNB1
CTNND1
EGFR
EP300
FARP2
FGFR1
IGF1R
INSR
IQGAP1
LMO7
MAP3K7
MAPK1
MAPK3
NECTIN3
PARD3
PTPN1
PTPRB
PTPRF
PTPRM
RAC1
RAC2
RHOA
SMAD3
SMAD4
SNAI1
SNAI2
TCF7L1
TGFBR1
TGFBR2
TJP1
VCL
WASF2
WASF3
WASL

TABLE 16
ADCY1
ADCY3
ADCY5
ADCY7
AGPAT2
AGPAT4
AKT1
AKT3
ARF1
ARF6
CXCL8
CYTH1
CYTH2
DGKD
DGKE
DGKG
DGKI
DGKZ
DNM3
EGFR
F2R
GAB1
GAB2
GNA13
GNAS
GRB2
INSR
KITLG
KRAS
LPAR6
MAP2K1
MAPK1
MAPK3
MTOR
NRAS
PDGFA
PDGFB
PDGFC
PDGFD
PDGFRA
PIK3CA
PIK3CB
PIK3R1
PIK3R3
PIP5K1C
PLCB1
PLCB2
PLCB3
PLCB4
PLCG1
PLCG2
PLD2
PLPP1
PLPP2
PLPP3
PRKCA
PTPN11
RALA
RALB
RALGDS
RAPGEF3
RAPGEF4
RHOA
RRAS
RRAS2
SHC1
SHC2
SHC3
SHC4
SOS1
SOS2
SPHK1
SPHK2
TSC1
TSC2

TABLE 17
ADCY1
ADCY3
ADCY5
ADCY7
AGPAT2
AGPAT4
AKT1
AKT3
ARF1
ARF6
CXCL8
CYTH1
CYTH2
DGKD
DGKE
DGKG
DGKI
DGKZ
DNM3
EGFR
F2R
GAB1
GAB2
GNA13
GNAS
GRB2
INSR
KITLG
KRAS
LPAR6
MAP2K1
MAPK1
MAPK3
MTOR
NRAS
PDGFA
PDGFB
PDGFC
PDGFD
PDGFRA
PIK3CA
PIK3CB
PIK3R1
PIK3R3
PIP5K1C
PLCB1
PLCB2
PLCB3
PLCB4
PLCG1
PLCG2
PLD2
PLPP1
PLPP2
PLPP3
PRKCA
PTPN11
RALA
RALB
RALGDS
RAPGEF3
RAPGEF4
RHOA
RRAS
RRAS2
SHC1
SHC2
SHC3
SHC4
SOS1
SOS2
SPHK1
SPHK2
TSC1
TSC2

TABLE 18

| | X141 | X142 | X143N | X144 | X145 | X146 | X147S | X148 | X149S | X150 | X151 | X152 |
|---|---|---|---|---|---|---|---|---|---|---|---|---|
| | Pr_T | Nd_L | d_CDl | Nd_C | Nd_C | Nd_F | m_CD | Nd_L | m_CD | Nd_C | Eu_C | Sm_C |
| | NFa | y6C | IB | CR2 | D4 | 4.80 | 169 | Y6G | 19 | D24 | D64 | D3 |
| B cells | 0 | 0.146 | 0.0624 | 0 | 0 | 0.0057 | 0 | 0.0096 | 0.6594 | 0.5406 | 0.0158 | 0 |
| | | 024 | 95 | | | 97 | | 41 | 54 | 23 | 37 | |
| Basop | 0 | 0.210 | 0.2271 | 0.0405 | 0 | 0.0441 | 0 | 0.0076 | 0 | 0.0468 | 0.0513 | 0 |
| hils | | 023 | 86 | 94 | | 79 | | 9 | | 42 | 9 | |
| CD4T | 0 | 0.200 | 0.0848 | 0.0669 | 0.868 | 0.1225 | 0 | 0.0077 | 0 | 0.0233 | 0.0174 | 0.793 |
| | | 052 | 15 | 64 | 711 | 58 | | 34 | | | 63 | 762 |
| CD8T | 0 | 0.629 | 0.1105 | 0.0245 | 0 | 0.0114 | 0 | 0.0005 | 0 | 0.0211 | 0.0129 | 0.600 |
| | | 259 | 64 | 26 | | 86 | | 99 | | 63 | 6 | 905 |
| eDCs | 0 | 0.206 | 0.2298 | 0.5721 | 0.018 | 0.0688 | 0 | 0.0336 | 0 | 0.7038 | 0.1580 | 0.004 |
| 1 | | 417 | 21 | 41 | 087 | 29 | | 05 | | 08 | 98 | 535 |
| eDCs | 0 | 0.216 | 0.8703 | 0.6329 | 0.039 | 0.0794 | 0 | 0.0305 | 0 | 0.0605 | 0.1550 | 0 |
| 2 | | 953 | 37 | 38 | 629 | | | 36 | | 32 | 07 | |
| ILC2 | 0 | 0.163 | 0.0528 | 0.0672 | 0 | 0 | 0 | 0 | 0 | 0.0118 | 0.0075 | 0 |
| | | 358 | 86 | 24 | | | | | | 09 | 3 | |
| ILC3 | 0 | 0.140 | 0.0671 | 0.0355 | 0.768 | 0.1198 | 0 | 0.0192 | 0 | 0.0257 | 0.0147 | 0 |
| | | 308 | 91 | 78 | 251 | 25 | | 72 | | 61 | 58 | |
| Macs | 0.013 | 0.838 | 0.7114 | 0.3171 | 0.124 | 0.5417 | 0.0436 | 0.0515 | 0 | 0.2559 | 0.6928 | 0.018 |
| 2 | 941 | 855 | 53 | 83 | 086 | 97 | 91 | 2 | | 86 | 87 | 173 |
| (Infl.) | | | | | | | | | | | | |
| Macs | 0 | 0.199 | 0.7515 | 0.2195 | 0.035 | 0.6261 | 0.0253 | 0.0306 | 0 | 0.0485 | 0.7390 | 0.008 |
| 3 | | 195 | 61 | 73 | 967 | 99 | 77 | 65 | | 38 | 2 | 045 |
| Macs | 0 | 0.214 | 0.7680 | 0.1881 | 0.564 | 0.7565 | 0.1017 | 0.0383 | 0 | 0.0598 | 0.8281 | 0.020 |
| 4 | | 815 | 09 | 3 | 437 | 97 | 1 | 33 | | 71 | 78 | 565 |
| Mono/ | 0 | 0.205 | 0.8011 | 0.5601 | 0.027 | 0.4610 | 0 | 0.0279 | 0 | 0.0416 | 0.7287 | 0.005 |
| Macs | | 218 | 69 | 93 | 383 | 83 | | 07 | | 06 | 44 | 794 |
| 1 | | | | | | | | | | | | |
| Mono | 0 | 0.866 | 0.6248 | 0.6226 | 0.024 | 0.0977 | 0 | 0.0238 | 0 | 0.0808 | 0.1621 | 0 |
| cytes | | 898 | 31 | 6 | 262 | 35 | | 83 | | 88 | 17 | |
| Neutro | 0.047 | 0.629 | 0.8807 | 0.4921 | 0.179 | 0.4625 | 0.0293 | 0.8832 | 0.0771 | 0.5995 | 0.6065 | 0.032 |
| phils 1 | 894 | 278 | 09 | 78 | 245 | 11 | 75 | 73 | 43 | 03 | 63 | 124 |
| Neutro | 0 | 0.624 | 0.7878 | 0.3516 | 0.067 | 0.1750 | 0 | 0.8943 | 0.0526 | 0.5799 | 0.0472 | 0 |
| phils 2 | | 492 | 82 | 83 | 181 | 96 | | 7 | 63 | | 32 | |
| NK-Ts | 0.956 | 0.413 | 0.6604 | 0.7495 | 0.186 | 0.6226 | 0 | 0.3719 | 0.4421 | 1 | 0.4140 | 0.617 |
| | 948 | 881 | 69 | 37 | 397 | 92 | | 93 | 81 | | 69 | 461 |
| NKs | 0 | 0.236 | 0.1654 | 0 | 0 | 0 | 0 | 0 | 0 | 0.0123 | 0.0094 | 0 |
| | | 178 | 55 | | | | | | | 43 | 31 | |
| pDCs | 0 | 0.537 | 0.2168 | 0.1959 | 0.093 | 0.0749 | 0 | 0.0166 | 0 | 0.0517 | 0.0502 | 0 |
| | | 336 | 14 | 13 | 889 | 8 | | | | 47 | 48 | |
| Plasma | 0 | 0.817 | 0.1956 | 0.1134 | 0.004 | 0.0355 | 0 | 0.0252 | 0.5930 | 0.5861 | 0.0362 | 0 |
| cells | | 411 | 13 | 31 | 576 | 17 | | 76 | 79 | 66 | 88 | |
| Pro/pr | 0 | 0.267 | 0.1583 | 0.1474 | 0.006 | 0.0400 | 0 | 0.0520 | 0.6759 | 0.5294 | 0.0400 | 0.010 |
| eB | | 3 | 43 | 72 | 131 | 31 | | 28 | 01 | 48 | 82 | 841 |
| cells | | | | | | | | | | | | |
| Red | 0 | 0.250 | 0.7242 | 0.2916 | 0.076 | 0.4669 | 0 | 0.0588 | 0 | 0.7591 | 0.6762 | 0.033 |
| blood | | 866 | 64 | 63 | 733 | 24 | | 23 | | 69 | 57 | 088 |
| cells | | | | | | | | | | | | |
| TCRgt | 0 | 0.150 | 0.0708 | 0.0814 | 0 | 0.0075 | 0 | 0.0022 | 0 | 0.0246 | 0.0225 | 0.841 |
| | | 48 | | 27 | | 25 | | 75 | | 54 | 72 | 834 |
| Tregs | 0 | 0.334 | 0.7116 | 0.3412 | 0.779 | 0.3594 | 0 | 0.0465 | 0 | 0.0924 | 0.5178 | 0.730 |
| | | 817 | 94 | 48 | 332 | 01 | | 62 | | 47 | 77 | 004 |
| Undefined | 0 | 0.283 | 0.1769 | 0.0560 | 0 | 0.0470 | 0 | 0.0147 | 0 | 0.0851 | 0.0771 | 0 |
| | | 121 | 52 | 21 | | 96 | | 37 | | 34 | 91 | |
| | X153 | X154S | X156G | X158 | X159 | X160G | X161 | X162 | X163 | X164 | X165 | X166 |
| | EuX | m_TER | d_TH | GdF | Tb_R | dSigle | Dy_Fc | Dy_C | Dy_C | Dy_C | Ho_T | Er_P |
| | CRI | 119 | Y1.2 | OXp3 | ORgt | c.H | ERI | D103 | D14 | D62L | Bet | DI |
| B | 0 | 0 | 0.0047 | 0.2635 | 0.0386 | 0.0192 | 0 | 0 | 0 | 0 | 0 | 0 |
| cells | | | 23 | 45 | 45 | 55 | | | | | | |
| Basop | 0.004 | 0 | 0.0422 | 0.3365 | 0.1037 | 0.0611 | 0.4251 | 0 | 0.009 | 0 | 0 | 0 |
| hits | 287 | | 68 | 29 | 67 | 24 | 86 | | 582 | | | |
| CD4 | 0.012 | 0.0135 | 0.6706 | 0.3019 | 0.0497 | 0.0460 | 0.0782 | 0 | 0 | 0 | 0.238 | 0 |
| T | 681 | 17 | 97 | 15 | 76 | 99 | 7 | | | | 488 | |
| CD8 | 0 | 0 | 0.7151 | 0.3141 | 0.0531 | 0.0441 | 0 | 0 | 0 | 0 | 0.624 | 0 |
| T | | | 25 | 92 | 52 | 41 | | | | | 937 | |
| eDCs | 0.975 | 0.0825 | 0.0684 | 0.4371 | 0.1609 | 0.1415 | 0.0742 | 0.9231 | 0.164 | 0.0521 | 0.125 | 0.02 |
| 1 | 201 | 39 | 36 | 71 | 66 | 09 | 83 | 93 | 222 | 51 | 9 | 2281 |
| eDCs | 0.007 | 0 | 0.0253 | 0.4052 | 0.3454 | 0.1073 | 0 | 0.0098 | 0.037 | 0 | 0.065 | 0 |
| 2 | 493 | | 42 | 5 | 99 | 28 | | 74 | 787 | | 428 | |
| ILC2 | 0 | 0 | 0.6897 | 0.2937 | 0.0925 | 0.0453 | 0 | 0.1237 | 0 | 0 | 0.008 | 0.01 |
| | | | 61 | 76 | 24 | 79 | | 39 | | | 787 | 9383 |
| ILC3 | 0 | 0 | 0.4160 | 0.3097 | 1 | 0.0879 | 0.0758 | 0.2268 | 0.005 | 0 | 0.038 | 9.31 |
| | | | 77 | 33 | | 56 | 23 | 39 | 89 | | 256 | E-05 |
| Macs | 0.109 | 0.0479 | 0.1380 | 0.5539 | 0.2552 | 0.1881 | 0.0777 | 0.0267 | 0.335 | 0 | 0.051 | 0.05 |
| 2 | 867 | 57 | 09 | 26 | 29 | 98 | 23 | 77 | 53 | | 022 | 6883 |
| (Infl.) | | | | | | | | | | | | |
| Macs | 0.103 | 0.0011 | 0.0763 | 0.4003 | 0.2156 | 0.0698 | 0.0227 | 0.0190 | 0.383 | 0 | 0.019 | 0 |
| 3 | 931 | 21 | 76 | 75 | 29 | 95 | 59 | 05 | 094 | | 403 | |
| Macs | 0.123 | 0.0106 | 0.0786 | 0.4371 | 0.2502 | 0.0676 | 0.0523 | 0.0367 | 0.215 | 0 | 0.029 | 0 |
| 4 | 834 | 85 | 38 | 88 | 34 | 32 | 77 | 16 | 817 | | 266 | |
| Mono/ | 0.063 | 0 | 0.0356 | 0.3943 | 0.2443 | 0.0840 | 0.0699 | 0.0259 | 0.717 | 0.0245 | 0.029 | 0 |
| Macs | 748 | | 24 | | 31 | 28 | 12 | 78 | 999 | 34 | 571 | |
| 1 | | | | | | | | | | | | |
| Monocytes | 0.001 | 0 | 0.0217 | 0.3878 | 0.1983 | 0.1245 | 0 | 0.1470 | 0.022 | 0.0441 | 0.029 | 0 |
| | 425 | | 82 | 99 | 61 | 14 | | 54 | 444 | 91 | 659 | |
| Neutrophils | 0.076 | 0.0700 | 0.1273 | 0.5541 | 0.3959 | 0.2228 | 0.0577 | 0.0296 | 0.226 | 0.5762 | 0.075 | 0.02 |
| | 427 | 43 | 65 | 15 | 56 | 19 | 11 | 62 | 578 | 17 | 494 | 7688 |
| 1 | | | | | | | | | | | | |
| Neutrophils | 0 | 0.0113 | 0.0359 | 0.3808 | 0.2570 | 0.0754 | 0 | 0 | 0.007 | 0.5955 | 0,000 | 0 |
| | | 42 | 59 | 54 | 69 | 38 | | | 388 | 59 | 539 | |
| 2 | | | | | | | | | | | | |
| NK- | 0.043 | 0.8247 | 0.4631 | 0.5390 | 0.7623 | 0.2083 | 0 | 0.3606 | 0.702 | 0.9519 | 0.463 | 0.18 |
| Ts | 041 | 62 | 9 | 98 | 4 | 25 | | 81 | 903 | 74 | 648 | 0886 |
| NKs | 0 | 0 | 0.2002 | 0.2965 | 0.0631 | 0.0276 | 0 | 0 | 0 | 0.1064 | 0.777 | 0 |
| | | | 93 | 78 | 04 | 12 | | | | 57 | 867 | |
| pDCs | 0.010 | 0 | 0.0690 | 0.4106 | 0.1431 | 0.6233 | 0 | 0.0024 | 0.021 | 0.0224 | 0.022 | 0 |
| | 262 | | 2 | 51 | 86 | 69 | | 66 | 514 | 45 | 386 | |
| Plasma | 0.007 | 0.0914 | 0.0414 | 0.4207 | 0.0885 | 0.0888 | 0 | 0 | 0.000 | 0 | 0.014 | 0.02 |
| cells | 561 | 54 | 8 | 47 | 21 | 06 | | | 528 | | 366 | 594 |
| Pro/pr | 0.023 | 0.0145 | 0.7031 | 0.5045 | 0.1648 | 0.2439 | 0 | 0.1947 | 0.030 | 0 | 0.069 | 0.06 |
| eB | 937 | 88 | 55 | 4 | 51 | 58 | | 69 | ill | | 459 | 0873 |
| cells | | | | | | | | | | | | |
| Red | 0.080 | 1 | 0.0968 | 0.4460 | 0.2422 | 0.1041 | 0.0381 | 0.0200 | 0.212 | 0.0108 | 0.034 | 0.04 |
| blood | 318 | | 4 | 12 | 12 | 68 | 59 | 36 | 181 | 03 | 083 | 1972 |
| cells | | | | | | | | | | | | |
| TCRg | 0.024 | 0.0243 | 0.8427 | 0.3180 | 1 | 0.0871 | 0 | 0.0177 | 0.010 | 0 | 0.122 | 0 |
| t | 757 | 78 | 43 | 74 | | 16 | | 01 | 909 | | 543 | |
| Tregs | 0.077 | 0.0547 | 0.6590 | 0.5171 | 0.2611 | 0.1682 | 0.1071 | 0.0477 | 0.152 | 0.0088 | 0.401 | 0.01 |
| | 596 | 35 | 27 | 06 | 72 | 73 | 1 | 13 | 598 | 01 | 388 | 801 |
| Undefined | 0.076 | 0 | 0.0844 | 0.4160 | 0.0935 | 0.0903 | 0 | 0 | 0.012 | 0 | 0.029 | 0.07 |
| | 252 | | 52 | 3 | 66 | 35 | | | 696 | | 263 | 9488 |
| | X167Er | X168E | X169T | X170E | X171Y | X172Y | X173Yb | X174 | X175L | X176 | X209B | |
| | GATA | r_CD8 | m_TC | r_NKl | b_CD4 | b_CD8 | _H.2Kb | Yb_IA | u_CDl | Yb_B2 | i_CDl | |
| | 3 | a | Rb | .1 | 4 | 6 | Db | IE | 27 | 20 | 1c | |
| B cells | 0 | 0 | 0 | 0 | 0.1542 | 0.0565 | 0.251315 | 0.6126 | 0.08607 | 0.4719 | 0 | |
| | | | | | 62 | 13 | | 9 | 1 | 94 | | |
| Basophils | 0 | 0 | 0 | 0 | 0.5727 | 0.1099 | 0.24452 | 0.1928 | 0 | 0 | 0.0631 | |
| | | | | | 93 | 08 | | 9 | | | 21 | |
| CD4T | 0.27677 | 0 | 0.8235 | 0 | 0.6983 | 0.3193 | 0.341174 | 0.2172 | 0.31049 | 0 | 0 | |
| | | | 16 | | 86 | 56 | | 99 | 3 | | | |
| CD8T | 0.26345 | 0.8112 | 0.6632 | 0 | 0.5792 | 0.4438 | 0.211897 | 0.2008 | 0.11016 | 0 | 0.0111 | |
| | 8 | 67 | 87 | | 53 | 99 | | 78 | 5 | | 78 | |
| eDCs 1 | 0 | 0 | 0 | 0 | 0.5380 | 0.4316 | 0.684761 | 0.7926 | 0.18343 | 0.2591 | 0.8673 | |
| | | | | | 28 | 74 | | 56 | 4 | 64 | 8 | |
| cDCs2 | 0 | 0 | 0 | 0 | 0.6908 | 0.2284 | 0.723806 | 0.8328 | 0.24355 | 0.3149 | 0.7979 | |
| | | | | | 23 | 77 | | 33 | 1 | 21 | 73 | |
| ILC2 | 0.90264 | 0.0641 | 0 | 0 | 0.7341 | 0.1552 | 0.460743 | 0.1562 | 0.66564 | 0 | 0 | |
| | | 78 | | | 64 | 71 | | 19 | | | | |
| ILC3 | 0.45579 | 0 | 0 | 0 | 0.9042 | 0.3829 | 0.53677 | 0.9029 | 0.94350 | 0.4693 | 0 | |
| | 6 | | | | 1 | 52 | | 15 | 1 | 91 | | |
| Macs 2 | 0 | 0 | 0 | 0 | 0.1394 | 0.4739 | 0.647562 | 0.7460 | 0.12249 | 0.1574 | 0.0555 | |
| (Infl.) | | | | | 03 | 48 | | 47 | 9 | 67 | 74 | |
| Macs 3 | 0 | 0 | 0 | 0 | 0.0984 | 0.5927 | 0.532491 | 0.8029 | 0.16829 | 0.2275 | 0.0337 | |
| | | | | | 53 | 22 | | 26 | 2 | 03 | 33 | |
| Macs 4 | 0 | 0 | 0 | 0 | 0.0733 | 0.6574 | 0.490502 | 0.5865 | 0.04199 | 0.0378 | 0.0510 | |
| | | | | | 2 | 74 | | 96 | 2 | 04 | 72 | |
| Mono/ | 0 | 0 | 0 | 0 | 0.3973 | 0.3624 | 0.644948 | 0.8590 | 0.24706 | 0.3352 | 0.4458 | |
| Macs 1 | | | | | 22 | 72 | | 57 | 7 | 67 | 77 | |
| Monoc | 0 | 0 | 0 | 0 | 0.6527 | 0.1700 | 0.283131 | 0.2412 | 0 | 0 | 0.0140 | |
| ytes | | | | | 02 | 76 | | 3 | | | 2 | |
| Neutro | 0.00175 | 0 | 0 | 0 | 0.7368 | 0.4402 | 0.503373 | 0.7294 | 0.20403 | 0.1958 | 0.0917 | |
| phils 1 | 1 | | | | 08 | 57 | | 26 | | 99 | 02 | |
| Neutro | 0 | 0 | 0 | 0 | 0.7203 | 0.1366 | 0.069216 | 0.1341 | 0.03982 | 0 | 0.0039 | |
| phils 2 | | | | | 74 | 5 | | 08 | 7 | | 51 | |
| NK-Ts | 0.32958 | 0.3207 | 0.2470 | 1 | 0.9784 | 0.4583 | 0.136316 | 0.2332 | 1 | 0.4260 | 0.5844 | |
| | 3 | 92 | 91 | | 1 | 04 | | 79 | | 82 | 35 | |
| NKs | 0.22093 | 0 | 0 | 0.3019 | 0.4878 | 0.0743 | 0.203643 | 0.1827 | 0 | 0 | 0.3697 | |
| | 6 | | | 62 | 97 | 84 | | 74 | | | 55 | |
| pDCs | 0 | 0 | 0 | 0 | 0.5095 | 0.1279 | 0.41297 | 0.4887 | 0.07632 | 0.1690 | 0.4591 | |
| | | | | | | 38 | | 25 | 7 | 16 | 01 | |
| Plasma | 0 | 0 | 0 | 0 | 0.1584 | 0.0614 | 0.454182 | 0.5864 | 0.03361 | 0.4139 | 0.0098 | |
| cells | | | | | 85 | 3 | | 78 | | 49 | 39 | |
| Pro/pre | 0.91519 | 0.1141 | 0 | 0 | 0.7633 | 0.2358 | 0.595826 | 0.6491 | 0.68822 | 0.5874 | 0.0235 | |
| B cells | 5 | 11 | | | 22 | 25 | | 61 | | 08 | 7 | |
| Red | 0 | 0 | 0 | 0 | 0.3990 | 0.3790 | 0.507751 | 0.7215 | 0.12038 | 0.1538 | 0.0804 | |
| blood | | | | | 83 | 08 | | 38 | 5 | 49 | 43 | |
| cells | | | | | | | | | | | | |
| TCRgt | 0.30181 | 0 | 0 | 0 | 0.8591 | 0.2740 | 0.241287 | 0.1795 | 0.78649 | 0 | 0 | |
| | 3 | | | | 47 | 48 | | 21 | 8 | | | |
| Tregs | 0.25141 | 0.0370 | 0.7560 | 0.0194 | 0.7177 | 0.5754 | 0.638128 | 0.7560 | 0.38593 | 0.2147 | 0.1292 | |
| | 3 | 91 | 99 | 26 | 21 | 47 | | 51 | | 02 | 66 | |
| Undefined | 0 | 0 | 0 | 0 | 0.2278 | 0.0796 | 0.381149 | 0.4253 | 0.01204 | 0.0079 | 0.0311 | |
| | | | | | 53 | 42 | | 8 | 8 | 5 | 37 | |

TABLE 19
P2ry12
Tmem119
Cx3cr1
Selplg
Serinc3
Mareks
Glul
Txnip
Hexb
Sparc
Csf1r
C1qa
C1qb
C1qc
Cst3
Ctss
O1fm13
P2ry13
Tgfbr1
Ctsb
Ctsd
Apoe
Lyz2
Tyrobp
Gnas
Fth1
B2m
Cstb
Timp2
H2.D1
Trem2
Axl
Cst7
Spp1
Ctsl
Lpl
Cd9
Csf1
Ccl6
Cd63
Itgax
Ank
Serpine2
Cadm1
Ctsz
Ctsa
Cd68
Cd52
Gusb
Hif1a

TABLE 20
3110056K07Rik
9030624G23Rik
Adat1
Anapc15
Atad3a
Bad
Bbs7
Camkmt
Ccdc57
Cyp2s1
Ddx39
Dnaaf5
Esrrg
Faml71a2
Fancd2
Gmppb
Hmces
Itih3
Kif21b
Micall2
Mrps27
Nalcn
Pex10
Pop4
Ptch2
Qrsl1
Rtel1
Septin14
Slc10a3
Slc39a6
Sox6
Stoml3
Taf6
Tbc1d5
Tle6
Tmem81
Tmppe
Trip12
Ttc9
Use1
Ust
Zan
Zfp58
ENSMUSG00000064622
ENSMUSG00000081854
ENSMUSG00000085636
ENSMUSG00000090673
ENSMUSG00000091275
ENSMUSG00000094842
ENSMUSG00000095352

TABLE 21
3110056K07Rik
9030624G23Rik
Adat1
Anapc15
Atad3a
Bad
Bbs7
Camkmt
Ccdc57
Cyp2s1
Ddx39
Dnaaf5
Esrrg
Fam171a2
Fancd2
Gmppb
Hmces
Itih3
Kif21b
Micall2
Mrps27
Nalcn
Pex10
Pop4
Ptch2
Qrsl1
Rtel1
Septin 14
Slc10a3
Slc39a6
Sox6
Stoml3
Taf6
Tbc1d5
Tle6
Tmem81
Tmppe
Trip12
Ttc9
Use1
Ust
Zan
Zfp58
ENSMUSG00000064622
ENSMUSG00000081854
ENSMUSG00000085636
ENSMUSG00000090673
ENSMUSG00000091275
ENSMUSG00000094842
ENSMUSG00000095352

TABLE 22
Abca1
Ahnak
Akap9
Dlc1
Dst
Egr1
Itga1
Kcnn3
Maf
Nfib
Prrcc
Reln
Sash1
Timp2

TABLE 23
Ank2
Ctnnd1
Ctsb
Mmr1
Ppfibp1
Scn3a
Smarca2
Stab1

TABLE 24
Arhgap31
Cdh11
Ctnna1
Ctnnd1
Egr1
Ep300
Foxp1
Igf1
Jmjd1c
Kmt2e
Lrp1
Map4
Nfib
Plce1
Podxl
Prrc2a
Reln
Shank3
Slc4a10
Smarca2
Sned1
Stab1
Tcf4
Tead1
Tmtc1
Tspan18
Ywhae
Zmiz1

TABLE 25
Ctnnd1
Egr1
Ep300
Foxp1
Kmt2e
Lrp1
Prrc2a
Smarca2
Stab1
Tcf4
Zmiz1

TABLE 26
Asap1
Foxp1
Kif1b
Lpp
Maf
Reln
Zfp3611
Zhx3
Zmiz1

TABLE 27
ADAM10
ADAR
AHNAK
BSG
CCDC50
DST
HDAC9
HSPA9
ITGA1
ITGA6
MAN2A1
MAP4K4
MPZL1
NFIB
NT5E
PAK2
PVR
RELN
SASH1
SQSTM1
TIMP2
ZYX

TABLE 28

| | cluster_2 | cluster_0 | cluster_1 | cluster_3 |
|---|---|---|---|---|
| Hmcn1 | 5.18948 | 7.335148 | 8.159985 | 6.875606 |
| Slc19a3 | 6.813669 | 7.387728 | 7.719819 | 7.865548 |
| Mpzl1 | 9.261161 | 9.205984 | 9.146622 | 8.749749 |
| itga6 | 7.235906 | 7.710421 | 8.22062 | 7.768691 |
| She | 7.463482 | 6.993214 | 7.172411 | 7.285977 |
| Sema3c | 7.470103 | 7.894566 | 7.659731 | 7.679211 |
| Scarb1 | 6.885622 | 7.718629 | 7.846623 | 7.649505 |
| Apoe | 8.356755 | 9.571595 | 9.017662 | 9.363265 |
| Beam | 7.668255 | 7.497221 | 7.395417 | 7.642855 |
| Psma1 | 7.528471 | 7.495766 | 7.434574 | 7.597206 |
| Isyna1 | 7.855183 | 8.124006 | 7.610811 | 7.573665 |
| Bsg | 12.00525 | 12.79916 | 13.02994 | 12.96478 |
| Sqstm1 | 8.930779 | 8.869301 | 8.633605 | 8.658331 |
| Tspan13 | 9.599012 | 9.972338 | 9.907196 | 9.216815 |
| Itga1 | 7.78763 | 7.571432 | 7.486232 | 7.557926 |
| Ptprg | 7.648599 | 7.619406 | 7.703569 | 7.790773 |
| Adamts1 | 7.8442 | 6.765857 | 7.070666 | 5.713259 |
| Lims2 | 7.2102 | 7.144381 | 7.006711 | 7.456291 |
| Egr1 | 7.60555 | 3.799432 | 4.321928 | 5.765459 |
| Ahnak | 7.882174 | 7.967756 | 8.559062 | 8.892917 |

TABLE 29
Top 10% highly expressed Alzheimer’s disease-associated genes that are expressed by LECs,
BECs and microglia

| | |
|---|---|
| mLECs | Adamts9, Aff1, Ap2a2, Ccdc50, Ckap5, Dgkz, Dmx11, Exoc4, Fat1, Fnbp4, Fxyd6, |
| | Gpc6, Kansl1, Kcnn3, Kdm3b, Madd, Man2a1, Map4k4, Nfic, Nr2f2, Nr3c2, Pcdh7, |
| | Pik3r1, Plekhg1, Prdm2, Rapgef6, Reln, Sash1, Sec24b, Slmpa, Sor11, Tcf712, Thsd7a, |
| | Tnxb, Top1, Trim56, Trip4, Usp6n1, Abca1, Adam10, Ahnak, Akap9, Cd2ap, Celf1, |
| | Dlc1, Dst, Efr3a, Egr1, Fermt2, Golim4, Gsk3b, Hmcn1, Itga1, Itga6, Nfib, Pak2, |
| | Parvb, Prrc2c, Ptprg, Rgl1, Serinc5, Sft2d2, She, Sppl2a, Tmem106b, Apoe, Bsg, |
| | Tspan13, Elmo1, Frmd4a, Maf and Timp2 |
| bBECs | Adamts1, Adar, Bcam, Cdc42se2, Clu, Gemin7, Hbegf, Hs3st1, Hspa9, Isyna1, Lims2, |
| | Mpzl1, Mtch2, Psmc3, Rora, Scarb1, Sema3c, Sik1, Slc19a3, Sqstm1, Abca1, Adam10, |
| | Ahnak, Akap9, Cd2ap, Celf1, Dlc1, Dst, Efr3a, Egr1, Fermt2, Golim4, Gsk3b, Hmcn1, |
| | Itga1, Itga6, Nfib, Pak2, Parvb, Prrc2c, Ptprg, Rgl1, Serinc5, Sft2d2, She, Sppl2a, |
| | Tmem106b, Crl1, Clptm1, Mef2c, Picalm, Psma1, Ssbp4, Apoe, Bsg and Tspan13 |
| Microglia | Abi3, Bin1, Ccr5, Cd33, Cycs, IL6ra, Inpp5d, Krit1, Ms4a6d, Sdf2l1, Sec11c, Siglech, |
| | Spi1, Tbxas1, Trem2, Ube2d2a, Vasp, Elmo1, Frmd4a, Maf, Timp2, Crl1, Clptm1, |
| | Mef2c, Picalm, Psma1, Ssbp4, Apoe, Bsg and Tspan13 |

Top 50 differentially expressed meningeal LEC genes between WT and 5xFAD LECs
GRID2, CDC25A, EVI2. 4930554G24RIK, ETFB, ADAMTS3, FAM219AOS, ENAH,
ENSMUSG00000085673, ENSMUSG00000054418, ENSMUSG00000075511, RNF5, ARG1,
RUNDC3B, 4921524J17RIK, ITGB2, CHRM3, GCDH, ENSMUSG00000053218,
ENSMUSG00000097358, CDKL2, RABGEF1, KCNRG, TIMP1, 4930404L05RIK, KIT, PPP2R1B,
EGLN3, B4GALT4, ENSMUSG00000069682, TFPI2, PAMR1, NOP10, SOX17, ZFP358, Mccc1,
MUM1L1, MIS12, ZFP869, BC031181, NPL, CYP2D22, TSSC4, LZIC, SUSD5,
ENSMUSG00000097020, TESK1, IBTK, HIST1H1E and ZBTB17
Meningeal LEC genes in pathways significantly changed between WT and 5xFAD mice

| | |
|---|---|
| Exocytosis | STAM, KIT, STX11, RAP1A, ABR, MILR1, LLGL1, RABGEF1, VAMP2, PIP5K1C, |
| pathway | RAB11FIP1, ITGB2, NR4A3, EXPH5, RAPGEF4, SYTL4, PPFIA2, MICAL3, |
| | EXOC8, and SCFD2 |
| Phospholipase | PDGFA, PTPN11, RAPGEF4, ADCY4, GABI, PLD1, CYTH3, KIT, SHC1, |
| D signaling | PIP5K1C, and PLCB3 |
| pathway | |

Differentially expressed genes uniquely expressed in meningeal lymphatics
S1PR2, EDN1, GPR82, GPR27, PTGDR, ADRB2
Differential genes in microglia of 5xFAD mice with intact or ablated meningeal lymphatics
Hexb, ApoE, H2-Aa, H2-Ab1, Cd74, H2-D1, and H-2Kd

What is claimed is:

1. A method of modulating an activity of a lymphatic endothelial cell (LEC), a brain myeloid cell (e.g., microglia (Mg)), an infiltrating leukocyte, and/or a brain blood vascular cell (e.g., a brain blood endothelial cell (bBEC)) in a subject in need thereof, wherein the activity is an alteration of gene expression in one or more genes, the method comprising
administering an effective amount of a flow modulator to the subject, wherein the flow modulator increases the fluid flow in the central nervous system (CNS) of the subject; and
administering an effective amount of a neurological therapeutic agent to the subject,
thereby modulating the activity of the LEC, brain myeloid cell, Mg, infiltrating leukocyte, brain blood vascular cell and/or bBEC in the subject.

2. The method of claim 1, wherein the alteration of gene expression is an increase in a level of gene expression of the one or more genes in Tables 2-29 as compared to a control level of gene expression of the one or more genes

3. The method of claim 2, wherein the level of gene expression of the one or more genes is increased at least 50%, at least 75%, at least 100%, at least 1.25 fold, at least 1.5-fold, at least 1.75-fold, or at least 2-fold as compared to the control level of gene expression of the one or more genes

4. The method of any one of claims 1-3, wherein the alteration of gene expression is a decrease in a level of gene expression of the one or more genes in Tables 2-29 as compared to a control level of gene expression of the one or more genes.

5. The method of claim 4, wherein the level of gene expression of the one or more genes is decreased at least 50%, at least 75%, at least 100%, at least 1.25 fold, at least 1.5-fold, at least 1.75-fold, or at least 2-fold as compared to the control level of gene expression of the one or more genes.

6. The method of any one of claims 2-5, wherein the control level is a level of the gene expression of the one or more genes in a healthy subject not having a neurological disease, or wherein the control level is an average level of gene expression of the one or more genes in a population of healthy subjects not having a neurological disease, or wherein the control level is a level of the gene expression of the one or more genes in an age-matched subject with intact and functional meningeal lymphatic vasculature and no underlying neurological disease, or wherein the control level is an average level of gene expression of the one or more genes in a population of age-matched subjects with intact and functional mengigeal lymphatic vasculature and no neurological disease.

7. The method of any one of claims 1-6, wherein the one or more genes is selected from the group consisting of:
Dst, Hmcn1, Rgl1, Prrc2c, Sft2d2, Itga6, Celf1, Sppl2a, Golim4, She, Abca1, Nfib, Akap9, Tmem106b, Dlc1, Adam10, Serinc5, Itga1, Ptprg, Fermt2, Efr3a, Parvb, Gsk3b, Pak2, Cd2ap, Egr1, and Ahnak;
Frmd4a, Maf, Timp2, and Elmo1;
Crl1, Clptm1, Picalm, Psma1, Ssbp4, and Mef2c;
Apoe Tspan13, and Bsg;
Abi3, Bin1, Ccr5, Cd33, Cycs, IL6ra, Inpp5d, Kritl, Ms4a6d, Sdf211, Sec11c, Siglech, Spi1, Tbxas1, Trem2, Ube2d2a, and Vasp;
Adamts1, Adar, Bcam, Cdc42se2, Clu, Gemin7, Hbegf, Hs3st1, Hspa9, Isyna1, Lims2, Mpzl1, Mtch2, Psmc3, Rora, Scarb1, Sema3c, Sik1, Slc19a3, and Sqstm1; and/or
Adamst9, Aff1, Aβ2a2, Ccdc50, Ckap5, Dgkz, Dmxl1, Exoc4, Fat1, Fnbp4, Fxyd6, Gpc6, Kansl1, Kcnn3, Kdm3b, Madd, Man2al, Map4k4, Nfic, Nr2f2, Nr3c2, Pcdh7, Pik3r1, Plekhg1, Prdm2, Rapgef6, Reln, Sash1, Sec24b, Slmpa, Sorl1, Tcf712, Thsd7a, Tnxb, Top1, Trim56, Trip4, and Usp6nl.

8. The method of any one of claims 1-6, wherein the one or more genes is selected from the group consisting of: GRID2, CDC25A, EVI2, 4930554G24RIK, ETFB, ADAMTS3, FAM219AOS, ENAH, ENSMUSG00000085673, ENSMUSG00000054418, ENSMUSG00000075511, RNF5, ARG1, RUNDC3B, 4921524J17RIK, ITGB2, CHRM3, GCDH, ENSMUSG00000053218, ENSMUSG00000097358, CDKL2, RABGEF1, KCNRG, TIMP1, 4930404L05RIK, KIT, PPP2R1B, EGLN3, B4GALT4, ENSMUSG00000069682, TFPI2, PAMR1, NOP10, SOX17, ZFP358, Mcccl, MUM1L1, MIS12, ZFP869, BC031181, NPL, CYP2D22, TSSC4, LZIC, SUSD5, ENSMUSG00000097020, TESK1, IBTK, HISTIHIE and ZBTB17.

9. The method of any one of claims 1-6, wherein the one or more genes is selected from the group consisting of: STAM, KIT, STX11, RAPlA, ABR, MILR1, LLGL1, RABGEF1, VAMP2, PIP5K1C, RABI IFIPI, ITGB2, NR4Aβ, EXPH5, RAPGEF4, SYTL4, PPFIA2, MICAL3, EXOC8, and SCFD2; and/or PDGFA, PTPN11, RAPGEF4, ADCY4, GAB1, PLD1, CYTH3, KIT, SHC1, PIP5K1C, and PLCB3.

10. The method of any one of claims 1-6, wherein the one or more genes is ApoE.

11. The method of any one of claims 1-6, wherein the one or more genes is selected from the group consisting of S1PR2, EDN1, GPR82, GPR27, PTGDR, and ADRB2.

12. The method of any one of claims 1-11, further comprising determining a level of gene expression of the one or more genes in the subject prior to administering the effective amount of the flow modulator and the effective amount of the neurological therapeutic agent to the subject.

13. The method of any one of claims 1-12, further comprising selecting a subject who would benefit from an increase in gene expression of the one or more genes in Tables 2-29 or a decrease in gene expression of the one or more genes in Tables 2-29.

14. The method of any one of claims 1-13, wherein the subject has a neurological disease, or is at risk for developing a neurological disease.

15. The method of any one of claims 1-13, further comprising selecting a subject that has a neurological disease, or is at risk for developing a neurological disease.

16. The method of claim 14 or claim 15, wherein the neurological disease is Alzheimer's Disease (AD).

17. The method of claim 16, wherein the subject has a risk factor for AD selected from the group consisting of: diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), age, familial AD, and a symptom of dementia; or a combination thereof.

18. The method of any one of claims 1-17, wherein the flow modulator is a VEGFR3 agonist or Fibroblast Growth Factor 2 (FGF2).

19. The method of any one of claims 1-18, wherein the neurological therapeutic agent is selected from the group consisting of a small molecule, a nucleic acid, a peptide, a protein, an antibody or antigen binding fragment thereof, a recombinant virus, a vaccine, and a cell.

20. The method of claim 19, wherein the neurological therapeutic agent comprises a small molecule.

21. The method of claim 20, wherein the small molecule is selected from the group consisting of Donepezil, Galantamine, Rivastigmine, Memantine, Lanabecestat, Atabecestat, Verubecestat, Elenbecestat, Semagacestat, Tarenflurbil, and Brexipiprazole.

22. The method of claim 19, wherein the neurological therapeutic agent comprises an antibody, or an antigen binding fragment thereof, that specifically binds to a protein or a peptide that forms pathological aggregate.

23. The method of claim 22, wherein the peptide or protein is selected from the group consisting of amyloid precursor protein, amyloid beta, fibrin, tau, apolipoprotein E (Apoe), alpha-synuclein, TDP43, and huntingtin.

24. The method of claim 23, wherein the protein is amyloid beta, and wherein the antibody or the antigen binding fragment thereof is selected from the group consisting of: bapineuzumab, gantenerumab, aducanumab, solanezumab, immunoglobulin, BAN2401, semorinemab, zagotenemab,crenezumab, and the antigen binding fragment thereof.

25. The method of claim 23, wherein the protein is amyloid beta, and wherein the antibody or the antigen binding fragment thereof comprises a HCDR1, a HCDR2, a HCDR3, a LCDR1, a LCDR2, and a LCDR3 of any one of bapineuzumab, gantenerumab, aducanumab, solanezumab, immunoglobulin, BAN2401, semorinemab, zagotenemab, crenezumab, or an antigen binding fragment thereof.

26. The method of claim 23, wherein the protein is tau, and wherein the antibody or the antigen binding fragment thereof is selected from the group consisting of Gosuranemab, Armanezumab, and an antigen binding fragment thereof.

27. The method of claim 23, wherein the protein is tau, and wherein the antibody or the antigen binding fragment thereof comprises a HCDR1, a HCDR2, a HCDR3, a LCDR1, a LCDR2, and a LCDR3 of any one of Gosuranemab, Armanezumab, or the antigen binding fragment thereof.

28. The method of claim 23, wherein the protein is alpha-synuclein, and wherein the antibody or the antigen binding fragment thereof is selected from the group consisting of BIIB054, PRX002/RG7935, prasinezumab, and the antigen binding fragment thereof.

29. The method of claim 28, wherein the protein is alpha-synuclein, and wherein the antibody or the antigen binding fragment thereof comprises a HCDR1, a HCDR2, a HCDR3, a LCDR1, a LCDR2, and a LCDR3 of any one of BIIB054, PRX002/RG7935, prasinezumab, or the antigen binding fragment thereof.

30. The method of any one of claims 1-29, wherein the flow modulator is a VEGFR3 agonist, said VEGFR3 agonist comprising VEGF-c, wherein the VEGF-c is administered by intra-cisterna magna (ICM) injection, and
wherein the neurological therapeutic agent comprises an antibody, or antigen-binding fragment thereof, that binds to amyloid beta, and wherein the antibody or antigen-binding fragment thereof is administered systemically.

31. A method of identifying a subject that has an enhanced risk of developing a neurological disease, comprising detecting an alteration in gene expression in one or more genes in Tables 2-29 in central nervous system prior to the onset of the neurological disease, thereby identifying the subject as having an enhanced risk of developing the neurological disease.

32. The method of claim 31, wherein the alteration in gene expression is in brain lymphatic endothelial cells (LECs), microglia (Mg), and/or brain blood endothelial cells (bBECs).

33. The method of claim 31, wherein the alteration in gene expression is in immune cells in the brain of the subject.

34. The method of claim 33, wherein the alteration in gene expression is in immune cells in brain cortices or meninges of the subject.

35. The method of any one of claims 31-34, wherein the gene is selected from the group consisting of Hexb, ApoE, H2-Aa, H2-Ab1, Cd74, H2-D1, and H-2Kd.

36. The method of any one of claims 32-34, wherein the brain LECs or immune cells are obtained from a biopsy of deep cervical lymph nodes or peripheral blood from the subject.

37. The method of claim 31, wherein the alteration in gene expression is in ear skin cells.

38. A method of identifying a subject that has an enhanced risk of developing neurological disease, comprising detecting an increase in a number of immune cells in central nervous system of the subject prior to the onset of the neurological disease, thereby identifying the subject as having an enhanced risk of developing the neurological disease.

39. The method of claim 38, wherein the increase in the number of immune cells is in brain cortices or meninges of the subject.

40. The method of claim 38 or 39, wherein the immune cells are CD45^{high} cells or H-2Kd expressing CD45^{int} cells.

41. The method of claim 38 or 39, wherein the immune cells are microglia or recruited lymphocytes from blood.

42. The method of claim 38 or 39, wherein the immune cells are selected from the group consisting of B cells, CD4⁺ T cells, CD8⁺ T cells, and type 3 innate lymphoid cells (ILC3s).

43. The method of any one of claims 38-42, wherein the number of immune cells is determined by in vivo fluorescence imaging.

44. A method of identifying a subject that has an enhanced risk of developing a neurological disease, comprising detecting one or more single nucleotide polymorphisms (SNPs) associated with one or more genes selected from the genes in Tables 2-29, thereby identifying the subject as having an enhanced risk of developing the neurological disease.

45. The method of claim 44, wherein the SNP is associated with a gene that is highly expressed in a lymphatic endothelial cell.

46. The method of claim 45, wherein the lymphatic endothelial cell is selected from the group consisting of a central nervous system lymphatic endothelial cell, a diaphragm lymphatic endothelial cell, and an ear skin endothelial cell.

47. The method of claim 45 or 46, wherein the gene that is highly expressed in the lymphatic endothelial cell has an average expression in the top 2^{nd}, 5^{th}, 10^{th}, or 25^{th} percentile out of all genes.

48. The method of claim 47, wherein the expression percentile is determined by RNA-seq data.

49. The method of any one of claims 45 to 48, wherein the gene is selected from the group consisting of the genes listed in FIG. 23.

50. The method of claim 49, wherein the gene is selected from the group consisting of Dst, Hmcn1, Rgl1, Prrc2c, Sft2d2, Itga6, Celf1, Sppl2a, Golim4, She, Abca1, Nfib, Akap9, Tmem106b, Dlc1, Adam10, Serinc5, Itga1, Ptprg, Fermt2, Efr3a, Parvb, Gsk3b, Pak2, Cd2ap, Egr1, and Ahnak.

51. The method of claim 49, wherein the gene is selected from the group consisting of Frmd4a, Maf, Timp2, and Elmo1.

52. The method of claim 49, wherein the gene is selected from the group consisting of Crl1, Clptm1, Picalm, Psma1, Ssbp4, and Mef2c.

53. The method of claim 49, wherein the gene is selected from the group consisting of Apoe Tspan13, and Bsg.

54. The method of any one of claims 1-53, wherein the subject is a human subject.

55. The method of claim 54, wherein the human subject is about 20 years old, about 30 years old, about 40 years old, about 50 years old, about 60 years old, about 70 years old, or about 80 years old.

56. The method of claim 55, wherein the human subject has been previously identified to have a risk of developing neurological disease.

57. The method of claim 56, wherein the human subject has been previously identified to have a risk of developing neurological disease by family history investigation or genetic screening.

58. The method of any one of claims 1-57, wherein the neurological disease is selected from the group consisting of AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CID), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, or a combination of two or more of any of the listed items.

59. The method of any one of claims 1-58, wherein the neurological disease is Alzheimer's disease.

60. A method of reducing the risk or delaying the onset of developing a neurological disease in a subject, comprising administering an effective amount of a neurological therapeutic agent to the subject prior to the onset of the neurological disease, thereby reducing the risk of developing the neurological disease in the subject, wherein the subject is identified to have an enhanced risk of developing a neurological disease using the method of any one of claims 31-53.

61. The method of claim 60, further comprising administering an effective amount of a flow modulator to a meningeal space of the subject.

62. The method of claim 60 or claim 61, wherein the neurological therapeutic agent reduces the number of immune cells in the brain.

63. A method of increasing clearance of a molecule from the central nervous system in a subject in need thereof, the method comprising:
administering an effective amount of a flow modulator to the subject by intra-cisterna magna (ICM) injection, wherein the flow modulator increases the fluid flow in the central nervous system of the subject;
and administering an effective amount of a neurological therapeutic agent to the subject by systemic administration,
thereby increasing the clearance of the molecule from the central nervous system of the subject.

64. A method of reducing an aggregate of a protein or peptide in the central nervous system of a subject in need thereof, the method comprising:
administering an effective amount of a flow modulator to the subject by intra-cisterna magna (ICM) injection, wherein the flow modulator increases the fluid flow in the central nervous system of the subject;
and administering an effective amount of a neurological therapeutic agent to the subject by systemic administration,
thereby reducing the aggregate of the protein or peptide in the subject.

65. A method of reducing a microglial inflammatory response in the central nervous system of a subject in need thereof, the method comprising:
administering an effective amount of a flow modulator to the subject by intra-cisterna magna (ICM) injection, wherein the flow modulator increases the fluid flow in the central nervous system of the subject;
and administering an effective amount of a neurological therapeutic agent to the subject by systemic administration,
thereby reducing the microglial inflammatory response in the central nervous system of the subject.

66. A method of reducing neurite dystrophy in the central nervous system of a subject in need thereof, the method comprising:
administering an effective amount of a flow modulator to the subject by intra-cisterna magna (ICM) injection, wherein the flow modulator increases the fluid flow in the central nervous system of the subject;
and administering an effective amount of a neurological therapeutic agent to the subject by systemic administration,
thereby reducing neurite dystrophy in the central nervous system of the subject.

67. A method of treating a neurological disease in a subject in need thereof, the method comprising:
administering an effective amount of a flow modulator to the subject by intra-cisterna magna (ICM) injection, wherein the flow modulator increases the fluid flow in the central nervous system of the subject;
and administering an effective amount of a neurological therapeutic agent to the subject by systemic administration,
thereby treating the neurological disease in the subject.

68. The method of any one of claims 63-67, wherein the flow modulator comprises a VEGFR3 agonist, optionally wherein the VEGFR3 agonist comprises a VEGF-c.

69. The method of any one of claims 63-68, wherein the neurological therapeutic agent is an antibody, or antigen-binding fragment thereof, optionally wherein the antibody, or antigen-binding fragment thereof, is an amyloid beta antibody, or antigen-binding fragment thereof.

70. The method of claim 69, wherein the amyloid beta antibody, or antigen-binding fragment thereof, is selected from the group consisting of: bapineuzumab, gantenerumab, aducanumab, solanezumab, immunoglobulin, BAN2401, semorinemab, zagotenemab,crenezumab, and an antigen binding fragment thereof.


SEQUENCE LISTING (5 sequences)

SEQ ID NO: 1 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 2 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 3 - protein, 419 aa, Homo sapiens [sequence omitted]

SEQ ID NO: 4 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN